(19) United States
(12) Patent Application Publication
(10) Pub. No.: US 2024/0239808 A1
(43) Pub. Date: Jul. 18, 2024

(54) INHIBITING UBIQUITIN-SPECIFIC PROTEASE 1 (USP1)

(71) Applicant: Forma Therapeutics, Inc., Watertown, MA (US)

(72) Inventors: Loren Berry, Arlington, MA (US); Alexandre Joseph Buckmelter, Action, MA (US)

(73) Assignee: Forma Therapeutics, Inc., Watertown, MA (US)

(21) Appl. No.: 18/286,053

(22) PCT Filed: Apr. 6, 2022
(86) PCT No.: PCT/US2022/023669

§ 371 Date: Oct. 6, 2023

Related U.S. Application Data
(60) Provisional application No. 63/171,796, filed on Apr. 7, 2021.

(51) Int. Cl. C07D 498/04; A61K 31/519; A61K 31/5365; A61K 31/5383; C07D 471/04; C07D 487/04; C07D 513/04; C07D 519/00

(52) U.S. Cl. CPC C07D 498/04; A61K 31/519; A61K 31/5365; A61K 31/5383; C07D 471/04; C07D 487/04; C07D 513/04; C07D 519/00

(57) ABSTRACT

The present disclosure provides compounds for inhibiting USP1, and related methods of preparing and using the compounds.

### CROSS REFERENCE TO RELATED APPLICATIONS

This application claims the benefit of and priority to U.S. provisional application No. 63/171,796, filed Apr. 7, 2021, which is herein incorporated by reference in its entirety.

### TECHNICAL FIELD

This disclosure provides compounds and related methods useful for inhibiting ubiquitin-specific protease 1 (USP1).

### BACKGROUND

USP1 is a cysteine isopeptidase of the USP subfamily of DUBs. Full-length human USP1 is composed of 785-amino acids, including a catalytic triad composed of Cys90, His593 and Asp751. USP1 deubiquitinates a variety of cellular targets involved in different processes related to cancer. For example, USP1 deubiquitinates PCNA (proliferating cell nuclear antigen), a key protein in translesion synthesis (TLS), and FANCD2 (Fanconi anemia group complementation group D2), a key protein in the Fanconi anemia (FA) pathway. These DNA damage response (DDR) pathways are essential for repair of DNA damage induced by DNA cross-linking agents such as cisplatin, mitomycin C, diepoxybutane, ionizing radiation and ultraviolet radiation.

USP1 is upregulated in BRCA1-mutant tumors and is synthetic lethal with BRCA1. BRCA-mutant and more broadly homologous recombination deficient (HRD) tumors are sensitive to PARP inhibitors (Mateo et al. 2019). Despite their effectiveness, resistance to PARP inhibitors occurs and leads to disease progression. One of the mechanisms of resistance to PARP inhibitors is restoration of replication fork stability. USP1 protects replication forks from collapse. Knockdown or inhibition of USP1 results in persistence of mono-ubiquitinated PCNA at the replication fork and cell death in BRCA1 deficient cells. In addition, inhibition of USP1 was antiproliferative in BRCA1-mutant cells resistant to PARP inhibitor suggesting that USP1 inhibitors could be useful in treating BRCA-mutant tumors resistant to PARP inhibitors.

USP1 affects other substrates beyond PCNA and FANCD2 and has been shown to impact epigenetic proteins, such as lysine-specific demethylase 4A (KDM4A) and enhancer of zeste homolog 2 (EZH2), as well as signaling pathways such as Fanconi anemia and PI3K/AKT. Different genetic contexts beyond BRCA mutations are therefore susceptible to drive dependency to USP1.

Inhibition of USP1 with small molecule inhibitors therefore has the potential to be a treatment for cancers, including BRCA mutant tumors, and other disorders. For this reason, there remains a considerable need for potent small molecule inhibitors of USP1.

### SUMMARY

The present disclosure provides compounds of Formula (I):

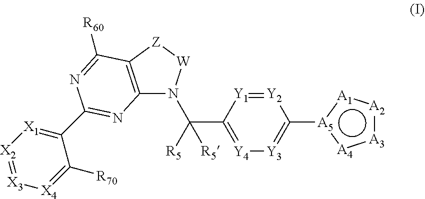

or pharmaceutically acceptable salts thereof,
wherein,
   X₁ is CR₆ or N; X₂ is CR₇ or N; X₃ is CR₈ or N; and X₄ is CR₉ or N;
   R₆ is hydrogen, halogen, (C₁-C₄)alkyl, (C₁-C₄)alkoxy, (C₃-C₆)cycloalkyl, —O—(C₃-C₆)cycloalkyl, or 4- to 6-membered heterocycloalkyl having 1-3 heteroatoms independently selected from N, O, and S, wherein the alkyl, cycloalkyl or heterocycloalkyl is optionally substituted with one or more substituents independently selected from halogen, hydroxyl, (C₁-C₄)alkoxy, or —NR_{b}R_{b};
   R₇, R₈ and R₉ are each independently hydrogen or halogen;
   R₇₀ is hydrogen, halogen, (C₁-C₄)alkyl, (C₁-C₄)alkoxy, (C₃-C₆)cycloalkyl, —O—(C₃-C₆)cycloalkyl, or 4- to 6-membered heterocycloalkyl having 1-3 heteroatoms independently selected from N, O, and S, wherein the alkyl, cycloalkyl or heterocycloalkyl is optionally substituted with one or more substituents independently selected from halogen, hydroxyl, (C₁-C₄)alkoxy, or —NR_{b}R_{b};
   R₆₀ is hydrogen, halogen, (C₁-C₄)alkyl, (C₁-C₄)alkoxy, (C₃-C₆)cycloalkyl, or 4- to 6-membered heterocycloalkyl having 1-3 heteroatoms independently selected from N, O, and S, wherein the alkyl, cycloalkyl or heterocycloalkyl is optionally substituted with one or more substituents independently selected from halogen, hydroxyl, (C₁-C₄)alkoxy, or —NR_{b}R_{b};
   Z and W are together selected to form a fused 5- or 6-membered ring selected from cycloalkyl, cycloalkenyl, heterocycloalkyl having 1-3 heteroatoms independently selected from N, O, or S, or heterocycloalkenyl having 1-3 heteroatoms independently selected from N, O, or S, wherein Z is selected from the group consisting of: —C(R₁₆)(R₁₆′)—, —C(R₂₀)(R₂₀′)—C(R₁₈)—*, —S—, —S—C(R₁₈)(R₁₈′)—*, —C(R₂₀)(R₂₀′)—C(R₁₈)(R₁₈′)—*, —C(R₁₈)(R₁₈′)—S—*, —N(R₁₄)—, —N(R₁₄)—C(R₁₈)(R₁₈′)—*, —N(R₁₄)—C(R₁₈)═*, —N═C(R₁₈)—*, —C(R₁₈)(R₁₈′)—N(R₁₄)—*, —O—, —O—C(R₁₈)(R₁₈′)—*, —C(R₁₈)(R₁₈′)—O—*, —O—C(R₁₈)═*, and —C(R₂₀)═C(R₁₈)—*, wherein * represents the point of attachment to W, and W is selected from the group consisting of: —(C═O)— ═C(R₉₀)—, and —C(R₁₀)(R₁₀′)—, provided that when Z is —N(R₁₄)—, W is not —(C═O)—; or
   Z and W are together selected to form a fused 5- or 6-membered heteroaryl ring, having 1-3 heteroatoms independently selected from N, O, or S, wherein Z is selected from the group consisting of —C(R₂₀)═*, —N═*, and —S—C(R₁₈)═*, wherein * represents the point of attachment to W; and W is selected from the group consisting of ═N— and ═C(R₉₀)—;
   R₉₀ is selected from the group consisting of hydrogen; hydroxyl; (C₁-C₄)alkyl optionally substituted with one or more halogen, hydroxyl or —N(R_{b})(R_{b}′); (C₃-C₆)cyclopropyl optionally substituted with one or more (C₁-C₄)alkyl; —O—(C₁-C₄)alkyl optionally substituted with one or more halogen; (C₁-C₄)alkyl-N(R_{b})(R_{b}′); —O—(C₃-C₆)cycloalkyl; and a 4- to 6-membered heterocycloalkyl having 1-3 heteroatoms independently selected from N, O, or S;
   R₁₀, R₁₀′, R₁₆, R₁₆′, R₁₈, R₁₈′, R₂₀, R₂₀′ are each independently selected from the group consisting of hydrogen, hydroxyl, (C₁-C₄)alkyl optionally substituted with one or more halogen, —O—(C₁-C₄)alkyl optionally substituted with one or more halogens, (C₁-C₄)alkyl-N(R_{b})(R_{b}′); or R₁₆ and R₁₆′, R₁₈ and R₁₈′, and R₂₀ and R₂₀′ each together form a spirocyclic 3- to 6-membered cycloalkyl optionally substituted with one or more Rₐ′;
   R₁₄ is selected from the group consisting of hydrogen and (C₁-C₄)alkyl;
   R₅ and R₅′ are each independently selected from hydrogen, halogen, (C₁-C₄)alkyl, —(C₁-C₄)alkyl-O—(C₁-C₄)alkyl, or —(C₁-C₄)alkyl-N(R_{b})(R_{b}′), wherein each alkyl is optionally substituted with one or more halogens; or R₅ and R₅′ together form a (C₃-C₆)cycloalkyl ring optionally substituted with one or more substituents independently selected from halogen or (C₁-C₄)alkyl;
   Y₁, Y₂, Y₃ and Y₄ are each independently —C(R_{y})— or N, wherein each R_{y} is independently hydrogen, halogen, or (C₁-C₄)alkyl;
   each of A₁, A₂, A₃, and A₄, is independently selected from the group consisting of C(R₂), N(R₁), O, and S;
   A₅ is N or CR₂;
   wherein A₁, A₂, A₃, A₄, A₅ together form a 5-membered heteroaryl ring;
wherein
   each R₁ is independently a bond, hydrogen, (C₁-C₄)alkyl, —O—(C₁-C₄)alkyl, 3- to 6-membered cycloalkyl, or 3- to 6-membered heterocycloalkyl having 1-3 heteroatoms independently selected from N, O, and S, wherein each (C₁-C₄)alkyl or —O—(C₁-C₄)alkyl is independently optionally substituted with one or more Rₐ, and each 3- to 6-membered cycloalkyl or 3- to 6-membered heterocycloalkyl is independently optionally substituted with one or more Rₐ′;
   each R₂ is independently a bond, hydrogen, (C₁-C₄)alkyl, —O—(C₁-C₄)alkyl, 3- to 6-membered cycloalkyl, or 3- to 6-membered heterocycloalkyl having 1-3 heteroatoms independently selected from N, O, and S, wherein each (C₁-C₄)alkyl or —O—(C₁-C₄)alkyl is independently optionally substituted with one or more Rₐ, and each 3- to 6-membered cycloalkyl or 3- to 6-membered heterocycloalkyl is independently optionally substituted with one or more Rₐ′; or
   two occurrences of R₂ on adjacent carbon atoms in A₁, A₂, A₃, or A₄, may together form a fused ring selected from a 5- to 6-membered heterocycloalkyl having 1-3 heteroatoms selected from N, O, or S or a 5- to 6-membered heteroaryl having 1-3 heteroatoms selected from N, O, or S, wherein each ring may independently be optionally substituted with one or more R_{a′};
   each Rₐ is independently selected from the group consisting of halogen, (C₁-C₄)alkyl, hydroxyl, —N(R_{b})(R_{b}′), (C₁-C₄)alkoxy optionally substituted with one or more R_{a′}, and 3- to 6-membered cycloalkyl optionally substituted with one or more R_{a′};
   each R_{a′} is independently halogen or (C₁-C₄)alkyl optionally substituted with one or more halogen, and
   each R_{b} and R_{b}′ is independently selected from the group consisting of hydrogen and (C₁-C₄)alkyl optionally substituted with one or more halogen.

In some embodiments, the compound is a compound of Formula (I) wherein Z is —S—C(R₁₈)(R₁₈′)—*, wherein * represents the point of attachment to W, and W is —C(R₁₀)(R₁₀′)—, such as a compound of Formula (IIa); or Z is —S—C(R₁₈)(R₁₈′)—*, wherein * represents the point of attachment to W, and W is —(C═O)—, such as a compound of Formula (IIIa):

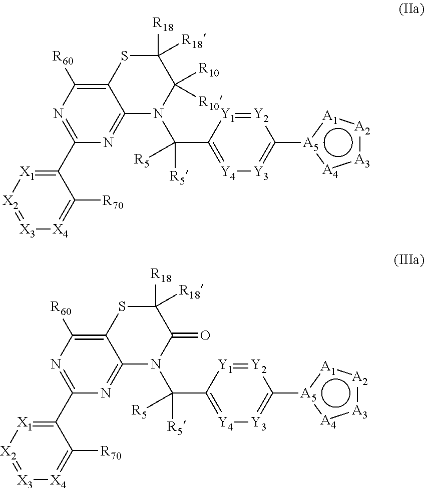

or a pharmaceutically acceptable salt thereof, wherein X₁, X₂, X₃, X₄, R₆₀, R₇₀, R₁₈, R₁₈′, R₁₀, R₁₀′, R₅, R₅′, Y₁, Y₂, Y₃, Y₄, A₁, A₂, A₃, A₄, and A₅ are each as described above with respect to Formula (I), and defined and described in classes and subclasses herein (e.g., with respect to any one or more of Formula (II)-(X)), both singly and in combination.

It will be appreciated that a reference to, e.g., Formula (II), includes each of, e.g., Formula (IIa), (IIb), and (IIc) in combination. This similarly applies to other formulae provided herein.

In some embodiments, the compound is a compound of Formula (I) wherein Z is —CH₂— and W is —CH₂—, such as a compound of Formula (Xa); or Z is —C(R₂₀)(R₂₀′)C(R₁₈)(R₁₈*)—*, wherein * represents the point of attachment to W, and W is —CH₂—, such as a compound of Formula (Xb):

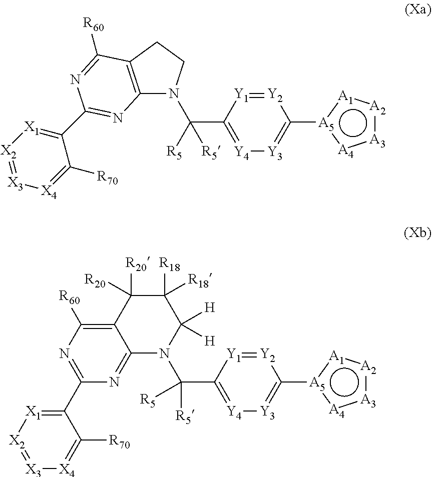

or a pharmaceutically acceptable salt thereof, wherein X₁, X₂, X₃, X₄, R₆₀, R₇₀, R₁₈, R₁₈′, R₂₀, R₂₀′, R₅, R₅′, Y₁, Y₂, Y₃, Y₄, A₁, A₂, A₃, A₄, and A₅ are each as described above with respect to Formula (I), and defined and described in classes and subclasses herein (e.g., with respect to any one or more of Formula (II)-(X)), both singly and in combination.

In some embodiments, the compound is a compound of Formula (I) wherein Z is —C(R₁₆)(R₁₆′)— and W is —(C═O)—, such as a compound of Formula (IX):

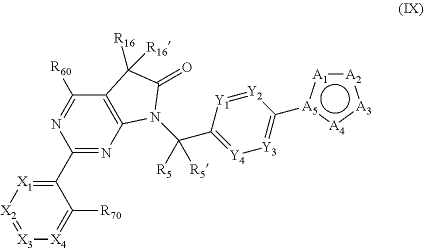

or a pharmaceutically acceptable salt thereof, wherein X₁, X₂, X₃, X₄, R₆₀, R₇₀, R₁₆, R₁₆′, R₅, R₅′, Y₁, Y₂, Y₃, Y₄, A₁, A₂, A₃, A₄, and A₅ are each as described above with respect to Formula (I), and defined and described in classes and subclasses herein (e.g., with respect to any one or more of Formula (II)-(X)), both singly and in combination.

In some embodiments, the compound is a compound of Formula (I) wherein Z is —N(R₁₄)—C(R₁₈)(R₁₈′)—*, wherein * represents the point of attachment to W, and W is —C(R₁₀)(R₁₀′)—, such as a compound of Formula (IIb); or Z is —N(R₁₄)—C(R₁₈)(R₁₈′)—*, wherein * represents the point of attachment to W, and W is —(C═O)— such as a compound of Formula (IIIb):

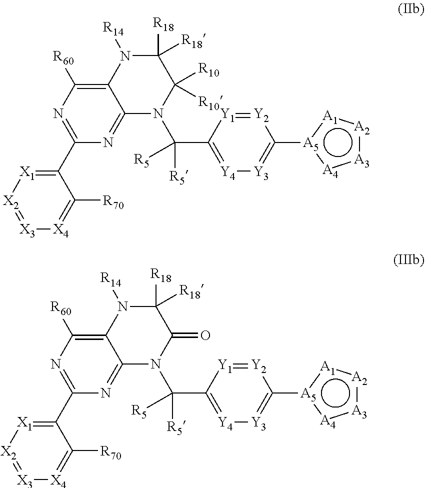

or a pharmaceutically acceptable salt thereof, wherein X₁, X₂, X₃, X₄, R₆₀, R₇₀, R₁₄, R₁₈, R₁₈′, R₁₀, R₁₀′, R₅, R₅′, Y₁, Y₂, Y₃, Y₄, A₁, A₂, A₃, A₄, and A₅ are each as described above with respect to Formula (I), and defined and described in classes and subclasses herein (e.g., with respect to any one or more of Formula (II)-(X)), both singly and in combination.

In some embodiments, the compound is a compound of Formula (I) wherein Z is —O—C(R₁₈)(R₁₈′)—*, wherein * represents the point of attachment to W, and W is —C(R₁₀)(R₁₀′)—, such as a compound of Formula (IIc); or wherein Z is —O—C(R₁₈)(R₁₈′)—*, wherein * represents the point of attachment to W, and W is —(C═O)—, such as a compound of Formula (IIIc):

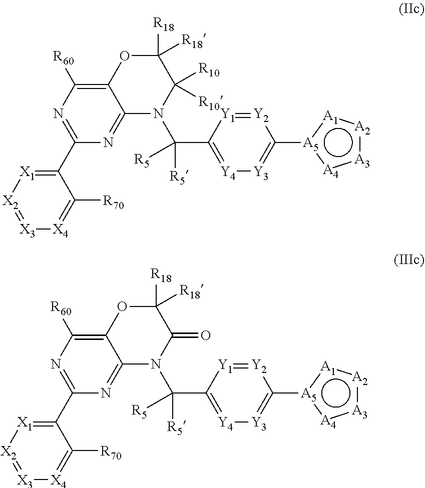

or a pharmaceutically acceptable salt thereof, wherein X₁, X₂, X₃, X₄, R₆₀, R₇₀, R₁₈, R₁₈, R₁₀, R₁₀′, R₅, R₅′, Y₁, Y₂, Y₃, Y₄, A₁, A₂, A₃, A₄, and A₅ are each as described herein with respect to Formula (I), and defined and described in classes and subclasses herein (e.g., with respect to any one or more of Formula (II)-(X)), both singly and in combination.

In some embodiments, the compound is a compound of Formula (I) wherein Z is —CH₂—O—*, wherein * represents the point of attachment to W, and W is —(C═O)—, such as a compound of Formula (IVa); or Z is —CH₂—N(R₁₄)—*, wherein * represents the point of attachment to W, and W is —(C═O)—, such as a compound of Formula (IVb); or Z is —CH₂—S—*, wherein * represents the point of attachment to W, and W is —(C═O)—, such as a compound of Formula (IVc):

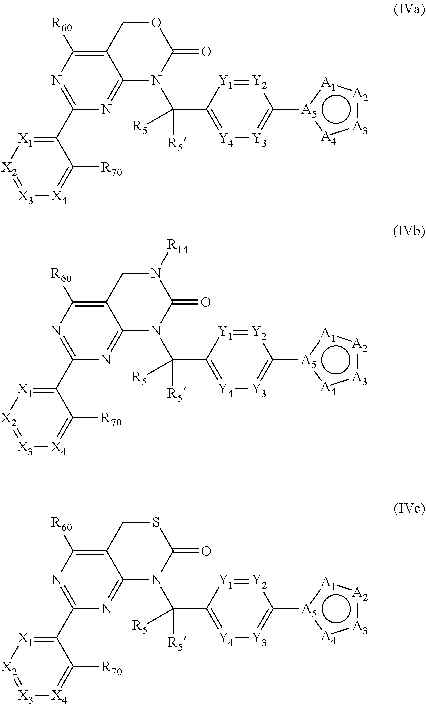

or a pharmaceutically acceptable salt thereof, wherein X₁, X₂, X₃, X₄, R₆₀, R₇₀, R₁₄, R₅, R₅′, Y₁, Y₂, Y₃, Y₄, A₁, A₂, A₃, A₄, and A₅ are each as described above with respect to Formula (I), and defined and described in classes and subclasses herein (e.g., with respect to any one or more of Formula (II)-(X)), both singly and in combination.

In some embodiments, the compound is a compound of Formula (I) wherein Z is —S— and W is —(C═O)—, such as a compound of Formula (Va); or Z is —O— and W is —(C═O)—, such as a compound of Formula (Vb):

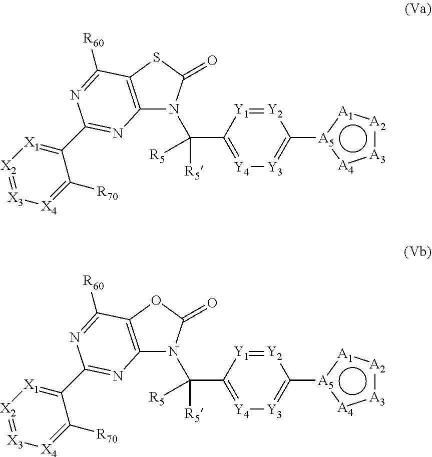

or a pharmaceutically acceptable salt thereof, wherein X₁, X₂, X₃, X₄, R₆₀, R₇₀, R₅, R₅′, Y₁, Y₂, Y₃, Y₄, A₁, A₂, A₃, A₄, and A₅ are each as described above with respect to Formula (I), and defined and described in classes and subclasses herein (e.g., with respect to any one or more of Formula (II)-(X)), both singly and in combination.

In some embodiments, the compound is a compound of Formula (I) wherein Z is —C(R₂₀)(R₂₀′)—C(R₁₈)(R₁₈′)—*, wherein * represents the point of attachment to W, and W is —(C═O)—, such as a compound of Formula (VIa); or Z is —C(R₂₀)═CH—*, wherein * represents the point of attachment to W, and W is —(C═O)—, such as a compound of Formula (VIb):

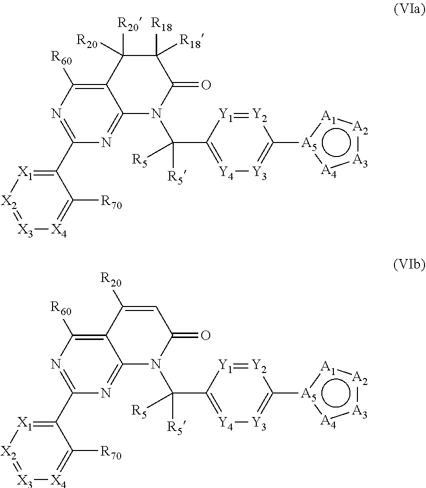

or a pharmaceutically acceptable salt thereof, wherein X₁, X₂, X₃, X₄, R₆₀, R₇₀, R₁₈, R₁₈′, R₂₀, R₂₀′, R₅, R₅′, Y₁, Y₂, Y₃, Y₄, A₁, A₂, A₃, A₄, and A₅ are each as described above with respect to Formula (I), and defined and described in classes and subclasses herein (e.g., with respect to any one or more of Formula (II)-(X)), both singly and in combination.

In some embodiments, the compound is a compound of Formula (I) wherein Z is —C(H)═ and W is ═N—, such as a compound of Formula (VII):

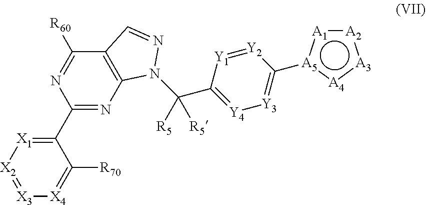

or a pharmaceutically acceptable salt thereof, wherein X₁, X₂, X₃, X₄, R₆₀, R₇₀, R₅, R₅′, Y₁, Y₂, Y₃, Y₄, A₁, A₂, A₃, A₄, and A₅ are each as described above with respect to Formula (I), and defined and described in classes and subclasses herein (e.g., with respect to any one or more of Formula (II)-(X)), both singly and in combination.

In some embodiments, the compound is a compound of Formula (I) wherein Z is —CH═ and W is ═C(R₉₀)—, such as a compound of Formula (VIIIa); or Z is —N═ and W is ═C(R₉₀)—, such as a compound of Formula (VIIIb):

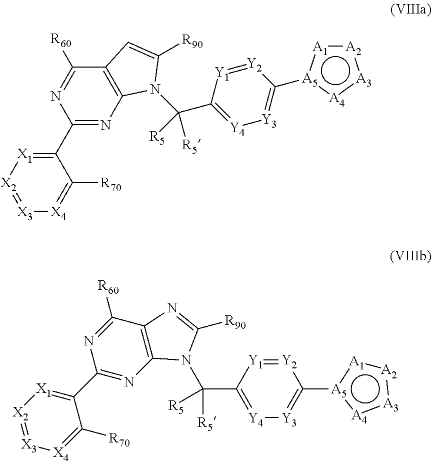

or a pharmaceutically acceptable salt thereof, wherein X₁, X₂, X₃, X₄, R₆₀, R₇₀, R₉₀, R₅, R₅′, Y₁, Y₂, Y₃, Y₄, A₁, A₂, A₃, A₄, and A₅ are each as described above with respect to Formula (I), and defined and described in classes and subclasses herein (e.g., with respect to any one or more of Formula (II)-(X)), both singly and in combination.

In some embodiments, the compound is a compound of Formula (VIIIb) wherein R₉₀ is —O—(C₁-C₄)alkyl (e.g., methoxy), such as a compound of Formula (VIIIc):

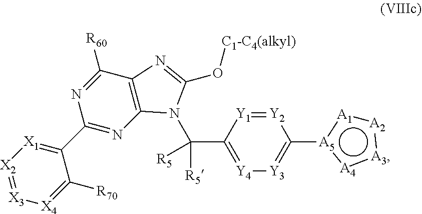

or a pharmaceutically acceptable salt thereof, wherein X₁, X₂, X₃, X₄, R₆₀, R₇₀, R₅, R₅′, Y₁, Y₂, Y₃, Y₄, A₁, A₂, A₃, A₄, and A₅ are each as described above with respect to Formula (I).

Another aspect of the application relates to a method of treating or preventing a disease or disorder associated with the inhibition of ubiquitin specific protease 1 (USP1). The method comprises administering to a patient in need of a treatment for diseases or disorders associated with modulation of ubiquitin specific protease 1 (USP1) an effective amount of a compound of any of Formulae (I)-(X), or a pharmaceutically acceptable salt, hydrate, solvate, prodrug, stereoisomer, or tautomer thereof. Another aspect of the application is directed to a method of inhibiting ubiquitin specific protease 1 (USP1). The method involves administering to a patient in need thereof an effective amount of a compound of any of Formulae (I)-(X), or a pharmaceutically acceptable salt, hydrate, solvate, prodrug, stereoisomer, or tautomer thereof.

Another aspect of the application is directed to pharmaceutical compositions comprising a compound of any of Formulae (I)-(X), or a pharmaceutically acceptable salt, hydrate, solvate, prodrug, stereoisomer, or tautomer thereof and a pharmaceutically acceptable carrier. The pharmaceutically acceptable carrier may further include an excipient, diluent, or surfactant.

Ultimately, the present application provides the medical community with compounds for development of new pharmaceutical compositions having the inhibition of USP1 enzymes as a mechanism of action. Another aspect of the present application relates to a compound of any of Formulae (I)-(X), or a pharmaceutically acceptable salt, hydrate, solvate, prodrug, stereoisomer, or tautomer thereof, for use in a method of treating or preventing a disease associated with inhibiting USP1. The present application provides inhibitors of USP1 that are therapeutic agents in the treatment of diseases such as cancer and other diseases associated with the modulation of ubiquitin specific protease 1 (USP1). The present application further provides methods of treating a disease or disorder associated with modulation of ubiquitin specific protease 1 (USP1) including, but not limited to, cancer comprising administering to a patient suffering from at least one of said diseases or disorders a compound of any of Formulae (I)-(X), or a pharmaceutically acceptable salt, hydrate, solvate, prodrug, stereoisomer, or tautomer thereof.

Another aspect of the application relates to a method of inhibiting or reducing DNA repair activity modulated by ubiquitin specific protease 1 (USP1). The method comprises administering to a patient in need thereof an effective amount of a compound of any of Formulae (I)-(X), or a pharmaceutically acceptable salt, hydrate, solvate, prodrug, stereoisomer, or tautomer thereof. Another aspect of the present application relates to a compound of any of Formulae (I)-(X), or a pharmaceutically acceptable salt, hydrate, solvate, prodrug, stereoisomer, or tautomer thereof, for use in a method of inhibiting or reducing DNA repair activity modulated by ubiquitin specific protease 1 (USP1). Another aspect of the present application relates to a compound of any of Formulae (I)-(X), or a pharmaceutically acceptable salt, hydrate, solvate, prodrug, stereoisomer, or tautomer thereof, for use in a method of treating or preventing a disease or disorder associated with DNA damage. Another aspect of the present application relates to the use of a compound of any of Formulae (I)-(X), or a pharmaceutically acceptable salt, hydrate, solvate, prodrug, stereoisomer, or tautomer thereof, in the manufacture of a medicament for treating or preventing a disease associated with inhibiting USP1. Another aspect of the application relates to a method of treating or preventing a disease or disorder associated with DNA damage. The method comprises administering to a patient in need of a treatment for diseases or disorders associated with DNA damage an effective amount of a compound of any of Formulae (I)-(X), or a pharmaceutically acceptable salt, hydrate, solvate, prodrug, stereoisomer, or tautomer thereof.

Another aspect of the application relates to a method of treating cancer. The method comprises administering to a patient in need thereof of a treatment for cancer an effective amount of a compound of any of Formulae (I)-(X), or a pharmaceutically acceptable salt, hydrate, solvate, prodrug, stereoisomer, or tautomer thereof. Another aspect of the present application relates to a compound of any of Formulae (I)-(X), or a pharmaceutically acceptable salt, hydrate, solvate, prodrug, stereoisomer, or tautomer thereof, for use in a method for treating or preventing cancer.

Another aspect of the present application relates to the use of a compound of any of Formulae (I)-(X), or a pharmaceutically acceptable salt, hydrate, solvate, prodrug, stereoisomer, or tautomer thereof, in the manufacture of a medicament for treating cancer.

Another aspect of the present application relates to the use of a compound of any of Formulae (I)-(X), or a pharmaceutically acceptable salt, hydrate, solvate, prodrug, stereoisomer, or tautomer thereof, in the manufacture of a medicament for inhibiting or reducing DNA repair activity modulated by ubiquitin specific protease 1 (USP1).

### BRIEF DESCRIPTION OF THE FIGURES

FIG. 1 is a table of exemplary Formula (Va), wherein the moiety

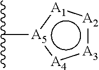

is

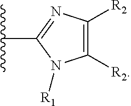

FIG. 2 is a table of exemplary compounds of Formula (Va), wherein R₇₀ is isopropyl and the moiety

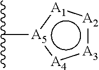

is

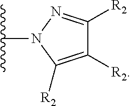

FIG. 3 is a table of exemplary compounds of Formula (Va), wherein R₇₀ is cyclopropyl and the moiety

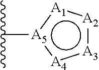

is

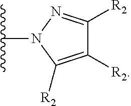

FIG. 4 is a table of exemplary compounds of Formula (Va).

FIG. 5 is a table of exemplary compounds of Formula (I), (IIa), (IIb), or (IIc).

FIG. 6 is a table of exemplary compounds of Formula (I), (IIIa), (IIIb), or (IIIc).

FIG. 7 is a table of exemplary compounds of Formula (I), (IVa), (IVb), or (IVc).

FIG. 8 is a table of exemplary compounds of Formula (Vb).

FIG. 9 is a table of exemplary compounds of Formula (VIa) or (VIb).

FIG. 10 is a table of exemplary compounds of Formula (VII).

FIG. 11 is a table of exemplary compounds of Formula (VIIIa), (VIIIb), or (VIIIc).

FIG. 12 is a table of exemplary compounds of Formula (I), (Xa) or (Xb).

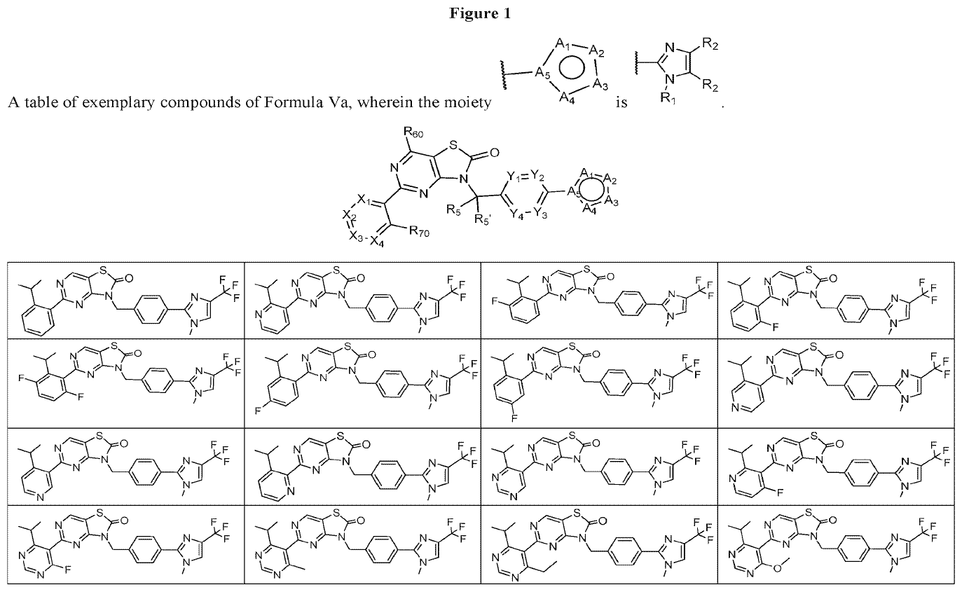

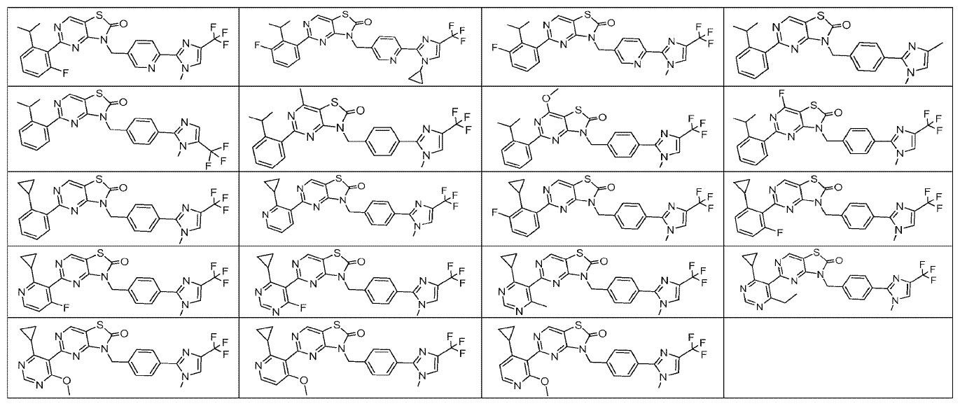

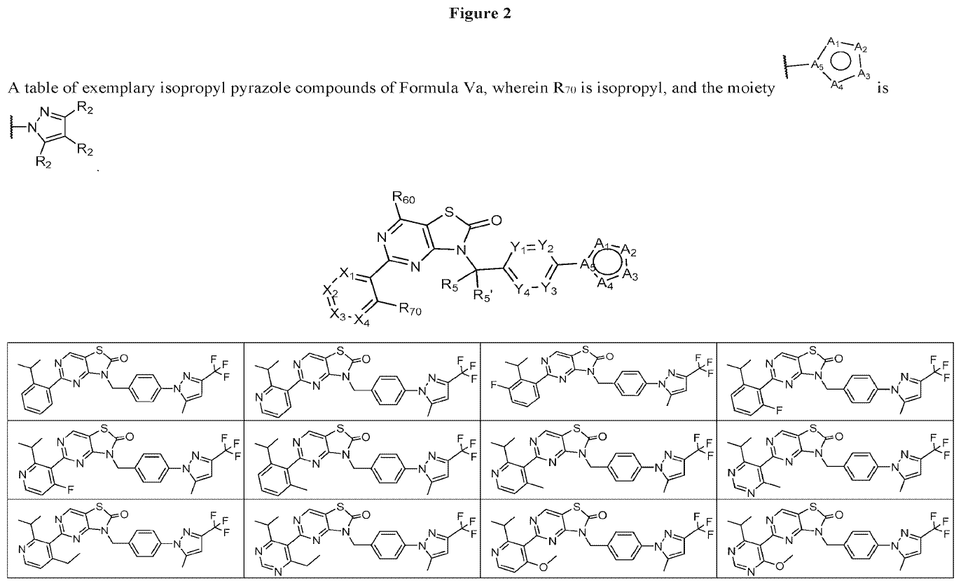

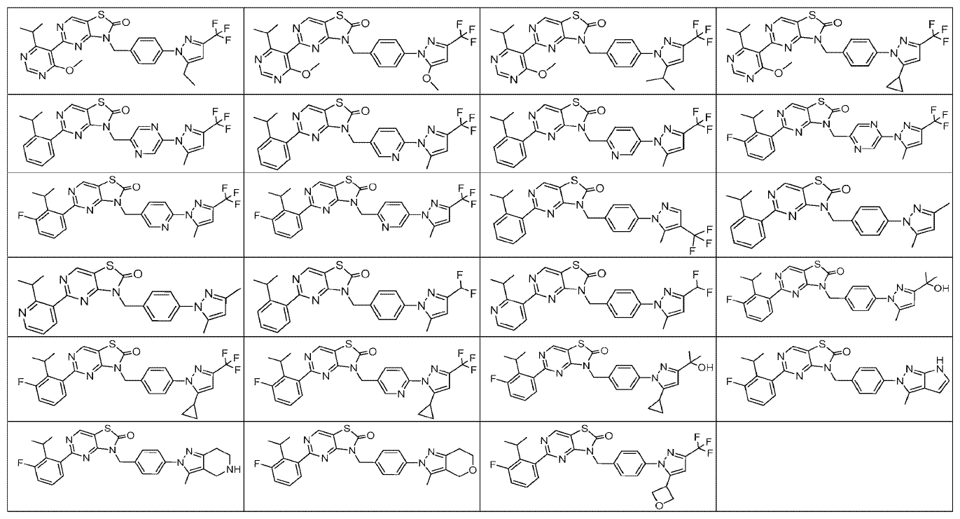

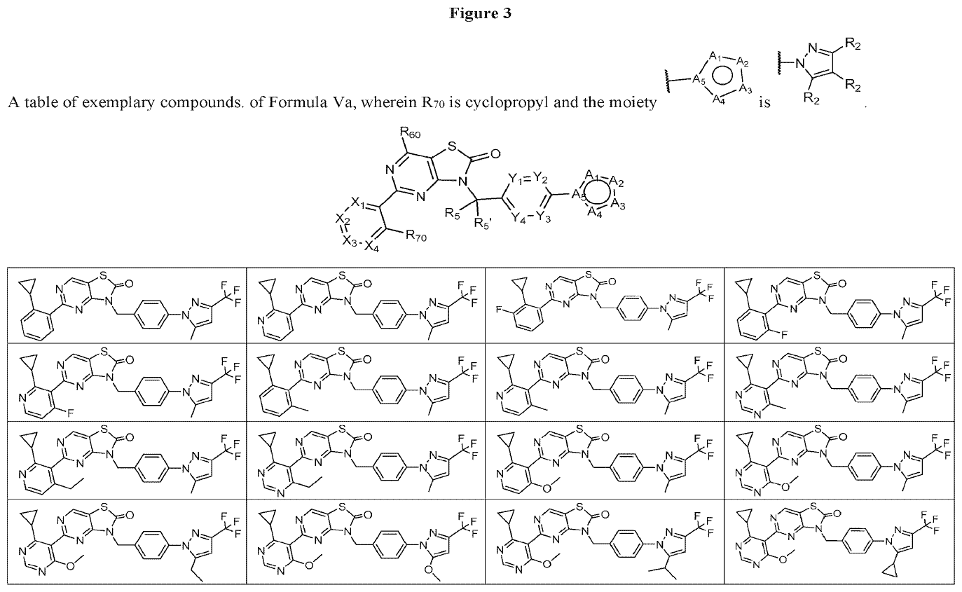

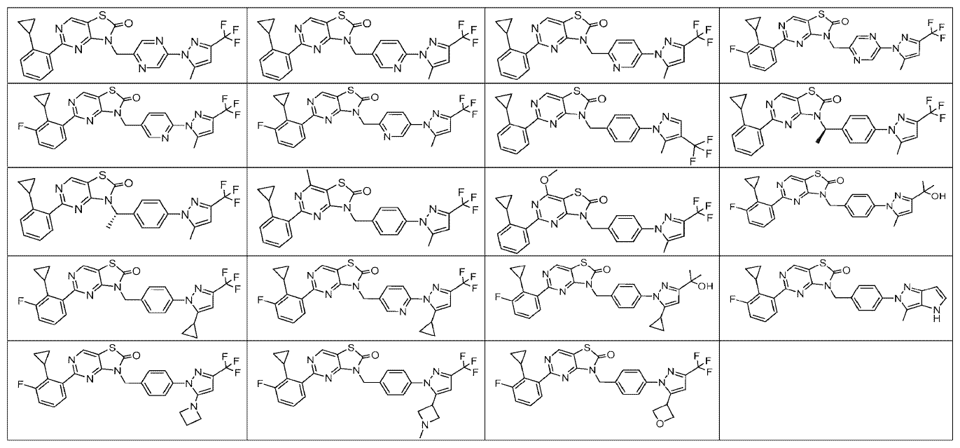

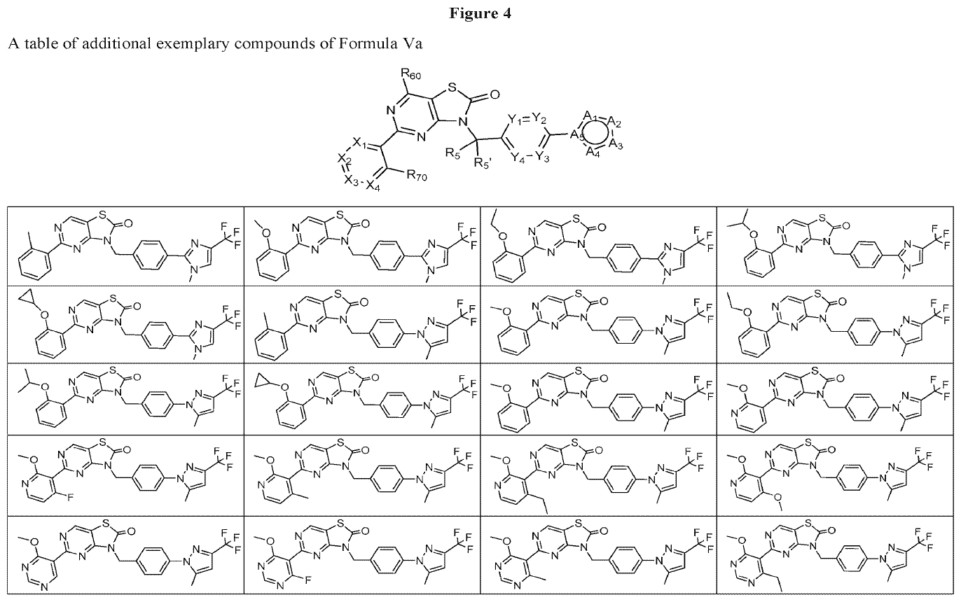

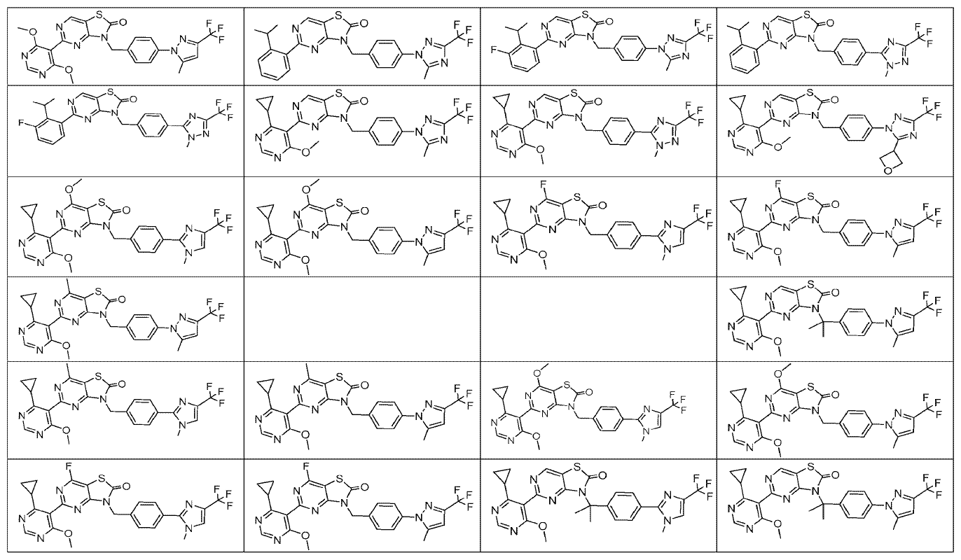

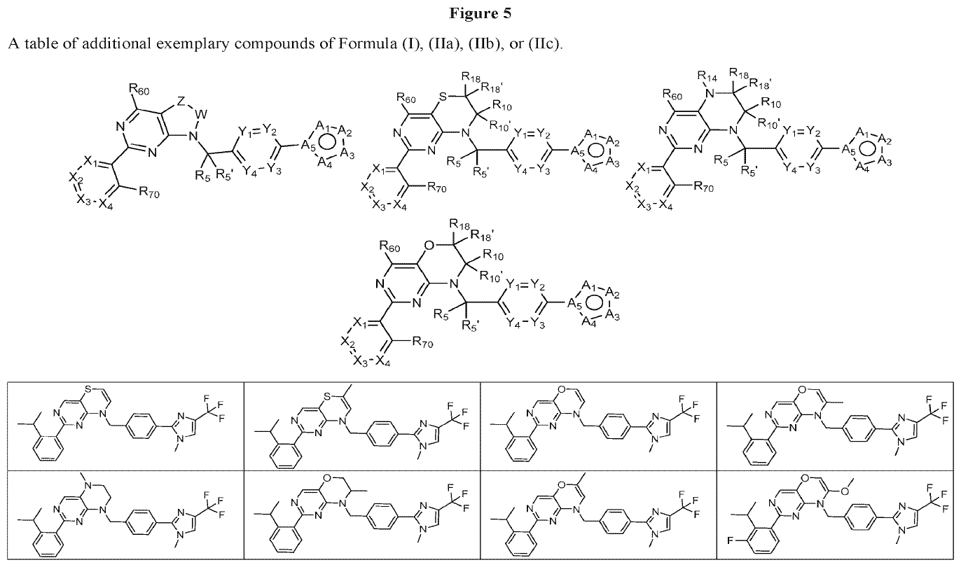

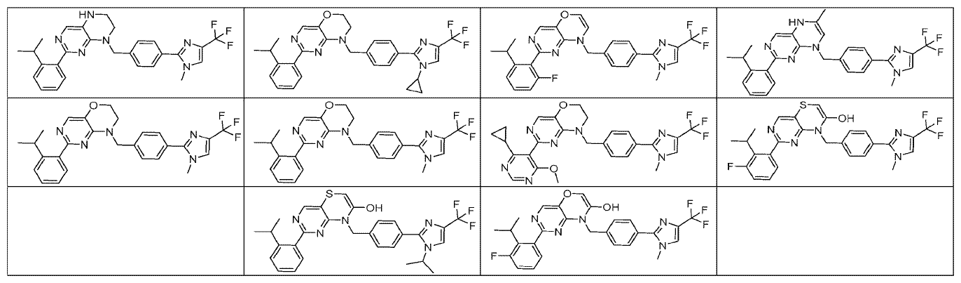

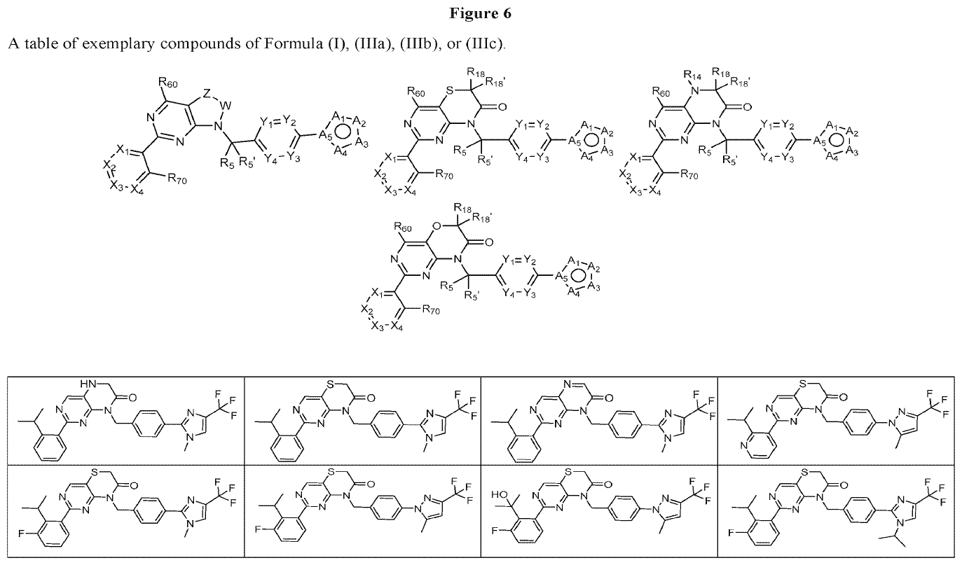

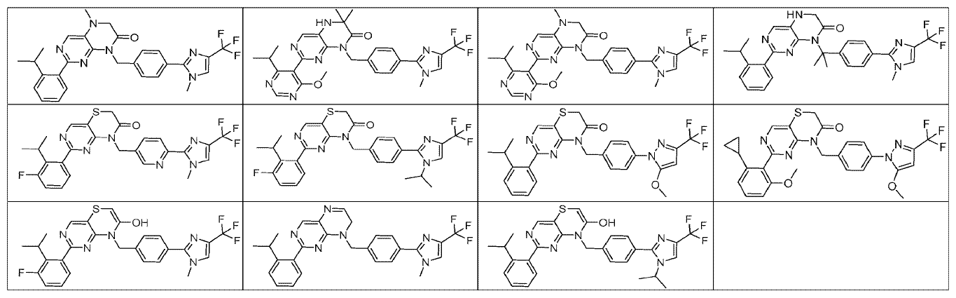

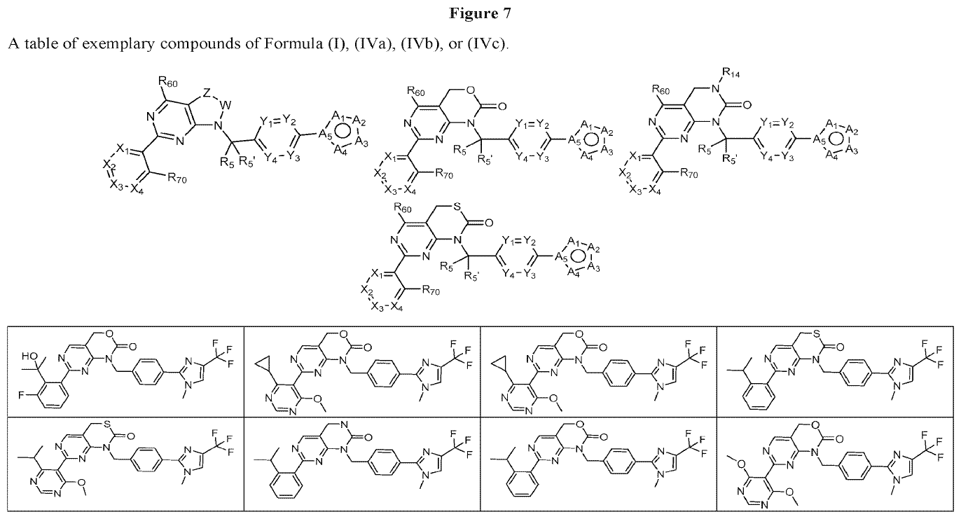

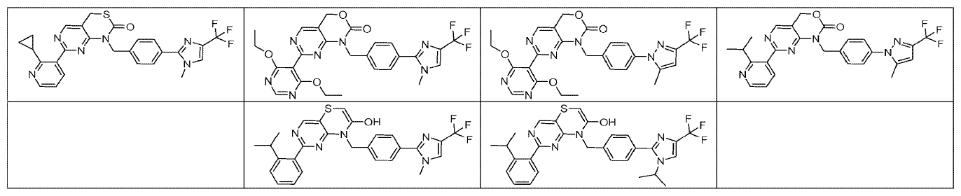

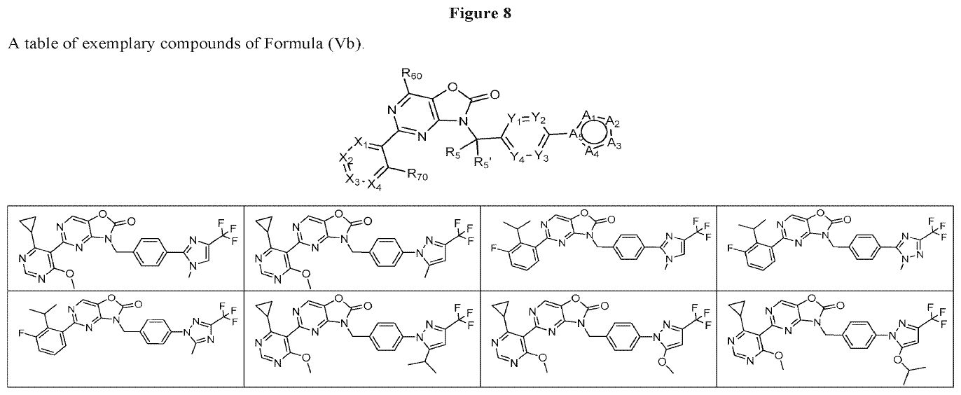

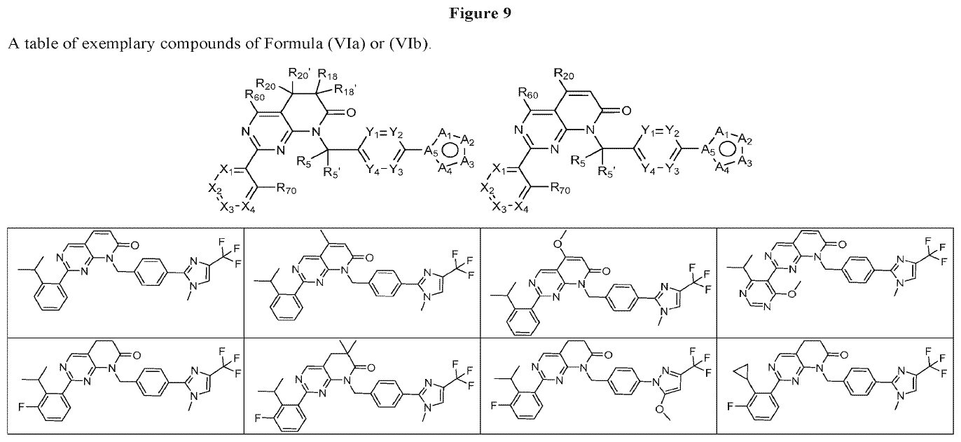

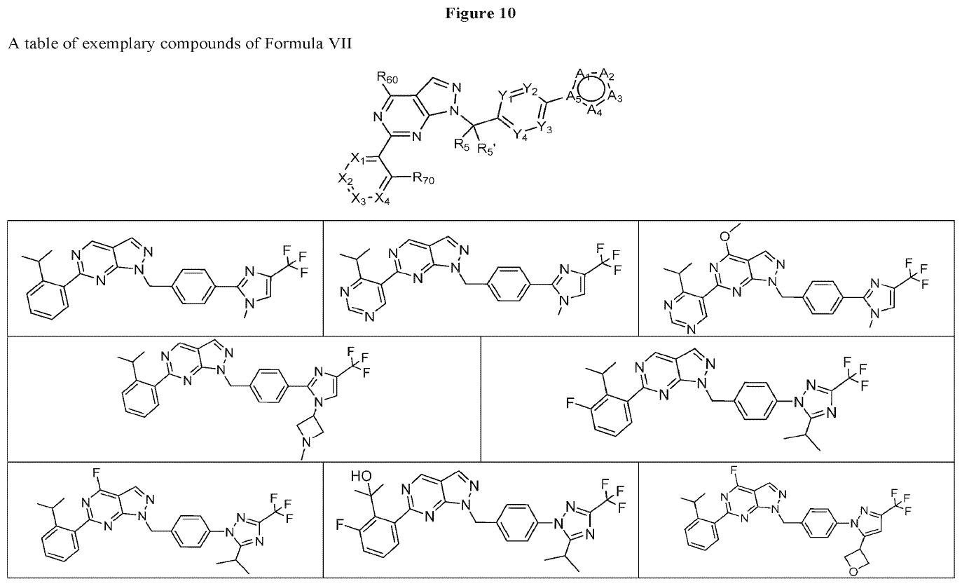

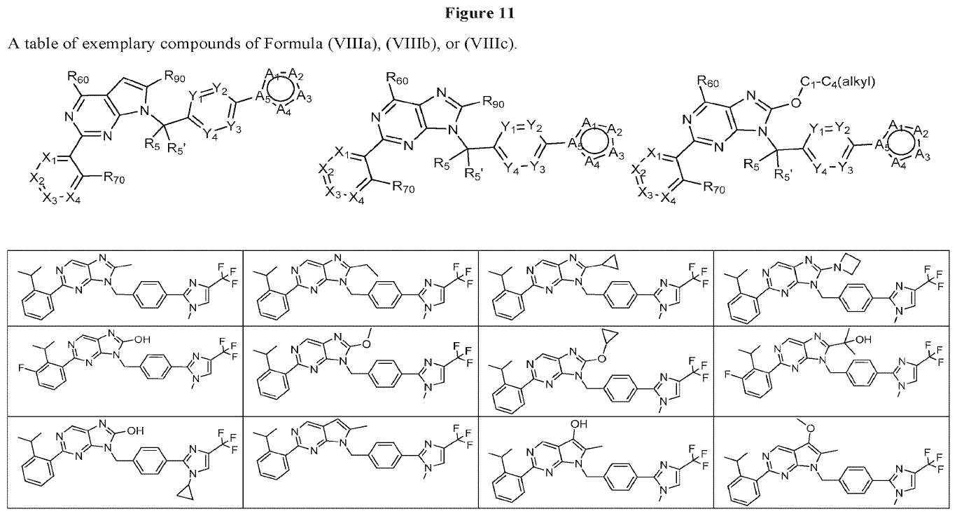

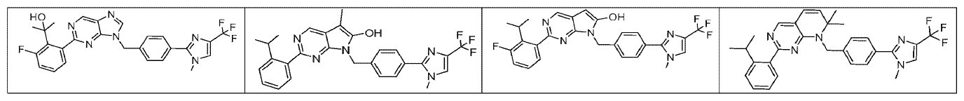

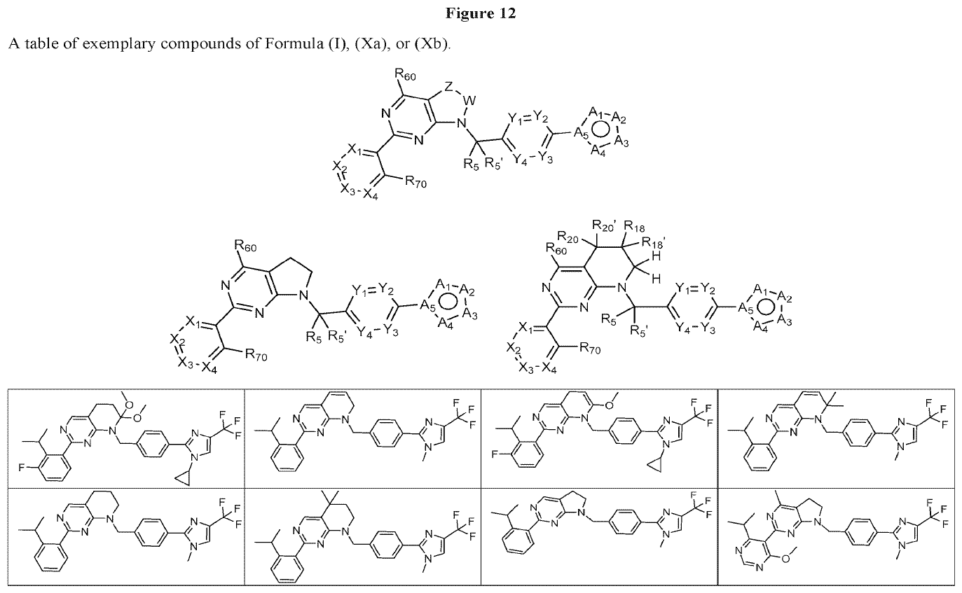

### DETAILED DESCRIPTION

The compounds of the present disclosure may be made by a variety of methods, including standard chemistry. Suitable synthetic routes are depicted in the examples given below.

The compounds of the present application can be prepared in a number of ways well known to those skilled in the art of organic synthesis. By way of example, compounds of the present application can be synthesized using the methods described below, together with synthetic methods known in the art of synthetic organic chemistry, or variations thereon as appreciated by those skilled in the art. Preferred methods include but are not limited to those methods described below. Compounds of the present application can be synthesized by following the steps outlined in the General Schemes below. Starting materials are either commercially available or made by known procedures in the reported literature or as illustrated.

The present disclosure provides compounds of Formula (I):

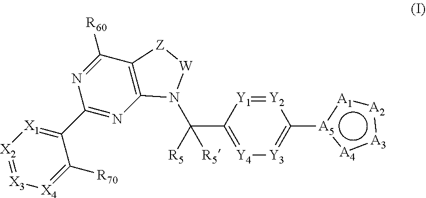

wherein, X₁, X₂, X₃, X₄, R₆₀, R₇₀, Z, W, R₅, R₅′, Y₁, Y₂, Y₃, Y₄, A₁, A₂, A₃, A₄, and A₅ are each as described herein, or a pharmaceutically acceptable salt thereof.

In some embodiments, X₁ is CR₆ or N; X₂ is CR₇ or N; X₃ is CR₈ or N; and X₄ is CR₉ or N; provided that no more than two of X₁, X₂, X₃ or X₄ are N and adjacent positions of X₁, X₂, X₃ or X₄ cannot both be N. In some embodiments, only one of X₁, X₂, X₃, X₄ is N. In some embodiments, only two of X₁, X₂, X₃, X₄ is N. In some embodiments, only two non-adjacent positions of X₁, X₂, X₃, X₄ are each N. In some embodiments, X₁ is CR₆, X₂ is CR₇, X₃ is CR₈, and X₄ is N. In some embodiments, X₁ is N; X₂ is CR₇; X₃ is CR₈; and X₄ is CR₉. In some embodiments, X₁ is CR₆; X₂ is N; X₃ is CR₈; and X₄ is CR₉. In some embodiments, X₁ is CR₆; X₂ is CR₇; X₃ is N; and X₄ is CR₉.

X₁ is CR₆ or N; X₂ is CR₇ or N; X₃ is CR₈ or N; and X₄ is CR₉ or N; provided that no more than two of X₁, X₂, X₃, or X₄ are N and adjacent positions of X₁, X₂, X₃, or X₄ cannot both be N.

In some embodiments, if X₂ is N, then X₁ is CR₆ and X₃ is CR₈; and if X₃ is N, then X₂ is CR₇ and X₄ is CR₉. In some embodiments, if X₂ is N, then X₁ and X₃ cannot be N; and if X₃ is N, then X₂ and X₄ cannot be N.

In some embodiments, R₆ is hydrogen; methyl or ethyl, wherein the methyl or ethyl is optionally substituted with one or more substituents independently selected from hydroxyl or halogen (e.g., F); cyclopropyl, cyclobutyl or cyclohexyl, wherein the cyclopropyl, cyclobutyl or cyclohexyl is optionally substituted with one or more substituents independently selected from methyl, hydroxyl, methoxy, —N(R_{b})(R_{b′}), or halogen (e.g., F); or a 4- to 6-membered heterocycloalkyl having 1-3 heteroatoms independently selected from N, O, or S, optionally substituted with one or more substituents independently selected from methyl, hydroxyl, methoxy, —N(R_{b})(R_{b}′), or halogen (e.g., F). In some embodiments, R₆ is selected from the group consisting of —H, —CH₃, —CH₂F, —CHF₂, —CF₃, —CH₂CH₃, —CH₂CH₂F, —CH₂CHF₂, —CH₂CF₃, —CH(CH₃)₂, —COH(CH₃)₂, cyclopropyl, cyclobutyl, —(CH₂)₂CH₃, —CH₂—O—CH₃, —CH₂—O—CH₂F, —CH₂—O—CHF₂, —CH₂—O—CF₃, —CH₂CH(CH₃)₂, —CH₂COH(CH₃)₂, methylcyclopropane, oxetane, azetidine, N-methylazetidine, cyclopentyl, cyclohexyl, —CH₂CH₂N-dimethyl, —O—CH₃, —O—CH₂F, —O—CHF₂, —O—CF₃, —O—CH₂CH₃, —O—CH₂CH₂F, —O—CH₂CHF₂, —O—CH₂CF₃, —O—CH(CH₃)₂, and —O— cyclopropyl.

In some embodiments, R₇, R₈ and R₉ are each independently hydrogen or halogen. In some embodiments, R₇, R₈ and R₉ are each independently hydrogen. In some embodiments, R₇, R₈ and R₉ are each independently hydrogen or F. In some embodiments, R₇, R₈ and R₉ are each independently hydrogen or Cl. In some embodiments, R₇, R₈ and R₉ are each independently hydrogen, F or Cl.

In some embodiments, R₆₀ is hydrogen, (C₁-C₄)alkyl, (C₃-C₆)cycloalkyl, or 4- to 6-membered heterocycloalkyl comprising one N or O heteroatom, wherein the alkyl, cycloalkyl or heterocycloalkyl is optionally substituted with one or more substituents independently selected from halogen, hydroxyl, (C₁-C₄)alkoxy, or —NR_{b}R_{b}. In some embodiments, R₆₀ is selected from the group consisting of: hydrogen, —CH₃, —CH₂F, —CHF₂, —CF₃, —CH₂CH₃, —CH₂CH₂F, —CH₂CHF₂, —CH₂CF₃, —CH(CH₃)₂, —COH(CH₃)₂, cyclopropyl, cyclobutyl, —(CH₂)₂CH₃, —CH₂—O—CH₃, —CH₂—O—CH₂F, —CH₂—O—CHF₂, —CH₂—O—CF₃, —CH₂CH(CH₃)₂, —CH₂COH(CH₃)₂, methylcyclopropane, oxetane, azetidine, N-methylazetidine, cyclopentyl, cyclohexyl, —CH₂CH₂N-dimethyl, —O—CH₃, —O—CH₂F, —O—CHF₂, —O—CF₃, —O—CH₂CH₃, —O—CH₂CH₂F, —O—CH₂CHF₂, —O—CH₂CF₃, —O—CH(CH₃)₂, and —O-cyclopropyl.

In some embodiments, R₇₀ is hydrogen, (C₁-C₄)alkyl, (C₃-C₆)cycloalkyl, or 4- to 6-membered heterocycloalkyl comprising one N or O heteroatom, wherein the alkyl, cycloalkyl or heterocycloalkyl is optionally substituted with one or more substituents independently selected from halogen, hydroxyl, (C₁-C₄)alkoxy, or —NR_{b}R_{b}. In some embodiments, R₇₀ is selected from the group consisting of: hydrogen, —CH₃, —CH₂F, —CHF₂, —CF₃, —CH₂CH₃, —CH₂CH₂F, —CH₂CHF₂, —CH₂CF₃, —CH(CH₃)₂, —COH(CH₃)₂, cyclopropyl, cyclobutyl, —(CH₂)₂CH₃, —CH₂—O—CH₃, —CH₂—O—CH₂F, —CH₂—O—CHF₂, —CH₂—O—CF₃, —CH₂CH(CH₃)₂, —CH₂COH(CH₃)₂, methylcyclopropane, oxetane, azetidine, N-methylazetidine, cyclopentyl, cyclohexyl, —CH₂CH₂N-dimethyl, —O—CH₃, —O—CH₂F, —O—CHF₂, —O—CF₃, —O—CH₂CH₃, —O—CH₂CH₂F, —O—CH₂CHF₂, —O—CH₂CF₃, —O—CH(CH₃)₂, and —O-cyclopropyl.

In some embodiments, R₅ and R₅′ are each hydrogen. In some embodiments, R₅ and R₅′ are each independently hydrogen or halogen (e.g., F). In some embodiments, R₅ and R₅′ are each independently hydrogen, F or Cl. In some embodiments, R₅ and R₅′ are each independently (C₁-C₄)alkyl optionally substituted with one or more F, —O—(C₁-C₄)alkyl optionally substituted with one or more substituents independently selected from F, —(C₁-C₄)alkyl-N(R_{b})(R_{b}′) wherein the alkyl is optionally substituted with one or more F. In some embodiments, R₅ and R₅′ are each independently hydrogen; methyl or ethyl each optionally substituted with one or more halogen; cyclopropyl optionally substituted with one or more halogen; or —O—(C₁-C₄)alkyl optionally substituted with one or more halogen. In some embodiments, R₅ and R₅′ are each independently hydrogen; methyl or ethyl each optionally substituted with one or more F; cyclopropyl optionally substituted with one or more F; or —O—(C₁-C₄)alkyl optionally substituted with one or more F. In some embodiments, R₅ and R₅′ are each independently hydrogen, —F, —CH₃, —CH₂CH₃, —O—CH₃, —O—CH₂CH₃, or —CH₂CH₂N-dimethyl. In some embodiments, R₅ and R₅′ are each independently hydrogen. In some embodiments, R₅ and R₅′ together form a spirocyclic cyclopropyl.

In some embodiments, R₅ and R₅′ together form a spirocyclic cyclopropyl, cyclobutyl or cyclohexyl optionally substituted with methyl or halogen. In some embodiments, R₅ and R₅′ together form a spirocyclic cyclopropyl or cyclobutyl optionally substituted with methyl or F. In some embodiments, R₅ and R₅′ together form a spirocyclic cyclopropyl, optionally substituted with methyl or F. In some embodiments, R₅ and R₅′ together form a spirocyclic cyclopropyl, optionally substituted with methyl. In some embodiments, R₅ and R₅′ together form a (C₃-C₆) cycloalkyl ring optionally substituted with one or more F or methyl.

In some embodiments, Y₁, Y₂, Y₃ and Y₄ are each independently —C(R_{y})— wherein R_{y} is as defined with respect to Formula (I) and described in classes and subclasses herein (e.g., with respect to any one or more of Formula (II)-(X)). In some embodiments, Y₁, Y₂, Y₃ and Y₄ are each independently —C(R_{y})— wherein each R_{y} is independently hydrogen. In some embodiments, Y₁, Y₂, Y₃ and Y₄ are each independently —C(R_{y})— wherein one, two, or three R_{y} groups are independently methyl optionally substituted with one or more F or Cl, and each remaining R_{y} is hydrogen. In some embodiments, Y₁ is —C(R_{y})—, where R_{y} is methyl optionally substituted with one or more F or Cl, and Y₂, Y₃ and Y₄ are each hydrogen. In some embodiments, Y₂ is —C(R_{y})—, where R_{y} is methyl optionally substituted with one or more F or Cl, and Y₁, Y₃ and Y₄ are each hydrogen. In some embodiments, Y₃ is —C(R_{y})—, where R_{y} is methyl optionally substituted with one or more F or Cl, and Y₁, Y₂ and Y₄ are each independently hydrogen. In some embodiments, Y₄ is —C(R_{y})—, where R_{y} is methyl optionally substituted with one or more F or Cl, and Y₁, Y₂ and Y₃ are each hydrogen. In some embodiments, Y₁, Y₂, Y₃ and Y₄ are each independently —C(R_{y})— wherein one R_{y} is F and each remaining R_{y} is hydrogen. In some embodiments, any one of Y₁, Y₂, Y₃ and Y₄ is N and each of the remaining Y₁, Y₂, Y₃ and Y₄ is independently —C(R_{y})— wherein each R_{y} is independently hydrogen or halogen (e.g., F or Cl). In some embodiments, any two of Y₁, Y₂, Y₃ and Y₄ is N and each of the remaining Y₁, Y₂, Y₃ and Y₄ is independently —C(R_{y})— wherein each R_{y} is independently hydrogen or halogen (e.g., F or Cl). In some embodiments, any one of Y₁ or Y₂, is N and the remaining one of Y₁ and Y₂, is —C(R_{y})— wherein R_{y} is hydrogen or halogen (e.g., F or Cl). In some embodiments, any one of Y₃, or Y₄, is N and the remaining one of Y₃ and Y₄, is —C(R_{y})—, wherein R_{y} is hydrogen or halogen (e.g., F or Cl). In some embodiments, any one of Y₁ or Y₂, is N, and the remaining one of Y₁ and Y₂, and each of Y₃ and Y₄ is independently —C(R_{y})— wherein each R_{y} is independently hydrogen or halogen (e.g., F or Cl). In some embodiments, any one of Y₃ or Y₄, is N and the remaining one of Y₃, and Y₄, and each of Y₁ and Y₂ is independently —C(R_{y})— wherein each R_{y} is independently hydrogen, halogen (e.g., F or Cl) or (C₁-C₄)alkyl (e.g., methyl). In some embodiments, any one of Y₃ or Y₄ is N and the remaining one of Y₃ and Y₄, and each of Y₁ and Y₂ is independently —C(R_{y})— wherein each R_{y} is methyl. In some embodiments, any one of Y₃ or Y₄ is N and the remaining one of Y₃ and Y₄, and each of Y₁ and Y₂ is independently —C(R_{y})—, wherein each R_{y} is hydrogen. In some embodiments, any one of Y₁ or Y₄ is N and the remaining one of Y₃ and Y₄, and each of Y₁ and Y₂ is independently —C(R_{y})—, wherein each R_{y} is methyl.

In some embodiments, A₁, A₂, A₃, A₄, and A₅ together form an optionally substituted 5-membered heteroaryl ring comprising one or more heteroatoms selected from the group consisting of N, O and S. In some embodiments, A₁, A₂, A₃, A₄, and A₅ together form an optionally substituted 5-membered heteroaryl ring comprising one or more nitrogen heteroatoms. In some embodiments, A₁, A₂, A₃, A₄, and A₅ together form an optionally substituted 5-membered heteroaryl ring comprising one or two nitrogen heteroatoms. In some embodiments, A₁, A₂, A₃, A₄, and A₅ together form an optionally substituted 5-membered heteroaryl ring with a total of one nitrogen heteroatom. In some embodiments, A₁, A₂, A₃, A₄, and A₅ together form an optionally substituted 5-membered heteroaryl ring with a total of two nitrogen heteroatoms. In some embodiments, A₁, A₂, A₃, A₄, and A₅ together form an optionally substituted 5-membered heteroaryl ring with a total of two non-adjacent nitrogen heteroatoms. In some embodiments, A₁, A₂, A₃, A₄, and A₅ together form an optionally substituted 5-membered heteroaryl ring and one or two of A₁, A₂, A₃, A₄, and A₅ is NR₁ and the remainder of A₁, A₂, A₃, A₄, and A₅ are each CR₂.

In some embodiments, each of A₁, A₂, A₃, A₄, and A₅ is selected from the group consisting of CR₂, NR₁, O, and S to form a 5-membered heteroaryl ring. In some embodiments, A₁ is N. In some embodiments, A₂ is CR₂. In some embodiments, A₃ is CR₂. In some embodiments, A₄ is NR₁. In some embodiments, A₅ is CR₂ or N. In some embodiments, A₁ is NR₁, wherein R₁ is a bond, and A₂ is CR₂, wherein R₂ is as defined with respect to Formula (I) and described in classes and subclasses herein (e.g., with respect to any one or more of Formula (II)-(X)). In some embodiments, A₁ is NR₁, wherein R₁ is a bond, and A₂ is CR₂, wherein R₂ is methyl optionally substituted with one or more F. In some embodiments, A₁ is NR₁, wherein R₁ is a bond, and A₂ is CR₂, wherein R₂ is —CF₃. In some embodiments, A₁ is NR₁, wherein R₁ is a bond, and A₃ is CR₂, wherein R₂ is as defined with respect to Formula (I) and described in classes and subclasses herein (e.g., with respect to any one or more of Formula (II)-(X)). In some embodiments, A₁ is NR₁, wherein R₁ is a bond, and A₃ is CH. In some embodiments, A₁ is NR₁, wherein R₁ is a bond, and A₂ is CR₂, wherein R₂ is methyl optionally substituted with one or more F, and A₃ is CR₂, wherein R₂ is as defined with respect to Formula (I) and described in classes and subclasses herein (e.g., with respect to any one or more of Formula (II)-(X)). In some embodiments, A₁ is NR₁, wherein R₁ is a bond, and A₂ is CF₃, and A₃ is CH. In some embodiments, A₁ is NR₁, wherein R₁ is a bond, and A₄ is NR₁, wherein R₁ is as defined with respect to Formula (I) and described in classes and subclasses herein (e.g., with respect to any one or more of Formula (II)-(X)). In some embodiments, A₁ is NR₁, wherein R₁ is a bond, and A₄ is NR₁, wherein R₁ is methyl, hydrogen or 3- to 6-membered cycloalkyl or heterocycloalkyl optionally substituted with one or more halogen or methyl. In some embodiments, A₁ is NR₁, wherein R₁ is a bond, and A₄ is NR₁, wherein R₁ is methyl or oxetane.

In some embodiments, A₁ is NR₁, wherein R₁ is a bond, and A₂ is NR₁ or CR₂. In some embodiments, A₃ is NR₁ or CR₂ if A₂ is —CH. In some embodiments, A₃ is CH, NH, O or S if A₂ is CR₂ and R₂ is not hydrogen, and A₄ is CR₂ and A₅ is C. In some embodiments, A₃ is CH, or NR₁, wherein R₁ is a bond, if A₂ is NR₁ or if A₄ is NR₁ or if A₅ is N. In some embodiments, A₄ is NR₁ or CR₂ and A₅ is N or C. In some embodiments, A₁ is NR₁, wherein R₁ is a bond, A₂ is NR₁ or CR₂, A₄ is NR₁ or CR₂ and A₅ is N or C. In some embodiments, A₁ is N, A₂ is CH, A₃ is NR₁ or CR₂, A₄ is NR₁ or CR₂ and A₅ is N or C. In some embodiments, A₁ is NR₁, wherein R₁ is a bond, A₂ is CR₂, A₃ is CH₂, NH, O or S, A₄ is CR₂ and A₅ is C. In some embodiments, A₁ is NR₁, wherein R₁ is a bond, A₂ is NR₁, A₃ is CH or N, A₄ is NR₁ or CR₂ and A₅ is C or N. In some embodiments, A₁ is NR₁, wherein R₁ is a bond, A₂ is NR₁ or CR₂, A₃ is CH or NR₁, wherein R₁ is a bond, A₄ is NR₁ and A₅ is C or N. In some embodiments, A₁ is NR₁, wherein R₁ is a bond, A₂ is NR₁ or CR₂, A₃ is CH or N, A₄ is NR₁ or CR₂ and A₅ is N.

In some embodiments, R₁ in A₁, A₂, A₃, A₄, and A₅ is a bond, hydrogen, methyl, ethyl, propyl (e.g., n-propyl or isopropyl), butyl (e.g., n-butyl or isobutyl), methoxy, ethoxy, propoxy, butoxy, cyclopropyl, cyclobutyl, cyclopentyl cyclohexyl, oxetane, or azetidine, wherein the methyl, ethyl, propyl (e.g., n-propyl or isopropyl), butyl (e.g., n-butyl or isobutyl), methoxy, ethoxy, propoxy, or butoxy is each optionally substituted with one or more Rₐ and the cyclopropyl, cyclobutyl, cyclopentyl, cyclohexyl, oxetane, or azetidine is each optionally substituted with one or more R_{a′} as defined with respect to Formula (I) and described in classes and subclasses herein (e.g., with respect to any one or more of Formula (II)-(X)). In some embodiments, R₁ in A₁, A₂, A₃, A₄, and A₅ is independently selected from the group consisting of: a bond, hydrogen, —CH₃, —CH₂F, —CHF₂, —CF₃, —CH₂CH₃, —CH₂CH₂F, —CH₂CHF₂, —CH₂CF₃, —CH(CH₃)₂, —COH(CH₃)₂, cyclopropyl, cyclobutyl, —(CH₂)₂CH₃, —CH₂—O—CH₃, —CH₂—O—CH₂F, —CH₂—O—CHF₂, —CH₂—O—CF₃, —CH₂CH(CH₃)₂, —CH₂COH(CH₃)₂, methylcyclopropane, oxetane, -azetidine, N-methylazetidine, cyclopentyl, cyclohexyl, and —CH₂CH₂N-dimethyl.

In some embodiments, R₂ in A₁, A₂, A₃, A₄, and A₅ is selected from the group consisting of: a bond, hydrogen, (C₁-C₄)alkyl, —O—(C₁-C₄)alkyl, 3-6 membered cycloalkyl, and 3- to 6-membered heterocycloalkyl having 1-3 heteroatoms independently selected from N, O, and S, wherein the (C₁-C₄)alkyl or —O—(C₁-C₄)alkyl is each optionally substituted with one or more Rₐ and the 3-6 membered cycloalkyl or 3- to 6-membered heterocycloalkyl is each optionally substituted with one or more R_{a′}. In some embodiments, R₂ in A₁, A₂, A₃, A₄, and A₅ is an oxygen-linked 3-6 member cycloalkyl or 3-6 member heterocycloalkyl or a nitrogen-linked 3-6 member cycloalkyl or 3-6 member heterocycloalkyl. In some embodiments, R₂ in A₁, A₂, A₃, A₄, and A₅ is independently selected from the group consisting of: a bond, hydrogen, —CH₃, —CH₂F, —CHF₂, —CF₃, —CH₂CH₃, —CH₂CH₂F, —CH₂CHF₂, —CH₂CF₃, —CH(CH₃)₂, —COH(CH₃)₂, cyclopropyl, cyclobutyl, —(CH₂)₂CH₃, —CH₂—O—CH₃, —CH₂—O—CH₂F, —CH₂—O—CHF₂, —CH₂—O—CF₃, —CH₂CH(CH₃)₂, —CH₂COH(CH₃)₂, methylcyclopropane, oxetane, azetidine, N-methylazetidine, cyclopentyl, cyclohexyl, —CH₂CH₂N-dimethyl, —O—CH₃, —O—CH₂F, —O—CHF₂, —O—CF₃, —O—CH₂CH₃, —O—CH₂CH₂F, —O—CH₂CHF₂, —O—CH₂CF₃, —O—CH(CH₃)₂, and —O-cyclopropyl.

In some embodiments, two occurrences of R₂ on adjacent carbon atoms in A₁, A₂, A₃, or A₄, may together form a fused ring selected from a 5- to 6-membered heterocycloalkyl having 1-3 heteroatoms selected from N, O, or S or a 5- to 6-membered heteroaryl having 1-3 heteroatoms selected from N, O, or S, wherein each ring may independently be optionally substituted with one or more R_{a′};

In some embodiments, A₁ is NR₁, wherein R₁ is a bond, A₂ is CR₂, wherein R₂ is methyl optionally substituted with one or more F, and A₄ is NR₁, wherein R₁ is as defined with respect to Formula (I) and described in classes and subclasses herein (e.g., with respect to any one or more of Formula (II)-(X)). In some embodiments, A₁ is NR₁, wherein R₁ is a bond, A₂ is CR₂, wherein R₂ is methyl optionally substituted with one or more F, A₃ is CR₂, and A₄ is NR₁, wherein R₁ and R₂ are each independently as defined with respect to Formula (I) and described in classes and subclasses herein (e.g., with respect to any one or more of Formula (II)-(X)). In some embodiments, A₁ is NR₁, wherein R₁ is a bond, A₂ is CF₃, A₃ is CH, and A₄ is NR₁, wherein R₁ is methyl or oxetane.

In some embodiments, each Rₐ in each R₁ or R₂ in A₁, A₂, A₃, A₄, and A₅ is independently selected from the group consisting of halogen, hydroxyl, —N(R_{b})(R_{b′}), (C₁-C₄)alkoxy optionally substituted with one or more R_{a′}, and 3- to 6-membered cycloalkyl optionally substituted with one or more R_{a′}. In some embodiments, each Rₐ in each R₁ or R₂ in A₁, A₂, A₃, A₄, and A₅ is independently selected from the group consisting of Cl, F, hydroxyl, —N(R_{b})(R_{b′}), methoxy or ethoxy optionally substituted with one or more R_{a′}, and cyclopropyl, cyclobutyl, or cyclohexyl optionally substituted with one or more Rₐ′. In some embodiments, each Rₐ in each R₁ or R₂ in A₁, A₂, A₃, A₄, and A₅ is independently selected from the group consisting of F, hydroxyl, —N(R_{b})(R_{b′}), methoxy optionally substituted with one or more R_{a′}, and cyclopropyl, optionally substituted with one or more Rₐ′.

In some embodiments, each Rₐ′ in each R₁ or R₂ in A₁, A₂, A₃, A₄, and A₅ is independently selected from the group consisting of halogen or (C₁-C₄)alkyl optionally substituted with one or more halogen. In some embodiments, each Rₐ′ in each R₁ or R₂ in A₁, A₂, A₃, A₄, and A₅ is independently selected from the group consisting of F, Cl or methyl, ethyl, propyl (e.g., n-propyl or isopropyl), or butyl (e.g., n-butyl or isobutyl) optionally substituted with one or more F or Cl. In some embodiments, each R_{a′} in each R₁ or R₂ in A₁, A₂, A₃, A₄, and A₅ is independently selected from the group consisting of F, or methyl, optionally substituted with one or more F.

In some embodiments, each R_{b} and R_{b}′ in each R₁ or R₂ in A₁, A₂, A₃, A₄, and A₅ is independently selected from the group consisting of hydrogen and (C₁-C₄)alkyl optionally substituted with one or more halogen. In some embodiments, each R_{b} and R_{b}′ in each R₁ or R₂ in A₁, A₂, A₃, A₄, and A₅ is independently selected from the group consisting of hydrogen, methyl, ethyl, propyl (e.g., n-propyl or isopropyl), or butyl (e.g., n-butyl or isobutyl) wherein each alkyl moiety is optionally substituted with one or more Cl or F. In some embodiments, each R_{b} and R_{b}′ in each R₁ or R₂ in A₁, A₂, A₃, A₄, and A₅ is independently selected from the group consisting of hydrogen, or methyl optionally substituted with one or more F. In some embodiments, each R_{b} and R_{b}′ in each R₁ or R₂ in A₁, A₂, A₃, A₄, and A₅ is methyl. In some embodiments, each R_{b} and R_{b}′ in each R₁ or R₂ in A₁, A₂, A₃, A₄, and A₅ is hydrogen.

In some embodiments, a compound provided herein is a compound wherein A₁ is NR₁, wherein R₁ is a bond, A₂ is CR₂ wherein R₂ is methyl optionally substituted with one or more F (e.g., CF₃), A₃ is CH, A₄ is NR₁ where R₁ is methyl or a 3-6 membered heteroaryl comprising an O heteroatom, and A₅ is C. In some embodiments, a compound provided herein is a compound wherein A₁ is CR₂ and R₂ is hydrogen or methyl optionally substituted with one or more F (e.g., CF₃) in A₁, A₂ is CR₂ wherein R₂ is hydrogen or methyl optionally substituted with one or more F (e.g., CF₃) in A₂, A₃ is CH, A₄ is NR₁, wherein R₁ is a bond, and A₅ is N. In some embodiments, a compound provided herein is a compound wherein A₁ is CH, A₂ is CH, A₃ is CH, A₄ is NR₁, wherein R₁ is a bond, and A₅ is N.

In some embodiments, the moiety

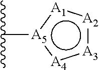

is selected from the group consisting of:

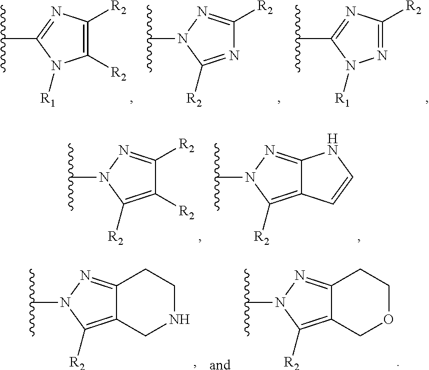

In some embodiments, Z and W are together selected to form an optionally substituted fused 5- or 6-membered ring selected from cycloalkyl, cycloalkenyl, heterocycloalkyl having 1-3 heteroatoms independently selected form N, O, or S, or heterocycloalkenyl having 1-3 heteroatoms independently selected form N, O, or S ring. In some embodiments, Z and W are together selected to form an optionally substituted fused 5- or 6-membered ring selected from cycloalkyl, cycloalkenyl, heterocycloalkyl having 1-3 heteroatoms independently selected form N, O, or S, or heterocycloalkenyl having 1-3 heteroatoms independently selected form N, O, or S ring, wherein Z is selected from the group consisting of: —C(R₁₆)(R₁₆′)—, —C(R₁₈)(R₁₈′)—, —C(R₂₀)(R₂₀′)—C(R₁₈)(R₁₈′)—*, —S—, —S—C(R₁₈)(R₁₈′)—*, —C(R₁₈)(R₁₈′)—S—*, —N(R₁₄)—, —N(R₁₄)—C(R₁₈)(R₁₈′)—*, —C(R₁₈)(R₁₈′)—N(R₁₄)—*, —O—, —O—C(R₁₈)(R₁₈′)—*, —C(R₁₈)(R₁₈′)—O—*, and —C(R₂₀)═C(R₁₈)—*, wherein * represents the point of attachment to W, and W is selected from the group consisting of —(C═O)— and —C(R₁₀)(R₁₀′)—. In some embodiments, Z and W are together selected to form a fused 5- or 6-membered heterocycloalkyl having 1-3 heteroatoms independently selected form N, O, or S, or heterocycloalkenyl having 1-3 heteroatoms independently selected form N, O, or S ring comprising one nitrogen heteroatom. In some embodiments, Z and W are together selected to form a fused 5- or 6-membered heterocycloalkyl having 1-3 heteroatoms independently selected form N, O, or S, or heterocycloalkenyl having 1-3 heteroatoms independently selected form N, O, or S ring comprising one nitrogen heteroatom and one S heteroatom.

In some embodiments, R₁₄ is hydrogen. In some embodiments, R₁₄ is (C₁-C₄)alkyl. In some embodiments, R₁₄ is methyl. In some embodiments, R₁₄ is ethyl, propyl (e.g., n-propyl or isopropyl), or butyl (e.g., n-butyl or isobutyl).

In some embodiments, Z and W are together selected to form an optionally substituted fused 5- or 6-membered heteroaryl ring having 1-3 heteroatoms independently selected form N, O, or S. In some embodiments, Z and W are together selected to form an optionally substituted fused 5- or 6-membered heteroaryl ring comprising one or two nitrogen heteroatoms. In some embodiments, Z and W are together selected to form an optionally substituted fused 5- or 6-membered heteroaryl ring having 1-3 heteroatoms independently selected form N, O, or S, wherein Z is selected from the group consisting of: —C(R₂₀)═, —N═, or —C(R₁₆)(R₁₆′)—; and W is selected from the group consisting of: ═N— and ═C(R₉₀)—.

In some embodiments, R₉₀ in W is selected from the group consisting of hydrogen; (C₁-C₄) alkyl optionally substituted with one or more halogen, hydroxyl or —N(R_{b})(R_{b′}); (C₃-C₆)cyclopropyl optionally substituted with one or more (C₁-C₄)alkyl; —O—(C₁-C₄) alkyl optionally substituted with one or more halogen; and (C₁-C₄) alkyl-N(R_{b})(R_{b′}), wherein R_{b} and R_{b′} are as defined herein with respect to Formula (I) and described in classes and subclasses herein (e.g., with respect to any one or more of Formula (II)-(X)).

In some embodiments, R₁₀, R₁₀′, R₁₆, R₁₆′, R₁₈, R₁₈, R₂₀, and R₂₀′ are each independently selected from the group consisting of hydrogen, (C₁-C₄)alkyl optionally substituted with one or more halogen, —O—(C₁-C₄)alkyl optionally substituted with one or more halogen, and (C₁-C₄)alkyl-N(R_{b})(R_{b′}); or R₁₆ and R₁₆′, R₁₈ and R₁₈′, and R₂₀ and R₂₀′ each together form a spirocyclic 3- to 6-membered cycloalkyl optionally substituted with one or more R_{a′}.

In some embodiments, a compound of Formula (I) can be a compound of Formula (IIa), Formula (IIb) or Formula (IIc). In some embodiments, the compound is a compound a compound of Formula (I) wherein Z is —S—C(R₁₈)(R₁₈′)—*, wherein * represents the point of attachment to W, and W is —C(R₁₀)(R₁₀′)—, such as a compound of Formula (IIa). In some embodiments, the compound is a compound a compound of Formula (I) wherein Z is —N(R₁₄)—C(R₁₈)(R₁₈′)—*, wherein * represents the point of attachment to W, and W is —C(R₁₀)(R₁₀′)—, such as a compound of Formula (IIb). In some embodiments, the compound is a compound of Formula (I) wherein Z is —O—C(R₁₈)(R₁₈′)—*, wherein * represents the point of attachment to W, and W is —C(R₁₀)(R₁₀′)—, such as a compound of Formula (IIc).

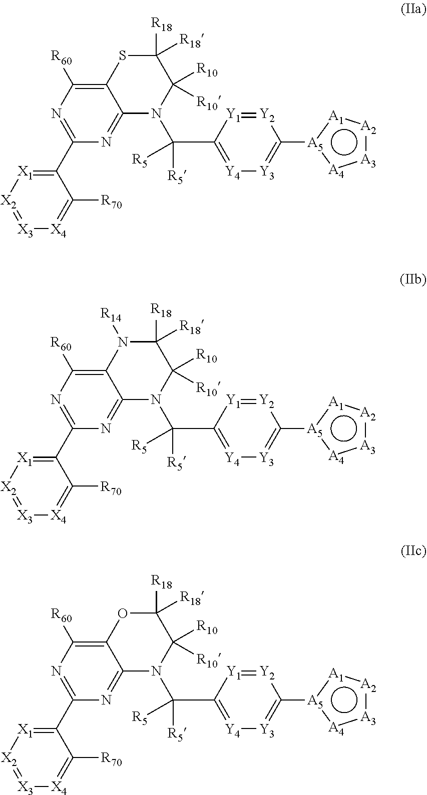

or a pharmaceutically acceptable salt thereof, wherein X₁, X₂, X₃, X₄, R₆₀, R₇₀, R₁₈, R₁₈′, R₁₀, R₁₀′, R₅, R₅′, Y₁, Y₂, Y₃, Y₄, A₁, A₂, A₃, A₄, and A₅ are each as described above with respect to Formula (I), and defined and described in classes and subclasses herein (e.g., with respect to any one or more of Formula (II)-(X)), both singly and in combination.

In some embodiments, a compound of Formula (IIa), Formula (IIb) or Formula (IIc) can be obtained using the method of General Scheme 1 below.

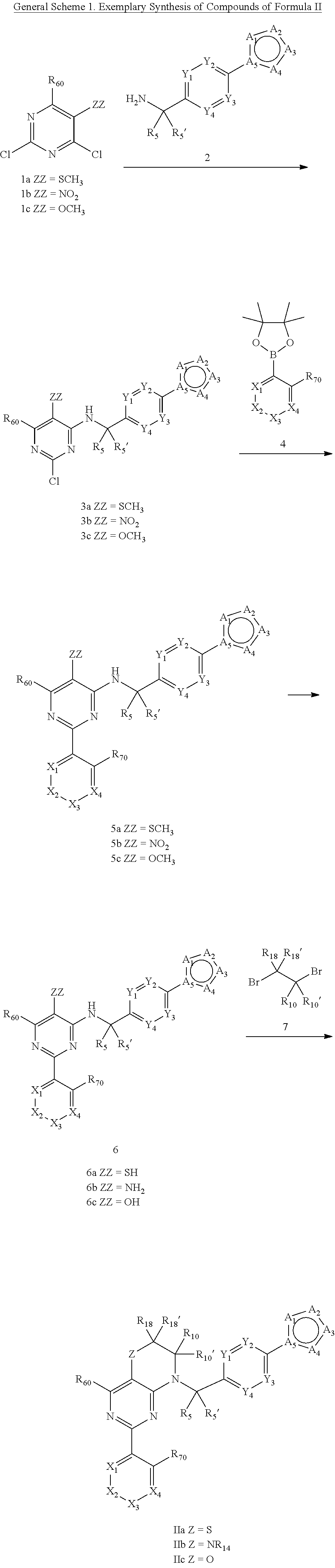

A general method of preparing compounds of Formula II is outlined in General Scheme 1. Amination of 1 with 2 using a base (i.e., potassium carbonate (K₂CO₃) in a solvent (i.e., DMF) yields 3. Coupling of 3 with an arylboronic acid/ester or heteroarylboronic acid/ester 4 using a catalytic amount of a palladium catalyst (e.g., Pd(dppf)Cl₂ CH₂Cl₂) and a base (e.g., potassium carbonate) in a solvent (e.g., 1,4-dioxane) at elevated temperature provides 5. Treatment of 5a (Z═SCH₃) with sodium methanethiolate in solvent (e.g., HMPA) at elevated temperature affords 6a (Z═SH). Reduction of 5b (Z═NO₂) using a metal (e.g., iron powder) and ammonium chloride in a solvent mixture (e.g., ethanol/THF/water) provides 6b (Z═NH₂). Treatment of 5c (Z═OCH₃) with borontribromide in solvent (e.g., DCM) affords 6c (Z═OH). Treatment of 6 with an optionally substituted 1,2-dibromoethane 7 provides the desired compound of Formula (II). Treatment of IIb when R₁₄═H with base (e.g., sodium hydride) and alkyl halide (e.g., methyl iodide) in solvent (e.g., DMF) provides desired compounds of Formula (IIb) where R₁₄=alkyl.

In some embodiments, a compound of Formula (I) can be a compound of Formula (IIIa), Formula (IIIb) or Formula (IIIc). In some embodiments, the compound is a compound a compound of Formula (I) wherein Z is —S—C(R₁₈)(R₁₈′)—*, wherein * represents the point of attachment to W, and W is —(C═O)—, such as a compound of Formula (IIIa). In some embodiments, the compound is a compound of Formula (I) wherein Z is —N(R₁₄)—C(R₁₈)(R₁₈′)—*, wherein * represents the point of attachment to W, and W is —(C═O)— such as a compound of Formula (IIIb). In some embodiments, the compound is a compound of Formula (I) wherein Z is —O—C(R₁₈)(R₁₈′)—*, wherein * represents the point of attachment to W, and W is —(C═O)—, such as a compound of Formula (IIIc).

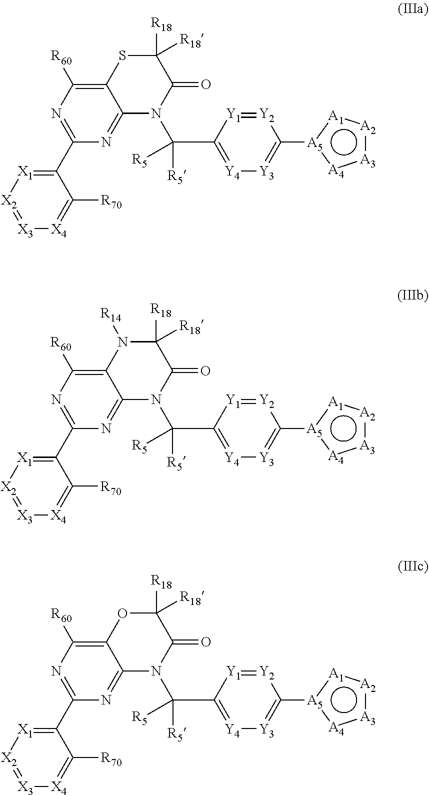

or a pharmaceutically acceptable salt thereof, wherein X₁, X₂, X₃, X₄, R₆₀, R₇₀, R₁₈, R₁₈′, R₁₀, R₁₀′, R₅, R₅′, Y₁, Y₂, Y₃, Y₄, A₁, A₂, A₃, A₄, and A₅ are each as described above with respect to Formula (I), and defined and described in classes and subclasses herein (e.g., with respect to any one or more of Formula (II)-(X)), both singly and in combination.

In some embodiments, a compound of Formula (I) that is a Formula (IIIa), Formula (IIIb) or Formula (IIIc) can be obtained using the method of General Scheme 2 below.

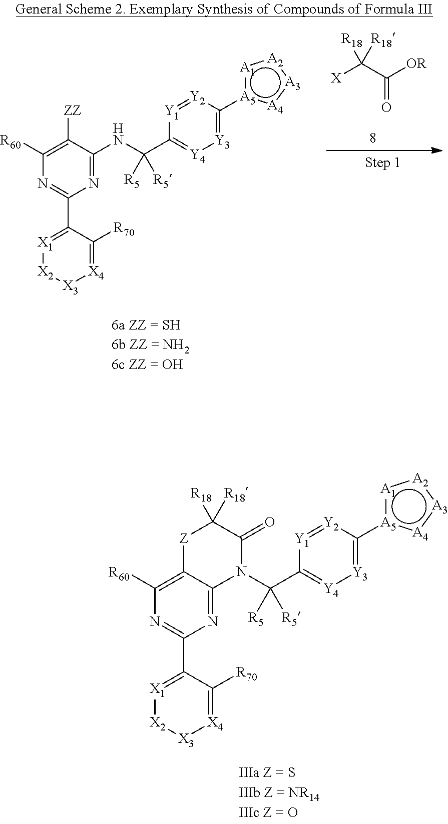

A general method of preparing compounds of Formula III is outlined in General Scheme 2. Alkylation of 6 with intermediate 8 wherein X is a halogen using a base (e.g., potassium carbonate) in a solvent (e.g., ACN) provides the desired compounds of Formula (III). Treatment of IIIb when R₁₄═H with base (e.g., sodium hydride) and alkyl halide (e.g., methyl iodide) in solvent (e.g., DMF) provides desired compounds of Formula (IIIb) where R₁₄=alkyl.

In some embodiments, a compound of Formula (I) can be a compound of Formula (IVa), Formula (IVb) or Formula (IVc). In some embodiments, the compound is a compound of Formula (I) wherein Z is —CH₂—O—*, wherein * represents the point of attachment to W, and W is —(C═O)—, such as a compound of Formula (IVa), or Z is —CH₂—N(R₁₄)—*, wherein * represents the point of attachment to W, and W is —(C═O)—, such as a compound of Formula (IVb), or Z is —CH₂—S—*, wherein * represents the point of attachment to W, and W is —(C═O)—, such as a compound of Formula (IVc).

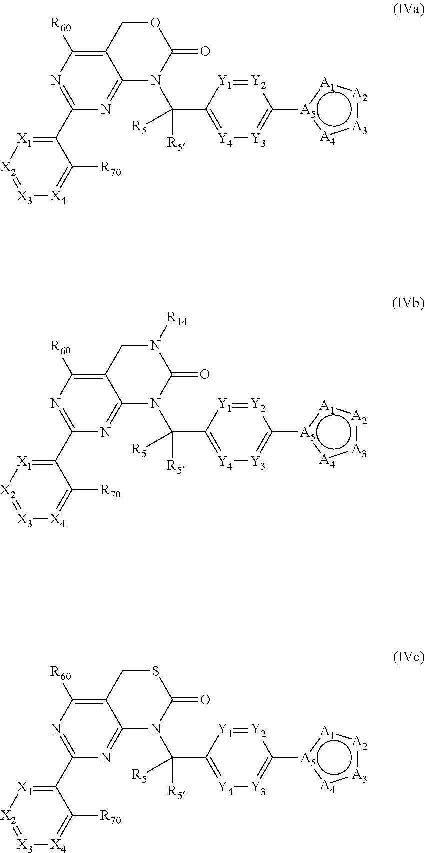

or a pharmaceutically acceptable salt thereof, wherein X₁, X₂, X₃, X₄, R₆₀, R₇₀, R₁₄, R₅, R₅′, Y₁, Y₂, Y₃, Y₄, A₁, A₂, A₃, A₄, and A₅ are each as described above with respect to Formula (I), and defined and described in classes and subclasses herein (e.g., with respect to any one or more of Formula (II)-(X)), both singly and in combination.

In some embodiments, a compound of Formula (I) that is a Formula (IVa), Formula (IVb), or Formula (IVc) can be obtained using the method of General Scheme 3 below.

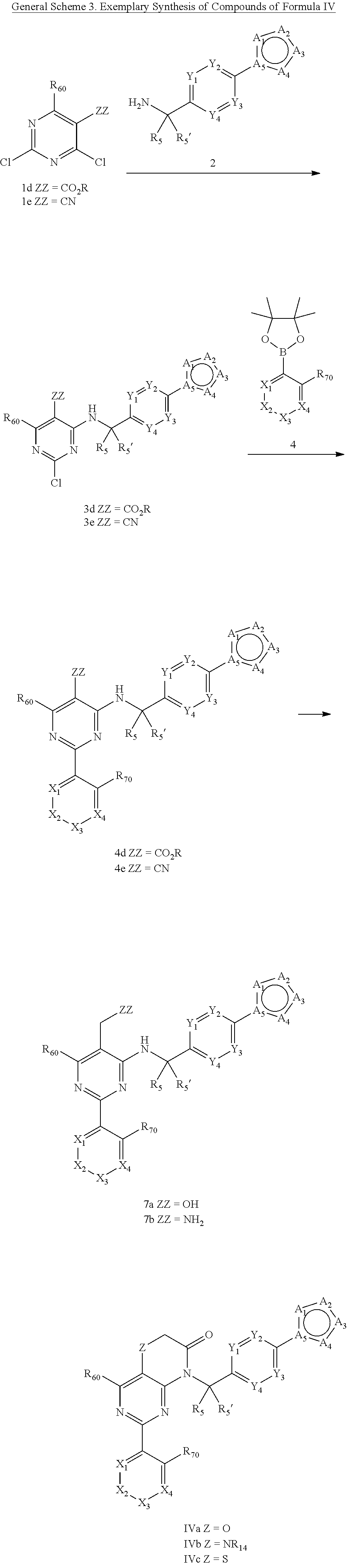

A general method of preparing compounds of Formula IV is outlined in General Scheme 3. Amination of 1d or 1e with 2 using a base (e.g., potassium carbonate (K₂CO₃) in a solvent (e.g., DMF) yields 3d or 3e. Coupling of 3d or 3e with an arylboronic acid/ester or heteroarylboronic acid/ester 4 using a catalytic amount of a palladium catalyst (e.g., Pd(dppf)Cl₂ CH₂Cl₂) and a base (e.g., potassium carbonate) in a solvent (e.g., 1,4-dioxane) at elevated temperature provides 4d or 4e. Treatment of 4d with a reducing agent (e.g., lithium aluminum hydride) in a solvent (e.g., THF) affords 7a. Treatment of 4e with hydrogen gas in the presence of a metal (e.g., Raney Ni) in a basic solvent (e.g., methanolic ammonia) affords 7b. Cyclization of 7a or 7b with CDI in a solvent (e.g., DCM) provides the compounds of Formula IVa or Formula IVb where R₁₄═H. Treatment of 7a with potassium ethylxanthate and hydrogen peroxide in a solvent (e.g., DMF) provides compounds of the desired Formula IVc. Treatment of compounds of Formula IVb (R₁₄═H) with base (e.g., sodium hydride) and alkyl halide (e.g., methyl iodide) in solvent (i.e., DMF) provides desired compounds of Formula IVc where R₁₄=alkyl.

In some embodiments, a compound of Formula (I) can be a compound of Formula (Va), Formula (Vb) or Formula (Vc). In some embodiments, the compound is a compound of Formula (I) wherein Z is —S— and W is —(C═O)—, such as a compound of Formula (Va); or Z is —O— and W is —(C═O)—, such as a compound of Formula (Vb):

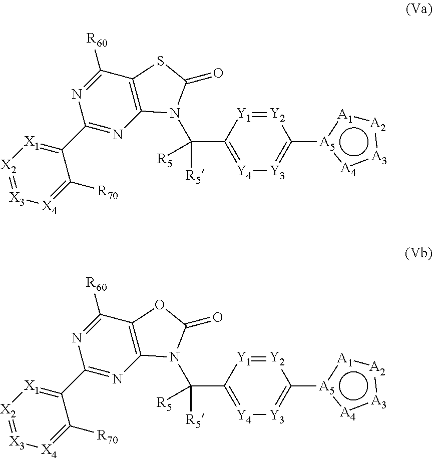

or a pharmaceutically acceptable salt thereof, wherein X₁, X₂, X₃, X₄, R₆₀, R₇, R₅, R₅′, Y₁, Y₂, Y₃, Y₄, A₁, A₂, A₃, A₄, and A₅ are each as described above with respect to Formula (I), and defined and described in classes and subclasses herein (e.g., with respect to any one or more of Formula (II)-(X)), both singly and in combination.

In some embodiments, a compound of Formula (I) that is a Formula (Va) can be obtained using the method of General Scheme 4 below.

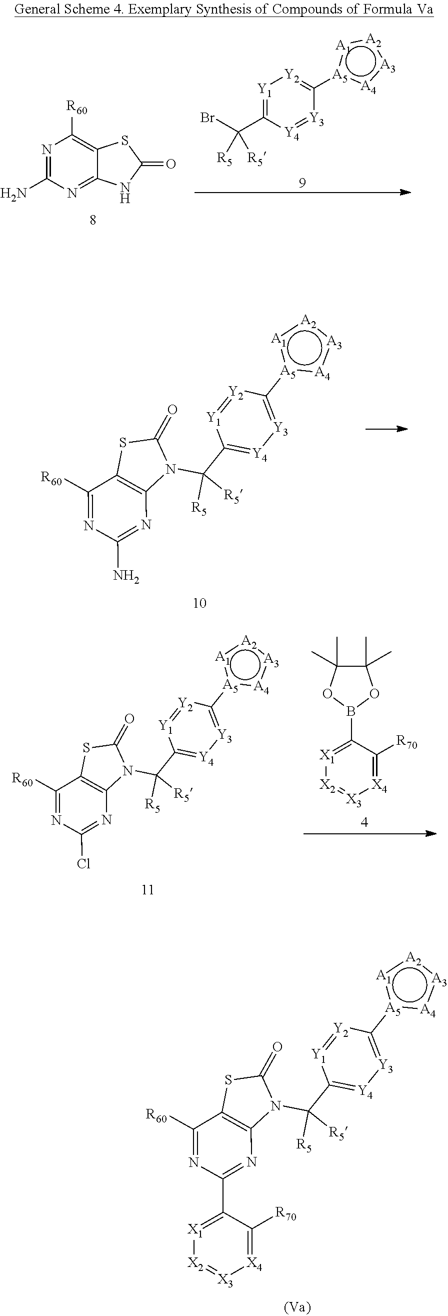

A general method of preparing compounds of Formula Va is outlined in General Scheme 4. Alkylation of 8 with 9 using a base (e.g., sodium hydride) in a solvent (e.g., DMF) yields 10. Treatment of 10 with hydrochloric acid and sodium nitrite and copper(I) chloride in water is a method that can be used to prepare 11. Coupling of 11 with an arylboronic acid/ester or heteroarylboronic acid/ester 4 using a catalytic amount of a palladium catalyst (e.g., Pd(dppf)Cl₂ CH₂Cl₂) and a base (e.g., potassium carbonate) in a solvent (e.g., 1,4-dioxane) at elevated temperature provides the desired compound of Formula (Va).

In some embodiments, a compound of Formula (I) that is a Formula (Vb) can be obtained using the method of General Scheme 5 below.

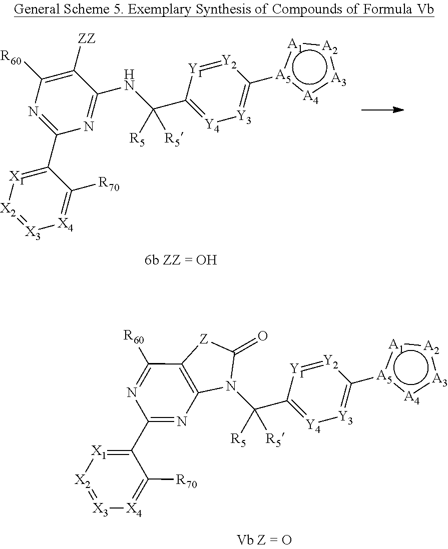

A general method of preparing compounds of Formula Vb is outlined in General Scheme 5. Treatment of 6b with CDI in a solvent (e.g., DCM) at elevated temperature provides the desired compound of Formula Vb.

In some embodiments, a compound of Formula (I) can be a compound of Formula (VIa), or Formula (VIb). In some embodiments, the compound is a compound of Formula (I) wherein Z is —C(R₂₀)(R₂₀′)—C(R₁₈)(R₁₈′)—*, wherein * represents the point of attachment to W, and W is —(C═O)—, such as a compound of Formula (VIa), or Z is —C(R₂₀)—CH—*, wherein * represents the point of attachment to W, and W is —(C═O)—, such as a compound of Formula (VIb):

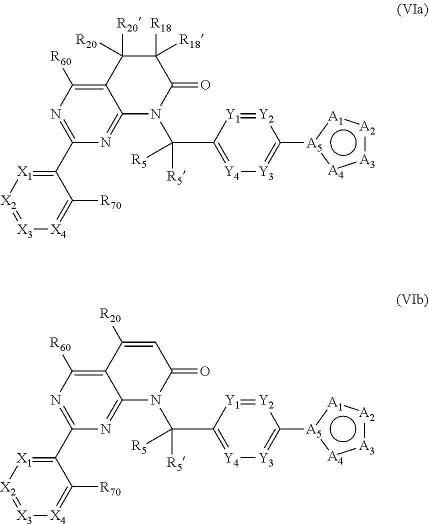

or a pharmaceutically acceptable salt thereof, wherein X₁, X₂, X₃, X₄, R₆₀, R₇₀, R₁₈, R₁₈′, R₂₀, R₂₀′, R₅, R₅′, Y₁, Y₂, Y₃, Y₄, A₁, A₂, A₃, A₄, and A₅ are each as described above with respect to Formula (I), and defined and described in classes and subclasses herein (e.g., with respect to any one or more of Formula (II)-(X)), both singly and in combination.

In some embodiments, a compound of Formula (I) that is a Formula (VIa) can be obtained using the method of General Scheme 6 below.

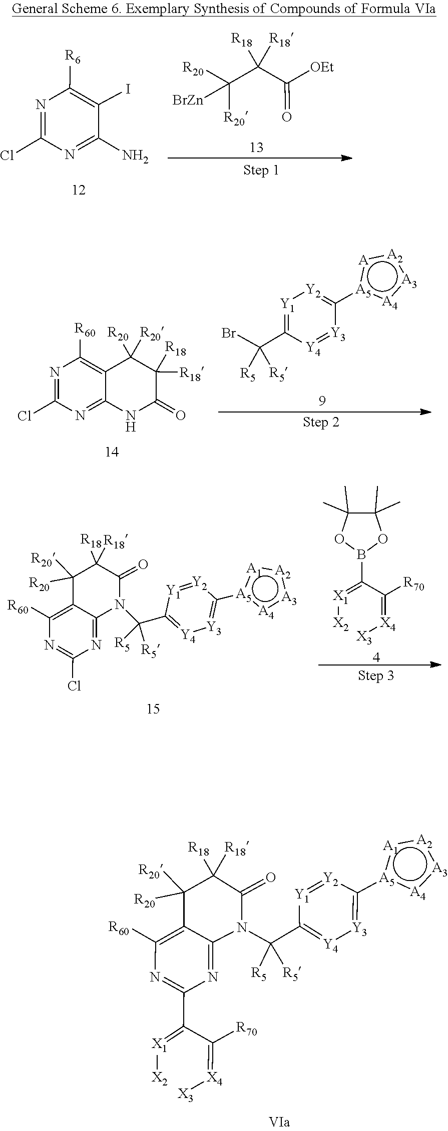

A general method of preparing compounds of Formula VIa is outlined in General Scheme 6. Treatment of 12 with organozinc reagent 13 using a catalytic amount of a palladium catalyst (e.g, tetrakis(triphenylphosphine)palladium(0)) in a solvent mixture (e.g., toluene/THF) yields 14. Alkylation of 14 with 9 using a base (e.g., cesium carbonate) in a solvent (e.g., DMF) affords 15. Coupling of 15 with an arylboronic acid/ester or heteroarylboronic acid/ester 4 using a catalytic amount of a palladium catalyst (e.g., Pd(dppf)Cl₂ CH₂Cl₂) and a base (e.g., potassium carbonate) in a solvent (e.g., 1,4-dioxane) at elevated temperature provides the desired compound of Formula VIa.

In some embodiments, a compound of Formula (I) that is a Formula (VIb) can be obtained using the method of General Scheme 7 below.

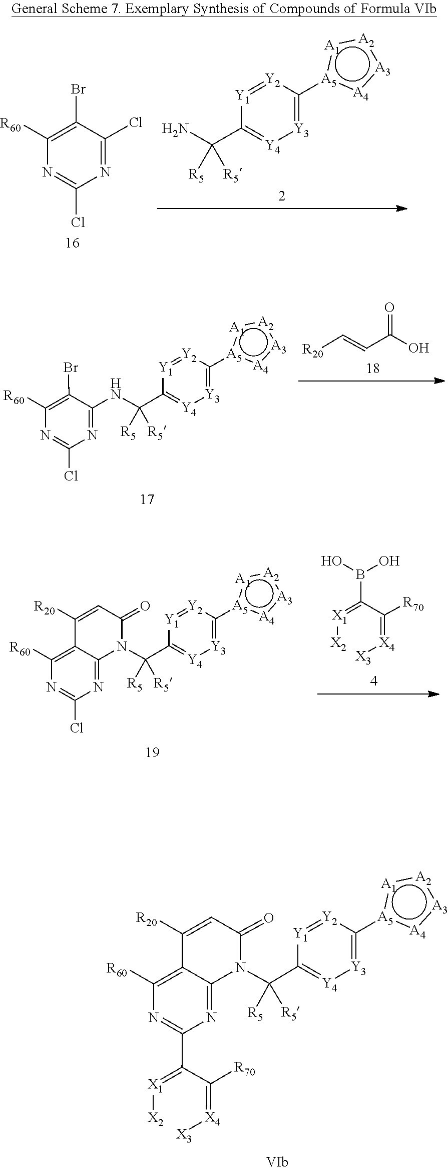

A general method of preparing compounds of Formula VIb is outlined in General Scheme 7. Alkylation of 16 with 9 using a base (e.g., potassium carbonate (K₂CO₃) in a solvent (e.g., DMF) yields 17. Treatment of 17 with a carboxylic acid such as 18 with a catalytic amount of a palladium catalyst (e.g., dichlorobis(tri-o-tolylphosphine)palladium(II)) and a base (e.g., DIEA) in a solvent (e.g., THF) at elevated temperature followed by addition of acetic anhydride under continued heating provides 19. Coupling of 19 with an arylboronic acid/ester or heteroarylboronic acid/ester 4 using a catalytic amount of a palladium catalyst (e.g., Pd(dppf)Cl₂CH₂Cl₂) and a base (e.g., potassium carbonate) in a solvent (e.g., 1,4-dioxane) at elevated temperature provides the desired compound of Formula (VIb).

In some embodiments, a compound of Formula (I) can be a compound of Formula (VII), or Formula (VIIIa). In some embodiments, the compound is a compound of Formula (I) wherein Z is —C(H)═ and W is ═N—, such as a compound of Formula (VII):

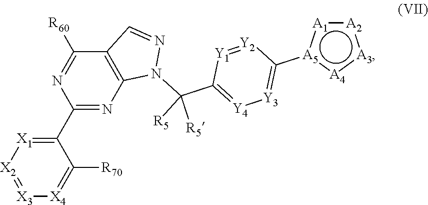

or a pharmaceutically acceptable salt thereof, wherein X₁, X₂, X₃, X₄, R₆₀, R₇₀, R₅, R₅′, Y₁, Y₂, Y₃, Y₄, A₁, A₂, A₃, A₄, and A₅ are each as described above with respect to Formula (I), and defined and described in classes and subclasses herein (e.g., with respect to any one or more of Formula (II)-(X)), both singly and in combination.

In some embodiments, the compound is a compound of Formula (I) wherein Z is —CH═ and W is ═C(R₉₀)—, such as a compound of Formula (VIIIa).

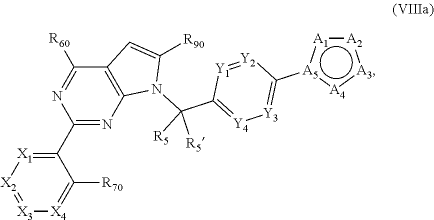

or a pharmaceutically acceptable salt thereof, wherein X₁, X₂, X₃, X₄, R₆₀, R₇₀, R₉₀, R₅, R₅′, Y₁, Y₂, Y₃, Y₄, A₁, A₂, A₃, A₄, and A₅ are each as described above with respect to Formula (I), and defined and described in classes and subclasses herein (e.g., with respect to any one or more of Formula (II)-(X)), both singly and in combination.

In some embodiments, a compound of Formula (I) that is a Formula (VIII) or a compound of Formula (VIIIa) can be obtained using the method of General Scheme 8 below.

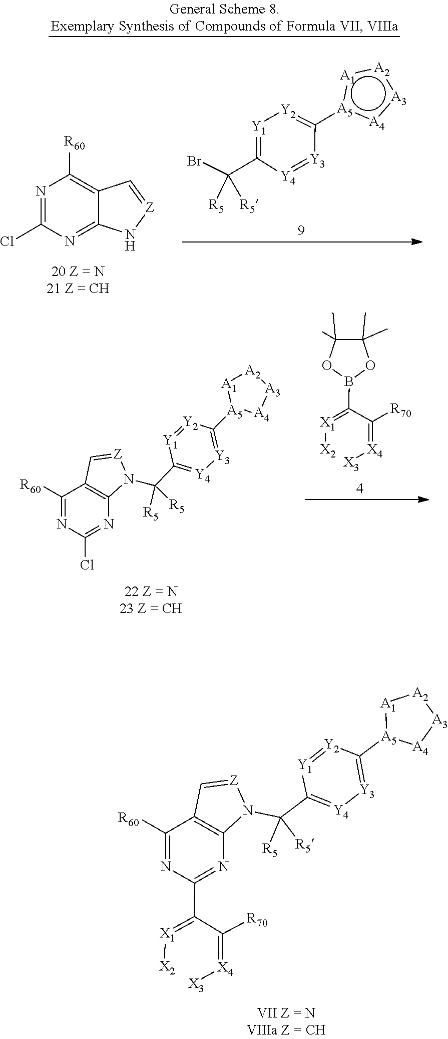

A general method of preparing compounds of Formula VII and VIIIa is outlined in General Scheme 8. Alkylation of 20 or 21 with 9 using a base (e.g., potassium carbonate) in a solvent (e.g., DMF) at elevated temperature yields 22 or 23, respectively. Coupling of 22 or 23 with an arylboronic acid/ester or heteroarylboronic acid/ester 4 using a catalytic amount of a palladium catalyst (e.g., Pd(dppf)Cl₂·CH₂Cl₂) and a base (e.g., potassium carbonate) in a solvent (e.g., 1,4-dioxane) at elevated temperature provides the desired compounds of Formula VII or VIIIa, respectively.

In some embodiments, a compound of Formula (I) can be a compound of Formula (VIIIb). In some embodiments, the compound is a compound of Formula (I) wherein Z is —N═ and W is ═C(R₉₀)—, such as a compound of Formula (VIIIb).

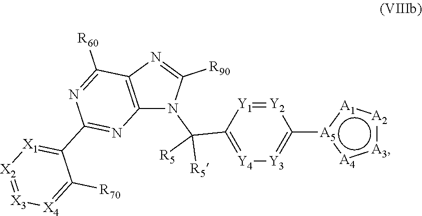

or a pharmaceutically acceptable salt thereof, wherein X₁, X₂, X₃, X₄, R₆₀, R₇₀, R₉₀, R₅, R₅′, Y₁, Y₂, Y₃, Y₄, A₁, A₂, A₃, A₄, and A₅ are each as described above with respect to Formula (I), and defined and described in classes and subclasses herein (e.g., with respect to any one or more of Formula (II)-(X)), both singly and in combination.

In some embodiments, a compound of Formula (I) that is a compound of Formula (VIIIb) can be obtained using the method of General Scheme 9 below.

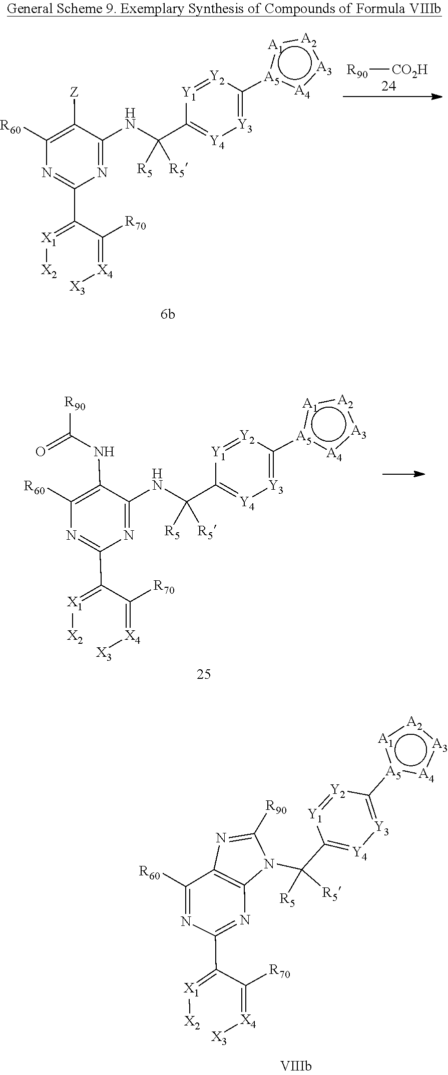

A general method of preparing compounds of Formula VIIIb is outlined in General Scheme 9. Treatment of 6b with a carboxylic acid 24, an activating reagent (e.g., HATU) and a base (e.g., DIEA) in a solvent (e.g., DMF) yields 25. Treatment of 25 in acidic solvent (e.g., acetic acid) at elevated temperature provides the desired compound of Formula (VIIIb).

In some embodiments, a compound of Formula (I) can be a compound of Formula (VIIIc). In some embodiments, the compound is a compound of Formula (VIIIb) wherein R₉₀ is —O—(C₁-C₄)alkyl (e.g., methoxy), such as a compound of Formula (VIIIc):

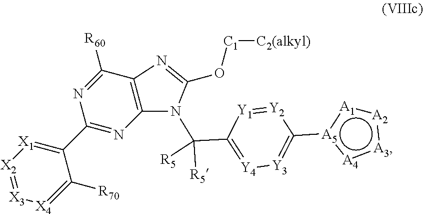

or a pharmaceutically acceptable salt thereof, wherein X₁, X₂, X₃, X₄, R₆₀, R₇₀, R₅, R₅′, Y₁, Y₂, Y₃, Y₄, A₁, A₂, A₃, A₄, and A₅ are each as described above with respect to Formula (I).

In some embodiments, a compound of Formula (I) that is a compound of Formula (VIIIc) can be obtained using the method of General Scheme 10 below.

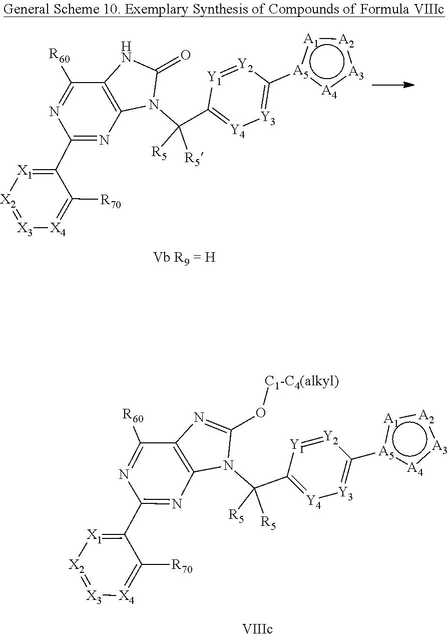

A general method of preparing compounds of Formula VIIIc is outlined in General Scheme 10. Treatment of compounds of Formula Vb when R₁₄═H with a base (e.g., sodium hydride) and an alkyl halide (e.g., methyl iodide) in solvent (e.g., DMF) provides compounds of Formula (VIIIc).

In some embodiments, the compound is a compound of Formula (I) wherein Z is —C(R₁₆)(R₁₆′)— and W is —(C═O)—, such as a compound of Formula (IX):

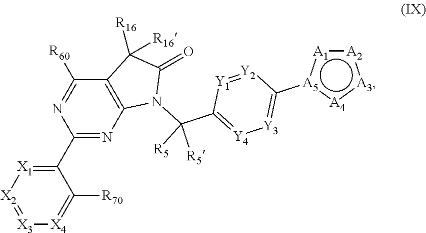

or a pharmaceutically acceptable salt thereof, wherein X₁, X₂, X₃, X₄, R₆₀, R₇₀, R₁₆, R₁₆′, R₅, R₅′, Y₁, Y₂, Y₃, Y₄, A₁, A₂, A₃, A₄, and A₅ are each as described above with respect to Formula (I), and defined and described in classes and subclasses herein (e.g., with respect to any one or more of Formula (II)-(X)), both singly and in combination.

In some embodiments, a compound of Formula (I) that is a compound of Formula (IX) can be obtained using the method of General Scheme 11 below.

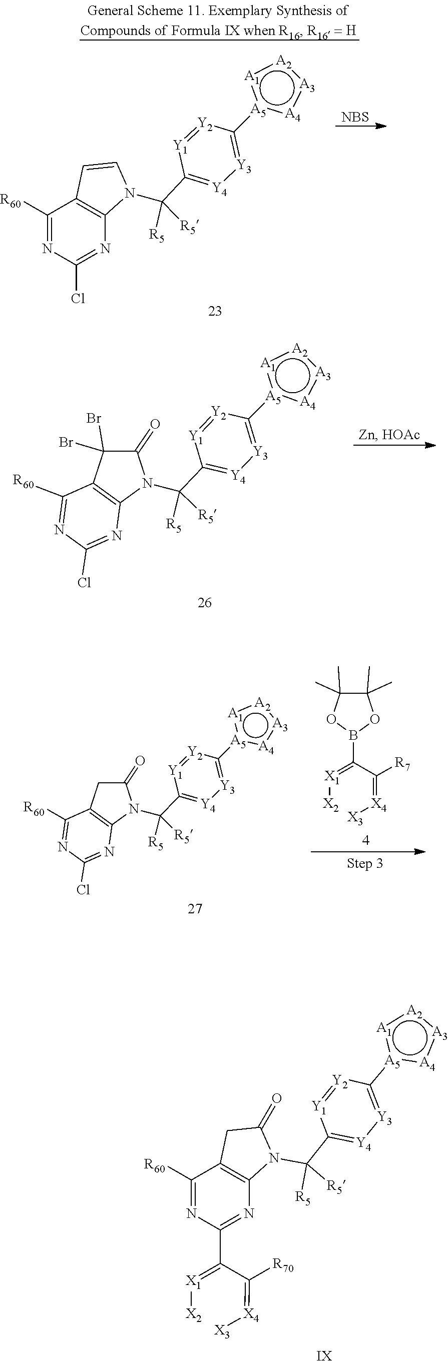

A general method of preparing compounds of Formula (IX) when R₁₆ and R₁₆′═H is outlined in General Scheme 11. Bromination of 23 with NBS in a solvent mixture (e.g., t-butanol/water) yields 26. Reduction of 26 using zinc in acetic acid affords 27. Coupling of 27 with an arylboronic acid/ester or heteroarylboronic acid/ester 4 using a catalytic amount of a palladium catalyst (e.g., Pd(dppf)Cl₂·CH₂Cl₂) and a base (e.g., potassium carbonate) in a solvent (e.g., 1,4-dioxane) at elevated temperature provides the desired compounds of Formula (IX).

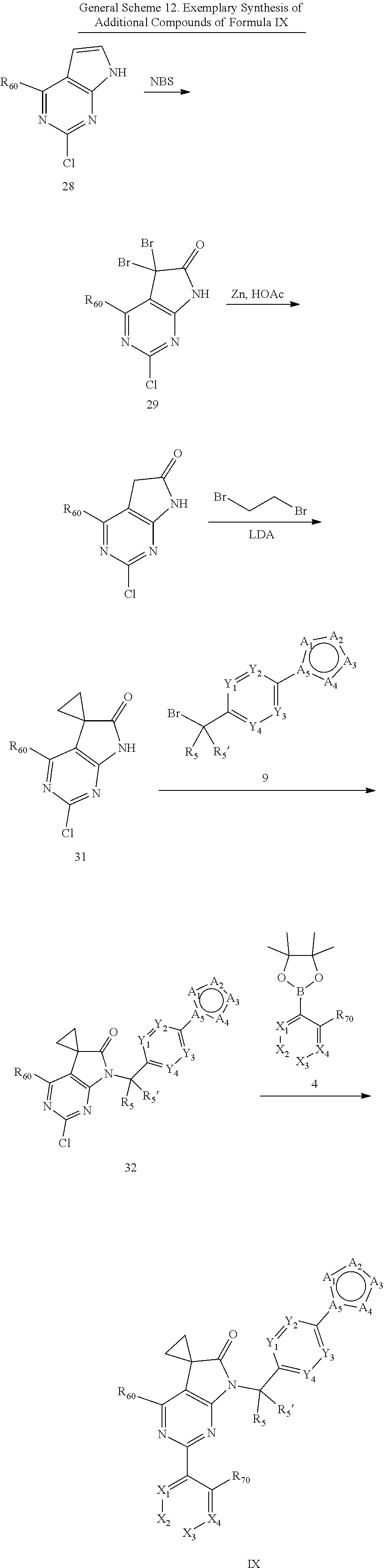

A general method of preparing compounds of Formula IX when R₁₆ and R_{16′} are taken together to form a cyclopropyl ring is outlined in General Scheme 12. Bromination of 28 with NBS in a solvent mixture (e.g., t-butanol/water) yields 29. Reduction of 29 using zinc in acetic acid affords 30. Alkylation of 30 with 1,2-dibromoethane using a base (e.g., potassium carbonate) in a solvent (e.g., DMF) at elevated temperature yields 31. Alkylation of 31 with 9 using a base (e.g., sodium hydride) in a solvent (e.g., DMF) yields 32. Coupling of 32 with an arylboronic acid/ester or heteroarylboronic acid/ester 4 using a catalytic amount of a palladium catalyst (e.g., Pd(dppf)Cl₂·CH₂Cl₂) and a base (e.g., potassium carbonate) in a solvent (e.g., 1,4-dioxane) at elevated temperature provides the desired compounds of Formula IX where R₁₆ and R_{16′} are taken together to form a cyclopropyl ring.

In some embodiments, the compound is a compound of Formula (I) wherein Z is —CH₂— and W is —CH₂—, such as a compound of Formula (Xa), or Z is —C(R₂₀)(R₂₀′)— and W is —CH₂—, such as a compound of Formula (Xb):

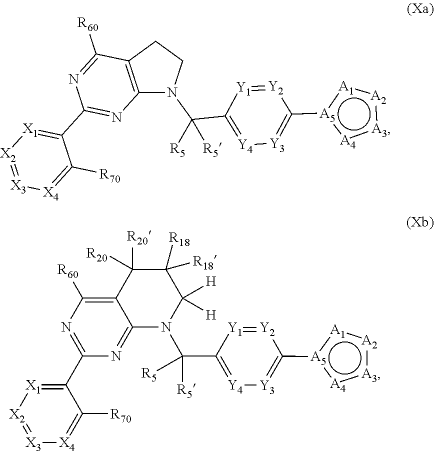

or a pharmaceutically acceptable salt thereof, wherein X₁, X₂, X₃, X₄, R₆₀, R₇₀, R₁₈, R₁₈′, R₂₀, R₂₀′, R₅, R₅′, Y₁, Y₂, Y₃, Y₄, A₁, A₂, A₃, A₄, and A₅ are each as described above with respect to Formula (I), and defined and described in classes and subclasses herein (e.g., with respect to any one or more of Formula (II)-(X)), both singly and in combination.

In some embodiments, a compound of Formula (I) that is a compound of Formula (Xa) or Formula (Xb) can be obtained using the method of General Scheme 12 below.

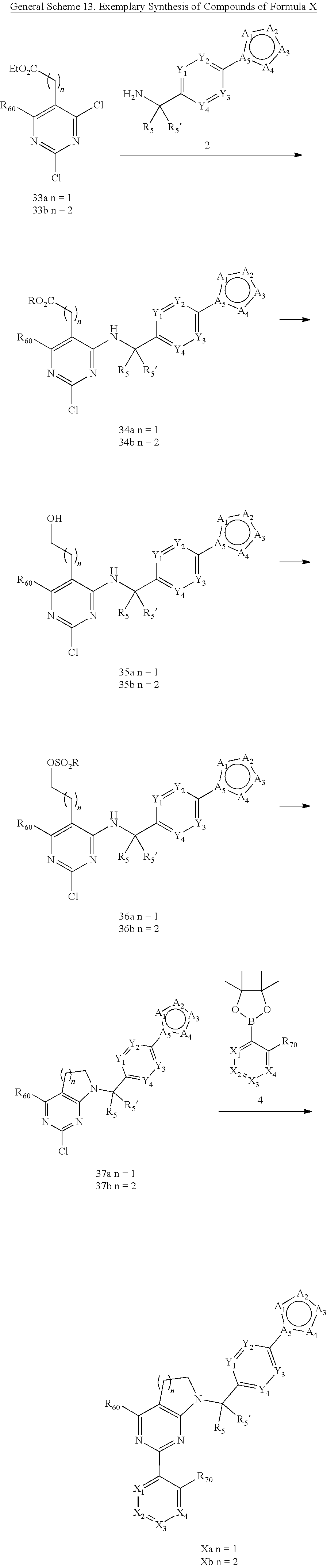

A general method of preparing compounds of Formula X is outlined in General Scheme 13. Amination of 33 with 2 using a base (e.g., K₂CO₃) in a solvent (e.g., DMF) yields 34. Treatment of 34 with a reducing agent (e.g., sodium borohydride) in a solvent (e.g., THF) affords 35. Treatment of 35 with an activating reagent (e.g., methanesulfonyl chloride) and a base (e.g., TEA) in a solvent (e.g., DCM) affords 36. Heating 36 with a base (e.g., DBU) in a solvent (e.g., DMF) at elevated temperature generates cyclized compound 37. Coupling of 37 with an arylboronic acid/ester or heteroarylboronic acid/ester 4 using a catalytic amount of a palladium catalyst (e.g., Pd(dppf)Cl₂·CH₂Cl₂) and a base (e.g., potassium carbonate) in a solvent (e.g., 1,4-dioxane) at elevated temperature provides compounds of Formula X.

The compounds of the present disclosure, i.e., compounds of Formula (I), or a pharmaceutically acceptable salt, enantiomer, hydrate, solvate, prodrug, isomer, or tautomer thereof, may be prepared by methods known in the art of organic synthesis as set forth in part by the following synthetic schemes. In the schemes described below, it is well understood that protecting groups for sensitive or reactive groups are employed where necessary in accordance with general principles or chemistry. Protecting groups are manipulated according to standard methods of organic synthesis (T. W. Greene and P. G. M. Wuts, “Protective Groups in Organic Synthesis”, Third edition, Wiley, New York 1999). These groups are removed at a convenient stage of the compound synthesis using methods that are readily apparent to those skilled in the art. The selection processes, as well as the reaction conditions and order of their execution, shall be consistent with the preparation of compounds of Formula (I).

Those skilled in the art will recognize if a stereocenter exists in the compounds of any of Formulae (I)-(X). Accordingly, the present disclosure includes both possible stereoisomers (unless specified in the synthesis) and includes not only racemic compounds but the individual enantiomers and/or diastereomers as well. When a compound is desired as a single enantiomer or diastereomer, it may be obtained by stereospecific synthesis or by resolution of the final product or any convenient intermediate. Resolution of the final product, an intermediate, or a starting material may be affected by any suitable method known in the art. See, for example, “Stereochemistry of Organic Compounds” by E. L. Eliel, S. H. Wilen, and L. N. Mander (Wiley-Interscience, 1994).

The compounds described herein may be made from commercially available starting materials or synthesized using known organic, inorganic, and/or enzymatic processes.

The disclosure also includes pharmaceutical compositions comprising one or more USP1 inhibitor compounds as described herein, or a pharmaceutically acceptable salt thereof, and a pharmaceutically acceptable excipient. In some embodiments, pharmaceutical compositions reported herein can be provided in a unit dosage form (e.g., capsule, tablet or the like). Pharmaceutical compositions comprising a compound provided herein can be provided in an oral dosage form such as a capsule or tablet. The oral dosage form optionally comprises one or more fillers, disintegrants, lubricants, glidants, anti-adherents and/or anti-statics. In some embodiments, an oral dosage form is prepared via dry blending. In some embodiments, an oral dosage form is a tablet and is prepared via dry granulation. For example, a USP1 inhibitor compound of the present disclosure can be dosed a therapeutically safe and effective frequency determined by clinical trials. The pharmaceutical compositions may be orally administered in any orally acceptable dosage form. Accordingly, a patient and/or subject can be selected for treatment using a compound described herein by first evaluating the patient and/or subject to determine whether the subject is diagnosed for a condition for which a suitable pharmaceutical composition comprising a USP1 inhibitor may be indicated, and if then administering to the subject a composition described herein.

In some embodiments, a USP1 inhibitor compound provided herein can be useful in the treatment of cancer including but not limited to DNA damage repair pathway deficient cancers. Additional examples of cancer include, but are not limited to, ovarian cancer, breast cancer (including triple negative breast cancer), non-small cell lung cancer (NSCLC), and osteosarcoma. The cancer can be BRCA1 or BRCA2 wildtype. The cancer can also be BRCA1 or BRCA2 mutant. The cancer can further be a PARP inhibitor resistant or refractory cancer, or a PARP inhibitor resistant or refractory BRCA1 or BRCA2-mutant cancer. In some embodiments, the compounds provided herein are useful for the development of therapies to treat a Poly (ADP-ribose) polymerase (“PARP”) inhibitor refractory or resistant cancer. In some embodiments, the cancer is a PARP inhibitor resistant or refractory BRCA1, BRCA2, or BRCA1 and BRCA2 mutant cancer. In some embodiments, the cancer is a PARP inhibitor resistant or refractory homologous recombination-deficient (HRD) driven cancer.

A pharmaceutical composition can comprise one or more compounds of Formula (I) including any compound disclosed in the examples below, as provided herein. In one example, an active pharmaceutical ingredient (API) can comprise a compound provided herein in a desired amount and concentration of the compound. Oral dosage forms comprising a compound provided herein can be prepared as a pharmaceutically acceptable formulation in a tablet, or in a capsule. The capsules can contain pharmaceutically acceptable excipients, and encapsulated capsules can be packaged in high-density polyethylene induction sealed bottles.

### EXAMPLES

The disclosure is further illustrated by the following examples and synthesis schemes, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

Unless otherwise indicated, the following abbreviations are used herein.

| | |
|---|---|
| Ac | acetyl |
| ACN | acetonitrile |
| amphos | Bis(di-tert-butyl(4-dimethylaminophenyl)phosphine) |
| Bn | benzyl |
| Bu | butyl |
| CDI | 1,1′-Carbonyldiimidazole |
| δ | chemical shift |
| DBU | 1,8-Diazabicyclo[5.4.0]undec-7-ene |
| DCE | 1,2-dichloroethane |
| DCM | dichloromethane |
| DIEA | N,N-diisopropylethylamine |
| DMF | N,N-dimethylformamide |
| DMSO | dimethylsulfoxide |
| dppf | 1,1′-bis(diphenylphosphino)ferrocene |
| EA | Ethyl Acetate |
| ES | electrospray |
| ESI | For LCMS Dataelectrospray ionization |
| equiv | equivalents |
| h | hour |
| ¹H NMR | proton nuclear magnetic resonance |
| HATU | 2-(3H-[1,2,3]triazolo[4,5-b]pyridin-3-yl)-1,1,3,3- |
| | tetramethylisouronium hexafluorophosphate |
| HPLC | high performance liquid chromatography |
| Hz | Hertz |
| LAH | Lithium aluminum hydride |
| LCMS | liquid chromatography/mass spectrometry |
| Me | methyl |
| min | minutes |
| M | molar |
| MS | mass spectrometry |
| Ms | mesyl |
| MW | Molecular weight |
| N | normal |
| NBS | N-bromosuccinimide |
| PE | petroleum ether |
| rt | room temperature |
| TEA | triethylamine |
| TFA | trifluoroacetic acid |
| THF | tetrahydrofuran |
| TLC | thin layer chromatography |
| XPhos | 2-Dicyclohexylphosphino-2′,4′,6′-triisopropylbiphenyl |
| XPhos- | methanesulfonato(2-dicyclohexylphosphino-2′,4′,6′- |
| Pd-G3 | triisopropyl-1,1′-biphenyl)(2′-amino-1,1′-biphenyl-2- |
| | yl)palladium(II) |

### Example 1: Synthesis of 5-(2-isopropylphenyl)-3-(4-(1-methyl-4-(trifluoromethyl)-1H-imidazol-2-yl)benzyl)thiazolo[4,5-d]pyrimidin-2(3H)-one (I-1)

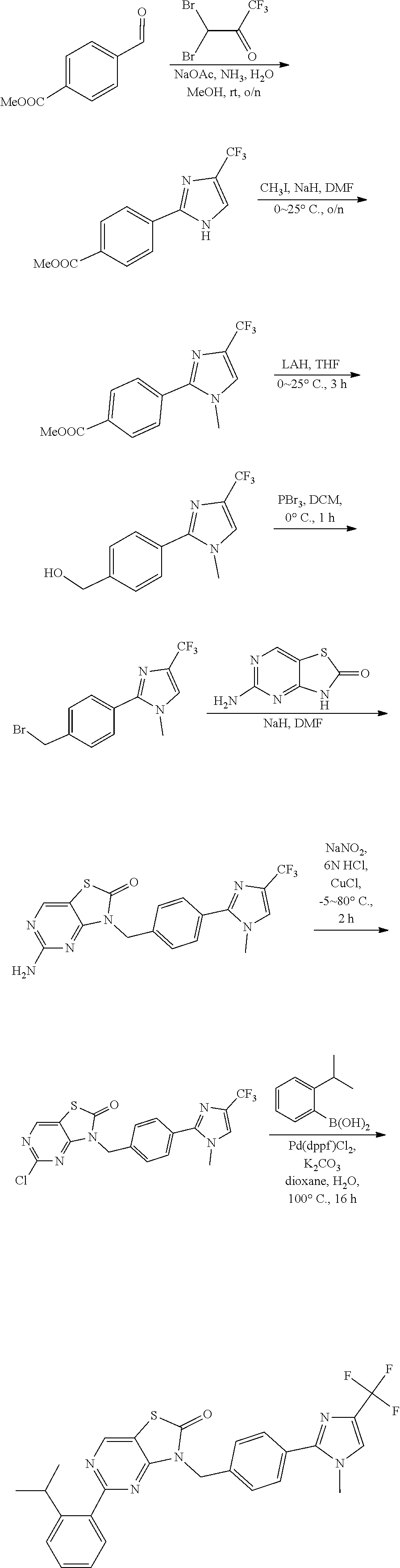

### Step 1. Synthesis of methyl 4-(4-(trifluoromethyl)-1H-imidazol-2-yl)benzoate

To a stirred solution of methyl 4-formylbenzoate (17.00 g, 98.38 mmol, 1.00 equiv) and 3,3-dibromo-1,1,1-trifluoropropan-2-one (55.89 g, 0.197 mmol, 2.00 equiv) in MeOH (500 mL) was added sodium acetate solution (16.99 g, 0.197 mmol, 2.00 equiv) and ammonia in water (100 mL) at 25° C. The resulting mixture was stirred for 18 h at 25° C. The resulting mixture was concentrated under vacuum. The residue was washed with water. The precipitated solids were collected by filtration and washed with water (3×500 mL). This resulted in methyl 4-[4-(trifluoromethyl)-1H-imidazol-2-yl]benzoate (22 g, 70.35%) as a yellow solid. LCMS (ES, m/z): 271 [M+H]⁺.

### Step 2. Synthesis of methyl 4-(1-methyl-4-(trifluoromethyl)-1H-imidazol-2-yl)benzoate

To a stirred solution of methyl 4-[4-(trifluoromethyl)-1H-imidazol-2-yl]benzoate (11.00 g, 34.603 mmol, 1.00 equiv) in DMF (100 mL) was added NaH (4.15 g, 103.80 mmol, 3.00 equiv, 60%) in portions at 0° C. The resulting mixture was stirred for 0.5 h at 0° C. To the above mixture was added CH₃I (17.00 g, 113.78 mmol, 3.29 equiv) dropwise at 0° C. The resulting mixture was stirred for additional 3 h at 25° C. The reaction was quenched by the addition of 1 N HCl (100 mL) at 0° C. The precipitated solids were collected by filtration and washed with water. The resulting solid was dried an oven. This resulted in methyl 4-[1-methyl-4-(trifluoromethyl)imidazol-2-yl]benzoate (9.8 g, 85.69%) as a yellow solid. LCMS (ES, m/z): 285 [M+H]⁺.

### Step 3. Synthesis of (4-(1-methyl-4-(trifluoromethyl)-1H-imidazol-2-yl)phenyl)methanol

To a stirred solution of methyl 4-[1-methyl-4-(trifluoromethyl)imidazol-2-yl]benzoate (9.80 g, 29.65 mmol, 1.00 equiv, 86%) in THF (50 mL) was added LiAlH₄ (2.12 g, 53.064 mmol, 1.79 equiv) in portions at 0° C. The resulting mixture was stirred for 3h at 25° C. The reaction was quenched with NH₄Cl (aq) at 25° C. The resulting mixture was extracted with EA (3×200 mL). The combined organic layers were washed with NaCl (2×100 mL), dried over anhydrous Na₂SO₄. After filtration, the filtrate was concentrated under reduced pressure. This resulted in [4-[1-methyl-4-(trifluoromethyl)imidazol-2-yl]phenyl]methanol (7 g, 72.79%) as a yellow oil. LCMS (ES, m/z): 257 [M+H]⁺.

### Step 4. Synthesis of 2-(4-(bromomethyl)phenyl)-1-methyl-4-(trifluoromethyl)-1H-imidazole

To a stirred solution of [4-[1-methyl-4-(trifluoromethyl)imidazol-2-yl]phenyl]methanol (7.00 g, 21.582 mmol, 1.00 equiv,) in DCM (50 mL) was added PBr₃ (18.50 g, 64.928 mmol, 3.01 equiv) dropwise at 0° C. The resulting mixture was stirred for 3 h at 25° C. The resulting mixture was concentrated under vacuum. The resulting mixture was washed with water. The precipitated solids were collected by filtration and dried over an oven. This resulted in 2-[4-(bromomethyl)phenyl]-1-methyl-4-(trifluoromethyl)imidazole (3.5 g, 43.19%) as a light yellow solid. LCMS (ES, m/z): 319, 321 [M+H]⁺.

### Step 5. Synthesis of 5-amino-3-(4-(1-methyl-4-(trifluoromethyl)-1H-imidazol-2-yl)benzyl)thiazolo[4,5-d]pyrimidin-2(3H)-one

To a stirred solution of 5-amino-3H-[1,3]thiazolo[4,5-d]pyrimidin-2-one (1.70 g, 8.694 mmol, 1.00 equiv) in DMF (20 mL) was added NaH (1.04 g, 26.002 mmol, 2.99 equiv, 60%) in portions at −10° C. The resulting mixture was stirred for 0.5 h at −10° C. Then 2-[4-(bromomethyl)phenyl]-1-methyl-4-(trifluoromethyl)imidazole (3.50 g, 10.419 mmol, 1.20 equiv, 95%) was added in and the mixture was stirred for 1 h at −10° C. The reaction was quenched by the addition of NH₄Cl (aq). The resulting mixture was extracted with EA (3×100 mL). The combined organic layers were washed with NaCl (2×100 mL), dried over anhydrous Na₂SO₄. After filtration, the filtrate was concentrated under reduced pressure. The residue was purified by silica gel column chromatography, eluted with EA/PE (1/2) to afford 5-amino-3-([4-[1-methyl-4-(trifluoromethyl)imidazol-2-yl]phenyl]methyl)-[1,3]thiazolo[4,5-d]pyrimidin-2-one (500 mg, 13.44%) as a light yellow solid. LCMS (ES, m/z): 407 [M+H]⁺.

### Step 6. Synthesis of 5-chloro-3-(4-(1-methyl-4-(trifluoromethyl)-1H-imidazol-2-yl)benzyl)thiazolo[4,5-d]pyrimidin-2(3H)-one

To a stirred solution of 5-amino-3-([4-[1-methyl-4-(trifluoromethyl)imidazol-2-yl]phenyl]methyl)-[1,3]thiazolo[4,5-d]pyrimidin-2-one (500 mg, 1.16 mmol, 1.00 equiv) in HCl (6M) (10.00 mL) was added NaNO₂ (150 mg, 2.06 mmol, 1.77 equiv) in water (0.5 mL) dropwise at −5° C. The resulting mixture was stirred for 10 min at −5° C. CuCl (347 mg, 3.50 mmol, 3.00 equiv) was added in. The resulting mixture was stirred for 2 h at 80° C. The mixture was allowed to cool down to 25° C. The resulting mixture was extracted with EA (3×50 mL). The combined organic layers were concentrated under reduced pressure. The residue was purified by silica gel column chromatography, eluted with EA/PE (1/3) to afford 5-chloro-3-([4-[1-methyl-4-(trifluoromethyl)imidazol-2-yl]phenyl]methyl)-[1,3]thiazolo[4,5-d]pyrimidin-2-one (200 mg, 38.18%) as a light yellow solid. LCMS (ES, m/z): 426, 428 [M+H]⁺.

### Step 7. Synthesis of 5-(2-isopropylphenyl)-3-(4-(1-methyl-4-(trifluoromethyl)-1H-imidazol-2-yl)benzyl)thiazolo[4,5-d]pyrimidin-2(3H)-one (I-1)

To a stirred mixture of 5-chloro-3-([4-[1-methyl-4-(trifluoromethyl)imidazol-2-yl]phenyl]methyl)-[1,3]thiazolo[4,5-d]pyrimidin-2-one (200 mg, 0.446 mmol, 1.00 equiv) and 2-isopropylphenylboronic acid (154 mg, 0.89 mmol, 2.00 equiv) in water (1 mL) and dioxane (10 mL) was added Pd(dppf)Cl₂ CH₂Cl₂ (72 mg, 0.089 mmol, 0.20 equiv,) and K₂CO₃ (154 mg, 1.11 mmol, 2.50 equiv). The resulting mixture was stirred for 16 h at 100° C. under N₂ atmosphere. The mixture was allowed to cool down to 25° C. The resulting mixture was concentrated under reduced pressure. The residue was purified by silica gel column chromatography, eluted with EA/PE (1/1). The crude product was purified by Prep-HPLC (Column: XBridge Shield RP18 OBD Column, 5 μm, 30×150 mm; Mobile Phase, A: water (containing 10 mmol/L NH₄HCO₃) and B: ACN (10% to 40% in 7 min); Flow rate: 60 mL/min; Detector: UV 254 nm). The product fractions were concentrated under vacuum to afford 5-(2-isopropylphenyl)-3-([4-[1-methyl-4-(trifluoromethyl)imidazol-2-yl]phenyl]methyl)-[1,3]thiazolo[4,5-d]pyrimidin-2-one (38.3 mg, 15.5%) as an off-white solid. LCMS (ES, m/z): 510.55 [M+H]⁺. 1H NMR (300 MHz, Methanol-d4) δ 8.88 (s, 1H), 7.73-7.43 (m, 8H), 7.30 (ddd, J=7.4, 6.4, 2.2 Hz, 1H), 5.37 (s, 2H), 3.77 (s, 3H), 3.52-3.41 (m, 1H), 1.19 (s, 3H), 1.17 (s, 3H).

### Example 2: Synthesis of 3-[8-([4-[1-methyl-4-(trifluoromethyl)-1H-imidazol-2-yl]phenyl]methyl)-6H,7H,8H-pyrimido[5,4-b][1,4]oxazin-2-yl]-2-(propan-2-yl)pyridine (I-2)

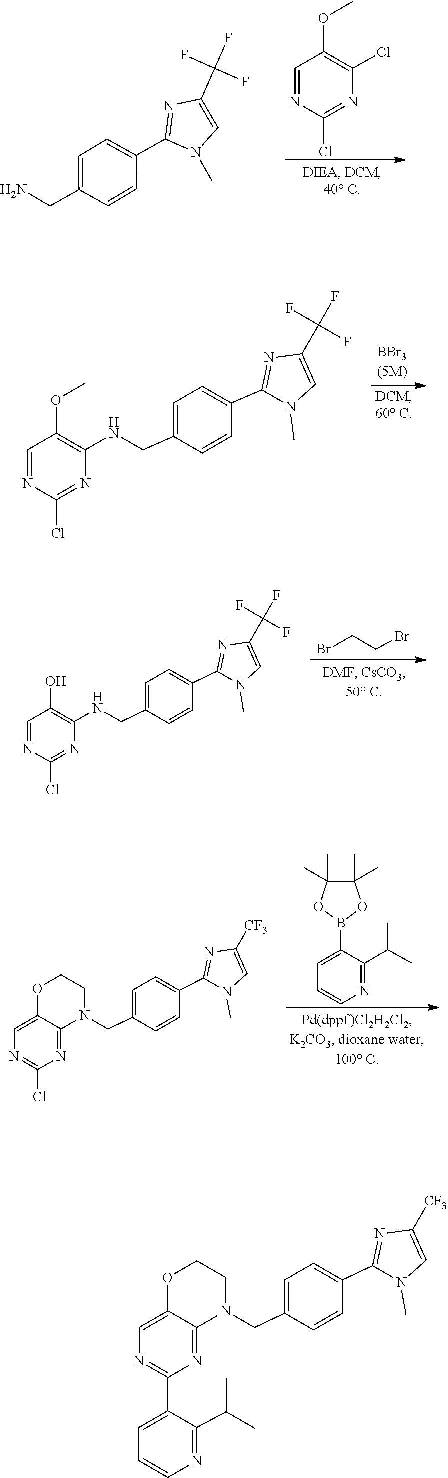

### Step 1. Synthesis of 2-chloro-5-methoxy-N-([4-[1-methyl-4-(trifluoromethyl)-1H-imidazol-2-yl]phenyl]methyl)pyrimidin-4-amine

To a stirred mixture of 2,4-dichloro-5-methoxypyrimidine (2.10 g, 11.75 mmol, 1.20 equiv) and DIEA (2.53 g, 19.59 mmol, 2.00 equiv) in DCM (30 mL) was added a solution of [4-[1-methyl-4-(trifluoromethyl)-1H-imidazol-2-yl]phenyl]methanamine (2.50 g, 9.79 mmol, 1.00 equiv) in DCM (10 mL) dropwise with stirring at 0° C. in 30 min. The resulting solution was stirred overnight at 17° C. The resulting mixture was concentrated under vacuum. The residue was applied onto a silica gel column (eluting with EA/petroleum ether 3/1) to afford 1.4 g (36%) of 2-chloro-5-methoxy-N-([4-[1-methyl-4-(trifluoromethyl)-1H-imidazol-2-yl]phenyl]methyl)pyrimidin-4-amine as a yellow solid. LC-MS (ESI) m/z 398.1 [M+H]⁺.

### Step 2. Synthesis of 2-chloro-4-[([4-[1-methyl-4-(trifluoromethyl)-1H-imidazol-2-yl]phenyl]methyl)amino]pyrimidin-5-ol

To a stirred mixture of 2-chloro-5-methoxy-N-([4-[1-methyl-4-(trifluoromethyl)-1H-imidazol-2-yl]phenyl]methyl)pyrimidin-4-amine (1.4 g, 3.52 mmol, 1.00 equiv) in DCM (10 mL) was added BBr₃ (10 mL). The resulting solution was stirred for 4 h at 60° C. After cooling to room temperature, the reaction mixture was poured in to ice/water, sodium hydroxide solution (1 mol/L) was employed to adjust the pH to 6-7, extracted with EA (100 ml×3). The organic layers were combined, washed with brine (100 mL×1), dried over anhydrous sodium sulfate and concentrated under vacuum. The residue was applied onto a silica gel column (eluting with DCM/methanol 10/1) to afford 1.1 g (81%) of 2-chloro-4-[([4-[1-methyl-4-(trifluoromethyl)-1H-imidazol-2-yl]phenyl]methyl)amino]pyrimidin-5-ol as a yellow solid. LC-MS (ESI) m/z 384 [M+H]⁺.

### Step 3. Synthesis of 2-[4-([2-chloro-6H,7H,8H-pyrimido[5,4-b][1,4]oxazin-8-yl]methyl)phenyl]-1-methyl-4-(trifluoromethyl)-1H-imidazole

Into a 8 mL round-bottom flask, was placed 2-chloro-4-[([4-[1-methyl-4-(trifluoromethyl)-1H-imidazol-2-yl]phenyl]methyl)amino]pyrimidin-5-ol (100 mg, 0.25 mmol, 1.00 equiv, 95%), 1,2-dibromoethane (140 mg, 0.75 mmol, 3.00 equiv), Cs₂CO₃ (403 mg, 1.24 mmol, 5.00 equiv), DMF (5 mL). The resulting solution was stirred for 3 h at 50° C. The reaction mixture was cooled to room temperature (20° C.). The resulting solution was diluted with 7 mL of water. The resulting solution was extracted with 2×7 mL of EA and the organic layers combined and concentrated under vacuum. The residue was purified by preparative TLC (EA:PE=1:1). This resulted in 80 mg (75%) of 2-[4-([2-chloro-6H,7H,8H-pyrimido[5,4-b][1,4]oxazin-8-yl]methyl)phenyl]-1-methyl-4-(trifluoromethyl)-1H-imidazole as yellow oil. LC-MS (ESI) m z 410 [M+H]⁺.

### Step 4. Synthesis of 3-[8-([4-[1-methyl-4-(trifluoromethyl)-1H-imidazol-2-yl]phenyl]methyl)-6H,7H,8H-pyrimido[5,4-b][1,4]oxazin-2-yl]-2-(propan-2-yl)pyridine (I-2)

Into a 25 mL round-bottom flask purged and maintained with an inert atmosphere of nitrogen, was placed 2-[4-([2-chloro-6H, 7H, 8H-pyrimido[5,4-b][1,4]oxazin-8-yl]methyl)phenyl]-1-methyl-4-(trifluoromethyl)-1H-imidazole (80 mg, 0.19 mmol, 1.00 equiv), 2-(propan-2-yl)-3-(tetramethyl-1,3,2-dioxaborolan-2-yl)pyridine (68.752 mg, 0.27 mmol, 1.50 equiv), Pd(dppf)Cl2·CH2Cl2 (15.145 mg, 0.02 mmol, 0.10 equiv), potassium carbonate (51.263 mg, 0.37 mmol, 2.00 equiv), dioxane (10 mL), water (3 mL). The resulting solution was stirred for 4 h at 100° C. The reaction mixture was cooled to rt (25° C.). The solids were filtered out. The filtrate was concentrated under vacuum. The residue was purified by preparative TLC (EA:PE=1:1). The crude product was purified by Prep-HPLC with the following conditions (Prep-HPLC-025): Column: XBridge Prep C18 OBD Column, 5 um, 19*150 mm; Mobile Phase A: Water (10 mMOL/L NH₄HCO₃), Mobile Phase B: ACN; Flow rate: 20 mL/min; Gradient: 35% B to 55% B in 7 min; 254 nm; Rt: Array min. This resulted in 45.6 mg (49%) of 3-[8-([4-[1-methyl-4-(trifluoromethyl)-1H-imidazol-2-yl]phenyl]methyl)-6H,7H,8H-pyrimido[5,4-b][1,4]oxazin-2-yl]-2-(propan-2-yl)pyridine as a white solid. LC-MS (ESI) m z 495.2 [M+H]^{+ 1}H NMR (400 MHZ, DMSO-d₆) δ 8.54-8.52 (m, 1H), 7.99 (s, 1H), 7.93 (s, 1H), 7.90-7.87 (m, 1H), 7.71-7.69 (m, 2H), 7.43-7.41 (m, 2H), 7.26-7.22 (m, 1H), 4.95 (s, 2H), 4.32-4.29 (m, 2H), 3.77 (s, 3H), 3.73-3.66 (m, 1H), 3.63-3.59 (m, 2H), 1.07 (d, J=6.4 Hz, 6H).

### Example 3: Synthesis of 8-([4-[1-methyl-4-(trifluoromethyl)-1H-imidazol-2-yl]phenyl]methyl)-2-[2-(propan-2-yl)phenyl]-6H,7H,8H-pyrimido[5,4-b][1,4]oxazin-7-one (I-3)

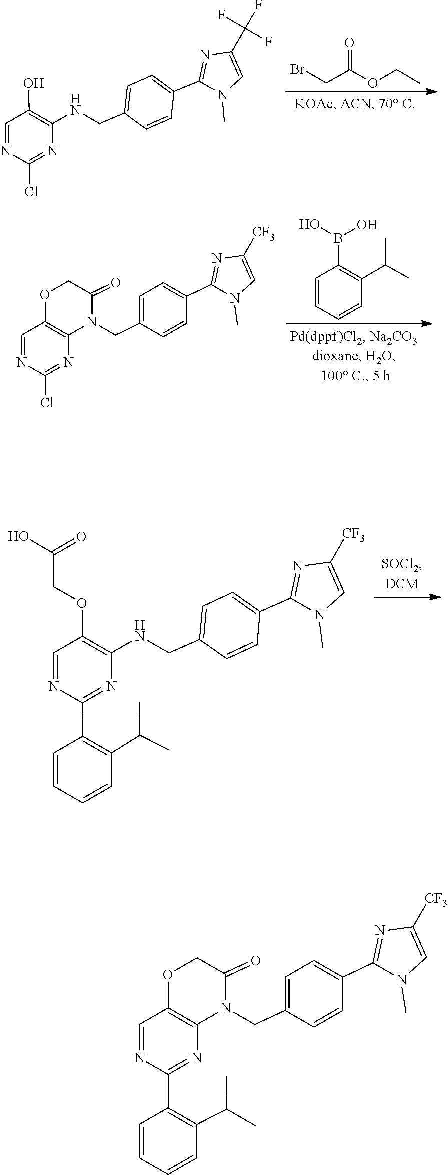

### Step 1. Synthesis of 2-chloro-8-([4-[1-methyl-4-(trifluoromethyl)-1H-imidazol-2-yl]phenyl]methyl)-6H,7H,8H-pyrimido[5,4-b][1,4]oxazin-7-one

To a stirred mixture of 2-chloro-4-[([4-[1-methyl-4-(trifluoromethyl)-1H-imidazol-2-yl]phenyl]methyl)amino]pyrimidin-5-ol (200 mg, 0.52 mmol, 1.00 equiv) and ethyl 2-bromoacetate (174 mg, 1.04 mmol, 2.00 equiv) in ACN (3 mL) was added KOAc (255 mg, 2.60 mmol, 5.00 equiv). The resulting solution was stirred overnight at 70° C. After cooling to room temperature, the solids were filtered out. The filtrate was concentrated under vacuum. The residue was purified by Prep-TLC (eluting with EA/ether petroleum 1/1) to afford 90 mg (41%) of 2-chloro-8-([4-[1-methyl-4-(trifluoromethyl)-1H-imidazol-2-yl]phenyl]methyl)-6H, 7H,8H-pyrimido[5,4-b][1,4]oxazin-7-one as a white solid. LC-MS (ESI) m/z 424 [M+H]⁺.

### Step 2. Synthesis of 2-([4-[([4-[1-methyl-4-(trifluoromethyl)-1H-imidazol-2-yl]phenyl]methyl)amino]-2-[2-(propan-2-yl)phenyl]pyrimidin-5-yl]oxy)acetic acid

To a stirred mixture of 2-chloro-8-([4-[1-methyl-4-(trifluoromethyl)-1H-imidazol-2-yl]phenyl]methyl)-6H,7H,8H-pyrimido[5,4-b][1,4]oxazin-7-one (90 mg, 0.21 mmol, 1.00 equiv) and [2-(propan-2-yl)phenyl]boronic acid (69 mg, 0.42 mmol, 2.00 equiv) in 1,4-dioxane (3 mL) and water (1 mL) was added Pd(dppf)Cl₂·CH₂Cl₂ (18 mg, 0.02 mmol, 0.10 equiv), sodium carbonate (45 mg, 0.42 mmol, 2.00 equiv), The resulting solution was stirred for 20 h at 100° C. The reaction mixture was cooled to room temperature. The solids were filtered out. The filtrate was concentrated under vacuum. The residue was purified by Prep-TLC (eluting with DCM/methanol 10/1) to afford 80 mg (72%) of 2-([4-[([4-[1-methyl-4-(trifluoromethyl)-1H-imidazol-2-yl]phenyl]methyl)amino]-2-[2-(propan-2-yl)phenyl]pyrimidin-5-yl]oxy)acetic acid as a white solid. LC-MS (ESI) m/z 510 [M+H]⁺.

### Step 3. Synthesis of 8-([4-[1-methyl-4-(trifluoromethyl)-1H-imidazol-2-yl]phenyl]methyl)-2-[2-(propan-2-yl)phenyl]-6H, 7H,8H-pyrimido[5,4-b][1,4]oxazin-7-one (I-3)

To a stirred mixture of 2-([4-[([4-[1-methyl-4-(trifluoromethyl)-1H-imidazol-2-yl]phenyl]methyl)amino]-2-[2-(propan-2-yl)phenyl]pyrimidin-5-yl]oxy)acetic acid (80 mg, 0.15 mmol, 1.00 equiv) in DCM (10 mL) was added thionyl chloride (54 mg, 0.45 mmol, 3.00 equiv) and DMF (0.05 mL, 0.65 mmol, 0.01 equiv). The resulting solution was stirred for 2 h at 50° C. The reaction mixture was cooled to room temperature. The resulting mixture was concentrated under vacuum. The crude product was purified by Prep-HPLC with the following conditions: Column, XBridge Prep C18 OBD Column, 19×150 mm 5 um; mobile phase, waters (0.05% TFA) and ACN (45.0% ACN up to 85.0% in 7 min); Detector, UV 254/220 nm. This resulted in 10.6 mg (14%) of 8-([4-[1-methyl-4-(trifluoromethyl)-1H-imidazol-2-yl]phenyl]methyl)-2-[2-(propan-2-yl)phenyl]-6H,7H,8H-pyrimido[5,4-b][1,4]oxazin-7-one as a white solid. LC-MS (ESI) m/z 508 [M+H]+ 1H-NMR: (300 MHz, DMSO, ppm): 8.53 (s, 1H), 7.93 (d, J=1.6 Hz, 1H), 7.67 (d, J=8.1 Hz, 2H), 7.49-7.39 (m, 5H), 7.27-7.21 (m, 1H), 5.31 (s, 2H), 5.06 (s, 2H), 3.76 (s, 3H), 3.47-3.42 (m, 1H), 1.01 (d, J=6.9 Hz, 6H).

### Example 4: Synthesis of 8-([4-[1-methyl-4-(trifluoromethyl)-1H-imidazol-2-yl]phenyl]methyl)-2-[2-(propan-2-yl)pyridin-3-yl]-6H,7H,8H-pyrimido[5,4-b][1,4]oxazin-7-one (I-4)

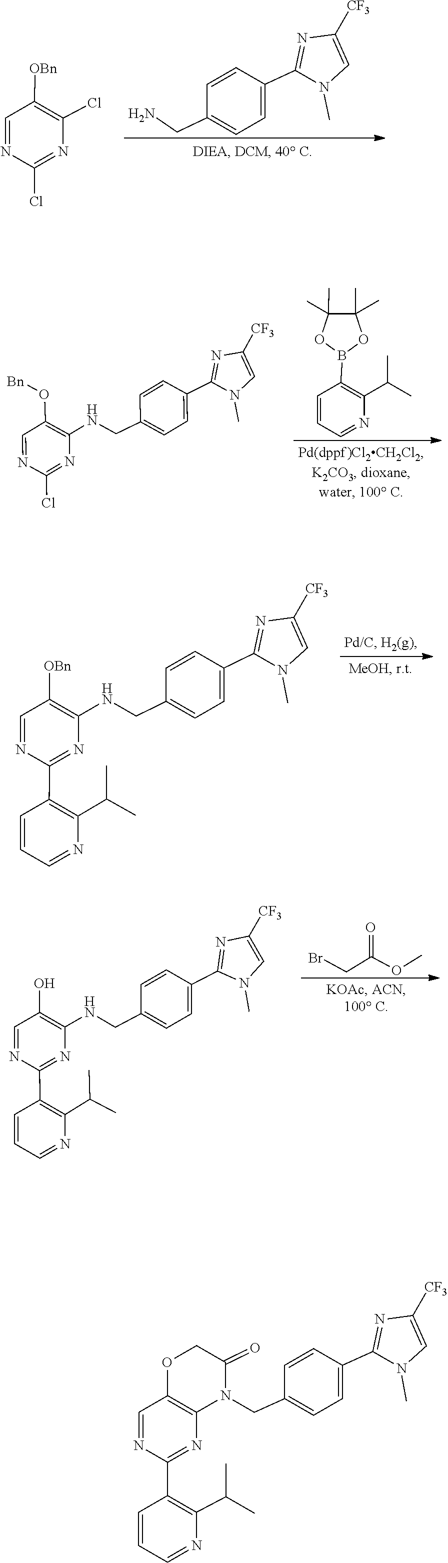

### Step 1. Synthesis of 5-(benzyloxy)-2-chloro-N-([4-[1-methyl-4-(trifluoromethyl)-1H-imidazol-2-yl]phenyl]methyl)pyrimidin-4-amine

Into a 50 mL round-bottom flask was placed 5-(benzyloxy)-2,4-dichloropyrimidine (1.86 g, 7.29 mmol, 1.10 equiv), DCM (20 mL), DIEA (1.71 g, 13.23 mmol, 2.00 equiv), [4-[1-methyl-4-(trifluoromethyl)-1H-imidazol-2-yl]phenyl]methanamine (1.7 g, 6.66 mmol, 1.00 equiv). The resulting solution was stirred for 3 h at 40° C. After cooling to room temperature, the resulting mixture was concentrated under vacuum. The residue was applied onto a silica gel column with EA/petroleum ether (2:3). This resulted in 900 mg (29%) of 5-(benzyloxy)-2-chloro-N-([4-[1-methyl-4-(trifluoromethyl)-1H-imidazol-2-yl]phenyl]methyl)pyrimidin-4-amine as a white solid. LC-MS (ESI) m/z 474.30 [M+H]+.

### Step 2. Synthesis of 5-(benzyloxy)-N-([4-[1-methyl-4-(trifluoromethyl)-1H-imidazol-2-yl]phenyl]methyl)-2-[2-(propan-2-yl)pyridin-3-yl]pyrimidin-4-amine

Into a 8 mL sealed tube purged and maintained with an inert atmosphere of nitrogen was placed 5-(benzyloxy)-2-chloro-N-([4-[1-methyl-4-(trifluoromethyl)-1H-imidazol-2-yl]phenyl]methyl)pyrimidin-4-amine (319 mg, 0.67 mmol, 1.00 equiv), 2-(propan-2-yl)-3-(tetramethyl-1,3,2-dioxaborolan-2-yl)pyridine (200 mg, 0.81 mmol, 1.20 equiv), Pd(dppf)Cl2 CH2Cl2 (55 mg, 0.07 mmol, 0.10 equiv), potassium carbonate (186 mg, 1.35 mmol, 2.00 equiv), 1,4-dioxane (1.5 mL), water (0.5 mL). The resulting solution was stirred overnight at 100° C. After cooling to room temperature, the resulting mixture was filtered and concentrated under vacuum. The residue was purified by Prep-TLC (eluting with DCM/methanol 10/1) to afford 200 mg (53%) of 5-(benzyloxy)-N-([4-[1-methyl-4-(trifluoromethyl)-1H-imidazol-2-yl]phenyl]methyl)-2-[2-(propan-2-yl)pyridin-3-yl]pyrimidin-4-amine as a pink solid. LC-MS (ESI) m/z 559.40 [M+H]+.

### Step 3. Synthesis of 4-[([4-[1-methyl-4-(trifluoromethyl)-1H-imidazol-2-yl]phenyl]methyl)amino]-2-[2-(propan-2-yl)pyridin-3-yl]pyrimidin-5-ol

Into a 50 mL round-bottom flask was placed 5-(benzyloxy)-N-([4-[1-methyl-4-(trifluoromethyl)-1H-imidazol-2-yl]phenyl]methyl)-2-[2-(propan-2-yl)pyridin-3-yl]pyrimidin-4-amine (100 mg, 0.18 mmol, 1.00 equiv), methanol (10 mL), palladium on carbon (10 mg, 10%). To the above hydrogen was introduced in. The resulting solution was stirred for 3 h at room temperature (22° C.). The reaction solution was filtered and concentrated. This resulted in 140 mg (crude) of 4-[([4-[1-methyl-4-(trifluoromethyl)-1H-imidazol-2-yl]phenyl]methyl)amino]-2-[2-(propan-2-yl)pyridin-3-yl]pyrimidin-5-ol as a white solid. LC-MS (ESI) m/z 469.35 [M+H]+.

### Step 4. Synthesis of 8-([4-[1-methyl-4-(trifluoromethyl)-1H-imidazol-2-yl]phenyl]methyl)-2-[2-(propan-2-yl)pyridin-3-yl]-6H,7H,8H-pyrimido[5,4-b][1,4]oxazin-7-one (I-4)

Into a 8 mL sealed tube was placed 4-[([4-[1-methyl-4-(trifluoromethyl)-1H-imidazol-2-yl]phenyl]methyl)amino]-2-[2-(propan-2-yl)pyridin-3-yl]pyrimidin-5-ol (84 mg, 0.18 mmol, 1.00 equiv), ethyl 2-bromoacetate (30 mg, 0.18 mmol, 1.00 equiv), KOAc (88 mg, 0.90 mmol, 5.00 equiv), ACN (2 mL). The resulting solution was stirred for 2 h at 30° C. The resulting solution was allowed to react with stirring overnight at 100° C. After cooling to room temperature, the resulting mixture was concentrated under vacuum. The residue was purified by prep-TLC with DCM/methanol (10/1). The crude product was purified by Prep-HPLC (Column: XBridge Shield RP18 OBD Prep Column, 130 A, 5 um, 19 mm×150 mm; Mobile phase: water (10 mmol NH4HCO3), MeCN (35% MeCN up to 85.0% over 7 min); Flow rate: 20 mL/min; Detector: 254 nm). This resulted in 9.3 mg (10%) of 8-([4-[1-methyl-4-(trifluoromethyl)-1H-imidazol-2-yl]phenyl]methyl)-2-[2-(propan-2-yl)pyridin-3-yl]-6H,7H,8H-pyrimido[5,4-b][1,4]oxazin-7-one as a white solid. LC-MS (ESI) m/z 509.20 [M+H]+ 1H NMR (300 MHz, CD3CN): δ 8.58-8.55 (m, 1H), 8.41 (s, 1H), 7.95-7.91 (m, 1H), 7.61 (d, J=8.4 Hz, 2H), 7.51 (s, 1H), 7.46 (d, J=8.4 Hz, 2H), 7.23-7.21 (m, 1H), 5.36 (s, 2H), 4.91 (s, 2H), 3.71 (s, 3H), 3.67-3.62 (m, 1H), 1.08 (d, J=6.6 Hz, 6H).

### Example 5: Synthesis of 6,6-dimethyl-8-([4-[1-methyl-4-(trifluoromethyl)-1H-imidazol-2-yl]phenyl]methyl)-2-[2-(propan-2-yl)pyridin-3-yl]-6H,7H,8H-pyrimido[5,4-b][1,4]oxazin-7-one (I-5)

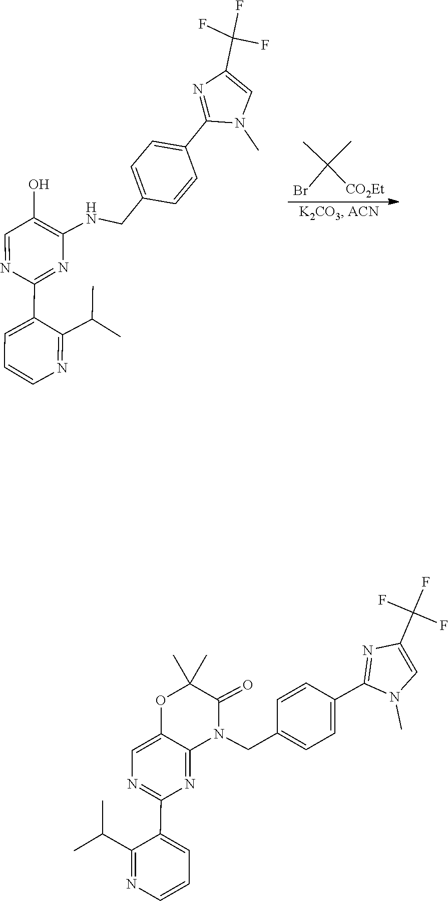

### Step 1. Synthesis of 6,6-dimethyl-8-([4-[1-methyl-4-(trifluoromethyl)-1H-imidazol-2-yl]phenyl]methyl)-2-[2-(propan-2-yl)pyridin-3-yl]-6H,7H,8H-pyrimido[5,4-b][1,4]oxazin-7-one (I-5)

Into a 8-mL vial, was placed 4-[([4-[1-methyl-4-(trifluoromethyl)-1H-imidazol-2-yl]phenyl]methyl)amino]-2-[2-(propan-2-yl)pyridin-3-yl]pyrimidin-5-ol (80 mg, 0.16 mmol, 1.00 equiv, 96%), ACN (2 mL), potassium carbonate (70.77 mg, 0.51 mmol, 3.12 equiv), ethyl 2-bromo-2-methylpropanoate (39.8 mg, 0.20 mmol, 1.25 equiv). The resulting solution was stirred for 48 h at 25° C. The resulting solution was diluted with 2 mL of water. The resulting solution was extracted with 3×2 mL of EA and the organic layers combined and dried over anhydrous sodium sulfate. The solids were filtered out. The resulting mixture was concentrated under vacuum. The crude product was purified by Prep-HPLC with the following conditions: Column, XBridge C18 OBD Prep Column, 5 μm, 19 mm×250 mm; mobile phase, water (10 mMOL/L NH4HCO3) and ACN (40.0% ACN up to 60.0% in 7 min); Detector, UV 254 nm. This resulted in 23.1 mg (26%) of 6,6-dimethyl-8-([4-[1-methyl-4-(trifluoromethyl)-1H-imidazol-2-yl]phenyl]methyl)-2-[2-(propan-2-yl)pyridin-3-yl]-6H,7H,8H-pyrimido[5,4-b][1,4]oxazin-7-one as an off-white solid. LC-MS-PH-FMA-PJ111-884-0: (ES, m/z): 537[M+H]+ H-NMR-PH-FMA-PJ111-884-0: (400 MHZ, Methanol-d4) δ 8.54-8.53 (m, 1H), 8.44 (s, 1H), 8.00-7.98 (m, 1H), 7.67 (s, 1H), 7.65-7.57 (m, 2H), 7.52-7.44 (m, 2H), 7.32-7.30 (m, 1H), 5.42 (s, 2H), 3.75 (s, 3H), 3.64-3.60 (m, 1H), 1.64 (s, 6H), 1.16 (d, J=6.8 Hz, 6H).

### Example 6: Synthesis of 3-methyl-1-([4-[1-methyl-4-(trifluoromethyl)-1H-imidazol-2-yl]phenyl]methyl)-7-[2-(propan-2-yl)phenyl]-1H,2H,3H,4H-[1,3]diazino[4,5-d]pyrimidin-2-one (I-6)

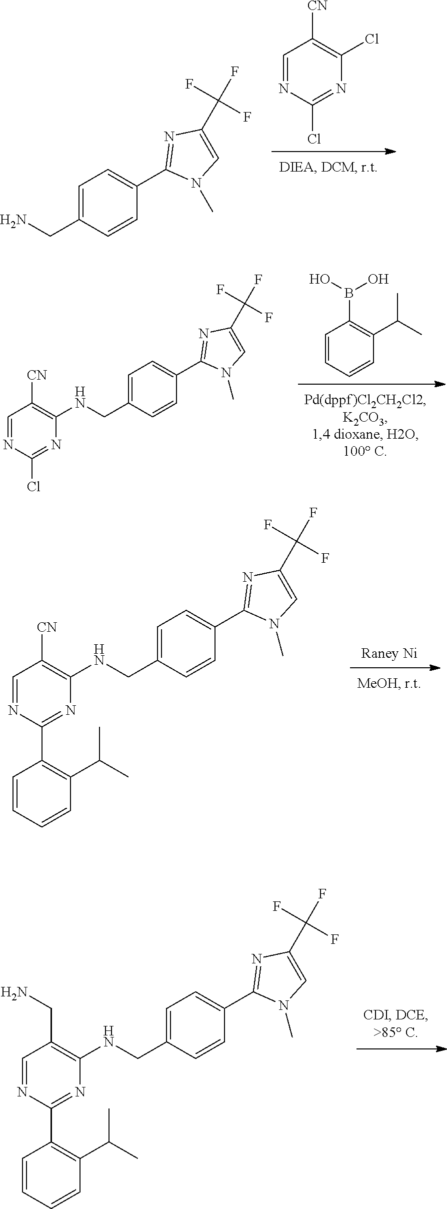

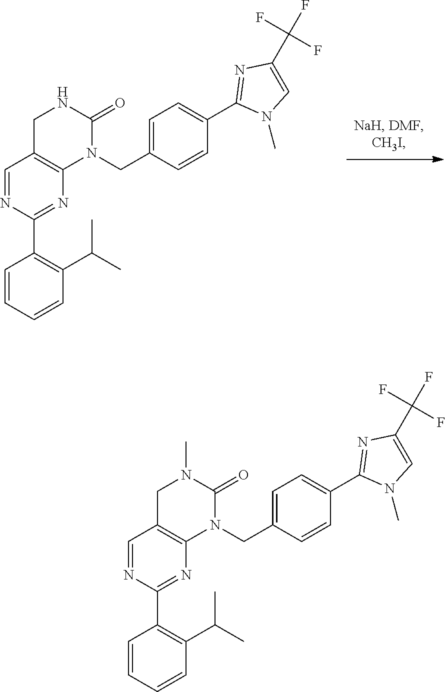 [SMILES: CC(C)c1ccccc1-c1ncc2c(n1)N(Cc1ccc(-c3nc(C(F)(F)F)cn3C)cc1)C(=O)NC2 ; CC(C)c1ccccc1-c1ncc2c(n1)N(Cc1ccc(-c3nc(C(F)(F)F)cn3C)cc1)C(=O)N(C)C2]

### Step 1. Synthesis of 2-chloro-4-[([4-[1-methyl-4-(trifluoromethyl)-1H-imidazol-2-yl]phenyl]methyl)amino]pyrimidine-5-carbonitrile

Into a 250 mL round-bottom flask, was placed 2,4-dichloropyrimidine-5-carbonitrile (2 g, 11.50 mmol, 1.01 equiv), DCM (60 mL). This was followed by the addition of DIEA (3 g, 23.21 mmol, 2.04 equiv) dropwise with stirring at 0° C. in 5 min. To this was added [4-[1-methyl-4-(trifluoromethyl)-1H-imidazol-2-yl]phenyl]methanamine (2.9 g, 11.36 mmol, 1.00 equiv). The resulting solution was stirred for 2 h at room temperature. The resulting mixture was concentrated under vacuum. The residue was applied onto a silica gel column with EA/petroleum ether (0/100-100/0). This resulted in 2.4 g (54%) of 2-chloro-4-[([4-[1-methyl-4-(trifluoromethyl)-1H-imidazol-2-yl]phenyl]methyl)amino]pyrimidine-5-carbonitrile as brown oil. LC-MS (ESI) m/z 393.2 [M+H]+ LC-MS (ESI) m/z 469.35 [M+H]+.

### Step 2. Synthesis of 4-[([4-[1-methyl-4-(trifluoromethyl)-1H-imidazol-2-yl]phenyl]methyl)amino]-2-[2-(propan-2-yl)phenyl]pyrimidine-5-carbonitrile

Into a 100 mL round-bottom flask purged and maintained with an inert atmosphere of nitrogen, was placed 2-chloro-4-[([4-[1-methyl-4-(trifluoromethyl)-1H-imidazol-2-yl]phenyl]methyl)amino]pyrimidine-5-carbonitrile (2.4 g, 6.11 mmol, 1.00 equiv), dioxane (50 mL), water (10 mL), [2-(propan-2-yl)phenyl]boronic acid (1.51 g, 9.21 mmol, 1.51 equiv), potassium carbonate (1.69 g, 12.23 mmol, 2.00 equiv), Pd(dppf)Cl2·CH2Cl2 (500 mg, 0.61 mmol, 0.10 equiv). The resulting solution was stirred for 18 h at 100° C. in an oil bath. The reaction mixture was cooled to room temperature with a water bath. The solids were filtered out. The resulting mixture was concentrated under vacuum. The residue was applied onto a silica gel column with EA/petroleum ether (0:100-70:30). This resulted in 2.24 g (77%) of 4-[([4-[1-methyl-4-(trifluoromethyl)-1H-imidazol-2-yl]phenyl]methyl)amino]-2-[2-(propan-2-yl)phenyl]pyrimidine-5-carbonitrile as brown oil. LC-MS (ESI) m/z 477.2 [M+H]+.

### Step 3. Synthesis of 5-(aminomethyl)-N-([4-[1-methyl-4-(trifluoromethyl)-1H-imidazol-2-yl]phenyl]methyl)-2-[2-(propan-2-yl)phenyl]pyrimidin-4-amine

Into a 250-mL round-bottom flask, was placed 4-[([4-[1-methyl-4-(trifluoromethyl)-1H-imidazol-2-yl]phenyl]methyl)amino]-2-[2-(propan-2-yl)phenyl]pyrimidine-5-carbonitrile (2.24 g, 4.70 mmol, 1.00 equiv), ammonia (7 M in methanol) (30 mL), Raney Ni (700 mg, 8.17 mmol, 1.74 equiv). To the above hydrogen was introduced in. The resulting solution was stirred for 3 days at room temperature (10° C.). The solids were filtered out. The resulting mixture was concentrated under vacuum. This resulted in 1.7 g (75%) of 5-(aminomethyl)-N-([4-[1-methyl-4-(trifluoromethyl)-1H-imidazol-2-yl]phenyl]methyl)-2-[2-(propan-2-yl)phenyl]pyrimidin-4-amine as a brown solid. LC-MS (ESI) m/z 480.1 [M+H]+.

### Step 4. Synthesis of 7-(2-isopropylphenyl)-1-([4-[1-methyl-4-(trifluoromethyl)imidazol-2-yl]phenyl]methyl)-3H,4H-pyrimido[4,5-d][1,3]diazin-2-one

To a stirred mixture of 5-(aminomethyl)-N-([4-[1-methyl-4-(trifluoromethyl)-1H-imidazol-2-yl]phenyl]methyl)-2-[2-(propan-2-yl)phenyl]pyrimidin-4-amine (50 mg, 0.10 mmol, 1.00 equiv) in DCE (1 mL) was added CDI (68 mg, 0.41 mmol, 4.00 equiv). The resulting mixture was stirred for 3 h at 90° C. Then the resulting mixture was concentrated and purified by Prep-HPLC (Column: XBridge Prep C18 OBD Column, 19*150 mm, 5 um; Mobile Phase A: water (0.05% TFA), Mobile Phase B: ACN; Flow rate: 20 mL/min; Gradient: 40% B to 70% B in 7 min, 70% B; Wave Length: 254/220 nm; Number Of Runs) to give 5 mg (9.5%) of 1-([4-[1-methyl-4-(trifluoromethyl)-1H-imidazol-2-yl]phenyl]methyl)-7-[2-(propan-2-yl)phenyl]-1H,2H,3H,4H-pyrimido[4,5-d][1,3]diazin-2-one as a white solid. LC-MS (ESI) m/z 507 [M+H]+.

### Step 5. Synthesis of 3-methyl-1-([4-[1-methyl-4-(trifluoromethyl)-1H-imidazol-2-yl]phenyl]methyl)-7-[2-(propan-2-yl)phenyl]-1H,2H,3H,4H-[1,3]diazino[4,5-d]pyrimidin-2-one (I-6)

Into a 8 mL vial, was placed 1-([4-[1-methyl-4-(trifluoromethyl)-1H-imidazol-2-yl]phenyl]methyl)-7-[2-(propan-2-yl)phenyl]-1H,2H,3H,4H-pyrimido[4,5-d][1,3]diazin-2-one (5 mg, 0.01 mmol, 1.00 equiv), DMF (0.2 mL). This was followed by the addition of sodium hydride (0.8 mg, 0.02 mmol, 2.00 equiv, 60%) at 0° C. The mixture was stirred for 30 min at 0° C. To this was added CH3I (1.5 mg, 0.01 mmol, 1.20 equiv). The resulting solution was stirred for 2 h at room temperature (19° C.). The reaction was then quenched by the addition of 0.2 ml of water. The crude product was purified by Prep-HPLC with the following conditions: Column, XBridge Shield RP18 OBD Column, 5 um, 19×150 mm; mobile phase, waters (0.1% FA) and ACN (45.0% ACN up to 70.0% in 7 min); Detector, UV 254 nm. This resulted in 1.5 mg (29%) of 3-methyl-1-([4-[1-methyl-4-(trifluoromethyl)-1H-imidazol-2-yl]phenyl]methyl)-7-[2-(propan-2-yl)phenyl]-1H,2H,3H,4H-[1,3]diazino[4,5-d]pyrimidin-2-one as a white solid. LC-MS (ESI) m/z 521 [M+H]+ 1H-NMR-PH-SDM-014-3R-13-0 (400 MHZ, DMSO-d6) δ (ppm): 8.45 (s, 1H), 7.65 (s, 1H), 7.56 (d, J=8.4 Hz, 2H), 7.45-7.38 (m, 5H), 7.24-7.20 (m, 1H), 5.37 (s, 2H), 4.64 (s, 2H), 3.74 (s, 3H), 3.39-3.35 (m, 1H), 3.09 (s, 3H), 1.04 (d, J=6.8 Hz, 6H).

### Example 7: Synthesis of 1-([4-[1-methyl-4-(trifluoromethyl)-1H-imidazol-2-yl]phenyl]methyl)-7-[2-(propan-2-yl)phenyl]-1H,2H, 4H-pyrimido[4,5-d][1,3]oxazin-2-one (I-7)

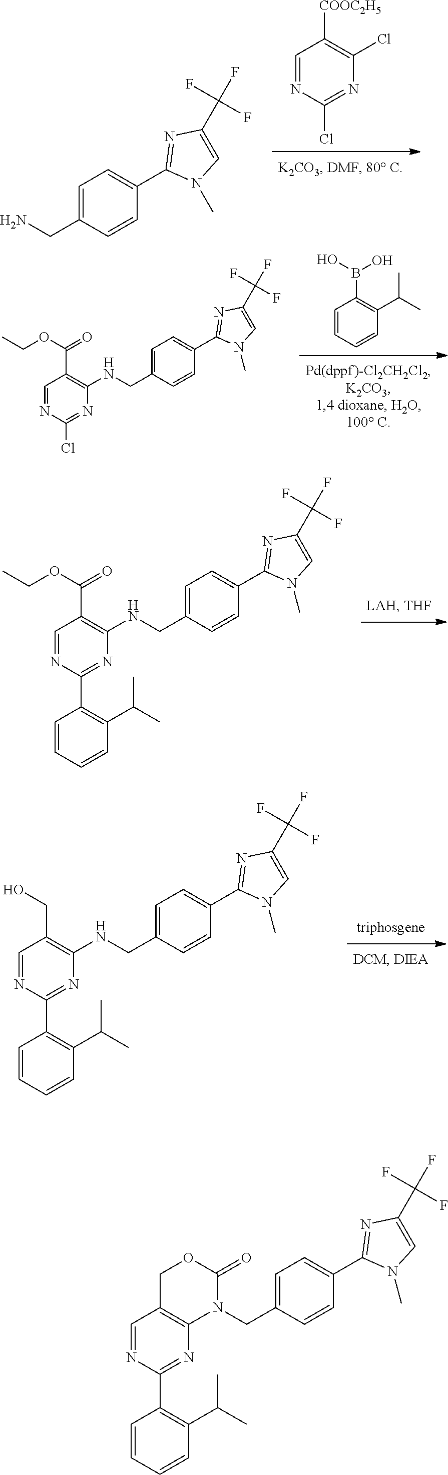

### Step 1. Synthesis of ethyl 2-chloro-4-[([4-[1-methyl-4-(trifluoromethyl)-1H-imidazol-2-yl]phenyl]methyl)amino]pyrimidine-5-carboxylate

Into a 100 mL round-bottom flask, was placed ethyl 2,4-dichloropyrimidine-5-carboxylate (2 g, 9.05 mmol, 1.00 equiv), [4-[1-methyl-4-(trifluoromethyl)-1H-imidazol-2-yl]phenyl]methanamine (2.31 g, 9.05 mmol, 1.00 equiv), DCM (20 mL). This was followed by the addition of DIEA (2.34 g, 18.11 mmol, 2.00 equiv) dropwise with stirring at 0° C. The resulting solution was stirred for 2 h at room temperature. The resulting solution was diluted with 50 mL of water. The resulting solution was extracted with 2×50 mL of DCM and the organic layers combined and dried over anhydrous sodium sulfate. The solids were filtered out. The resulting mixture was concentrated under vacuum. The residue was applied onto a silica gel column with EA/PE (1/100-1/1). This resulted in 1.6 g (40%) of ethyl 2-chloro-4-[([4-[1-methyl-4-(trifluoromethyl)-1H-imidazol-2-yl]phenyl]methyl)amino]pyrimidine-5-carboxylate as yellow oil. LCMS (ES, m/z): 440 [M+H]+.

### Step 2. Synthesis of ethyl 4-[([4-[1-methyl-4-(trifluoromethyl)-1H-imidazol-2-yl]phenyl]methyl)amino]-2-[2-(propan-2-yl)phenyl]pyrimidine-5-carboxylate

Into a 100 mL round-bottom flask purged and maintained with an inert atmosphere of nitrogen, was placed ethyl 2-chloro-4-[([4-[1-methyl-4-(trifluoromethyl)-1H-imidazol-2-yl]phenyl]methyl)amino]pyrimidine-5-carboxylate (1.6 g, 3.64 mmol, 1.00 equiv), [2-(propan-2-yl)phenyl]boronic acid (782 mg, 4.77 mmol, 1.00 equiv), Pd(dppf)Cl2·CH2Cl2 (389 mg, 0.48 mmol, 0.10 equiv), potassium carbonate (1.32 g, 9.55 mmol, 2.00 equiv), dioxane (25 mL), water (5 mL). The resulting solution was stirred for 3 h at 80° C. in an oil bath. The reaction mixture was cooled to room temperature. The solids were filtered out. The filter cakes were washed with 2×50 mL of EA. The filtrate was washed with 100 mL of brine. The mixture was dried over anhydrous sodium sulfate and concentrated under vacuum. The residue was applied onto a silica gel column with EA/hexane (1/100-1/1). This resulted in 1 g (53%) of ethyl 4-[([4-[1-methyl-4-(trifluoromethyl)-1H-imidazol-2-yl]phenyl]methyl)amino]-2-[2-(propan-2-yl)phenyl]pyrimidine-5-carboxylate as yellow oil. LCMS (ES, m/z): 524 [M+H]+.

### Step 3. Synthesis of [4-[([4-[1-methyl-4-(trifluoromethyl)-1H-imidazol-2-yl]phenyl]methyl)amino]-2-[2-(propan-2-yl)phenyl]pyrimidin-5-yl]methanol

Into a 50 mL 3-necked round-bottom flask purged and maintained with an inert atmosphere of nitrogen, was placed ethyl 4-[([4-[1-methyl-4-(trifluoromethyl)-1H-imidazol-2-yl]phenyl]methyl)amino]-2-[2-(propan-2-yl)phenyl]pyrimidine-5-carboxylate (500 mg, 0.96 mmol, 1.00 equiv), tetrahydrofuran (15 mL). This was followed by the addition of LAH (54 mg, 1.55 mmol, 1.50 equiv) in several batches at 0° C. The resulting solution was stirred for 2 h at 0° C. The reaction was then quenched by the addition of 300 mg of sodium sulfate decahydrate. The solids were filtered out. The filter cake was washed with 10 mL of methanol. The combined filter was concentrated under vacuum. This resulted in 200 mg (43%) of [4-[([4-[1-methyl-4-(trifluoromethyl)-1H-imidazol-2-yl]phenyl]methyl)amino]-2-[2-(propan-2-yl)phenyl]pyrimidin-5-yl]methanol as a white solid. LCMS (ES, m/z): 482 [M+H]+.

### Step 4. Synthesis of 1-([4-[1-methyl-4-(trifluoromethyl)-1H-imidazol-2-yl]phenyl]methyl)-7-[2-(propan-2-yl)phenyl]-1H,2H, 4H-pyrimido[4,5-d][1,3]oxazin-2-one (I-7)

Into a 8 mL round-bottom flask, was placed [4-[([4-[1-methyl-4-(trifluoromethyl)-1H-imidazol-2-yl]phenyl]methyl)amino]-2-[2-(propan-2-yl)phenyl]pyrimidin-5-yl]methanol (60 mg, 0.12 mmol, 1.00 equiv), DCM (1 mL), DIEA (81 mg, 0.63 mmol, 5.00 equiv). This was followed by the addition of a solution of ditrichloromethyl carbonate (111 mg, 0.37 mmol, 3.00 equiv) in dichloromethane (1 mL) dropwise with stirring at 0° C. The resulting solution was stirred for 1 h at 0° C. The reaction was then quenched by the addition of 2 mL of water. The resulting solution was extracted with 2×2 mL of dichloromethane and the organic layers combined and concentrated under vacuum. The crude product was purified by Prep-HPLC with the following conditions (Prep-HPLC-025): Column, XBridge Prep Shield RP18 OBD Column, 19×150 mm, 5 um-13 nm; mobile phase, water with 0.05% NH4HCO3 and ACN (35% ACN up to 81% in 10 min); Detector, 220/254 nm. This resulted in 11.8 mg (19%) of 1-([4-[1-methyl-4-(trifluoromethyl)-1H-imidazol-2-yl]phenyl]methyl)-7-[2-(propan-2-yl)phenyl]-1H,2H, 4H-pyrimido[4,5-d][1,3]oxazin-2-one as a white solid. LC-MS (ESI) m/z 508.2 [M+H]+ 1H NMR (300 MHz, CD3OD) δ 8.61 (s, 1H), 7.69 (s, 1H), 7.64-7.61 (m, 2H), 7.54-7.49 (m, 3H), 7.45-7.44 (m, 2H), 7.30-7.25 (m, 1H), 5.56 (s, 2H), 5.43 (s, 2H), 3.77 (s, 3H), 3.46-3.42 (m, 1H), 1.11 (d, J=6.9 Hz, 6H).

### Example 8: Synthesis of 1-([4-[1-methyl-4-(trifluoromethyl)-1H-imidazol-2-yl]phenyl]methyl)-7-[2-(propan-2-yl)pyridin-3-yl]-1H,2H, 4H-pyrimido[4,5-d][1,3]oxazin-2-one (I-8)

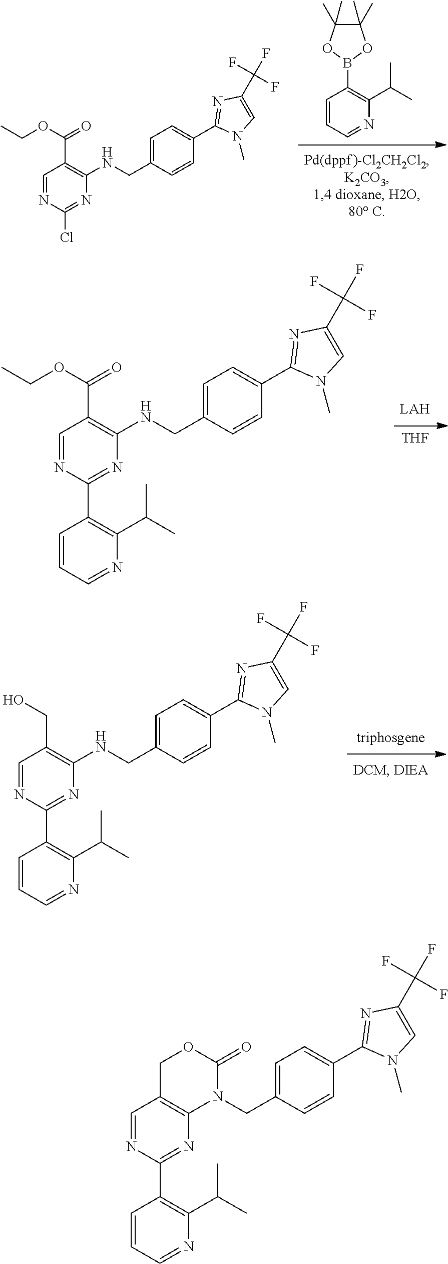 [SMILES: CCOC(=O)c1cnc(Cl)nc1NCc1ccc(-c2nc(C(F)(F)F)cn2C)cc1 ; CC(C)c1ncccc1B1OC(C)(C)C(C)(C)O1 ; CCOC(=O)c1cnc(-c2cccnc2C(C)C)nc1NCc1ccc(-c2nc(C(F)(F)F)cn2C)cc1 ; "THF" ; CC(C)c1ncccc1-c1ncc(CO)c(NCc2ccc(-c3nc(C(F)(F)F)cn3C)cc2)n1 ; CC(C)c1ncccc1-c1ncc2c(n1)N(Cc1ccc(-c3nc(C(F)(F)F)cn3C)cc1)C(=O)OC2]

### Step 1. Synthesis of ethyl 4-[([4-[1-methyl-4-(trifluoromethyl)-1H-imidazol-2-yl]phenyl]methyl)amino]-2-[2-(propan-2-yl)pyridin-3-yl]pyrimidine-5-carboxylate

Into a 100-mL round-bottom flask purged and maintained with an inert atmosphere of nitrogen, was placed ethyl 2-chloro-4-[([4-[1-methyl-4-(trifluoromethyl)-1H-imidazol-2-yl]phenyl]methyl)amino]pyrimidine-5-carboxylate (550 mg, 1.25 mmol, 1.00 equiv), 2-(propan-2-yl)-3-(tetramethyl-1,3,2-dioxaborolan-2-yl)pyridine (309.45 mg, 1.25 mmol, 1.00 equiv), Pd(dppf)Cl2·CH2Cl2 (102.23 mg, 0.13 mmol, 0.10 equiv), potassium carbonate (345.78 mg, 2.50 mmol, 2.00 equiv), water (2 mL), dioxane (10 mL). The resulting solution was stirred for 3 h at 80° C. The reaction mixture was cooled to room temperature. The resulting mixture was concentrated under vacuum. The residue was applied onto a silica gel column with EA/petroleum ether (0˜35%). This resulted in 520 mg (79%) of ethyl 4-[([4-[1-methyl-4-(trifluoromethyl)-1H-imidazol-2-yl]phenyl]methyl)amino]-2-[2-(propan-2-yl)pyridin-3-yl]pyrimidine-5-carboxylate as yellow oil. LC-MS-PH-FMA-PJ111-805-2: (ES, m/z): 524[M+H]+.

### Step 2. Synthesis of [4-[([4-[1-methyl-4-(trifluoromethyl)-1H-imidazol-2-yl]phenyl]methyl)amino]-2-[2-(propan-2-yl)pyridin-3-yl]pyrimidin-5-yl]methanol

Into a 100-mL round-bottom flask, was placed ethyl 4-[([4-[1-methyl-4-(trifluoromethyl)-1H-imidazol-2-yl]phenyl]methyl)amino]-2-[2-(propan-2-yl)pyridin-3-yl]pyrimidine-5-carboxylate (520 mg, 0.99 mmol, 1.00 equiv), LAH (38.2 mg, 1.09 mmol, 1.10 equiv), tetrahydrofuran (10 mL). The resulting solution was stirred for 2 h at 0° C. The reaction was then quenched by the addition of sodium sulfate decahydrate. The solids were filtered out. The resulting mixture was concentrated under vacuum. This resulted in 260 mg (crude) of [4-[([4-[1-methyl-4-(trifluoromethyl)-1H-imidazol-2-yl]phenyl]methyl)amino]-2-[2-(propan-2-yl)pyridin-3-yl]pyrimidin-5-yl]methanol as yellow oil. LC-MS-PH-FMA-PJ111-805-3: (ES, m/z): 482[M+H]+.

### Step 3. Synthesis of 1-([4-[1-methyl-4-(trifluoromethyl)-1H-imidazol-2-yl]phenyl]methyl)-7-[2-(propan-2-yl)pyridin-3-yl]-1H,2H, 4H-pyrimido[4,5-d][1,3]oxazin-2-one (I-8)

Into a 100-mL round-bottom flask purged and maintained with an inert atmosphere of nitrogen, was placed [4-[([4-[1-methyl-4-(trifluoromethyl)-1H-imidazol-2-yl]phenyl]methyl)amino]-2-[2-(propan-2-yl)pyridin-3-yl]pyrimidin-5-yl]methanol (50 mg, 0.10 mmol, 1.00 equiv), ditrichloromethyl carbonate (92.4 mg, 0.31 mmol, 3.00 equiv), dichloromethane (8 mL), DIEA (66.9 mg, 0.52 mmol, 5.00 equiv). The resulting solution was stirred overnight at 20° C. The reaction was then quenched by the addition of 10 mL of methanol. The resulting mixture was concentrated under vacuum. The residue was applied onto a prep-TLC plate and eluted with dichloromethane/methanol (20:1). The crude product was purified by Prep-HPLC with the following conditions: Column, XBridge C18 OBD Prep Column, 100A, 5 μm, 19 mm×250 mm; mobile phase, water (10 mmOL/L NH4HCO3) and ACN (hold 40.0% ACN in 12 min); Detector, UV 220/254 nm. This resulted in 18.3 mg (35%) of 1-([4-[1-methyl-4-(trifluoromethyl)-1H-imidazol-2-yl]phenyl]methyl)-7-[2-(propan-2-yl)pyridin-3-yl]-1H,2H, 4H-pyrimido[4,5-d][1,3]oxazin-2-one as a white solid. LC-MS-PH-FMA-PJ111-805-0: (ES, m/z): 508[M+H]+ H-NMR: (400 MHZ, Methanol-d4) δ 8.63 (s, 1H), 8.56 (d, J=4.8 Hz, 1H), 7.99-7.97 (m, 1H), 7.67 (s, 1H), 7.64-7.58 (m 2H), 7.51-7.47 (m, 2H), 7.32-7.29 (m, 1H), 5.54 (s, 2H), 5.41 (s, 2H), 3.75 (s, 3H), 3.65-3.58 (m, 1H), 1.14 (d, J=6.8 Hz, 6H.

### Example 9: Synthesis 3-[7-([4-[1-methyl-4-(trifluoromethyl)-1H-imidazol-2-yl]phenyl]methyl)-7H-pyrrolo[2,3-d]pyrimidin-2-yl]-2-(propan-2-yl)pyridine (I-9)

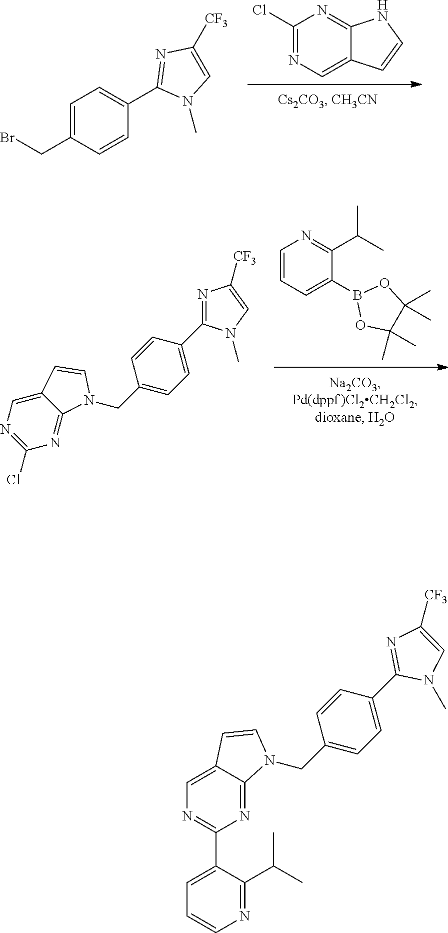

### Step 1. Synthesis of 2-[4-([2-chloro-7H-pyrrolo[2,3-d]pyrimidin-7-yl]methyl)phenyl]-1-methyl-4-(trifluoromethyl)-1H-imidazole

To a stirred mixture of 2-chloro-7H-pyrrolo[2,3-d]pyrimidine (241 mg, 1.57 mmol, 1.00 equiv) and 2-[4-(bromomethyl)phenyl]-1-methyl-4-(trifluoromethyl)-1H-imidazole (500 mg, 1.57 mmol, 1.00 equiv) in ACN (10 mL) was added Cs₂CO₃ (766 mg, 2.35 mmol, 1.50 equiv). The resulting solution was stirred for 1 h at 80° C. The reaction mixture was cooled to 25° C. The solids were filtered out. The resulting mixture was concentrated under vacuum. The residue was applied onto a silica gel column with PE/EA (0˜30%). This resulted in 600 mg (93%) of 2-[4-([2-chloro-7H-pyrrolo[2,3-d]pyrimidin-7-yl]methyl)phenyl]-1-methyl-4-(trifluoromethyl)-1H-imidazole as a white solid. LC/MS (ES, m/z): 392, 394 [M+H]+.

### Step 2. Synthesis of 3-[7-([4-[1-methyl-4-(trifluoromethyl)-1H-imidazol-2-yl]phenyl]methyl)-7H-pyrrolo[2,3-d]pyrimidin-2-yl]-2-(propan-2-yl)pyridine (I-9)

To a stirred mixture of 2-[4-([2-chloro-7H-pyrrolo[2,3-d]pyrimidin-7-yl]methyl)phenyl]-1-methyl-4-(trifluoromethyl)-1H-imidazole (100 mg, 0.23 mmol, 1.00 equiv) and 2-(propan-2-yl)-3-(tetramethyl-1,3,2-dioxaborolan-2-yl)pyridine (76 mg, 0.31 mmol, 1.205 equiv) in dioxane (5 mL) and water (1 mL) was added Pd(dppf)Cl₂ CH₂Cl₂ (21 mg, 0.03 mmol, 0.101 equiv), potassium carbonate (105 mg, 0.76 mmol, 2.98 equiv). The resulting solution was stirred overnight at 80° C. The reaction mixture was cooled to 25° C. The resulting solution was diluted with 20 mL of water. The resulting solution was extracted with 3×20 mL of EA and the organic layers combined and concentrated under vacuum. The residue was applied onto a silica gel column with EA/petroleum ether (1:1). The crude product was purified by Prep-HPLC with the following conditions (Column, XBridge C18 OBD Prep Column, 100A, 5 um, 19 mm×250 mm; Mobile phase, water (10 mmol/L NH₄HCO₃) and ACN (41.0% ACN up to 61.0% in 7 min); Detector, UV 254 nm). This resulted in 21 mg of 3-[7-([4-[1-methyl-4-(trifluoromethyl)-1H-imidazol-2-yl]phenyl]methyl)-7H-pyrrolo[2,3-d]pyrimidin-2-yl]-2-(propan-2-yl)pyridine as a white solid. LCMS (ESI) m/z 477 [M+H]^{+ 1}H NMR (400 MHZ, DMSO-d₆) δ 9.18 (s, 1H), 8.61-8.60 (m, 1H), 8.06-8.04 (m, 1H), 7.90 (s, 1H), 7.84 (d, J=3.6 Hz, 1H), 7.69-7.67 (m, 2H), 7.38-7.36 (m, 2H), 7.34-7.31 (m, 1H), 6.75 (d, J=3.6 Hz, 1H), 5.60 (s, 2H), 3.75 (s, 3H), 3.73-3.68 (m, 1H) 1.16 (d, J=6.4 Hz, 6H).

### Example 10: Synthesis of 3-[1-([4-[1-methyl-4-(trifluoromethyl)-1H-imidazol-2-yl]phenyl]methyl)-1H-pyrazolo[3,4-d]pyrimidin-6-yl]-2-(propan-2-yl)pyridine (I-10)

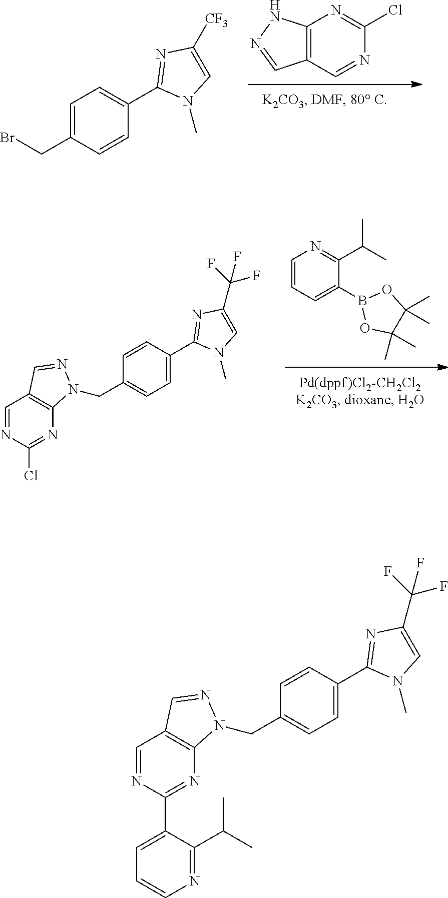

### Step 1. Synthesis of 2-[4-([6-chloro-1H-pyrazolo[3,4-d]pyrimidin-1-yl]methyl)phenyl]-1-methyl-4-(trifluoromethyl)-1H-imidazole

Into a 100-mL round-bottom flask, was placed 6-chloro-1H-pyrazolo[3,4-d]pyrimidine (600 mg, 3.88 mmol, 1.00 equiv), DMF (15 mL), potassium carbonate (1.07 g, 7.74 mmol, 1.99 equiv), 2-[4-(bromomethyl)phenyl]-1-methyl-4-(trifluoromethyl)-1H-imidazole (1.49 g, 4.67 mmol). The resulting solution was stirred for 3 h at 80° C. in an oil bath. The solids were filtered out. The resulting mixture was concentrated under vacuum. The residue was applied onto a silica gel column with EA/petroleum ether (100-0%). This resulted in 250 mg (16%) of 2-[4-([6-chloro-1H-pyrazolo[3,4-d]pyrimidin-1-yl]methyl)phenyl]-1-methyl-4-(trifluoromethyl)-1H-imidazole as a white solid. And 250 mg (16%) of 2-[4-([6-chloro-2H-pyrazolo[3,4-d]pyrimidin-2-yl]methyl)phenyl]-1-methyl-4-(trifluoromethyl)-1H-imidazole as a white solid. LC-MS (ESI) m/z 393.1 [M+H]+.

### Step 2. Synthesis of 3-[1-([4-[1-methyl-4-(trifluoromethyl)-1H-imidazol-2-yl]phenyl]methyl)-1H-pyrazolo[3,4-d]pyrimidin-6-yl]-2-(propan-2-yl)pyridine (I-10)

Into a 100-mL round-bottom flask purged and maintained with an inert atmosphere of nitrogen, was placed 2-[4-([6-chloro-1H-pyrazolo[3,4-d]pyrimidin-1-yl]methyl)phenyl]-1-methyl-4-(trifluoromethyl)-1H-imidazole (250 mg, 0.64 mmol, 1.00 equiv), 2-(propan-2-yl)-3-(tetramethyl-1,3,2-dioxaborolan-2-yl)pyridine (189 mg, 0.76 mmol, 1.20 equiv), potassium carbonate (176 mg, 1.27 mmol, 2.00 equiv), dioxane (15 mL), water (3 mL), Pd(dppf)Cl2 Cl2Cl2 (52 mg, 0.06 mmol, 0.100 equiv). The resulting solution was stirred overnight at 105° C. in an oil bath. The reaction mixture was cooled. The solids were filtered out. The resulting mixture was concentrated under vacuum. The residue was applied onto a silica gel column with EA/petroleum ether (90-0%). The crude product was purified by Prep-HPLC with the following conditions (Column, XBridge Shield RP18 OBD Column, 5 μm, 19×150 mm; Mobile phase, water (10 mmol/L NH₄HCO₃) and ACN (35.0% ACN up to 75.0% in 7 min); Detector, UV 220 nm). This resulted in 47.7 mg (16%) of 3-[1-([4-[1-methyl-4-(trifluoromethyl)-1H-imidazol-2-yl]phenyl]methyl)-1H-pyrazolo[3,4-d]pyrimidin-6-yl]-2-(propan-2-yl)pyridine as a white solid. LC-MS (ESI) m/z 478.0 [M+H]+ 1H NMR (300 MHZ, DMSO-d6) δ 9.51 (s, 1H), 8.73-8.64 (m, 1H), 8.51 (s, 1H), 8.19-8.09 (m, 1H), 7.91 (s, 1H), 7.71 (d, J=8.1 Hz, 2H), 7.41 (d, J=8.4 Hz, 2H), 7.107.35 (m, 1H), 5.79 (s, 2H), 3.74 (s, 3H), 3.73-3.65 (m, 1H), 1.21 (d, J=6.6 Hz, 6H).

### Example 11: Synthesis 3′-([4-[1-methyl-4-(trifluoromethyl)-1H-imidazol-2-yl]phenyl]methyl)-5′-[2-(propan-2-yl)pyridin-3-yl]-2′,3′-dihydrospiro[cyclopropane-1,1′-pyrrolo[2,3-d]pyrimidine]-2′-one (I-11)

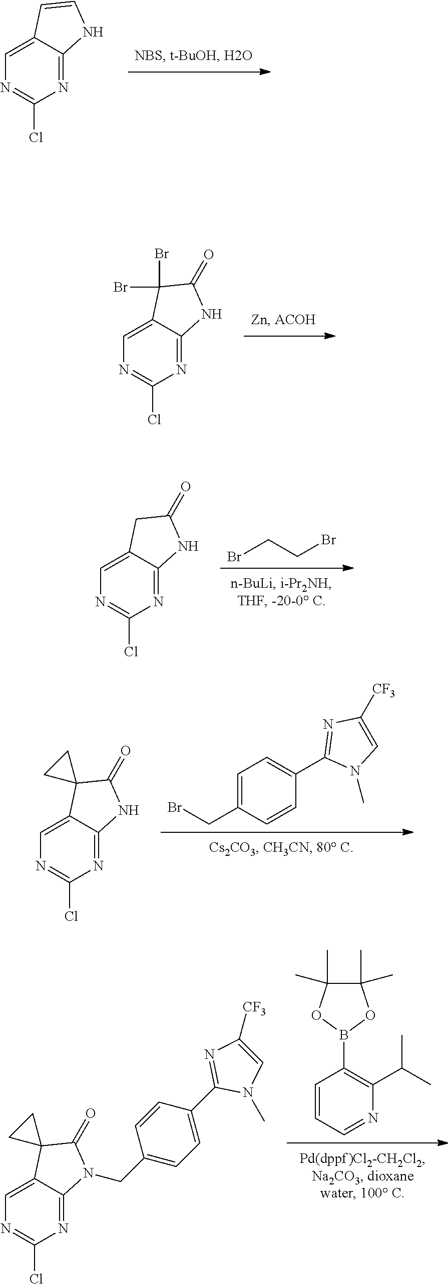

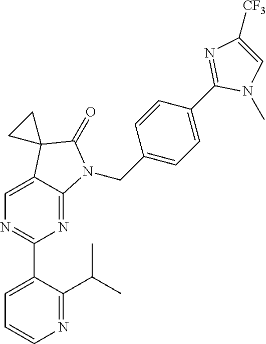

### Step 1. Synthesis of 5,5-dibromo-2-chloro-5H,6H, 7H-pyrrolo[2,3-d]pyrimidin-6-one

Into a 1000-mL roundbottom flask, was placed 2-chloro-7H-pyrrolo[2,3-d]pyrimidine (7 g, 45.58 mmol, 1.00 equiv), tert-Butanol (150 mL), water (40 mL). This was followed by the addition of NBS (48.55 g, 272.78 mmol, 6.00 equiv) in several batches at 0° C. The resulting solution was stirred overnight at room temperature. The resulting solution was diluted with 200 mL of water. The resulting solution was extracted with 2×200 mL of EA and the organic layers combined. The organic layers were washed with brine (200 mL) and dried over sodium sulfate. The solids were filtered out. The resulting mixture was concentrated under vacuum. This resulted in 13 g (87%) of 5,5-dibromo-2-chloro-5H,6H, 7H-pyrrolo[2,3-d]pyrimidin-6-one as a yellow solid. LC-MS (ESI) m/z 326[M+H]+.

### Step 2. Synthesis of 2-chloro-5H,6H, 7H-pyrrolo[2,3-d]pyrimidin-6-one

Into a 500-mL round-bottom flask, was placed 5,5-dibromo-2-chloro-5H,6H, 7H-pyrrolo[2,3-d]pyrimidin-6-one (8 g, 24.44 mmol, 1.00 equiv), AcOH (80 mL), tetrahydrofuran (20 mL). This was followed by the addition of Zn (9.5 g, 145.24 mmol, 6.00 equiv) in several batches at 0° C. The resulting solution was stirred for 3 h at room temperature (20° C.). The solids were filtered out. The filtrate was concentrated under vacuum. The residue was applied onto a silica gel column with EA/petroleum ether (50-100%). This resulted in 3 g (72%) of 2-chloro-5H,6H, 7H-pyrrolo[2,3-d]pyrimidin-6-one as yellow oil. LC-MS (ESI) m/z 170 [M+H]+.

### Step 3. Synthesis of 5′-chloro-2′,3′-dihydrospiro[cyclopropane-1,1′-pyrrolo[2,3-d]pyrimidine]-2′-one

Into a 50-mL 3-necked round-bottom flask purged and maintained with an inert atmosphere of nitrogen, was placed 2-chloro-5H,6H,7H-pyrrolo[2,3-d]pyrimidin-6-one (500 mg, 2.80 mmol, 1.00 equiv), tetrahydrofuran (6 mL), i-Pr2NH (594 mg, 5.87 mmol, 2.00 equiv). This was followed by the addition of n-BuLi (2.5 M) (4.7 mL, 11.7 mmol, 4.00 equiv) dropwise with stirring at −40° C. The above mixture was stirred for 0.5 h at −40° C. and warmed to 0° C. To this was added 1,2-dibromoethane (1.66 g, 8.84 mmol, 3.00 equiv) dropwise with stirring at 0° C. The resulting solution was stirred overnight at 20° C. The reaction mixture was cooled to 0° C. The reaction was then quenched by the addition of 20 mL of NH₄Cl (sat., aq.). The resulting solution was extracted with 2×20 mL of EA and the organic layers combined and concentrated under vacuum. The residue was purified by preparative TLC (EA:PE=1:1). This resulted in 50 mg (9%) of 5′-chloro-2′,3′-dihydrospiro[cyclopropane-1,1′-pyrrolo[2,3-d]pyrimidine]-2′-one as a brown solid. LC-MS (ESI) m/z 195.9 [M+H]+.

### Step 4. Synthesis of 5′-chloro-3′-([4-[1-methyl-4-(trifluoromethyl)-1H-imidazol-2-yl]phenyl]methyl)-2′,3′-dihydrospiro[cyclopropane-1,1′-pyrrolo[2,3-d]pyrimidine]-2′-one

Into a 8-mL vial, was placed 5′-chloro-2′,3′-dihydrospiro[cyclopropane-1,1′-pyrrolo[2,3-d]pyrimidine]-2′-one (100 mg, 0.49 mmol, 1.00 equiv, 95%), 2-[4-(bromomethyl)phenyl]-1-methyl-4-(trifluoromethyl)-1H-imidazole (164 mg, 0.49 mmol, 1.00 equiv, 95%), Cs2CO3 (334 mg, 1.03 mmol, 2.00 equiv), ACN (3 mL). The resulting solution was stirred for 30 min at 80° C. The reaction mixture was cooled to 25° C. The solids were filtered out. The filtrate was concentrated under vacuum. The residue was purified by preparative TLC (EA:PE=1:1). This resulted in 40 mg (18%) of 5′-chloro-3′-([4-[1-methyl-4-(trifluoromethyl)-1H-imidazol-2-yl]phenyl]methyl)-2′,3′-dihydrospiro[cyclopropane-1,1′-pyrrolo[2,3-d]pyrimidine]-2′-one as a white solid. LC-MS (ESI) m/z 434.2 [M+H]+.

### Step 5. Synthesis of 3′-([4-[1-methyl-4-(trifluoromethyl-)1H-imidazol-2-yl]phenyl]methyl)-5′-[2-(propan-2-yl)pyridin-3-yl]-2′,3′-dihydrospiro[cyclopropane-1,1′-pyrrolo[2,3-d]pyrimidine]-2′-one (I-11)

Into a 25-mL round-bottom flask purged and maintained with an inert atmosphere of nitrogen, was placed 5′-chloro-3′-([4-[1-methyl-4-(trifluoromethyl)-1H-imidazol-2-yl]phenyl]methyl)-2′,3′-dihydrospiro[cyclopropane-1,1′-pyrrolo[2,3-d]pyrimidine]-2′-one (50 mg, 0.11 mmol, 1.00 equiv), 2-(propan-2-yl)-3-(tetramethyl-1,3,2-dioxaborolan-2-yl)pyridine (43 mg, 0.17 mmol, 1.50 equiv, 95%), Pd(dppf)Cl2 CH2Cl2 (9.4 mg, 0.01 mmol, 0.10 equiv), sodium carbonate (24 mg, 0.23 mmol, 2.00 equiv), dioxane (10 mL), water (2 mL). The resulting solution was stirred for 3 h at 100° C. The reaction mixture was cooled 25° C. The mixture was filtered through a Celite pad. The filtrate was concentrated under vacuum. The residue was purified by preparative TLC (EA:PE=1:1). The crude product was purified by Prep-HPLC with the following conditions (Prep-HPLC-025): Column: XBridge Prep C18 OBD Column 19×150 mm 5 um; Mobile Phase A: water (0.05% ammonia in water), Mobile Phase B: ACN; Flow rate: 20 mL/min; Gradient: 25% B to 55% B in 7 min; 254 nm. This resulted in 9.4 mg (16%) of 3′-([4-[1-methyl-4-(trifluoromethyl)-1H-imidazol-2-yl]phenyl]methyl)-5′-[2-(propan-2-yl)pyridin-3-yl]-2′,3′-dihydrospiro[cyclopropane-1,1′-pyrrolo[2,3-d]pyrimidine]-2′-one as a white solid. LC-MS (ESI) m/z 519.3 [M+H]+ 1H NMR (400 MHZ, CD3OD) δ 8.60-8.59 (m, 1H), 8.35 (s, 1H), 8.04-8.01 (m, 1H), 7.69 (s, 1H), 7.65 (d, J=8.0 Hz, 2H), 7.58 (d, J=8.4 Hz, 2H), 7.38-7.35 (m, 1H), 5.59 (s, 2H), 3.77 (s, 3H), 3.66-3.59 (m, 1H), 1.99-1.88 (m, 4H), 1.23 (d, J=6.8 Hz, 6H).

### Example 12: Synthesis of 5-(2-isopropylphenyl)-3-(4-(1-methyl-4-(trifluoromethyl)-1H-imidazol-2-yl)benzyl)oxazolo[4,5-d]pyrimidin-2(3H)-one (I-12)

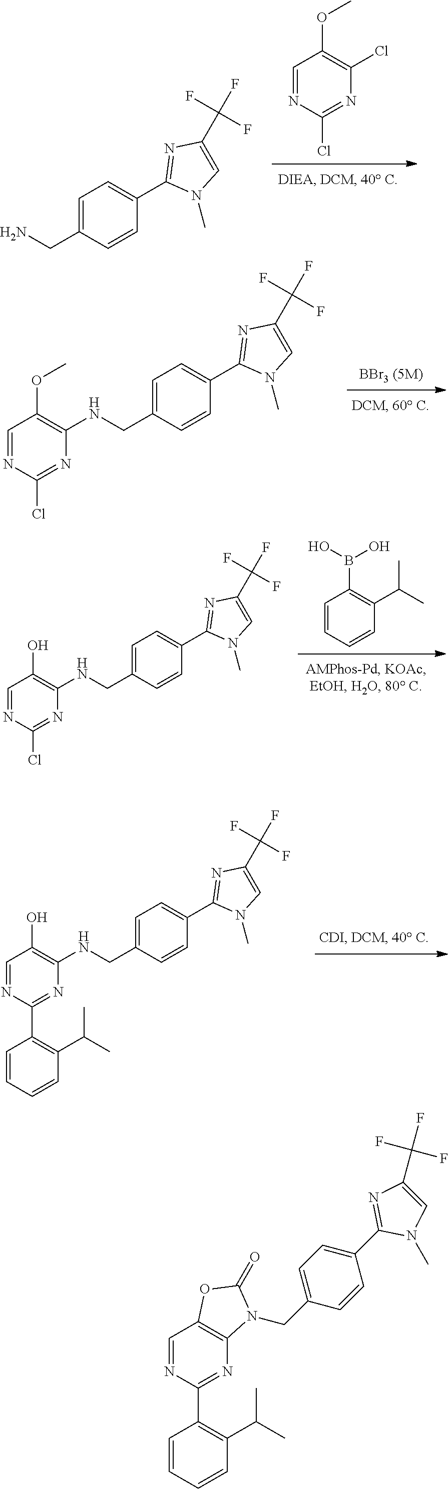

### Step 1. Synthesis of 2-chloro-5-methoxy-N-([4-[1-methyl-4-(trifluoromethyl)-1H-imidazol-2-yl]phenyl]methyl)pyrimidin-4-amine

Into a 250 mL round-bottom flask was placed 2,4-dichloro-5-methoxypyrimidine (2.10 g, 11.75 mmol, 1.20 equiv), dichloromethane (30 mL), DIEA (2.53 g, 19.59 mmol, 2.00 equiv). This was followed by the addition of a solution of [4-[1-methyl-4-(trifluoromethyl)-1H-imidazol-2-yl]phenyl]methanamine (2.5 g, 9.79 mmol, 1.00 equiv) in dichloromethane (10 mL) dropwise with stirring at 0° C. in 30 min. The resulting solution was stirred overnight at 17° C. The resulting mixture was concentrated under vacuum. The residue was applied onto a silica gel column (eluting with EA/petroleum ether 3/1) to afford 1.4 g (36%) of 2-chloro-5-methoxy-N-([4-[1-methyl-4-(trifluoromethyl)-1H-imidazol-2-yl]phenyl]methyl)pyrimidin-4-amine as a yellow solid. LC-MS (ESI) m/z 398.1 [M+H]+.

### Step 2. Synthesis of 2-chloro-4-[([4-[1-methyl-4-(trifluoromethyl)-1H-imidazol-2-yl]phenyl]methyl)-amino]pyrimidin-5-ol

Into a 250 mL round-bottom flask was placed 2-chloro-5-methoxy-N-([4-[1-methyl-4-(trifluoromethyl)-1H-imidazol-2-yl]phenyl]methyl)pyrimidin-4-amine (1.4 g, 3.52 mmol, 1.00 equiv), dichloromethane (10 mL), BBr₃ (10 mL). The resulting solution was stirred for 4 h at 60° C. After cooling to room temperature, the reaction mixture was poured into ice/water, sodium hydroxide solution (1 mol/L) was employed to adjust the pH to 6-7, extracted with EA (100 ml×3). The organic layers were combined, washed with brine (100 mL×2), dried over anhydrous sodium sulfate and concentrated under vacuum. The residue was applied onto a silica gel column (eluting with dichloromethane/methanol 10/1) to afford 1.1 g (81%) of 2-chloro-4-[([4-[1-methyl-4-(trifluoromethyl)-1H-imidazol-2-yl]phenyl]methyl)amino]pyrimidin-5-ol as a yellow solid. LC-MS (ESI) m/z 384.0 [M+H]+.

### Step 3. Synthesis of 4-[([4-[1-methyl-4-(trifluoromethyl)-1H-imidazol-2-yl]phenyl]methyl)amino]-2-[2-(propan-2-yl)phenyl]pyrimidin-5-ol

Into a 8 mL sealed tube purged and maintained with an inert atmosphere of nitrogen, was placed 2-chloro-4-[([4-[1-methyl-4-(trifluoromethyl)-1H-imidazol-2-yl]phenyl]methyl)amino]pyrimidin-5-ol (50 mg, 0.13 mmol, 1.00 equiv), [2-(propan-2-yl)phenyl]boronic acid (107 mg, 0.65 mmol, 5.00 equiv), Pd(amphos)C12 (9 mg, 0.01 mmol, 0.10 equiv), KOAc (28 mg, 0.29 mmol, 2.20 equiv), ethanol (2 mL), water (0.5 mL). The resulting solution was stirred for 2 h at 80° C. with microwave. After cooling to room temperature, the reaction mixture was concentrated under vacuum. The residue was applied onto a silica gel column (eluting with dichloromethane/methanol 7/3) to afford 41 mg (67%) of 4-[([4-[1-methyl-4-(trifluoromethyl)-1H-imidazol-2-yl]phenyl]methyl)amino]-2-[2-(propan-2-yl)phenyl]pyrimidin-5-ol as a colorless solid. LC-MS (ESI) m/z 468.2 [M+H]+.

### Step 4. Synthesis of 5-(2-isopropylphenyl)-3-(4-(1-methyl-4-(trifluoromethyl)-1H-imidazol-2-yl)benzyl)oxazolo[4,5-d]pyrimidin-2(3H)-one (I-12)

Into a 8-mL sealed tube, was placed 4-[([4-[1-methyl-4-(trifluoromethyl)-1H-imidazol-2-yl]phenyl]methyl)amino]-2-[2-(propan-2-yl)phenyl]pyrimidin-5-ol (20 mg, 0.04 mmol, 1.00 equiv), DCE (2 mL), ditrichloromethyl carbonate (25 mg, 0.08 mmol, 2.00 equiv). The resulting solution was stirred for 3 h at 70° C. After cooling to room temperature, the reaction mixture was concentrated under vacuum. The residue was purified by Prep-HPLC with the following conditions (Column: XBridge C18 OBD Prep Column, 130 Å, 5 μm, 19 mm×250 mm; Mobile phase: water (0.1% FA), MeCN (56.0% MeCN over 11 min); Flow rate: 20 mL/min; Detector: 254 nm). This resulted in 8.4 mg (39.8%) of 5-(2-isopropylphenyl)-3-(4-(1-methyl-4-(trifluoromethyl)-1H-imidazol-2-yl)benzyl)oxazolo[4,5-d]pyrimidin-2(3H)-one as a white solid. LC-MS (ESI) m/z 494.2 [M+H]+. ¹H NMR (300 MHz, CD3OD-d4) δ 8.57 (s, 1H), 7.71-7.68 (m, 5H), 7.55-7.45 (m, 3H), 7.31-7.26 (m, 1H), 5.21 (s, 2H), 3.78 (s, 3H), 3.43-3.33 (m, 1H), 1.21 (d, J=6.3 Hz, 6H).

### Example 13: Synthesis of 3-([4-[1-methyl-4-(trifluoromethyl)-1H-imidazol-2-yl]phenyl]methyl)-5-[2-(propan-2-yl)phenyl]-2H,3H-[1,3]oxazolo[4,5-d]pyrimidin-2-one (I-13)

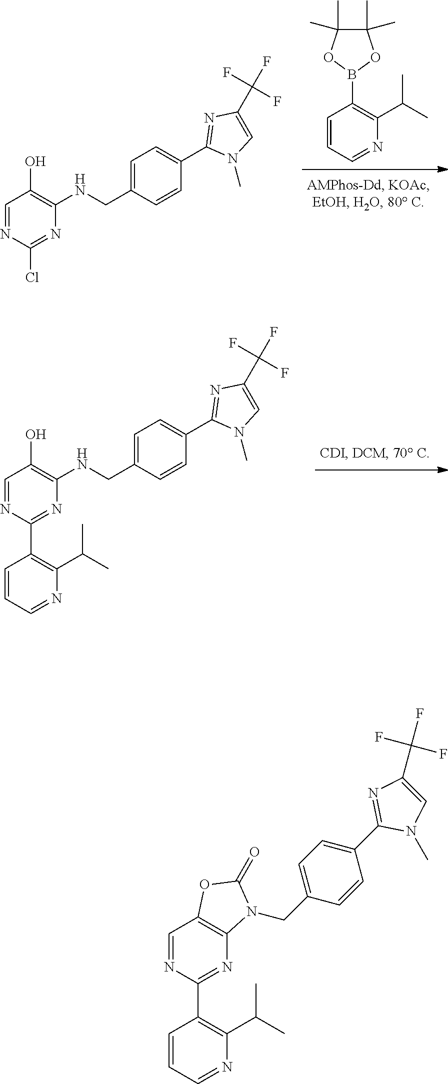

### Step 1. Synthesis of 4-[([4-[1-methyl-4-(trifluoromethyl)-1H-imidazol-2-yl]phenyl]methyl)amino]-2-[2-(propan-2-yl)pyridin-3-yl]pyrimidin-5-ol

Into a 5 mL sealed tube purged and maintained with an inert atmosphere of nitrogen was placed 2-chloro-4-[([4-[1-methyl-4-(trifluoromethyl)-1H-imidazol-2-yl]phenyl]methyl)amino]pyrimidin-5-ol (150 mg, 0.39 mmol, 1.00 equiv), 2-(propan-2-yl)-3-(tetramethyl-1,3,2-dioxaborolan-2-yl)pyridine (289 mg, 1.17 mmol, 3.00 equiv), Pd(amphos)Cl2 (28 mg, 0.04 mmol, 0.10 equiv), KOAc (84 mg, 0.86 mmol, 2.20 equiv), ethanol (3 mL), water (0.6 mL). The final reaction mixture was irradiated with microwave radiation for 2 h at 80° C. The reaction mixture was cooled to room temperature. The resulting mixture was concentrated under vacuum. The residue was applied onto a silica gel column with dichloromethane/methanol (20:1). This resulted in 40 mg (22%) of 4-[([4-[1-methyl-4-(trifluoromethyl)-1H-imidazol-2-yl]phenyl]methyl)amino]-2-[2-(propan-2-yl)pyridin-3-yl]pyrimidin-5-ol as a yellow solid. LC-MS(ESI) 469.3 [M+H]+.

### Step 2. Synthesis of 3-([4-[1-methyl-4-(trifluoromethyl)-1H-imidazol-2-yl]phenyl]methyl)-5-[2-(propan-2-yl)phenyl]-2H,3H-[1,3]oxazolo[4,5-d]pyrimidin-2-one (I-13)

Into a 8 mL vial, was placed 4-[([4-[1-methyl-4-(trifluoromethyl)-1H-imidazol-2-yl]phenyl]methyl)amino]-2-[2-(propan-2-yl)phenyl]pyrimidin-5-ol (20 mg, 0.04 mmol, 1.00 equiv), DCE (2 mL), CDI (20.8 mg, 0.13 mmol, 3.00 equiv). The resulting solution was stirred overnight at 70° C. in an oil bath. The reaction mixture was cooled to room temperature and concentrated under vacuum. The residue was purified by Prep-HPLC with the following conditions (Column:XBridge Prep C18 OBD Column, 19×150 mm 5 um; Mobile phase, water (10 mmOL/L NH₄HCO₃) and ACN (40.0% ACN up to 70.0% in 7 min); Detector: 254/220 nm. This resulted in 2.1 mg (10%) of 3-([4-[1-methyl-4-(trifluoromethyl)-1H-imidazol-2-yl]phenyl]methyl)-5-[2-(propan-2-yl)phenyl]-2H,3H-[1,3]oxazolo[4,5-d]pyrimidin-2-one as a white solid. LC-MS (ESI) m/z 495.2 [M+H]+ 1H NMR (400MHZ, DMSO-d6) δ 8.79 (s, 1H), 8.62 (s, 1H), 7.97-7.95 (m, 2H), 7.72-7.70 (d, J=8.0 Hz, 2H), 7.56 (d, J=8.4 Hz, 2H), 7.33-7.31 (m, 1H), 5.11 (s, 2H), 3.75 (s, 3H), 3.62-3.56 (m, 1H), 1.14 (d, J=6.80 Hz, 6H).

### Example 14: Synthesis of 8-(azetidin-3-yl)-2-[2-(propan-2-yl)phenyl]-9-[[4-(1H-pyrazol-1-yl)phenyl]methyl]-9H-purine (I-14)

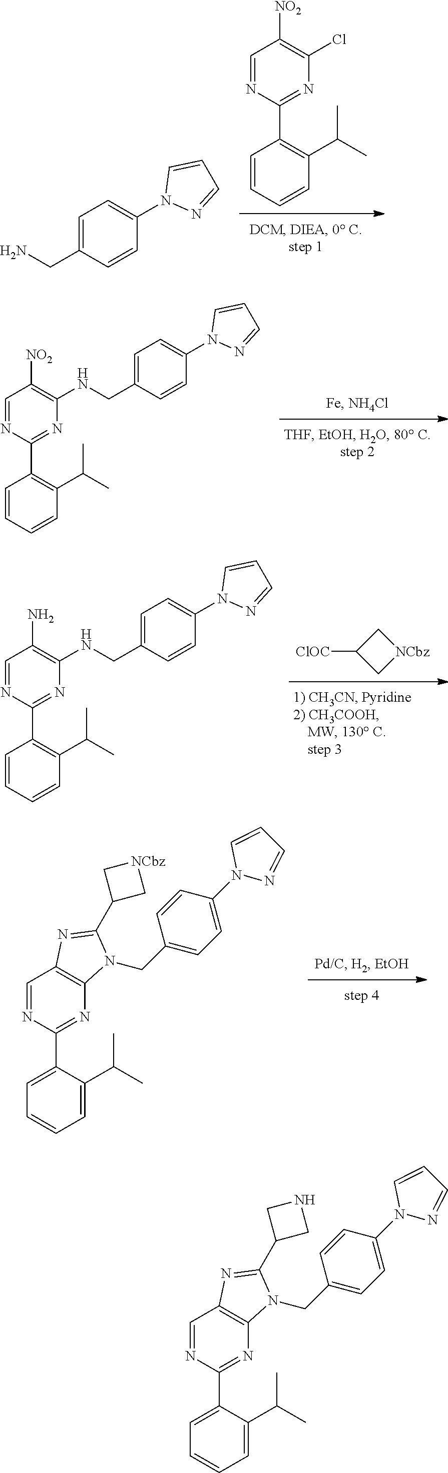

### Step 1. Synthesis of 5-nitro-2-[2-(propan-2-yl)phenyl]-N-[[4-(1H-pyrazol-1-yl)phenyl]methyl]pyrimidin-4-amine

Into a 100 mL round-bottom flask, was placed 4-chloro-5-nitro-2-[2-(propan-2-yl)phenyl]pyrimidine (800 mg, 2.88 mmol, 1 equiv), DIEA (1.1 g, 8.51 mmol, 3.00 equiv), dichloromethane (10 mL). This was followed by the addition of a solution of [4-(1H-pyrazol-1-yl)phenyl]methanamine (500 mg, 2.89 mmol, 1.00 equiv) in dichloromethane (10 mL) dropwise with stirring. The resulting solution was stirred for 2 h at 0° C. in a water/ice bath. The resulting mixture was concentrated under vacuum. The residue was applied onto a silica gel column with EA/PE (0-100%). This resulted in 800 mg (67%) of 5-nitro-2-[2-(propan-2-yl)phenyl]-N-[[4-(1H-pyrazol-1-yl)phenyl]methyl]pyrimidin-4-amine as a yellow solid. LC-MS (ESI) m/z 415.1[M+H]+.

### Step 2. Synthesis of 2-[2-(propan-2-yl)phenyl]-4-N-[[4-(1H-pyrazol-1-yl)phenyl]methyl]pyrimidine-4,5-diamine

Into a 100 mL round-bottom flask, was placed 5-nitro-2-[2-(propan-2-yl)phenyl]-N-[[4-(1H-pyrazol-1-yl)phenyl]methyl]pyrimidin-4-amine (800 mg, 1.93 mmol, 1.00 equiv), Fe (541 mg, 9.69 mmol, 5.00 equiv), NH4Cl (205 mg, 3.83 mmol, 2.00 equiv), tetrahydrofuran (7 mL), ethanol (7 mL), water (5 mL). The resulting solution was stirred for 1 h at 80° C. in an oil bath. The reaction mixture was cooled to room temperature. The mixture was filtered through a celite pad. The resulting solution was diluted with 20 mL of water. The resulting solution was extracted with 3×20 mL of EA. The organic layers were combined. The mixture was dried over anhydrous sodium sulfate. The solids were filtered out. The resulting mixture was concentrated under vacuum. This resulted in 600 mg (81%) of 2-[2-(propan-2-yl)phenyl]-4-N-[[4-(1H-pyrazol-1-yl)phenyl]methyl]pyrimidine-4,5-diamine as a yellow solid. LC-MS (ESI) m/z 385.0[M+H]+.

### Step 3. Synthesis of benzyl 3-[2-[2-(propan-2-yl)phenyl]-9-[[4-(1H-pyrazol-1-yl)phenyl]methyl]-9H-purin-8-yl]azetidine-1-carboxylate

Into a 100-mL round-bottom flask, was placed a solution of 2-[2-(propan-2-yl)phenyl]-4-N-[[4-(1H-pyrazol-1-yl)phenyl]methyl]pyrimidine-4,5-diamine (300 mg, 0.78 mmol, 1.00 equiv) in ACN (10 mL), pyridine (618 mg, 7.81 mmol, 10.01 equiv). This was followed by the addition of a solution of benzyl 3-(carbonochloridoyl)azetidine-1-carboxylate (216 mg, 0.85 mmol, 1.09 equiv) in ACN (20 mL) dropwise with stirring at room temperature in 1 h. The mixture was concentrated under vacuum. To this was added acetic acid (15 mL). The resulting solution was stirred for 1 h at 130° C. in a microwave reactor. The resulting solution was extracted with 20 mL of EA and the organic layers combined and dried over anhydrous sodium sulfate. The solids were filtered out. The resulting mixture was concentrated under vacuum. The residue was applied onto a TLC with EA/petroleum ether (1/1). This resulted in 200 mg (44%) of benzyl 3-[2-[2-(propan-2-yl)phenyl]-9-[[4-(1H-pyrazol-1-yl)phenyl]methyl]-9H-purin-8-yl]azetidine-1-carboxylate as a yellow solid. LC-MS−: (ES, m/z): 584[M+H]+.

### Step 4. Synthesis of 8-(azetidin-3-yl)-2-[2-(propan-2-yl)phenyl]-9-[[4-(1H-pyrazol-1-yl)phenylmethyl]-9H-purine (I-14)

Into a 100-mL round-bottom flask, was placed 7-[2-[2-(propan-2-yl)phenyl]-9-[[4-(1H-pyrazol-1-yl)phenyl]methyl]-9H-purin-8-yl]-3-oxa-5-azabicyclo[7.3.1]trideca-1(13), 9,11-trien-4-one (150 mg, 0.26 mmol, 1.00 equiv), ethanol (30 mL), Pd/C (100 mg, 10%). To the above H2 (g) was introduced in. The resulting solution was stirred for 35 min at room temperature. The solids were filtered out. The resulting mixture was concentrated under vacuum. The crude product was purified by Prep-HPLC with the following conditions: Column, XBridge BEH C18 OBD Prep Column, 5×19 mm; mobile phase, Mobile Phase A: water with 0.05% NH4HCO3, Mobile Phase B: ACN; Flow rate: 20 mL/min; Gradient: 25% B to 45% B in 10 min; Detector, 254&220 nm. This resulted in 4.0 mg (3%) of 8-(azetidin-3-yl)-2-[2-(propan-2-yl)phenyl]-9-[[4-(1H-pyrazol-1-yl)phenyl]methyl]-9H-purine as an off-white solid. LC-MS: (ES, m/z): 450 [M+H]+ 1H-NMR-PH-FMA-PJ111-517-0: (400 MHZ, CD3OD-d4, ppm) δ 9.12 (s, 1H), 8.20 (s, 1H), 7.74-7.70 (m, 3H), 7.57-7.55 (m, 1H), 7.47-7.41 (m, 2H), 7.34-7.26 (m, 3H), 6.51-6.50 (m, 1H), 5.56 (s, 2H), 4.45-4.37 (m, 1H), 4.08-4.04 (m, 2H), 3.79-3.75 (m, 2H), 3.38-3.32 (m, 1H), 1.16 (d, J=6.8 Hz, 6H).

### Example 15: Synthesis of 8-(oxetan-3-yl)-2-[2-(propan-2-yl)phenyl]-9-[[4-(1H-pyrazol-1-yl)phenyl methyl]-9H-purine (I-15)

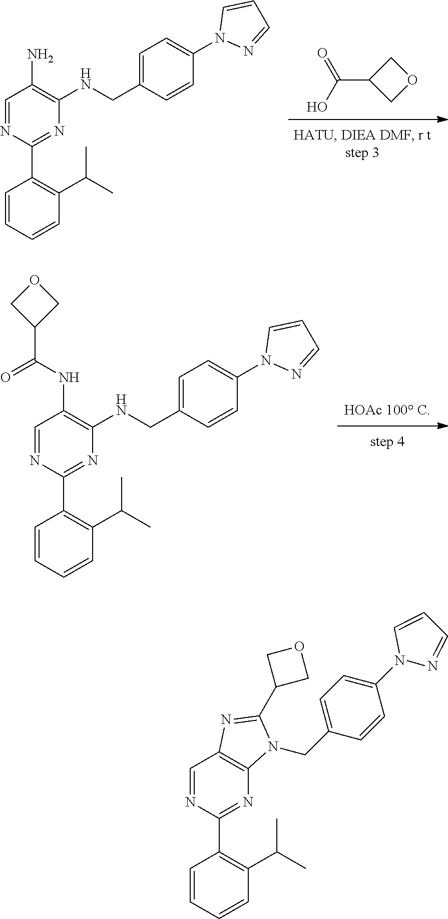

### Step 1. Synthesis of N-[2-[2-(propan-2-yl)phenyl]-4-([[4-(1H-pyrazol-1-yl)phenyl]methyl]amino)pyrimidin-5-yl]oxetane-3-carboxamide

Into a 25 mL round-bottom flask, was placed 2-[2-(propan-2-yl)phenyl]-4-N-[[4-(1H-pyrazol-1-yl)phenyl]methyl]pyrimidine-4,5-diamine (100 mg, 0.26 mmol, 1.00 equiv), HATU (98 mg, 0.26 mmol, 1.00 equiv), DIEA (100 mg, 0.77 mmol, 3.00 equiv), oxetane-3-carboxylic acid (80 mg, 0.78 mmol, 3 equiv), DMF (3 mL). The resulting solution was stirred overnight at room temperature. The reaction was then quenched by the addition of NaHCO3 (s). The resulting solution was diluted with 12 mL. The resulting solution was extracted with 3×10 mL of EA. The organic layers were combined. The mixture was dried over anhydrous sodium sulfate. The solids were filtered out. The resulting mixture was concentrated under vacuum. The residue was purified by preparative TLC EA/PE (0-100%). The crude product was purified by Prep-HPLC with the following Column: XBridge Prep C18 OBD Column 19×150 mm 5 μm; Mobile Phase A: water (10 mmol/l NH₄HCO₃), Mobile Phase B: ACN; Flow rate: 20 mL/min; Gradient: 35% B to 55% B in 7 min; 254 220 nm. This resulted in 50 mg (20%) of N-[2-[2-(propan-2-yl)phenyl]-4-([[4-(1H-pyrazol-1-yl)phenyl]methyl]amino)pyrimidin-5-yl]oxetane-3-carboxamide as yellow oil. LC-MS (ESI) m/z 469.3[M+H]+.

### Step 2. Synthesis of 8-(oxetan-3-yl)-2-[2-(propan-2-yl)phenyl]-9-[[4-(1H-pyrazol-1-yl)phenyl]methyl]-9H-purine (I-15)

Into a 25 mL round-bottom flask, was placed N-[2-[2-(propan-2-yl)phenyl]-4-([[4-(1H-pyrazol-1-yl)phenyl]methyl]amino)pyrimidin-5-yl]oxetane-3-carboxamide (45 mg, 0.10 mmol, 1.00 equiv), HOAc (3 mL). The resulting solution was stirred for 4 h at 100° C. in an oil bath. The reaction mixture was cooled to room temperature. The resulting mixture was concentrated under vacuum. The pH value of the solution was adjusted to 7-8 with NH3 in methanol (7 mol/L). The crude product was purified by Prep-HPLC with the following conditions: Column, XBridge Prep C18 OBD Column, 5 μm, 19×150 mm; mobile phase, water (10 mmol/l NH4HCO3) and ACN (15.0% ACN up to 95.0% in 5 min); Detector, UV 254 220 nm. This resulted in 13.8 mg (32%) of 8-(oxetan-3-yl)-2-[2-(propan-2-yl)phenyl]-9-[[4-(1H-pyrazol-1-yl)phenyl]methyl]-9H-purine as a white solid. LC-MS (ESI) m/z 451.0[M+H]⁺ 1H NMR (300 MHz, Methanol-d4) δ 9.13 (s, 1H), 8.18 (d, J=2.4 Hz, 1H), 7.71-7.67 (m, 3H), 7.55-7.52 (m, 1H), 7.45-7.38 (m, 2H), 7.30-7.23 (m, 3H), 6.49-6.47 (m, 1H), 5.50 (s, 2H), 4.93-4.85 (m, 4H), 4.79-4.70 (m, 1H), 3.37-3.33 (m, 1H), 1.14 (d, J=6.9 Hz, 6H).

### Example 16: Synthesis of 7-([4-[1-methyl-4-(trifluoromethyl)-1H-imidazol-2-yl]phenyl]methyl)-2-[2-(propan-2-yl)pyridin-3-yl]-5H,6H,7H-pyrrolo[2,3-d]pyrimidin-6-one (I-16)

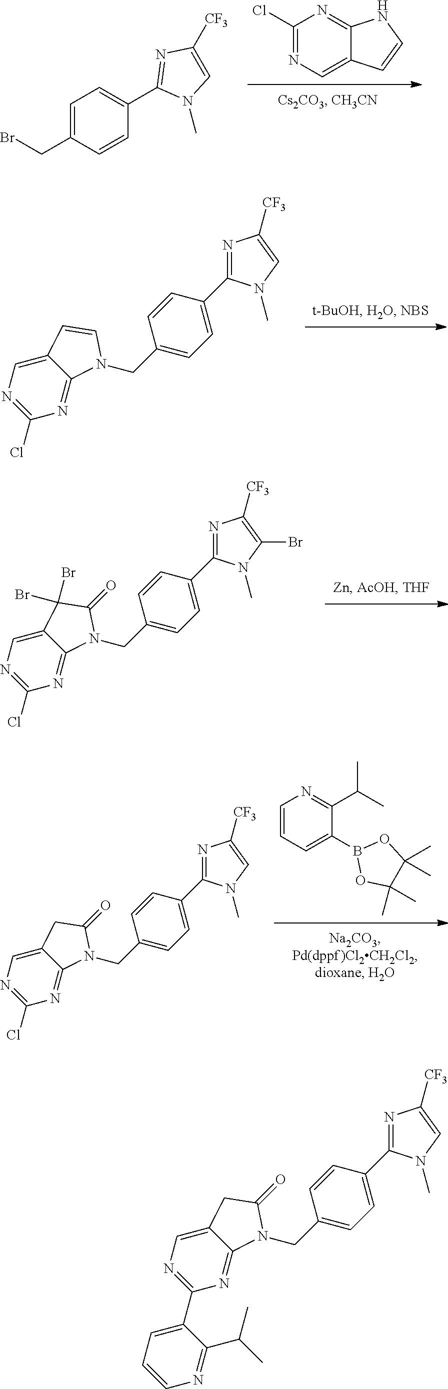

### Step 1. Synthesis of 2-[4-([2-chloro-7H-pyrrolo[2,3-d]pyrimidin-7-yl]methyl)phenyl]-1-methyl-4-(trifluoromethyl)-1H-imidazole

To a stirred mixture of 2-chloro-7H-pyrrolo[2,3-d]pyrimidine (241 mg, 1.57 mmol, 1.00 equiv) and 2-[4-(bromomethyl)phenyl]-1-methyl-4-(trifluoromethyl)-1H-imidazole (500 mg, 1.57 mmol, 1.00 equiv) in ACN (10 mL), was added Cs2CO3 (766 mg, 2.35 mmol, 1.50 equiv). The resulting solution was stirred for 1 h at 80° C. The reaction mixture was cooled to 25° C. The solids were filtered out. The resulting mixture was concentrated under vacuum. The residue was applied onto a silica gel column with PE/EA (0˜30%). This resulted in 600 mg (93%) of 2-[4-([2-chloro-7H-pyrrolo[2,3-d]pyrimidin-7-yl]methyl)phenyl]-1-methyl-4-(trifluoromethyl)-1H-imidazole as a white solid. LCMS (ES, m/z): 392, 394 [M+H]+.

### Step 2. Synthesis of 5,5-dibromo-7-([4-[5-bromo-1-methyl-4-(trifluoromethyl)-1H-imidazol-2-yl]phenyl]methyl)-2-chloro-5H,6H,7H-pyrrolo[2,3-d]pyrimidin-6-one

To a stirred mixture of 2-[4-([2-chloro-7H-pyrrolo[2,3-d]pyrimidin-7-yl]methyl)phenyl]-1-methyl-4-(trifluoromethyl)-1H-imidazole (600 mg, 1.53 mmol, 1.00 equiv) in tert-Butanol (15 mL) and water (3 mL), was added NBS (1.36 g, 7.64 mmol, 5.00 equiv). The resulting solution was stirred overnight at 80° C. The resulting mixture was concentrated under vacuum. The resulting solution was diluted with 15 mL of water. The resulting solution was extracted with 2×30 mL of EA and the organic layers combined and dried over sodium sulfate. The solids were filtered out. The resulting mixture was concentrated under vacuum. The residue was applied onto a silica gel column with PE/EA (0-40%). This resulted in 1 g (96%) of 5,5-dibromo-7-([4-[5-bromo-1-methyl-4-(trifluoromethyl)-1H-imidazol-2-yl]phenyl]methyl)-2-chloro-5H,6H, 7H-pyrrolo[2,3-d]pyrimidin-6-one as a white solid. LCMS (ES, m/z): 642 [M+H]+.

### Step 3. Synthesis of 2-chloro-7-([4-[1-methyl-4-(trifluoromethyl)-1H-imidazol-2-yl]phenyl]methyl)-5H,6H,7H-pyrrolo[2,3-d]pyrimidin-6-one

To a stirred mixture of 5,5-dibromo-7-([4-[5-bromo-1-methyl-4-(trifluoromethyl)-1H-imidazol-2-yl]phenyl]methyl)-2-chloro-5H,6H,7H-pyrrolo[2,3-d]pyrimidin-6-one (500 mg, 0.78 mmol, 1.00 equiv) in AcOH (10 mL), tetrahydrofuran (5 mL) was added zinc dust (151 mg, 2.31 mmol, 3.00 equiv) in several batches at 0° C. The resulting solution was stirred overnight at room temperature (20° C.). The solids were filtered out. The resulting mixture was concentrated under vacuum. The residue was purified by preparative TLC (EA:PE=1:3). This resulted in 300 mg (95%) of 2-chloro-7-([4-[1-methyl-4-(trifluoromethyl)-1H-imidazol-2-yl]phenyl]methyl)-5H,6H, 7H-pyrrolo[2,3-d]pyrimidin-6-one as a yellow solid. LCMS (ES, m/z): 408 [M+H]+.

### Step 4. Synthesis of 7-([4-[1-methyl-4-(trifluoromethyl)-1H-imidazol-2-yl]phenyl]methyl)-2-[2-(propan-2-yl)pyridin-3-yl]-5H,6H,7H-pyrrolo[2,3-d]pyrimidin-6-one (I-16)

To a stirred mixture of 2-chloro-7-([4-[1-methyl-4-(trifluoromethyl)-1H-imidazol-2-yl]phenyl]methyl)-5H,6H,7H-pyrrolo[2,3-d]pyrimidin-6-one (50 mg, 0.12 mmol, 1.00 equiv), 2-(propan-2-yl)-3-(tetramethyl-1,3,2-dioxaborolan-2-yl)pyridine (45 mg, 0.18 mmol, 1.50 equiv) in dioxane (10 mL), water (2 mL) was added Pd(dppf)Cl2 CH2Cl2 (10 mg, 0.01 mmol, 0.10 equiv), sodium carbonate (26 mg, 0.25 mmol, 2.00 equiv), The resulting solution was stirred for 2 h at 100° C. The reaction mixture was cooled to 25° C. The mixture was filtered through a Celite pad. The resulting mixture was concentrated under vacuum. The residue was purified by preparative TLC (EA:PE=1:1). The crude product was purified by Prep-HPLC with the following conditions (Prep-HPLC-025): Column: XBridge C18 OBD Prep Column, 100 A, 5 um, 19 mm×250 mm; Mobile Phase A:waters (0.05% ammonia in water), Mobile Phase B: ACN; Flow rate: 20 mL/min; Gradient: 35% B to 65% B in 8 min; 254 nm. This resulted in 6.9 mg (11%) of 7-([4-[1-methyl-4-(trifluoromethyl)-1H-imidazol-2-yl]phenyl]methyl)-2-[2-(propan-2-yl)pyridin-3-yl]-5H,6H,7H-pyrrolo[2,3-d]pyrimidin-6-one as a greenish solid. LCMS (ES, m/z): 493 [M+H]+ H-NMR-PH-FMA-PJ111-811-0: (300 MHz, DMSO, ppm): δ 8.63-8.61 (m, 1H), 8.59 (s, 1H), 7.99-7.96 (m, 1H), 7.93-7.92 (m, 2H), 7.69 (d, J=8.4 Hz, 2H), 7.48 (d, J=8.4 Hz, 2H), 7.35-7.31 (m, 1H), 5.00 (s, 2H), 3.89 (s, 2H), 3.76 (s, 3H), 3.64-3.60 (1H, m), 1.11 (d, J=6.6 Hz, 6H).

### Example 17: Synthesis of 8-methoxy-9-([4-[1-methyl-4-(trifluoromethyl)-1H-imidazol-2-yl]phenyl]methyl)-2-[2-(propan-2-yl)pyridin-3-yl]-9H-purine (I-17)

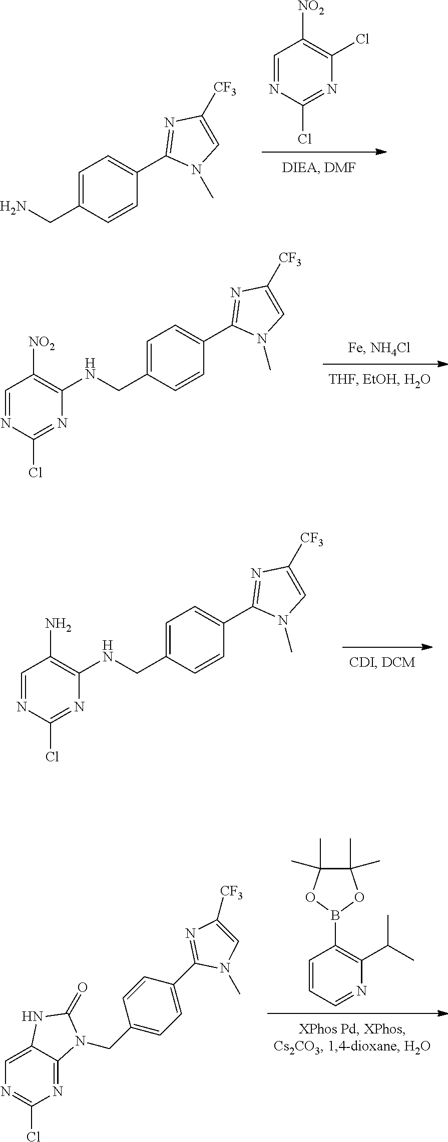 [SMILES: Cn1cc(C(F)(F)F)nc1-c1ccc(CN)cc1 ; O=[N+]([O-])c1cnc(Cl)nc1Cl ; Cn1cc(C(F)(F)F)nc1-c1ccc(CNc2nc(Cl)ncc2[N+](=O)[O-])cc1 ; Cn1cc(C(F)(F)F)nc1-c1ccc(CNc2nc(Cl)ncc2N)cc1 ; Cn1cc(C(F)(F)F)nc1-c1ccc(Cn2c(=O)[nH]c3cnc(Cl)nc32)cc1 ; CC(C)c1ncccc1B1OC(C)(C)C(C)(C)O1]

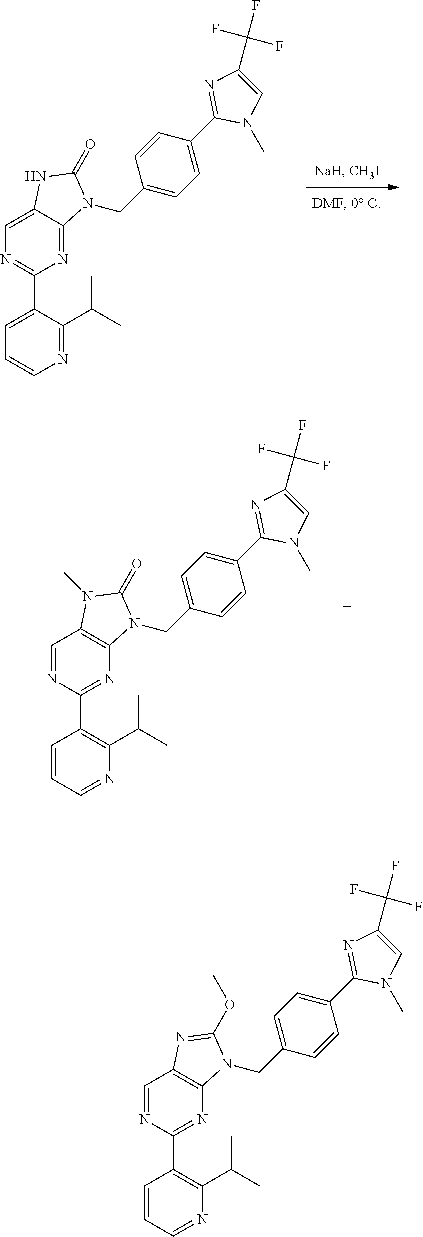 [SMILES: CC(C)c1ncccc1-c1ncc2[nH]c(=O)n(Cc3ccc(-c4nc(C(F)(F)F)cn4C)cc3)c2n1 ; CC(C)c1ncccc1-c1ncc2c(n1)n(Cc1ccc(-c3nc(C(F)(F)F)cn3C)cc1)c(=O)n2C ; COc1nc2cnc(-c3cccnc3C(C)C)nc2n1Cc1ccc(-c2nc(C(F)(F)F)cn2C)cc1]

### Step 1. Synthesis of 2-chloro-N-([4-[1-methyl-4-(trifluoromethyl)imidazol-2-yl]phenyl]methyl)-5-nitropyrimidin-4-amine

A mixture of 1-[4-[1-methyl-4-(trifluoromethyl)imidazol-2-yl]phenyl]methanamine (79 g, 278 mmol), 2,4-dichloro-5-nitropyrimidine (64.5 g, 333.5 mmol) and DIEA (107.8 g, 835.3 mmol) in DMF (1100 mL) was stirred for 1 h at 25° C. The reaction was quenched by the addition of water (1500 mL) at room temperature. The resulting mixture was extracted with EA (3×2000 mL). The combined organic layers were washed with brine (3×1500 mL), dried over anhydrous Na2SO4. After filtration, the filtrate was concentrated under reduced pressure. The residue was purified by silica gel column chromatography, eluted with PE/EA (1:1) to afford 2-chloro-N-([4-[1-methyl-4-(trifluoromethyl)imidazol-2-yl]phenyl]methyl)-5-nitropyrimidin-4-amine (55 g, 43%) as a yellow solid. LCMS (ES, m/z): 413 [M+H]+.

### Step 2. Synthesis of 2-chloro-N4-([4-[1-methyl-4-(trifluoromethyl)imidazol-2-yl]phenylmethyl)pyrimidine-4,5-diamine

To a stirred mixture of 2-chloro-N-([4-[1-methyl-4-(trifluoromethyl)imidazol-2-yl]phenyl]methyl)-5-nitropyrimidin-4-amine (55 g, 119.6 mmol) and Fe (33.4 g, 599.5 mmol) in THF (450 mL) and EtOH (450 mL) was added NH4Cl (12.7 g, 239.9 mmol) in water (90 mL) at room temperature. The resulting mixture was stirred for 1 h at 80° C. The mixture was allowed to cool down to room temperature. The resulting mixture was filtered, the filter cake was washed with EA (3×1000 mL). The filtrate was concentrated under reduced pressure. This resulted in 2-chloro-N4-([4-[1-methyl-4-(trifluoromethyl)imidazol-2-yl]phenyl]methyl)pyrimidine-4,5-diamine (52 g, 94%) as a brown solid. LCMS (ES, m/z): 383 [M+H]+.

### Step 3. Synthesis of 2-chloro-9-([4-[1-methyl-4-(trifluoromethyl)imidazol-2-yl]phenyl]methyl)-7H-purin-8-one

To a stirred mixture of 2-chloro-N4-([4-[1-methyl-4-(trifluoromethyl)imidazol-2-yl]phenyl]methyl)pyrimidine-4,5-diamine (52 g, 122.2 mmol) in DCM (610 mL) was added CDI (79.4 g, 488.8 mmol) in portions at 25° C. The resulting mixture was stirred for 1 h at 40° C. The reaction was quenched by the addition of water (400 mL) at room temperature. The resulting mixture was extracted with CH2Cl2 (3×800 mL). The combined organic layers were washed with brine (800 mL), dried over anhydrous Na2SO4. After filtration, the filtrate was concentrated under reduced pressure. The residue was purified by silica gel column chromatography, eluted with PE/EA (1:4) to afford 2-chloro-9-([4-[1-methyl-4-(trifluoromethyl)imidazol-2-yl]phenyl]methyl)-7H-purin-8-one (29 g, 43%) as a yellow solid. LCMS (ES, m/z): 409 [M+H]+.

### Step 4. Synthesis of 2-(2-isopropylpyridin-3-yl)-9-([4-[1-methyl-4-(trifluoromethyl)imidazol-2-yl]phenyl]methyl)-7H-purin-8-one

To a mixture of 2-chloro-9-([4-[1-methyl-4-(trifluoromethyl)imidazol-2-yl]phenyl]methyl)-7H-purin-8-one (29.0 g, 63.8 mmol) and 2-isopropyl-3-(4,4,5,5-tetramethyl-1,3,2-dioxaborolan-2-yl)pyridine (23.5 g, 95.1 mmol) in dioxane (1000 mL) and water (200 mL) were added Cs2CO3 (52.0 g, 159.6 mmol) and XPhos (12.1 g, 25.5 mmol), XPhos Pd G3 (10.8 g, 12.7 mmol). The resulting mixture was stirred for 16 h at 90° C. under nitrogen atmosphere. The mixture was allowed to cool down to room temperature. The resulting mixture was filtered, the filter cake was washed with EA (3×2000 mL). The filtrate was concentrated under reduced pressure. The resulting mixture was diluted with water (1000 mL). The resulting mixture was extracted with EA (3×2000 mL). The combined organic layers were washed with brine (1000 mL), dried over anhydrous Na2SO4. After filtration, the filtrate was concentrated under reduced pressure. The residue was purified by reverse flash chromatography with the following conditions: column, C18 silica gel; mobile phase, water (containing 0.05% TFA), ACN (5% to 50% gradient in 60 min); detector, UV 254 nm. The residue was purified by reverse flash chromatography with the following conditions: column, C18 silica gel; mobile phase, water (containing 6.5 mM NH4HCO3+NH4OH), ACN (0% to 50% gradient in 60 min); detector, UV 254 nm. The product fractions were lyophilized to afford 2-(2-isopropylpyridin-3-yl)-9-([4-[1-methyl-4-(trifluoromethyl)imidazol-2-yl]phenyl]methyl)-7H-purin-8-one (5.098 g, 16%) as a white solid. 1H-NMR (CD3OD, 400 MHZ) δ (ppm): 8.56-8.54 (m, 1H), 8.38 (s, 1H), 8.00-7.97 (m, 1H), 7.67-7.57 (m, 5H), 7.35-7.31 (m, 1H), 5.22 (s, 2H), 3.74 (s, 3H), 3.61-3.54 (m, 1H). LCMS (ES, m/z): 494 [M+H]+.

### Step 5. Synthesis of 8-methoxy-9-([4-[1-methyl-4-(trifluoromethyl)-1H-imidazol-2-yl]phenyl]methyl)-2-[2-(propan-2-yl)pyridin-3-yl]-9H-purine (I-17)

Into a 25-mL round-bottom flask, was placed 9-([4-[1-methyl-4-(trifluoromethyl)-1H-imidazol-2-yl]phenyl]methyl)-2-[2-(propan-2-yl)pyridin-3-yl]-8,9-dihydro-7H-purin-8-one (100 mg, 0.20 mmol, 1.00 equiv), DMF (1 mL). This was followed by the addition of sodium hydride (10.5 mg, 0.26 mmol, 1.30 equiv, 60%) in several batches at 0° C. then stirred at 0° C. for 0.5 h. To this was added iodomethane (43.2 mg, 0.30 mmol). The resulting solution was stirred for 5 h at room temperature. The reaction was then quenched by the addition of 2 mL of water. The resulting solution was extracted with 2×3 mL of EA and the organic layers combined and dried over anhydrous sodium sulfate and concentrated under vacuum. The crude product was purified by Prep-HPLC with the following conditions: Column, XBridge Prep C18 OBD Column19*150 mm 5 μmC-0013; mobile phase, water (0.05% TFA)/ACN; Detector, uv254,220. Gradient: 20% ACN to 40% ACN in 7 min. This resulted in 34.7 mg (34%) of 7-methyl-9-([4-[1-methyl-4-(trifluoromethyl)-1H-imidazol-2-yl]phenyl]methyl)-2-[2-(propan-2-yl)pyridin-3-yl]-8,9-dihydro-7H-purin-8-one as a white solid. And 3.6 mg (4%) of 8-methoxy-9-([4-[1-methyl-4-(trifluoromethyl)-1H-imidazol-2-yl]phenyl]methyl)-2-[2-(propan-2-yl)pyridin-3-yl]-9H-purine as a white solid. LC-MS−: (ES, m/z): 508.3[M+H]+. ¹H-NMR-PH-FMA-PJ111-772-0A: (300 MHZ, Methanol-d4): δ 8.57-8.54 (m, 1H), 8.51 (s, 1H), 8.01-7.98 (m, 1H), 7.61 (s, 4H), 7.52 (s, 1H), 7.35-7.31 (m, 1H), 5.26 (s, 2H), 3.77 (s, 3H), 3.62-3.57 (m, 1H), 3.54 (s, 3H), 1.22 (d, J=6.9 Hz, 6H).

### Example 18: Synthesis of 7-([4-[1-methyl-4-(trifluoromethyl)-1H-imidazol-2-yl]phenyl]methyl)-2-[2-(propan-2-yl)phenyl]-5H,6H,7H-pyrrolo[2,3-d]pyrimidin-6-one (I-18)

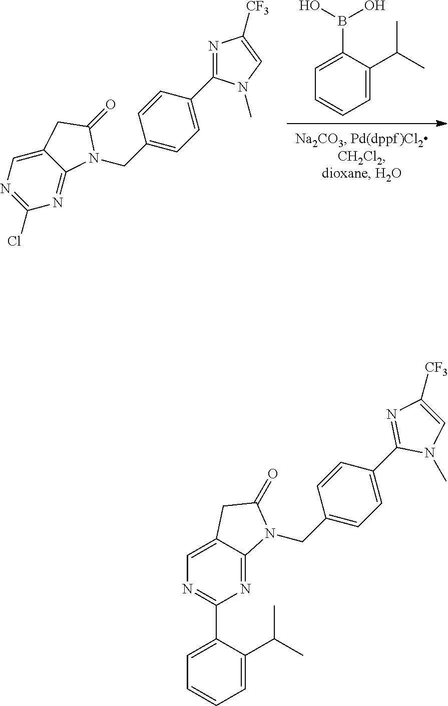 [SMILES: Cn1cc(C(F)(F)F)nc1-c1ccc(CN2C(=O)Cc3cnc(Cl)nc32)cc1 ; CC(C)c1ccccc1B(O)O ; CC(C)c1ccccc1-c1ncc2c(n1)N(Cc1ccc(-c3nc(C(F)(F)F)cn3C)cc1)C(=O)C2]

### Step 1. Synthesis of 7-([4-[1-methyl-4-(trifluoromethyl)-1H-imidazol-2-yl]phenyl]methyl)-2-[2-(propan-2-yl)phenyl]-5H,6H,7H-pyrrolo[2,3-d]pyrimidin-6-one (I-18)

Into a 25 mL round-bottom flask purged and maintained with an inert atmosphere of nitrogen, was placed 2-chloro-7-([4-[1-methyl-4-(trifluoromethyl)-1H-imidazol-2-yl]phenyl]methyl)-5H,6H,7H-pyrrolo[2,3-d]pyrimidin-6-one (50 mg, 0.12 mmol, 1.00 equiv), [2-(propan-2-yl)phenyl]boronic acid (20 mg, 0.12 mmol, 1.00 equiv), sodium carbonate (26 mg, 0.25 mmol, 2.00 equiv), Pd(dppf)Cl2·CH2Cl2 (10 mg, 0.01 mmol, 0.10 equiv), dioxane (4 mL), water (1 mL). The resulting solution was stirred for 2 h at 100° C. The reaction mixture was cooled to 25° C. The mixture was filtered through a Celite pad. The resulting mixture was concentrated under vacuum. The residue was purified by preparative TLC (EA:PE=1:1). The crude product was purified by Prep-HPLC with the following conditions (Column: XBridge Prep Shield RP18 OBD Column, 19×150 mm, 5 um-13 nm; Mobile phase, water with 0.05% NH4HCO3 and ACN (30% ACN up to 60% in 8 min); Detector: 254 nm). This resulted in 10.1 mg (16%) of 7-([4-[1-methyl-4-(trifluoromethyl)-1H-imidazol-2-yl]phenyl]methyl)-2-[2-(propan-2-yl)phenyl]-5H,6H, 7H-pyrrolo[2,3-d]pyrimidin-6-one as a brown solid. LC-MS (ESI) m/z 492.2 [M+H]+ 1H NMR (400 MHZ, CD3OD) δ8.48 (s, 1H), 7.70-7.58 (m, 5H), 7.48-7.43 (m, 3H), 7.30-7.26 (m, 1H), 5.10 (s, 2H), 3.77 (s, 3H), 3.41-3.36 (m, 3H), 1.16 (d, J=6.8 Hz, 6H).

### Example 19: Synthesis of 7-([4-[1-(oxetan-3-yl)-4-(trifluoromethyl)-1H-imidazol-2-yl]phenylmethyl)-2-[2-(propan-2-yl)pyridin-3-yl]-5H,6H,7H-pyrrolo[2,3-d]pyrimidin-6-one (I-19)

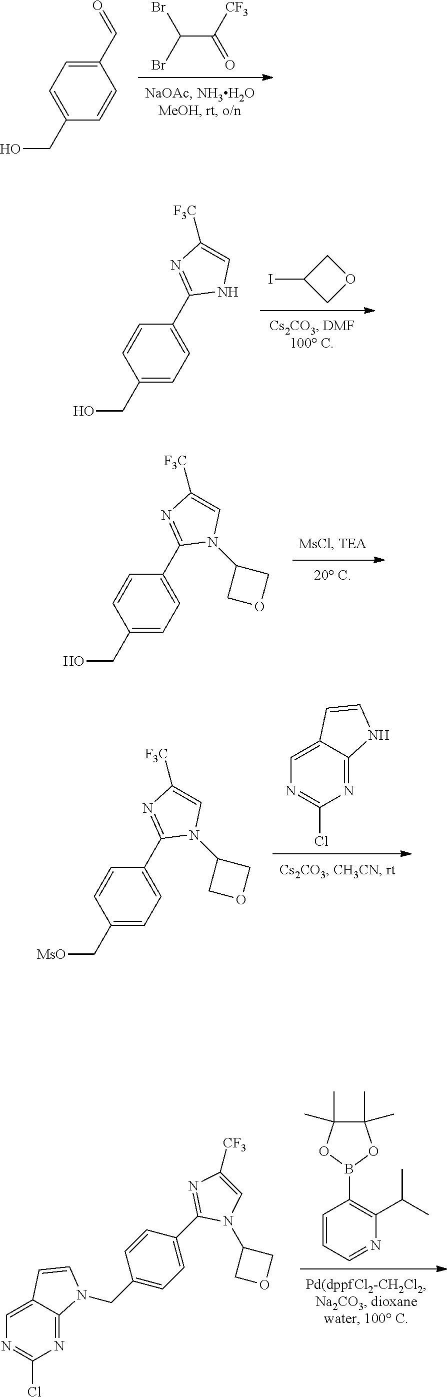

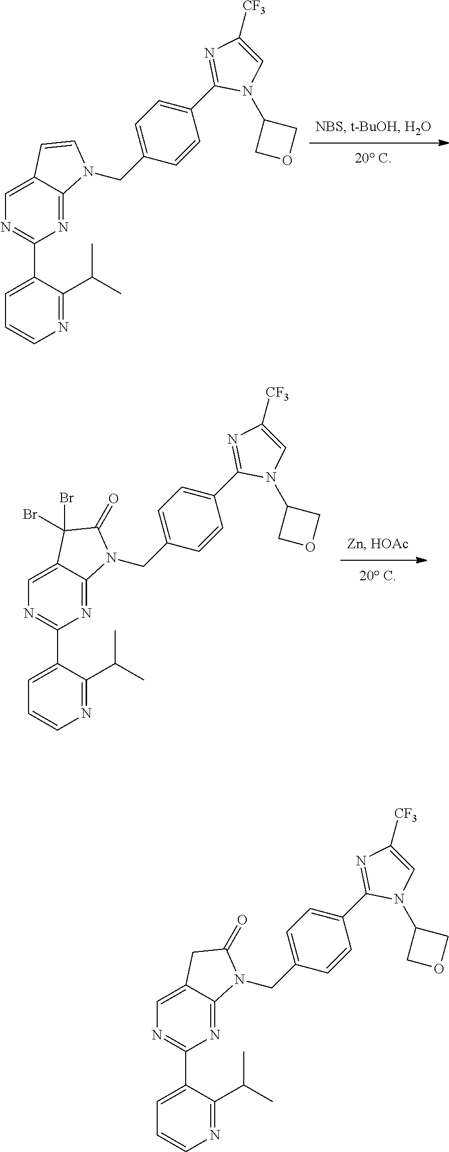 [SMILES: CC(C)c1ncccc1-c1ncc2ccn(Cc3ccc(-c4nc(C(F)(F)F)cn4C4COC4)cc3)c2n1 ; CC(C)c1ncccc1-c1ncc2c(n1)N(Cc1ccc(-c3nc(C(F)(F)F)cn3C3COC3)cc1)C(=O)C2(Br)Br ; CC(C)c1ncccc1-c1ncc2c(n1)N(Cc1ccc(-c3nc(C(F)(F)F)cn3C3COC3)cc1)C(=O)C2]

### Step 1. Synthesis of [4-[4(trifluoromethyl)-1H-imidazol-2-yl]phenyl]methanol

Into a 500-mL round-bottom flask, was placed 3,3-dibromo-1,1,1-trifluoropropan-2-one (24.7 g, 90.62 mmol, 1.25 equiv), water (40 mL, 2.22 mol). This was followed by the addition of NaOAc (15 g, 182.85 mmol, 2.52 equiv). The above mixture was stirred for 2 h at 100° C. and allow cooled to rt. Into another 500-mL round-bottom flask was added 4-(hydroxymethyl)benzaldehyde (10 g, 72.72 mmol, 1.00 equiv, 99%), methanol (157 mL) and ammonia (47 mL). The above mixture was stirred for 2 h at rt and was then added to the first cooled mixture. The resulting solution was stirred overnight at room temperature. The resulting mixture was concentrated under vacuum. The solids were filtered out. The crude product was slurried from EA/PE in the ratio of 1/3. This resulted in 7 g (37%) of [4-[4(trifluoromethyl)-1H-imidazol-2-yl]phenyl]methanol as a yellow solid. LC-MS (ESI) m/z 243 [M+H]+.

### Step 2. Synthesis of [4-[1-(oxetan-3-yl)-4-(trifluoromethyl)-1H-imidazol-2-yl]phenyl]methanol

Into a 100 mL round-bottom flask, was placed [4-[4-(trifluoromethyl)-1H-imidazol-2-yl]phenyl]methanol (1.5 g, 6.19 mmol, 1.00 equiv), 3-iodooxetane (1.14 g, 6.20 mmol, 1.20 equiv), Cs2CO3 (4.04 g, 12.40 mmol, 2.01 equiv), DMF (20 mL). The resulting solution was stirred for 12 h at 110° C. The reaction mixture was cooled to room temperature. The resulting solution was diluted with 30 mL of water. The resulting solution was extracted with 2×30 mL of EA and the organic layers combined and dried over anhydrous sodium sulfate. The solids were filtered out. The resulting mixture was concentrated under vacuum. This resulted in 300 mg (16%) of [4-[1-(oxetan-3-yl)-4-(trifluoromethyl)-1H-imidazol-2-yl]phenyl]methanol as a yellow solid. LC-MS (ESI) m/z 299 [M+H]+.

### Step 3. Synthesis of [4-[1-(oxetan-3-yl)-4-(trifluoromethyl)-1H-imidazol-2-yl]phenyl]methyl methane sulfonate

Into a 8 mL vial, was placed a solution of [4-[1-(oxetan-3-yl)-4-(trifluoromethyl)-1H-imidazol-2-yl]phenyl]methanol (100 mg, 0.32 mmol, 1.00 equiv) in dichloromethane (2 mL), TEA (48 mg, 0.47 mmol, 1.50 equiv). This was followed by the addition of methanesulfonyl chloride (40.135 mg, 0.35 mmol, 1.10 equiv) at 0° C. The resulting solution was stirred for 30 min at room temperature (20° C.). The resulting solution was diluted with 2 mL of water. The resulting solution was extracted with 2×2 mL of dichloromethane and the organic layers combined and dried over anhydrous Na2SO4 and concentrated under vacuum. This resulted in 80 mg (62%) of [4-[1-(oxetan-3-yl)-4-(trifluoromethyl)-1H-imidazol-2-yl]phenyl]methyl methane sulfonate as yellow oil. LC-MS (ESI) m/z 377 [M+H]+.

### Step 4. Synthesis of -[4-([2-chloro-7H-pyrrolo[2,3-d]pyrimidin-7-yl]methyl)phenyl]-1-(oxetan-3-yl)-4-(trifluoromethyl)-1H-imidazole

Into a 8 mL vial, was placed 2-chloro-7H-pyrrolo[2,3-d]pyrimidine (53 mg, 0.35 mmol, 1.00 equiv), [4-[1-(oxetan-3-yl)-4-(trifluoromethyl)-1H-imidazol-2-yl]phenyl]methyl methanesulfonate (130 mg, 0.32 mmol, 1.00 equiv), Cs2CO3 (225 mg, 0.69 mmol, 2.00 equiv), ACN (3 mL). The resulting solution was stirred for 2 h at room temperature (20° C.). The resulting solution was diluted with 5 mL of water. The resulting solution was extracted with 2×5 mL of EA and the organic layers combined and concentrated under vacuum. The residue was purified by preparative TLC (EA:PE=1:3). This resulted in 80 mg (51%) of 2-[4-([2-chloro-7H-pyrrolo[2,3-d]pyrimidin-7-yl]methyl)phenyl]-1-(oxetan-3-yl)-4-(trifluoromethyl)-1H-imidazole as a white solid. LC-MS (ESI) m/z 434 [M+H]+.

### Step 5. Synthesis of 3-[7-([4-[1-(oxetan-3-yl)-4-(trifluoromethyl)-1H-imidazol-2-yl]phenyl]methyl)-7H-pyrrolo[2,3-d]pyrimidin-2-yl]-2-(propan-2-yl)pyridine

Into a 25 mL round-bottom flask purged and maintained with an inert atmosphere of nitrogen, was placed 2-[4-([2-chloro-7H-pyrrolo[2,3-d]pyrimidin-7-yl]methyl)phenyl]-1-(oxetan-3-yl)-4-(trifluoromethyl)-1H-imidazole (80 mg, 0.18 mmol, 1.00 equiv), 2-(propan-2-yl)-3-(tetramethyl-1,3,2-dioxaborolan-2-yl)pyridine (80 mg, 0.31 mmol, 1.50 equiv), Pd(dppf)Cl2 CH2Cl2 (15 mg, 0.02 mmol, 0.10 equiv), sodium carbonate (39 mg, 0.37 mmol, 2.00 equiv), dioxane (10 mL), water (3 mL). The resulting solution was stirred for 4 h at 100° C. The reaction mixture was cooled to room temperature (25° C.). The solids were filtered out. The filtrate was concentrated under vacuum. The residue was purified by preparative TLC (EA:PE=1:1). This resulted in 70 mg (73%) of 3-[7-([4-[1-(oxetan-3-yl)-4-(trifluoromethyl)-1H-imidazol-2-yl]phenyl]methyl)-7H-pyrrolo[2,3-d]pyrimidin-2-yl]-2-(propan-2-yl)pyridine as yellow oil.

### Step 6. Synthesis of 5,5-dibromo-7-([4-[1-(oxetan-3-yl)-4-(trifluoromethyl)-1H-imidazol-2-yl]phenyl]methyl)-2-[2-(propan-2-yl)pyridin-3-yl]-5H,6H,7H-pyrrolo[2,3-d]pyrimidin-6-one

Into a 25 mL round-bottom flask, was placed 3-[7-([4-[1-(oxetan-3-yl)-4-(trifluoromethyl)-1H-imidazol-2-yl]phenyl]methyl)-7H-pyrrolo[2,3-d]pyrimidin-2-yl]-2-(propan-2-yl)pyridine (70 mg, 0.13 mmol, 1.00 equiv), tert-Butanol (5 mL), water (2 mL), NBS (68 mg, 0.38 mmol, 3.00 equiv). The resulting solution was stirred for 6 h at room temperature (20° C.). The resulting solution was diluted with 5 mL of water. The resulting solution was extracted with 2×5 mL of EA and the organic layers combined and dried over anhydrous sodium sulfate and concentrated under vacuum. The residue was purified by preparative TLC (EA:PE=2:1). This resulted in 90 mg (96%) of 5,5-dibromo-7-([4-[1-(oxetan-3-yl)-4-(trifluoromethyl)-1H-imidazol-2-yl]phenyl]methyl)-2-[2-(propan-2-yl)pyridin-3-yl]-5H,6H,7H-pyrrolo[2,3-d]pyrimidin-6-one as a yellow solid. LC-MS (ESI) m/z 693 [M+H]+.

### Step 7. Synthesis of 7-([4-[1-(oxetan-3-yl)-4-(trifluoromethyl)-1H-imidazol-2-yl]phenyl]methyl)-2-[2-(propan-2-yl)pyridin-3-yl]-5H,6H,7H-pyrrolo[2,3-d]pyrimidin-6-one (I-19)

Into a 8 mL vial, was placed a solution of 5,5-dibromo-7-([4-[1-(oxetan-3-yl)-4-(trifluoromethyl)-1H-imidazol-2-yl]phenyl]methyl)-2-[2-(propan-2-yl)pyridin-3-yl]-5H,6H,7H-pyrrolo[2,3-d]pyrimidin-6-one (90 mg, 0.12 mmol, 1.00 equiv) in tetrahydrofuran (1 mL), acetic acid (1 mL), Zn (42 mg, 0.64 mmol, 5.00 equiv). The resulting solution was stirred for 2 h at room temperature (20° C.). The solids were filtered out. The filtrate was concentrated under vacuum. The crude product was purified by Prep-HPLC with the following conditions: Column: XBridge C18 OBD Prep Column, 100 A, 5 μm, 19 mm×250 mm; Mobile Phase A:water (10 mmol/L NH4HCO3), Mobile Phase B: ACN; Flow rate: 20 mL/min; Gradient: 35% B to 55% B in 7 min;

254 nm; Rt: 7 min. This resulted in 2.7 mg (4%) of 7-([4-[1-(oxetan-3-yl)-4-(trifluoromethyl)-1H-imidazol-2-yl]phenyl]methyl)-2-[2-(propan-2-yl)pyridin-3-yl]-5H,6H, 7H-pyrrolo[2,3-d]pyrimidin-6-one as a light yellow solid. LC-MS (ESI) m/z 535.2 [M+H]+ 1H NMR (400 MHZ, CD3OD) δ 8.61-8.59 (m, 1H), 8.53 (s, 1H), 8.31 (s, 1H), 8.03-8.01 (m, 1H), 7.59 (d, J=8.4 Hz, 2H), 7.48 (d, J=8.4 Hz, 2H), 7.38-7.35 (m, 1H), 5.55-5.48 (m, 1H), 5.11 (s, 2H), 4.98-4.95 (m, 4H), 4.86-4.83 (m, 2H), 3.65-3.58 (m, 1H), 1.23 (d, J=6.8 Hz, 6H).

### Example 20: Synthesis of 2-chloro-8-([4-[1-methyl-4-(trifluoromethyl)-1H-imidazol-2-yl]phenyl]methyl)-5H,6H,7H,8H-pyrido[2,3-d]pyrimidin-7-one (I-20)

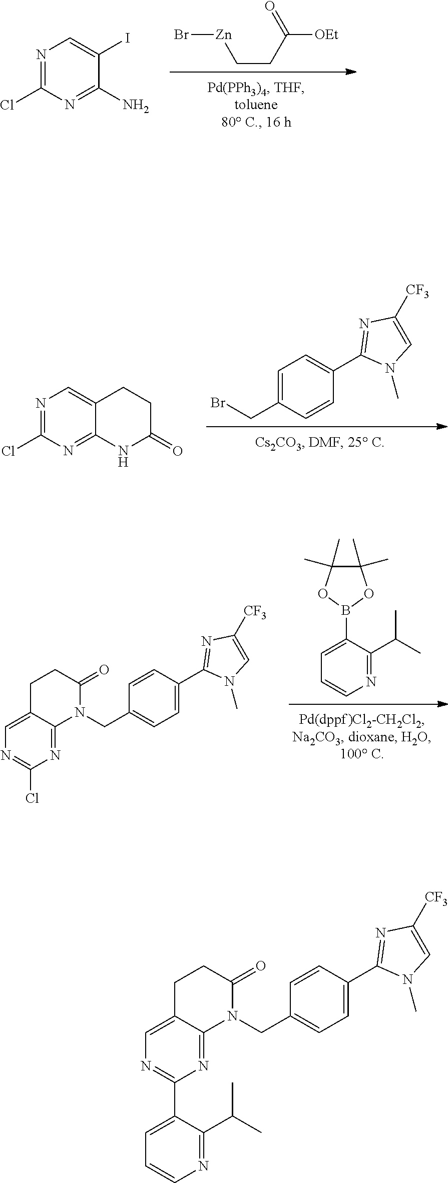

### Step 1. Synthesis of 2-chloro-5H,6H,7H,8H-pyrido[2,3-d]pyrimidin-7-one

Into a 50 mL 3-necked round-bottomed flask purged and maintained with an inert atmosphere of nitrogen, was placed a solution of 2-chloro-5-iodopyrimidin-4-amine (1 g, 3.91 mmol, 1.00 equiv) in toluene (20 mL), Pd(PPh3)4 (454 mg, 0.39 mmol, 0.10 equiv), ethyl 3-(bromozincio)propanoate (0.5 M in THF) (24 mL, 11.73 mmol, 3.00 equiv). The resulting solution was stirred overnight at 80° C. The reaction mixture was cooled to rt. The resulting mixture was concentrated under vacuum. The residue was applied onto a TLC with EA/petroleum ether (0-100%). The residue was purified by preparative TLC (DCM:MeOH=20:1). This resulted in 160 mg (20%) of 2-chloro-5H,6H,7H,8H-pyrido[2,3-d]pyrimidin-7-one as a light yellow solid. LC-MS (ESI) m/z 184 [M+H]+.

### Step 2. Synthesis of 2-chloro-8-([4-[1-methyl-4-(trifluoromethyl)-1H-imidazol-2-yl]phenyl]methyl)-5H,6H,7H,8H-pyrido[2,3-d]pyrimidin-7-one

Into a 50 mL round-bottom flask, was placed 2-chloro-5H,6H,7H,8H-pyrido[2,3-d]pyrimidin-7-one (80 mg, 0.40 mmol, 1.00 equiv), 2-[4-(bromomethyl)phenyl]-1-methyl-4-(trifluoromethyl)-1H-imidazole (139 mg, 0.41 mmol, 1.03 equiv), Cs2CO3 (285 mg, 0.86 mmol, 2.14 equiv), DMF (8 mL). The resulting solution was stirred for 5 h at 25° C. The resulting solution was diluted with 20 mL of water, extracted with 3×20 mL of EA and the organic layers were combined and concentrated under vacuum. The residue was applied onto a silica gel column with EA/petroleum ether (1:2). The collected fractions were combined and concentrated under vacuum. This resulted in 40 mg (24%) of 2-chloro-8-([4-[1-methyl-4-(trifluoromethyl)-1H-imidazol-2-yl]phenyl]methyl)-5H,6H,7H,8H-pyrido[2,3-d]pyrimidin-7-one as yellow oil. LC-MS (ESI) m/z 422 [M+H]+.

### Step 3. Synthesis of 8-([4-[1-methyl-4-(trifluoromethyl)-1H-imidazol-2-yl]phenyl]methyl)-2-[2-(propan-2-yl)pyridin-3-yl]-5H,6H,7H,8H-pyrido[2,3-d]pyrimidin-7-one (I-20)

Into a 25 mL round-bottom flask purged and maintained with an inert atmosphere of nitrogen, was placed 2-chloro-8-([4-[1-methyl-4-(trifluoromethyl)-1H-imidazol-2-yl]phenyl]methyl)-5H,6H,7H,8H-pyrido[2,3-d]pyrimidin-7-one (50 mg, 0.12 mmol, 1.00 equiv), 2-(propan-2-yl)-3-(tetramethyl-1,3,2-dioxaborolan-2-yl)pyridine (35 mg, 0.14 mmol, 1.00 equiv), Pd(dppf)Cl2 CH2Cl2 (9.6 mg, 0.01 mmol, 1.00 equiv), sodium carbonate (25 mg, 0.24 mmol, 1.00 equiv), dioxane (10 mL), water (3 mL). The resulting solution was stirred for 2 h at 100° C. The reaction mixture was cooled. The resulting solution was diluted with 15 mL of EA. The mixture was filtered through a Celite pad. The resulting mixture was concentrated under vacuum. The residue was purified by preparative TLC (EA:PE=1:3). The crude product was purified by Prep-HPLC with the following conditions (Prep-HPLC-025): Column: XBridge Shield RP18 OBD Column, 5 μm, 19*150 mm; Mobile Phase A: water (10 mmol/L NH4HCO3), Mobile Phase B: ACN; Flow rate: 20 mL/min; Gradient: 35% B to 65% B in 7 min; 254 nm; Rt: 6 min. This resulted in 24.7 mg (41%) of 8-([4-[1-methyl-4-(trifluoromethyl)-1H-imidazol-2-yl]phenyl]methyl)-2-[2-(propan-2-yl)pyridin-3-yl]-5H,6H,7H,8H-pyrido[2,3-d]pyrimidin-7-one as a white solid. LC-MS (ESI) m/z 507.2 [M+H]+ 1H NMR (400 MHZ, CD3OD) δ 8.61 (s, 1H), 8.56-8.54 (m, 1H), 7.95-7.92 (m, 1H), 7.66-7.65 (m, 1H), 7.58-7.56 (m, 2H), 7.44-7.42 (m, 2H), 7.33-7.30 (m, 1H), 5.44 (s, 2H), 3.74 (s, 3H), 3.63-3.56 (m, 1H), 3.14-3.10 (m, 2H), 2.94-2.90 (m, 2H), 1.11 (d, J=6.8 Hz, 6H).

### Example A: Ubitquin-Rhodamine 110 Assay for USP1 Activity

The HTS assay was performed in a final volume of 20 μL in assay buffer containing 20 mM Tris-HCl (pH 8.0, (1M Tris-HCl, pH 8.0 solution; Corning 46-031-CM)), 2 mM CaCl₂) (1M Calcium Chloride solution; Sigma #21114) 1 mM GSH (L-Glutathione reduced; Sigma #G4251), 0.01% Prionex (0.22 μM filtered, Sigma #G-0411), and 0.01% Triton X-100. Stock compound solutions were stored at −20° C. as 10 mM in DMSO. Up to 1 month prior to the assay, 2 mM test compounds were pre-dispensed into assay plates (Black, low volume; Corning #3820) and frozen at −20° C. Pre-stamped assay plates were allowed to come to room temperature on the day of the assay. For the screen, 100 nL of 2 mM was pre-dispensed for a final screening concentration of 10 μM (DMSO_{(fc)}=0.5%). The final concentration of the enzyme (USP1, construct USP1 (1-785, GG670, 671AA)/UAF1 (1-677)-Flag; Viva) in the assay was 100 pM. Final substrate (Ub-Rh110; Ubiquitin-Rhodamine 110, R&D Systems #U-555) concentration was 25 nM with [Ub-Rh110]<<Km. 10 μL of 2× enzyme was added to assay plates (pre-stamped with compound) either simultaneously with 2×Ub-Rh110 or preincubated with USP1 40 minutes prior to the addition of 10 μL of 2×Ub-Rh110 to compound plates. Plates were incubated stacked for 45 minutes at room temperature before fluorescence was read on the Envision (Excitation at 485 nm and Emission at 535 nm; Perkin Elmer) or on the PheraSTAR (Excitation at 485 nm and Emission at 535 nm; BMG Labtech).

For follow-up IC₅₀ studies, each assay was performed in a final volume of 15 μL in assay buffer containing 20 mM Tris-HCl (pH 8.0, (1M Tris-HCl, pH 8.0 solution; Corning 46-031-CM)), 1 mM GSH (L-Glutathione reduced; Sigma #G4251), 0.03% BGG (0.22 μM filtered, Sigma, #G7516-25G), and 0.01% Triton X-100 (Sigma, #T9284-10L)). Nanoliter quantities of either an 8-point or 10-point, 3-fold serial dilution in DMSO were pre-dispensed into assay plates (Perkin Elmer, ProxiPlate-384 F Plus, #6008269) for a final test concentration range of either 25 μM to 11 nM or 25 μM to 1.3 nM, respectively. The final concentration of the enzyme (USP1, construct USP1 (1-785, GG670, 671AA)/UAF1 (1-677)-Flag; Viva) in the assay was 25 pM. Final substrate (Ub-Rh110; Ubiquitin-Rhodamine 110, R&D Systems #U-555) concentration was 25 nM with [Ub-Rh110]<<Km. 5 μL of 2× enzyme was added to assay plates (pre-stamped with compound) preincubated with USP1 for 30 minutes and then 5 μL of 2×Ub-Rh110 was added to assay plates. Plates were incubated stacked for 20 minutes at room temperature before 5 μL of stop solution (final concentration of 10 mM citric acid in assay buffer (Sigma, #251275-500G)). Fluorescence was read on the Envision (Excitation at 485 nm and Emission at 535 nm; Perkin Elmer) or on the PheraSTAR (Excitation at 485 nm and Emission at 535 nm; BMG Labtech).

For both assay formats data were reported as percent inhibition compared with control wells based on the following equation: % inh=[1−((FLU−Ave_{Low})/(Ave_{High}−Ave_{Low}))]×100 where FLU=measured Fluorescence, Ave_{Low}=average Fluorescence of no enzyme control (n=16), and Ave_{High}=average Fluorescence of DMSO control (n=16). IC₅₀ values were determined by curve fitting of the standard 4 parameter logistic fitting algorithm included in the Activity Base software package: IDBS XE Designer Model205. Data is fitted using the Levenburg Marquardt algorithm.

As set forth in the Table below, IC₅₀ values are defined as follows: ≤0.010 μM (+++); >0.010 μM and ≤0.1 μM (++); >0.1 μM and <1.0 μM (+).

Table of Compound Activities

| IC50 | | | |
|---|---|---|---|
| (μM) | Name | Structure | Ex # |
| +++ | 5-(2-isopropylphenyl)-3-(4- (1-methyl-4- (trifluoromethyl)-1H- imidazol-2- yl)benzyl)thiazolo[4,5- d]pyrimidin-2(3H)-one | 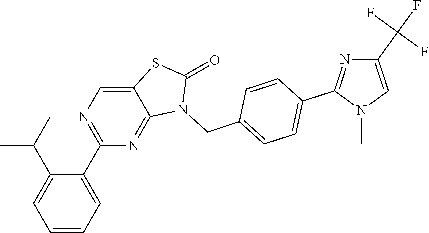 [SMILES: CC(C)c1ccccc1-c1ncc2sc(=O)n(Cc3ccc(-c4nc(C(F)(F)F)cn4C)cc3)c2n1] | I-1 |
| + | 8-(azetidin-3-yl)-2-[2- (propan-2-yl)phenyl]-9-[[4- (1H-pyrazol-1- yl)phenyl]methyl]-9H- purine | 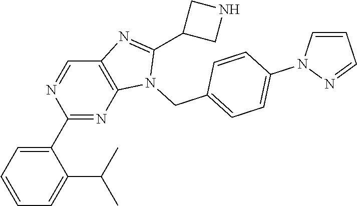 [SMILES: CC(C)c1ccccc1-c1ncc2nc(C3CNC3)n(Cc3ccc(-n4cccn4)cc3)c2n1] | I-14 |
| + | 8-(oxetan-3-yl)-2-[2- (propan-2-yl)phenyl]-9-[[4- (1H-pyrazol-1- yl)phenyl]methyl]-9H- purine | 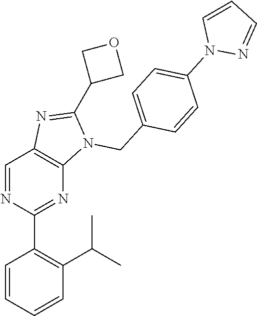 [SMILES: CC(C)c1ccccc1-c1ncc2nc(C3COC3)n(Cc3ccc(-n4cccn4)cc3)c2n1] | I-15 |
| ++ | 3-methyl-1-([4-[1-methyl-4- (trifluoromethyl)-1H- imidazol-2- yl]phenyl]methyl)-7-[2- (propan-2-yl)phenyl]- 1H,2H,3H,4H- [1,3]diazino[4,5- d]pyrimidin-2-one | 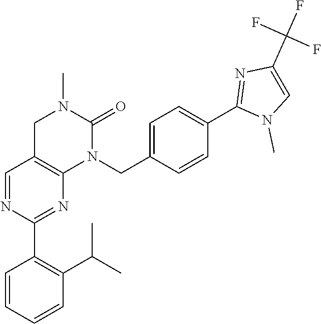 [SMILES: CC(C)c1ccccc1-c1ncc2c(n1)N(Cc1ccc(-c3nc(C(F)(F)F)cn3C)cc1)C(=O)N(C)C2] | I-6 |
| + | 8-methoxy-9-([4-[1-methyl- 4-(trifluoromethyl)-1H- imidazol-2- yl]phenyl]methyl)-2-[2- (propan-2-yl)pyridin-3-yl]- 9H-purine | 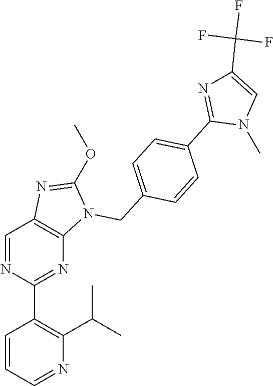 [SMILES: COc1nc2cnc(-c3cccnc3C(C)C)nc2n1Cc1ccc(-c2nc(C(F)(F)F)cn2C)cc1] | I-17 |
| +++ | 5-(2-isopropylphenyl)-3-(4- (1-methyl-4- (trifluoromethyl)-1H- imidazol-2- yl)benzyl)oxazolo[4,5- d]pyrimidin-2(3H)-one | 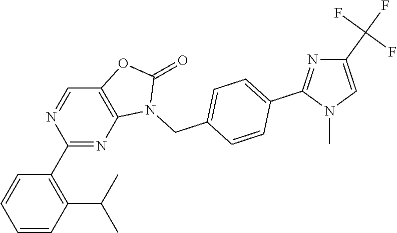 [SMILES: CC(C)c1ccccc1-c1ncc2oc(=O)n(Cc3ccc(-c4nc(C(F)(F)F)cn4C)cc3)c2n1] | I-12 |
| +++ | 1-([4-[1-methyl-4- (trifluoromethyl)-1H- imidazol-2- yl]phenyl]methyl)-7-[2- (propan-2-yl)phenyl]- 1H,2H,4H-pyrimido[4,5- d][1,3]oxazin-2-one | 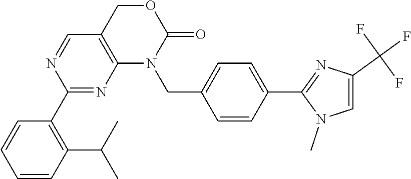 [SMILES: CC(C)c1ccccc1-c1ncc2c(n1)N(Cc1ccc(-c3nc(C(F)(F)F)cn3C)cc1)C(=O)OC2] | I-7 |
| ++ | 1-([4-[1-methyl-4- (trifluoromethyl)-1H- imidazol-2- yl]phenyl]methyl)-7-[2- (propan-2-yl)pyridin-3-yl]- 1H,2H,4H-pyrimido[4,5- d][1,3]oxazin-2-on | 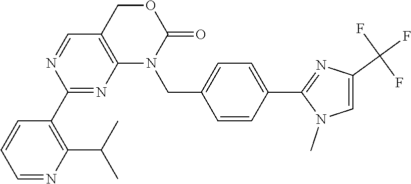 [SMILES: CC(C)c1ncccc1-c1ncc2c(n1)N(Cc1ccc(-c3nc(C(F)(F)F)cn3C)cc1)C(=O)OC2] | I-8 |
| + | 3-([4-[1-methyl-4- (trifluoromethyl)-1H- imidazol-2- yl]phenyl]methyl)-5-[2- (propan-2-yl)phenyl]- 2H,3H-[1,3]oxazolo[4,5- d]pyrimidin-2-one | 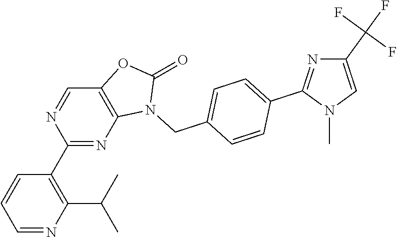 [SMILES: CC(C)c1ncccc1-c1ncc2oc(=O)n(Cc3ccc(-c4nc(C(F)(F)F)cn4C)cc3)c2n1] | I-13 |
| ++ | 7-([4-[1-methyl-4- (trifluoromethyl)-1H- imidazol-2- yl]phenyl]methyl)-2-[2- (propan-2-yl)pyridin-3-yl]- 5H,6H,7H-pyrrolo[2,3- d]pyrimidin-6-one | 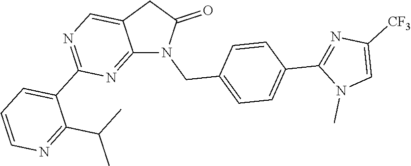 [SMILES: CC(C)c1ncccc1-c1ncc2c(n1)N(Cc1ccc(-c3nc(C(F)(F)F)cn3C)cc1)C(=O)C2] | I-16 |
| +++ | 7-([4-[1-methyl-4- (trifluoromethyl)-1H- imidazol-2- yl]phenyl]methyl)-2-[2- (propan-2-yl)phenyl]- 5H,6H,7H-pyrrolo[2,3- d]pyrimidin-6-one | 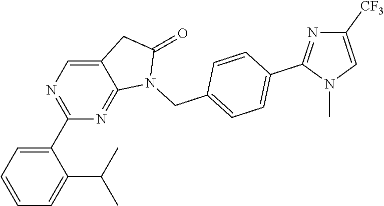 [SMILES: CC(C)c1ccccc1-c1ncc2c(n1)N(Cc1ccc(-c3nc(C(F)(F)F)cn3C)cc1)C(=O)C2] | I-18 |
| +++ | 8-([4-[1-methyl-4- (trifluoromethyl)-1H- imidazol-2- yl]phenyl]methyl)-2-[2- (propan-2-yl)phenyl]- 6H,7H,8H-pyrimido[5,4- b][1,4]oxazin-7-one | 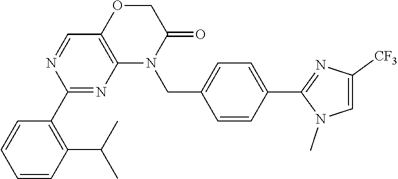 [SMILES: CC(C)c1ccccc1-c1ncc2c(n1)N(Cc1ccc(-c3nc(C(F)(F)F)cn3C)cc1)C(=O)CO2] | I-3 |
| ++ | 3′-([4-[1-methyl-4- (trifluoromethyl)-1H- imidazol-2- yl]phenyl]methyl)-5′-[2- (propan-2-yl)pyridin-3-yl]- 2′,3′- dihydrospiro[cyclopropane- 1,1′-pyrrolo[2,3- d]pyrimidine]-2′-one | 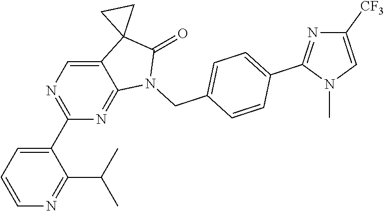 [SMILES: CC(C)c1ncccc1-c1ncc2c(n1)N(Cc1ccc(-c3nc(C(F)(F)F)cn3C)cc1)C(=O)C21CC1] | I-11 |
| +++ | 3-[7-([4-[1-methyl-4- (trifluoromethyl)-1H- imidazol-2- yl]phenyl]methyl)-7H- pyrrolo[2,3-d]pyrimidin-2- yl]-2-(propan-2-yl)pyridine | 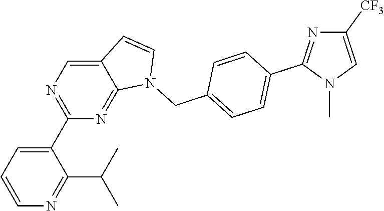 [SMILES: CC(C)c1ncccc1-c1ncc2ccn(Cc3ccc(-c4nc(C(F)(F)F)cn4C)cc3)c2n1] | I-9 |
| + | 7-([4-[1-(oxetan-3-yl)-4- (trifluoromethyl)-1H- imidazol-2- yl]phenyl]methyl)-2-[2- (propan-2-yl)pyridin-3-yl]- 5H,6H,7H-pyrrolo[2,3- d]pyrimidin-6-one | 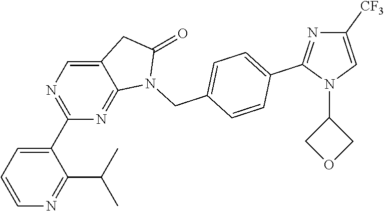 [SMILES: CC(C)c1ncccc1-c1ncc2c(n1)N(Cc1ccc(-c3nc(C(F)(F)F)cn3C3COC3)cc1)C(=O)C2] | I-19 |
| ++ | 2-chloro-8-([4-[1-methyl-4- (trifluoromethyl)-1H- imidazol-2- yl]phenyl]methyl)- 5H,6H,7H,8H-pyrido[2,3- d]pyrimidin-7-one | 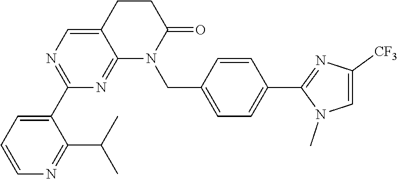 [SMILES: CC(C)c1ncccc1-c1ncc2c(n1)N(Cc1ccc(-c3nc(C(F)(F)F)cn3C)cc1)C(=O)CC2] | I-20 |
| +++ | 3-[8-([4-[1-methyl-4- (trifluoromethyl)-1H- imidazol-2- yl]phenyl]methyl)- 6H,7H,8H-pyrimido[5,4- b][1,4]oxazin-2-yl]-2- (propan-2-yl)pyridine | 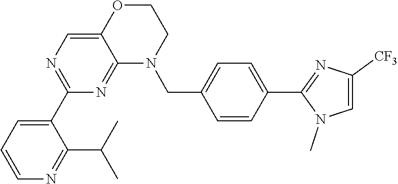 [SMILES: CC(C)c1ncccc1-c1ncc2c(n1)N(Cc1ccc(-c3nc(C(F)(F)F)cn3C)cc1)CCO2] | I-2 |
| ++ | 8-([4-[1-methyl-4- (trifluoromethyl)-1H- imidazol-2- yl]phenyl]methyl)-2-[2- (propan-2-yl)pyridin-3-yl]- 6H,7H,8H-pyrimido[5,4- b][1,4]oxazin-7-one | 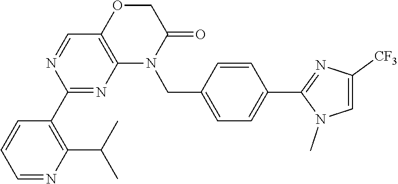 [SMILES: CC(C)c1ncccc1-c1ncc2c(n1)N(Cc1ccc(-c3nc(C(F)(F)F)cn3C)cc1)C(=O)CO2] | I-4 |
| ++ | 3-[1-([4-[1-methyl-4- (trifluoromethyl)-1H- imidazol-2- yl]phenyl]methyl)-1H- pyrazolo[3,4-d]pyrimidin-6- yl]-2-(propan-2-yl)pyridine | 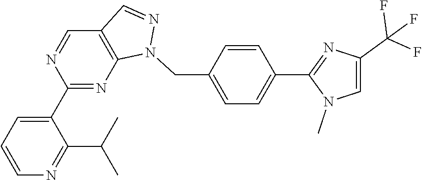 [SMILES: CC(C)c1ncccc1-c1ncc2cnn(Cc3ccc(-c4nc(C(F)(F)F)cn4C)cc3)c2n1] | I-10 |
| ++ | 6,6-dimethyl-8-([4-[1- methyl-4-(trifluoromethyl)- 1H-imidazol-2- yl]phenyl]methyl)-2-[2- (propan-2-yl)pyridin-3-yl]- 6H,7H,8H-pyrimido[5,4- b][1,4]oxazin-7-one | 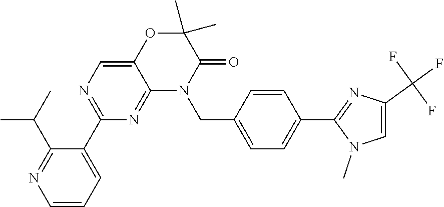 [SMILES: CC(C)c1ncccc1-c1ncc2c(n1)N(Cc1ccc(-c3nc(C(F)(F)F)cn3C)cc1)C(=O)C(C)(C)O2] | I-5 |
| +++ | 5-(4-cyclopropyl-6- methoxypyrimidin-5-yl)-3- (4-(5-methyl-3- (trifluoromethyl)-1H- pyrazol-1- yl)benzyl)thiazolo[4,5- d]pyrimidin-2(3H)-one | 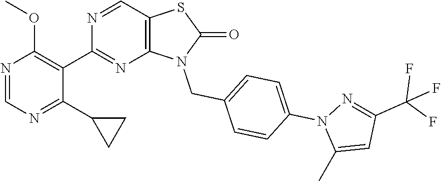 [SMILES: COc1ncnc(C2CC2)c1-c1ncc2sc(=O)n(Cc3ccc(-n4nc(C(F)(F)F)cc4C)cc3)c2n1] | I-21 |
| +++ | 5-(4-cyclopropyl-6- methoxypyrimidin-5-yl)-3- (4-(1-methyl-4- (trifluoromethyl)-1H- imidazol-2- yl)benzyl)thiazolo[4,5- d]pyrimidin-2(3H)-one | 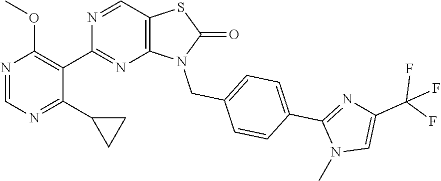 [SMILES: COc1ncnc(C2CC2)c1-c1ncc2sc(=O)n(Cc3ccc(-c4nc(C(F)(F)F)cn4C)cc3)c2n1] | I-22 |

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

We claim:

1. A compound of Formula (I) 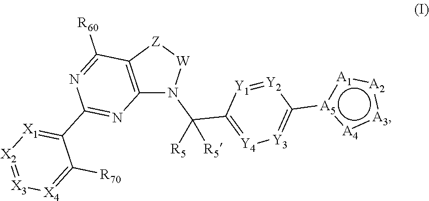
or a pharmaceutically acceptable salt thereof,
wherein,
X₁ is CR₆ or N; X₂ is CR₇ or N; X₃ is CR₈ or N; and X₄ is CR₉ or N;
R₆ is hydrogen, halogen, (C₁-C₄)alkyl, (C₁-C₄)alkoxy, (C₃-C₆)cycloalkyl, —O—(C₃-C₆)cycloalkyl, or 4- to 6-membered heterocycloalkyl having 1-3 heteroatoms independently selected from N, O, and S, wherein the alkyl, cycloalkyl or heterocycloalkyl is optionally substituted with one or more substituents independently selected from halogen, hydroxyl, (C₁-C₄)alkoxy, or —NR_{b}R_{b};
R₇, R₈ and R₉ are each independently hydrogen or halogen;
R₇₀ is hydrogen, halogen, (C₁-C₄)alkyl, (C₁-C₄)alkoxy, (C₃-C₆)cycloalkyl, —O—(C₃-C₆)cycloalkyl, or 4- to 6-membered heterocycloalkyl having 1-3 heteroatoms independently selected from N, O, and S, wherein the alkyl, cycloalkyl or heterocycloalkyl is optionally substituted with one or more substituents independently selected from halogen, hydroxyl, (C₁-C₄)alkoxy, or —NR_{b}R_{b};
R₆₀ is hydrogen, halogen, (C₁-C₄)alkyl, (C₁-C₄)alkoxy, (C₃-C₆)cycloalkyl, or 4- to 6-membered heterocycloalkyl having 1-3 heteroatoms independently selected from N, O, and S, wherein the alkyl, cycloalkyl or heterocycloalkyl is optionally substituted with one or more substituents independently selected from halogen, hydroxyl, (C₁-C₄)alkoxy, or —NR_{b}R_{b};
Z and W are together selected to form a fused 5- or 6-membered ring selected from cycloalkyl, cycloalkenyl, heterocycloalkyl having 1-3 heteroatoms independently selected from N, O, or S, or heterocycloalkenyl having 1-3 heteroatoms independently selected from N, O, or S, wherein Z is selected from the group consisting of: —C(R₁₆)(R₁₆′)—, —C(R₂₀)(R₂₀′)—C(R₁₈)(R₁₈′)—*, —C(R₂₀)(R₂₀′)—C(R₁₈)═*, —S—, —S—C(R₁₈)(R₁₈′)—*, —C(R₁₈)(R₁₈′)—S—*, —N(R₁₄)—, —N(R₁₄)—C(R₁₈)(R₁₈′)—*, —N(R₁₄)—C(R₁₈)═*, —N═C(R₁₈)—*, —C(R₁₈)(R₁₈′)—N(R₁₄)—*, —O—, —O—C(R₁₈)(R₁₈′)—*, —C(R₁₈)(R₁₈′)—O—*, —O—C(R₁₈)═*, and —C(R₂₀)═C(R₁₈)—*, wherein * represents the point of attachment to W, and W is selected from the group consisting of: —(C═O)— ═C(R₉₀)—, and —C(R₁₀)(R₁₀′)—, provided that when Z is —N(R₁₄)—, W is not —(C═O)—; or
Z and W are together selected to form a fused 5- or 6-membered heteroaryl ring, having 1-3 heteroatoms independently selected from N, O, or S, wherein Z is selected from the group consisting of —C(R₂₀)═*, —N═*, and —S—C(R₁₈)═*, wherein * represents the point of attachment to W; and W is selected from the group consisting of ═N— and ═C(R₉₀)—;
R₉₀ is selected from the group consisting of hydrogen; hydroxyl; (C₁-C₄)alkyl optionally substituted with one or more halogen, hydroxyl or —N(R_{b})(R_{b}′); (C₃-C₆)cyclopropyl optionally substituted with one or more (C₁-C₄)alkyl; —O—(C₁-C₄) alkyl optionally substituted with one or more halogen; (C₁-C₄)alkyl-N(R_{b})(R_{b}′); —O—(C₃-C₆)cycloalkyl; and a 4- to 6-membered heterocycloalkyl having 1-3 heteroatoms independently selected from N, O, or S;
R₁₀, R₁₀′, R₁₆, R₁₆′, R₁₈, R₁₈′, R₂₀, R₂₀′ are each independently selected from the group consisting of hydrogen, hydroxyl, (C₁-C₄)alkyl optionally substituted with one or more halogen, —O—(C₁-C₄) alkyl optionally substituted with one or more halogens, (C₁-C₄)alkyl-N(R_{b})(R_{b}′); or R₁₆ and R₁₆′, R₁₈ and R₁₈′, and R₂₀ and R₂₀′ each together form a spirocyclic 3- to 6-membered cycloalkyl optionally substituted with one or more Rₐ′;
R₁₄ is selected from the group consisting of hydrogen and (C₁-C₄)alkyl;
R₅ and R₅′ are each independently selected from hydrogen, halogen, (C₁-C₄)alkyl, —(C₁-C₄)alkyl-O—(C₁-C₄) alkyl, or —(C₁-C₄)alkyl-N(R_{b})(R_{b}′), wherein each alkyl is optionally substituted with one or more halogens; or R₅ and R₅′ together form a (C₃-C₆)cycloalkyl ring optionally substituted with one or more substituents independently selected from halogen or (C₁-C₄)alkyl;
Y₁, Y₂, Y₃ and Y₄ are each independently —C(R_{y})— or N, wherein each R_{y} is independently hydrogen, halogen, or (C₁-C₄)alkyl;
each of A₁, A₂, A₃, and A₄, is independently selected from the group consisting of C(R₂), N(R₁), O, and S;
A₅ is N or CR₂;
wherein A₁, A₂, A₃, A₄, A₅ together form a 5-membered heteroaryl ring;
wherein
each R₁ is independently a bond, hydrogen, (C₁-C₄)alkyl, —O—(C₁-C₄)alkyl, 3- to 6-membered cycloalkyl, or 3- to 6-membered heterocycloalkyl having 1-3 heteroatoms independently selected from N, O, and S, wherein each (C₁-C₄)alkyl or —O—(C₁-C₄)alkyl is independently optionally substituted with one or more Rₐ, and each 3- to 6-membered cycloalkyl or 3- to 6-membered heterocycloalkyl is independently optionally substituted with one or more R_{a′};
each R₂ is independently a bond, hydrogen, (C₁-C₄)alkyl, —O—(C₁-C₄)alkyl, 3- to 6-membered cycloalkyl, or 3- to 6-membered heterocycloalkyl having 1-3 heteroatoms independently selected from N, O, and S, wherein each (C₁-C₄)alkyl or —O—(C₁-C₄)alkyl is independently optionally substituted with one or more Rₐ, and each 3- to 6-membered cycloalkyl or 3- to 6-membered heterocycloalkyl is independently optionally substituted with one or more R_{a′}; or
two occurrences of R₂ on adjacent carbon atoms in A₁, A₂, A₃, or A₄, may together form a fused ring selected from a 5- to 6-membered heterocycloalkyl having 1-3 heteroatoms selected from N, O, or S or a 5- to 6-membered heteroaryl having 1-3 heteroatoms selected from N, O, or S, wherein each ring may independently be optionally substituted with one or more R_{a′};
each Rₐ is independently selected from the group consisting of halogen, (C₁-C₄)alkyl, hydroxyl, —N(R_{b})(R_{b}′), (C₁-C₄)alkoxy optionally substituted with one or more R_{a′}, and 3- to 6-membered cycloalkyl optionally substituted with one or more R_{a′};
each R_{a′} is independently halogen or (C₁-C₄)alkyl optionally substituted with one or more halogen, and
each R_{b} and R_{b}′ is independently selected from the group consisting of hydrogen and (C₁-C₄)alkyl optionally substituted with one or more halogen.
provided that:
when Z is —N═ or —C(R₂₀)═, wherein R₂₀ is hydrogen or (C₁-C₄)alkyl optionally substituted with one or more halogen;
then W is not ═N— or —C(R₉₀)—, wherein R₉₀ is hydrogen or (C₁-C₄)alkyl optionally substituted with one or more halogen.

2. A compound of Formula (I) 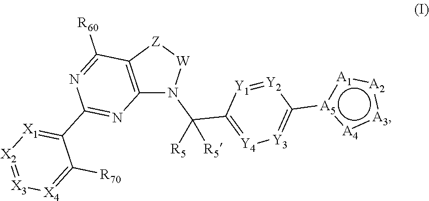
or a pharmaceutically acceptable salt thereof,
wherein,
X₁ is CR₆ or N; X₂ is CR₇ or N; X₃ is CR₈ or N; and X₄ is CR₉ or N;
R₆ is hydrogen, halogen, (C₁-C₄)alkyl, (C₁-C₄)alkoxy, (C₃-C₆)cycloalkyl, —O—(C₃-C₆)cycloalkyl, or 4- to 6-membered heterocycloalkyl having 1-3 heteroatoms independently selected from N, O, and S, wherein the alkyl, cycloalkyl or heterocycloalkyl is optionally substituted with one or more substituents independently selected from halogen, hydroxyl, (C₁-C₄)alkoxy, or —NR_{b}R_{b′};
R₇, R₈ and R₉ are each independently hydrogen or halogen;
R₇₀ is hydrogen, halogen, (C₁-C₄)alkyl, (C₁-C₄)alkoxy, (C₃-C₆)cycloalkyl, —O—(C₃-C₆)cycloalkyl, or 4- to 6-membered heterocycloalkyl having 1-3 heteroatoms independently selected from N, O, and S, wherein the alkyl, cycloalkyl or heterocycloalkyl is optionally substituted with one or more substituents independently selected from halogen, hydroxyl, (C₁-C₄)alkoxy, or —NR_{b}R_{b′};
R₆₀ is hydrogen, halogen, (C₁-C₄)alkyl, (C₁-C₄)alkoxy, (C₃-C₆)cycloalkyl, or 4- to 6-membered heterocycloalkyl having 1-3 heteroatoms independently selected from N, O, and S, wherein the alkyl, cycloalkyl or heterocycloalkyl is optionally substituted with one or more substituents independently selected from halogen, hydroxyl, (C₁-C₄)alkoxy, or —NR_{b}R_{b′};
Z and W are together selected to form a fused 5- or 6-membered heteroaryl ring, having 1-3 heteroatoms independently selected from N, O, or S, wherein Z is selected from the group consisting of —C(R₂₀)═* or —N═*, wherein * represents the point of attachment to W; and W is selected from the group consisting of ═N— and ═C(R₉₀)—;
R₉₀ is selected from the group consisting of hydrogen or (C₁-C₄)alkyl optionally substituted with one or more halogen;
R₂₀ is hydrogen or (C₁-C₄)alkyl optionally substituted with one or more halogen;
R₅ and R₅′ are each independently selected from hydrogen, halogen, (C₁-C₄)alkyl, —(C₁-C₄)alkyl-O—(C₁-C₄)alkyl, or —(C₁-C₄)alkyl-N(R_{b})(R_{b}′), wherein each alkyl is optionally substituted with one or more halogens; or R₅ and R₅′ together form a (C₃-C₆)cycloalkyl ring optionally substituted with one or more substituents independently selected from halogen or (C₁-C₄)alkyl;
Y₁, Y₂, Y₃ and Y₄ are each independently —C(R_{y})— or N, wherein each R_{y} is independently hydrogen, halogen, or (C₁-C₄)alkyl;
each of A₁, A₂, A₃, and A₄, is independently selected from the group consisting of C(R₂), N(R₁), O, and S;
A₅ is N or CR₂;
wherein A₁, A₂, A₃, A₄, A₅ together form a 5-membered heteroaryl ring;
wherein
each R₁ is independently a bond, hydrogen, (C₁-C₄)alkyl, —O—(C₁-C₄)alkyl, 3- to 6-membered cycloalkyl, or 3- to 6-membered heterocycloalkyl having 1-3 heteroatoms independently selected from N, O, and S, wherein each (C₁-C₄)alkyl or —O—(C₁-C₄)alkyl is independently optionally substituted with one or more Rₐ, and each 3- to 6-membered cycloalkyl or 3- to 6-membered heterocycloalkyl is independently optionally substituted with one or more R_{a′};
each R₂ is independently a bond, hydrogen, (C₁-C₄)alkyl, —O—(C₁-C₄)alkyl, 3- to 6-membered cycloalkyl, or 3- to 6-membered heterocycloalkyl having 1-3 heteroatoms independently selected from N, O, and S, wherein each (C₁-C₄)alkyl or —O—(C₁-C₄)alkyl is independently optionally substituted with one or more Rₐ, and each 3- to 6-membered cycloalkyl or 3- to 6-membered heterocycloalkyl is independently optionally substituted with one or more R_{a′}; or
two occurrences of R₂ on adjacent carbon atoms in A₁, A₂, A₃, or A₄, may together form a fused ring selected from a 5- to 6-membered heterocycloalkyl having 1-3 heteroatoms selected from N, O, or S or a 5- to 6-membered heteroaryl having 1-3 heteroatoms selected from N, O, or S, wherein each ring may independently be optionally substituted with one or more R_{a′};
each R^{a} is independently selected from the group consisting of halogen, (C₁-C₄)alkyl, hydroxyl, —N(R_{b})(R_{b}′), (C₁-C₄)alkoxy optionally substituted with one or more Rₐ′, and 3- to 6-membered cycloalkyl optionally substituted with one or more Rₐ;
each R_{a′} is independently halogen or (C₁-C₄)alkyl optionally substituted with one or more halogen, and
each R_{b} and R_{b}′ is independently selected from the group consisting of hydrogen and (C₁-C₄)alkyl optionally substituted with one or more halogen.
provided that:
the compound is not:
6-(3-methoxypyridin-4-yl)-1-(4-(1-methyl-4-(trifluoromethyl)-1H-imidazol-2-yl)benzyl)-1H-pyrazolo[3,4-d]pyrimidine;
1-(4-(1-methyl-4-(trifluoromethyl)-1H-imidazol-2-yl)benzyl)-6-(2-(methylsulfonyl)-phenyl)-1H-pyrazolo[3,4-d]pyrimidine;
1-(4-(1-methyl-4-(trifluoromethyl)-1H-imidazol-2-yl)benzyl)-6-(2-methyl-6-(methylsulfonyl)phenyl)-1H-pyrazolo[3,4-d]pyrimidine;
6-(2-(ethylsuffonyl)phenyl)-1-(4-(1-methyl-4-(trifluoromethyl)-1H-imidazol-2-yl)benzyl)-1H-pyrazolo[3,4-d]pyrimidine;
1-(4-(3-(difluoromethyl)-5-methyl-1H-pyrazol-1-yl)benzyl)-6-(2-isopropylpyridin-3-yl)-1H-pyrazolo[3,4-d]pyrimidine;
6-(2-methoxy-4-methylpyridin-3-yl)-1-(4-(1-methyl-4-(trifluoromethyl)-1H-imidazol-2-yl)benzyl)-1H-pyrazolo[3,4-d]pyrimidine;
6-(4-methoxy-6-methylpyrimidin-5-yl)-1-(4-(1-methyl-4-(trifluoromethyl)-1H-imidazol-2-yl)benzyl)-1H-pyrazolo[3,4-d]pyrimidine;
6-(2-isopropylpyridin-3-yl)-1-(4-(5-methyl-3-(trifluoromethyl)-1H-pyrazol-1-yl)benzyl)-1H-pyrazolo[3,4-d]pyrimidine;
6-(2,6-dimethoxyphenyl)-1-(4-(1-methyl-4-(trifluoromethyl)-1H-imidazol-2-yl)benzyl)-1H-pyrazolo[3,4-d]pyrimidine;
6-(2-methoxyphenyl)-1-(4-(1-methyl-4-(trifluoromethyl)-1H-imidazol-2-yl)benzyl)-1H-pyrazolo[3,4-d]pyrimidine;
6-(2-isopropylphenyl)-1-(4-(5-methyl-3-(trifluoromethyl)-1H-pyrazol-1-yl)benzyl)-1H-pyrazolo[3,4-d]pyrimidine;
6-(2-methoxy-6-methylphenyl)-1-(4-(1-methyl-4-(trifluoromethyl)-1H-imidazol-2-yl)benzyl)-1H-pyrazolo[3,4-d]pyrimidine;
2-methoxy-3-(1-(4-(1-methyl-4-(trifluoromethyl)-1H-imidazol-2-yl)benzyl)-1H-pyrazolo[3,4-d]pyrimidin-6-yl)isonicotinonitrile;
2-(2-isopropylphenyl)-9-(4-(1-methyl-4-(trifluoromethyl)-1H-imidazol-2-yl)benzyl)-9H-purine;
6-(4-cyclopropyl-6-methoxypyrimidin-5-yl)-1-(4-(1-methyl-4-(trifluoromethyl)-1H-imidazol-2-yl)benzyl)-1H-pyrazolo[3,4-d]pyrimidine;
6-(2-isopropylpyridin-3-yl)-1-(4-(1-methyl-4-(trifluoromethyl)-1H-imidazol-2-yl)benzyl)-1H-pyrazolo[3,4-d]pyrimidine (Example 9); 1-(4-(1-methyl-4-(trifluoromethyl)-1H-imidazol-2-yl)benzyl)-6-(2-methyl-6-(methylthio)phenyl)-1H-pyrazolo[3,4-d]pyrimidine;
6-(4-cyclopropyl-6-methoxypyrimidin-5-yl)-1-(4-(5-methyl-3-(trifluoromethyl)-1H-pyrazol-1-yl)benzyl)-1H-pyrazolo[3,4-d]pyrimidine;
6-(2-cyclopropyl-6-methoxyphenyl)-1-(4-(1-methyl-4-(trifluoromethyl)-1H-imidazol-2-yl)benzyl)-1H-pyrazolo[3,4-d]pyrimidine;
6-(2-isopropylphenyl)-1-(4-(1-(1-methylazetidin-3-yl)-4-(trifluoromethyl)-1H-imidazol-2-yl)benzyl)-1H-pyrazolo[3,4-d]pyrimidine;
6-(2-isopropylphenyl)-1-(1-(4-(1-methyl-4-(trifluoromethyl)-1H-imidazol-2-yl)phenyl)ethyl)-1H-pyrazolo[3,4-d]pyrimidine;
6-(2-isopropylphenyl)-4-methyl-1-(4-(1-methyl-4-(trifluoromethyl)-1H-imidazol-2-yl)benzyl)-1H-pyrazolo[3,4-d]pyrimidine;
6-(3-fluoro-2-isopropylphenyl)-1-(4-(1-methyl-4-(trifluoromethyl)-1H-imidazol-2-yl)benzyl)-1H-pyrazolo[3,4-d]pyrimidine;
6-(2-isopropylphenyl)-3-methyl-1-(4-(1-methyl-4-(trifluoromethyl)-1H-imidazol-2-yl)benzyl)-1H-pyrazolo[3,4-d]pyrimidine;
6-(2-isopropylphenyl)-1-(4-(1-methyl-4-(trifluoromethyl)-1H-imidazol-2-yl)benzyl)-1H-pyrazolo[3,4-d]pyrimidine;
6-(4-(tert-butyl)-6-methoxypyrimidin-5-yl)-1-(4-(1-methyl-4-(trifluoromethyl)-1H-imidazol-2-yl)benzyl)-1Ii-pyrazolo[3,4-d]pyrimidine;
6-(4-(2-fluoropropan-2-yl)-6-methoxypyrimidin-5-yl)-1-(4-(1-methyl-4-(trifluoromethyl)-1H-imidazol-2-yl)benzyl)-1H-pyrazolo[3,4-d]pyrimidine;
6-(4-methoxy-6-(1-methylcyclopropyl)pyrimidin-5-yl)-1-(4-(1-methyl-4-(trifluoromethyl)-1H-imidazol-2-yl)benzyl)-1H-pyrazolo[3,4-d]pyrimidine;
6-(4-cyclobutyl-6-methoxypyrimidin-5-yl)-1-(4-(1-methyl-4-(trifluoromethyl)-1II-imidazol-2-yl)benzyl)-1H-pyrazolo[3,4-d]pyrimidine;
(S)-6-(4-cyclopropyl-6-methoxypyrimidin-5-yl)-1-(1-(4-(1-methyl-4-(trifluoromethyl)-1H-imidazol-2-yl)phenyl)ethyl)-1H-pyrazolo[3,4-d]pyrimidine;
(R)-6-(4-cyclopropyl-6-methoxypyrimidin-5-yl)-1-(1-(4-(1-methyl-4-(trifluoromethyl)-1H-imidazol-2-yl)phenyl)ethyl)-1H-pyrazolo[3,4-d]pyrimidine;
6-(4-cyclopropyl-6-ethoxypyrimidin-5-yl)-1-(4-(1-methyl-4-(trifluoromethyl)-1H-imidazol-2-yl)benzyl)-1H-pyrazolo[3,4-d]pyrimidine; 6-(4-cyclopropyl-6-isopropoxypyrimidin-5-yl)-1-(4-(1-methy{-4-(trifluoromethyl)-1H-imidazol-2-yl)benzyl)-1H-pyrazolo[3,4-d]pyrimidine;
6-cyclopropyl-5-(1-(4-(1-methyl-4-(trifluoromethyl)-1H-imidazol-2-yl)benzyl)-1H-pyrazolo[3,4-d]pyrimidin-6-yl)pyrimidine-4-carbonitrile;
6-cyclopropyl-N,N-dimethyl-5-(1-(4-(1-methyl-4-(trifluoromethyl)-1H-imidazol-2-yl)benzyl)-1H-pyrazolo[3,4-d]pyrimidin-6-yl)pyrimidin-4-amine;
6-(4,6-dimethoxypyrimidin-5-yl)-1-(4-(1-methyl-4-(trifluoromethyl)-1H-imidazol-2-yl)benzyl)-1H-pyrazolo[3,4-d]pyrimidine;
6-(4-cyclopropyl-6-methoxypyrimidin-5-yl)-1-(4-(1-ethyl-4-(trifluoromethyl)-1H-imidazol-2-yl)benzyl)-1H-pyrazolo[3,4-d]pyrimidine;
6-(4-isopropyl-6-methylpyrimidin-5-yl)-1-(4-(5-methyl-3-(trifluoromethyl)-1H-pyrazol-1-yl)benzyl)-1H-pyrazolo[3,4-d]pyrimidine;
1-(4-(5-methyl-3-(trifluoromethyl)-1H-pyrazol-1-yl)benzyl)-6-(4-methyl-6-(methylthio)pyrimidin-5-yl)-1H-pyrazolo[3,4-d]pyrimidine;
2-(4-cyclopropyl-6-methoxypyrimidin-5-yl)-8-methyl-9-(4-(5-methyl-3-(trifluoromethyl)-1H-pyrazol-1-yl)benzyl)-9H-purine;
1-(4-(5-methyl-3-(trifluoromethyl)-1H-pyrazol-1-yl)benzyl)-6-(4-methyl-6-(methylsulfonyl)pyrimidin-5-yl)-1H-pyrazolo[3,4-d]pyrimidine;
6-(4-(2-methoxyethoxy)-6-methylpyrimidin-5-yl)-1-(4-(5-methyl-3-(trifluoromethyl)-1H-pyrazol-1-yl)benzyl)-1H-pyrazolo[3,4-d]pyrimidine;
6-(1-isopropyl-4-methyl-1H-pyrazol-5-yl)-1-(4-(5-methyl-3-(trifluoromethyl)-1H-pyrazol-1-yl)benzyl)-1H-pyrazolo[3,4-d]pyrimidine;
6-(1-isopropyl-4-methoxy-1H-pyrazol-5-yl)-1-(4-(5-methyl-3-(trifluoromethyl)-1H-pyrazol-1-yl)benzyl)-1H-pyrazolo[3,4-d]pyrimidine;
6-(4-cyclopropyl-1-isopropyl-1H-pyrazol-5-yl)-1-(4-(5-methyl-3-(trifluoromethyl)-1H-pyrazol-1-yl)benzyl)-1H-pyrazolo[3,4-d]pyrimidine;
2-(4-cyclopropyl-6-methoxypyrimidin-5-yl)-7-(4-(1-methyl-4-(trifluoromethyl)-1H-imidazol-2-yl)benzyl)-7H-pyrrolo[2,3-d]pyrimidine;
5-(4-cyclopropyl-6-methoxypyrimidin-5-yl)-3-(4-(1-methyl-4-(trifluoromethyl)-1H-imidazol-2-yl)benzyl)-3H-[1,2,3]triazolo[4,5-d]pyrimidine;
6-(4,6-diethoxypyrimidin-5-yl)-1-(4-(1-ethyl-4-(trifluoromethyl)-1H-imidazol-2-yl)benzyl)-1H-pyrazolo[3,4-d]pyrimidine;
6-(4-cyclopropyl-6-methoxypyrimidin-5-yl)-1-(4-(1-(trifluoromethyl)-5,6,7,8-tetrahydroimidazo[L5-a]pyrazin-3-yl)benzyl)-1H-pyrazolo[3,4-d]pyrimidine;
6-(4-cyclopropyl-6-methoxypyrimidin-5-yl)-1-(4-(1-methyl-5-(trifluoromethyl)-1H-1,2,4-triazol-3-yl)benzyl)-1H-pyrazolo[3,4-d]pyrimidine;
6-(1-cyclopropyl-4-methoxy-1H-pyrazol-5-yl)-1-(4-(1-ethyl-4-(trifluoromethyl)-1H-imidazol-2-yl)benzyl)-1H-pyrazolo[3,4-d]pyrimidine;
6-(4-cyclopropyl-6-methoxypyrimidin-5-yl)-1-((6-(5-methoxy-3-(trifluoromethyl)-1H-pyrazol-1-yl)pyridin-3-yl)methyl)-1H-pyrazolo[3,4-d]pyrimidine;
(R)-6-(4-cyclobutyl-6-methoxypyrimidin-5-yl)-1-(1-(4-(1-ethyl-4-(trifluoromethyl)-1H-imidazol-2-yl)phenyl)ethyl)-1H-pyrazolo[3,4-d]pyrimidine;
(S)-6-(4-cyclobutyl-6-methoxypyrimidin-5-yl)-1-(1-(4-(1-ethyl-4-(trifluoromethyl)-1H-imidazol-2-yl)phenyl)ethyl)-1H-pyrazolo[3,4-d]pyrimidine;
(R)-6-(4-cyclopropyl-6-methoxypyrimidin-5-yl)-1-(1-(4-(1-ethyl-4-(trifluoromethyl)-1H-imidazol-2-yl)phenyl)ethyl)-1H-pyrazolo[3,4-d]pyrimidine;
(S)-6-(4-cyclopropyl-6-methoxypyrimidin-5-yl)-1-(1-(4-(1-ethyl-4-(trifluoromethyl)-1H-imidazol-2-yl)phenyl)ethyl)-1H-pyrazolo[3,4-d]pyrimidine;
6-(4-cyclopropyl-6-methoxypyrimidin-5-yl)-1-(4-(1-methyl-2-(trifluoromethyl)-1H-imidazol-4-yl)benzyl)-1H-pyrazolo[3,4-d]pyrimidine;
6-(4-(2-fluoropropan-2-yl)-6-methoxypyrimidin-5-yl)-1-(4-(1-methyl-4-(trifluoromethyl)-1H-imidazol-2-yl)benzyl)-1H-pyrazolo[3,4-d]pyrimidine;
6-(4-cyclopropyl-6-methoxypyrimidin-5-yl)-1-(4-(5-ethoxy-3-(trifluoromethyl)-1H-pyrazol-1-yl)benzyl)-1H-pyrazolo[3,4-d]pyrimidine;
6-(4-cyclopropyl-6-methoxypyrimidin-5-yl)-1-((6-(1-ethyl-4-(trifluoromethyl)-1H-imidazol-2-yl)pyridin-3-yl)methyl)-1H-pyrazolo[3,4-d]pyrimidine;
1-(4-((6-(4-cyclopropyl-6-methoxypyrimidin-5-yl)-1H-pyrazolo[3,4-d]pyrimidin-1-yl)methyl)phenyl)-5-methyl-1H-pyrazole-3-carbonitrile;
6-(4-cyclopropyl-1-ethyl-1H-pyrazol-5-yl)-1-(4-(1-ethyl-4-(trifluoromethyl)-1H-imidazol-2-yl)benzyl)-1H-pyrazolo[3,4-d]pyrimidine;
6-(4-cyclopropyl-6-(methoxy-d3)pyrimidin-5-yl)-1-(4-(1-ethyl-4-(trifluoromethyl)-1H-imidazol-2-yl)benzyl)-1H-pyrazolo[3,4-d]pyrimidine; 6-cyclopropyl-5-(1-(4-(1-methyl-4-(trifluoromethyl)-1H-imidazol-2-yl)benzyl)-1H-pyrazolo[3,4-d]pyrimidin-6-yl)pyrimidine-4-carbonitrile;
6-(4-cyclopropoxy-6-cyclopropylpyrimidin-5-yl)-1-(4-(1-ethyl-4-(trifluoromethyl)-1H-imidazol-2-yl)benzyl)-1H-pyrazolo[3,4-d]pyrimidine;
6-(4-cyclobutoxy-6-cyclopropylpyrimidin-5-yl)-1-(4-(1-ethyl-4-(trifluoromethyl)-1H-imidazol-2-yl)benzyl)-1H-pyrazolo[3,4-d]pyrimidine;
6-(4-cyclopropyl-6-(difluoromethoxy)pyrimidin-5-yl)-1-(4-(1-ethyl-4-(trifluoromethyl)-1H-imidazol-2-yl)benzyl)-1H-pyrazolo[3,4-d]pyrimidine;
6-(4-cyclopropyl-6-methoxy-2-methy]pyrimidin-5-yl)-1-(4-(1-methyl-4-(trifluoromethyl)-1H-imidazol-2-yl)benzyl)-1H-pyrazolo[3,4-d]pyrimidine;
6-(4-cyclopropyl-6-methoxypyrimidin-5-yl)-1-(4-(1-(oxetan-3-yl)-4-(trifluoromethyl)-1H-imidazol-2-yl)benzyl)-1H-pyrazolo[3,4-d]pyrimidine;
6-(4-(tert-butyl)-6-methoxypyrimidin-5-yl)-1-(4-(1-methyl-4-(trifluoromethyl)-1H-imidazol-2-yl)benzyl)-1H-pyrazolo[3,4-d]pyrimidine;
6-(4-(tert-butoxy)-6-cyclopropylpyrimidin-5-yl)-1-(4-(1-ethyl-4˜(trifluoromethyl)-1H-imidazol-2-yl)benzyl)-1H-pyrazolo[3,4-d]pyrimidine;
6-(4-cyclopropyl-6-(2-fluoroethoxy)pyrimidin-5-yl)-1-(4-(1-ethyl-4-(trifluoromethyl)-1H-imidazol-2-yl)benzyl)-1H-pyrazolo[3,4-d]pyrimidine;
6-(4-cyclopropyl-6-methoxypyrimidin-5-yl)-1-(4-(1-(methyl-d3)-4-(trifluoromethyl)-1H-imidazol-2-yl)benzyl)-1H-pyrazolo[3,4-d]pyrimidine;
1-(4-(1-cyclobutyl-4-(trifluoromethyl)-1H-imidazol-2-yl)benzyl)-6-(4-cyclopropyl-6-methoxypyrimidin-5-yl)-1H-pyrazolo[3,4-d]pyrimidine;
6-(4,6-dimethoxypyrimidin-5-yl)-1-(4-(1-ethyl-4-(trifluoromethyl)-1H-imidazol-2-yl)benzyl)-1H-pyrazolo[3,4-d]pyrimidine;
6-(4-cyclopropyl-6-methoxypyrimidin-5-y{)-1-(4-(1-isopropyl-4-(trifluoromethyl)-1H-imidazol-2-yl)benzyl)-1H-pyrazolo[3,4-d]pyrimidine;
1-(4-(1-cyclopropyl-4-(trifluoromethyl)-1H-imidazol-2-yl)benzyl)-6-(4-cyclopropyl-6-methoxypyrimidin-5-yl)-1H-pyrazolo[3,4-d]pyrimidine;
6-(4-cyclopropyl-6-methoxypyrimidin-5-yl)-1-(4-(1-(2-fluoroethyl)-4-(trifluoromethyl)-1H-imidazol-2-yl)benzyl)-1H-pyrazolo[3,4-d]pyrimidine;
6-(4-cyclopropyl-6-methoxypyrimidin-5-yl)-1-(4-(4-(trifluoromethyl)-1H-imidazol-2-yl)benzyl)-1H-pyrazolo[3,4-d]pyrimidine; 6-(4-cyclopropyl-6-methoxypyrimidin-5-yl)-1-(3-fluoro-4-(1-methyl-4-(trifluoromethyl)-1H-imidazol-2-yl)benzyl)-1H-pyrazolo[3,4-d]pyrimidine;
6-(4-cyclopropyl-6-methoxypyrimidin-5-yl)-1-(2-fluoro-4-(1-methyl-4-(trifluoromethyl)-1H-imidazol-2-yl)benzyl)-1H-pyrazolo[3,4-d]pyrimidine;
6-(2,4-dimethoxy-6-methylpyrimidin-5-yl)-1-(4-(1-methyl-4-(trifluoromethyl)-1H-imidazol-2-yl)benzyl)-1H-pyrazolo[3,4-d]pyrimidine;
6-(4-cyclopropyl-6-methoxypyrimidin-5-yl)-1-(4-(5-methoxy-3-(trifluoromethyl)-1H-pyrazol-1-yl)benzyl)-1H-pyrazolo[3,4-d]pyrimidine;
6-(4-cyclopropyl-6-(2-methoxyethoxy)pyrimidin-5-yl)-1-(4-(1-methyl-4-(trifluoromethyl)-1H-imidazol-2-yl)benzyl)-1H-pyrazolo[3,4-d]pyrimidine;
6-(4-cyclopropyl-6-(2-methoxy ethoxy)pyrimidin-5-yl)-1-(4-(5-methyl-3-(trifluoromethyl)-1H-pyrazol-1-yl)benzyl)-1H-pyrazolo[3,4-d]pyrimidine;
6-(4-methoxy-6-(1-methylcyclopropyl)pyrimidin-5-yl)-1-(4-(1-methyl-4-(trifluoromethyl)-1H-imidazol-2-yl)benzyl)-1H-pyrazolo[3,4-d]pyrimidine;
6-(1-isopropyl-4-methyl-1H-pyrazol-5-yl)-1-(4-(5-methyl-3-(trifluoromethyl)-1H-pyrazol-1-yl)benzyl)-1H-pyrazolo[3,4-d]pyrimidine;
(S)-6-(4-cyclopropyl-6-methoxypyrimidin-5-yl)-1-(1-(4-(1-methyl-4-(trifluoromethyl)-1H-imidazol-2-yl)phenyl)ethyl)-1H-pyrazolo[3,4-d]pyrimidine;
(R)-6-(4-cyclopropyl-6-methoxypyrimidin-5-yl)-1-(1-(4-(1-methyl-4-(trifluoromethyl)-1H-imidazol-2-yl)phenyl)ethyl)-1H-pyrazolo[3,4-d]pyrimidine;
6-(4-cyclobutyl-6-methoxypyrimidin-5-yl)-1-(4-(1-methyl-4-(trifluoromethyl)-1H-imidazol-2-yl)benzyl)-1H-pyrazolo[3,4-d]pyrimidine;
6-(4-cyclopropyl-6-isopropoxypyrimidin-5-yl)-1-(4-(1-methyl-4-(trifluoromethyl)-1H-imidazol-2-yl)benzyl-1H-pyrazolo[3,4-d]pyrimidine;
6-(4-isopropyl-6-methylpyrimidin-5-yl)-1-(4-(5-methyl-3-(trifluoromethyl)-1H-pyrazol-1-yl)benzyl)-1H-pyrazolo[3,4-d]pyrimidine;
6-(4-cyclopropyl-6-ethoxypyrimidin-5-yl)-1-(4-(1-methyl-4-(trifluoromethyl)-1H-imidazol-2-yl)benzyl)-1H-pyrazolo[3,4-d]pyrimidine;
N,N,6-trimethyl-5-(1-(4-(5-methyl-3-(trifluoromethyl)-1H-pyrazol-1-yl)benzyl)-1H-pyrazolo[3,4-d]pyrimidin-6-yl)pyrimidin-4-amine;
1-(4-(5-methyl-3-(trifluoromethyl)-1H-pyrazol-1-yl)benzyl)-6-(4-methyl-6-(methylthio)pyrimidin-5-yl)-1H-pyrazolo[3,4-d]pyrimidine; 6-(4-cyclopropyl-6-methoxypyrimidin-5-yl)-1-(4-(1-ethyl-4-(trifluoromethyl)-1H-imidazol-2-yl)benzyl)-1H-pyrazolo[3,4-d]pyrimidine;
6-(4,6-dimethoxypyrimidin-5-yl)-1-(4-(1-methyl-4-(trifluoromethyl)-1H-imidazol-2-yl)benzyl)-1H-pyrazolo[3,4-d]pyrimidine;
6-(4-(2-methoxyethoxy)-6-methylpyrimidin-5-yl)-1-(4-(5-methyl-3-(trifluoromethyl)-1H-pyrazol-1-yl)benzyl)-1H-pyrazolo[3,4-d]pyrimidine;
6-(4-cyclopropyl-6-methoxypyrimidin-5-yl)-1-(4-(1-(1-methylazetidin-3-yl)-4-(trifluoromethyl)-1H-imidazol-2-yl)benzyl)-1H-pyrazolo[3,4-d]pyrimidine;
6-(4-methoxy-6-methylpyrimidin-5-yl)-1-(4-(5-methyl-3-(trifluoromethyl)-1H-pyrazol-1-yl)benzyl)-1H-pyrazolo[3,4-d]pyrimidine;
3-(1-(4-(5-methyl-3-(trifluoromethyl)-1H-pyrazol-1-yl)benzyl)-1H-pyrazolo[3,4-d]pyrimidin-6-yl)picolinonitrile;
6-(4-cyclopropyl-6-methoxypyrimidin-5-yl)-1-(4-(3-(difluoromethyl)-5-methyl-1H-pyrazol-1-yl)benzyl)-1H-pyrazolo[3,4-d]pyrimidine;
6-cyclopropyl-N-methyl-5-(1-(4-(1-methyl-4-(trifluoromethyl)-1H-imidazol-2-yl)benzyl)-1H-pyrazolo[3,4-d]pyrimidin-6-yl)pyrimidin-4-amine;
1-(4-(5-methoxy-3-(trifluoromethyl)-1H-pyrazol-1-yl)benzyl)-6-(4-methoxy-6-methylpyrimidin-5-yl)-1H-pyrazolo[3,4-d]pyrimidine;
6-(4-cyclopropyl-6-((tetrahydrofuran-3-yl)oxy)pyrimidin-5-yl)-1-(4-(1-methyl-4-(trifluoromethyl)-1H-imidazol-2-yl)benzyl)-1H-pyrazolo[3,4-d]pyrimidine;
6-(4-(benzyloxy)-6-cyclopropylpyrimidin-5-yl)-1-(4-(1-ethyl-4-(trifluoromethyl)-1H-imidazol-2-yl)benzyl-1H-pyrazolo[3,4-d]pyrimidine;
6-(4-cyclopropyl-6-methoxypyrimidin-5-yl)-1-(4-(3-(difluoromethyl)-1-methyl-1H-1,2,4-triazol-5-yl)benzyl)-1H-pyrazolo[3,4-d]pyrimidine;
6-(4-(benzyloxy)-6-cyclopropylpyrimidin-5-yl)-1-(4-(1-methyl-4-(trifluoromethyl)-1H-imidazol-2-yl)benzyl)-1H-pyrazolo[3,4-d]pyrimidine;
6-(4-cyclopropyl-6-methoxypyrimidin-5-yl)-1-(4-(5-methoxy-1-methyl-1H-1,2,4-triazol-3-yl)benzyl)-1H-pyrazolo[3,4-d]pyrimidine;
2-(2-(4-((6-(4-cyclopropyl-6-methoxypyrimidin-5-yl)-1H-pyrazolo[3,4-d]pyrimidin-1-yl)methyl)phenyl)-4-(trifluoromethyl)-1H-imidazol-1-y 1)-N,N-dimethylacetamide;
6-cyclopropyl-N,N-dimethyl-5-(1-(4-(1-methyl-4-(trifluoromethyl)-1H-imidazol-2-yl)benzyl)-1H-pyrazolo[3,4-d]pyrimidin-6-yl)pyrimidin-4-amine; 6-(4-cyclopropyl-6-methoxypyrimidin-5-yl)-1-(4-(5-(difluoromethyl)-1-methyl-1H-1,2,4-triazol-3-yl)benzyl)-1H-pyrazolo[3,4-d]pyrimidine;
6-(4-cyclopropyl-6-(pyrrolidin-1-yl)pyrimidin-5-yl)-1-(4-(1-methyl-4-(trifluoromethyl)-1H-imidazol-2-yl)benzyl)-1H-pyrazolo[3,4-d]pyrimidine;
6-(4-(azetidin-1-yl)-6-cyclopropylpyrimidin-5-yl)-1-(4-(1-methyl-4-(trifluoromethyl)-1H-imidazol-2-yl)benzyl)-1H-pyrazolo[3,4-d]pyrimidine; and
5-(4-cyclopropyl-6-methoxypyrimidin-5-yl)-3-(4-(1-methyl-4-(trifluoromethyl)-1H-imidazol-2-yl)benzyl)-3H-[1,2,3]triazolo[4,5-d]pyrimidine,
6-(4-cyclopropyl-6-methoxypyrimidin-5-yl)-1-(4-(1-ethyl-4-(trifluoromethyl)-1H-imidazol-2-yl)benzyl)-3-(methylsulfonyl)-1H-pyrazolo[3,4-d]pyrimidine;
6-(4-cyclopropyl-6-methoxypyrimidin-5-yl)-1-(4-(1-ethyl-4-(trifluoromethyl)-1H-imidazol-2-yl)benzyl)-3-(methylthio)-1H-pyrazolo[3,4-d]pyrimidine;
6-(4-cyclopropyl-6-methoxypyrimidin-5-yl)-1-(4-(1-ethyl-4-(trifluoromethyl)-1H-imidazol-2-yl)benzyl)-3-((4-methoxybenzyl)thio)-1H-pyrazolo[3,4-d]pyrimidine;
(S)-1-(1-(4-(5-bromo-1-methyl-4-(trifluoromethyl)-1H-imidazol-2-yl)phenyl)ethyl)-6-(4-cyclopropyl-6-methoxypyrimidin-5-yl)-3-methoxy-1H-pyrazolo[3,4-d]pyrimidine;
2-(4-cyclopropyl-6-methoxypyrimidin-5-yl)-7-(4-(1-isopropyl-4-(trifluoromethyl)-1H-imidazol-2-yl)benzyl)-7H-pyrrolo[2,3-d]pyrimidine;
2-(4-cyclopropyl-6-methoxypyrimidin-5-yl)-9-(4-(1-isopropyl-4-(trifluoromethyl)-1H-imidazol-2-yl)benzyl)-8-(methoxymethyl)-9H-purine;
6-(4-cyclopropyl-6-methoxypyrimidin-5-yl)-1-(4-(1-ethyl-4-(trifluoromethyl)-1H-imidazol-2-yl)benzyl)-3-methoxy-1H-pyrazolo[3,4-d]pyrimidine;
2-(4-cyclopropyl-6-methoxypyrimidin-5-yl)-8-methyl-9-(4-(1-methyl-4-(trifluoromethyl)-1H-imidazol-2-yl)benzyl)-9H-purine;
8-cyclopropyl-2-(4-cyclopropyl-6-methoxypyrimidin-5-yl)-9-(4-(5-methyl-3-(trifluoromethyl)-1H-pyrazol-1-yl)benzyl)-9H-purine;
8-cyclobutyl-2-(4-cyclopropyl-6-methoxypyrimidin-5-yl)-9-(4-(5-methyl-3-(trifluoromethyl)-1H-pyrazol-1-yl)benzyl)-9H-purine;
2-(4-cyclopropyl-6-methoxypyrimidin-5-yl)-8-isopropyl-9-(4-(5-methyl-3-(trifluoromethyl)-1H-pyrazol-1-yl)benzyl)-9H-purine; 2-(4-cyclopropyl-6-methoxypyrimidin-5-yl)-8-ethyl-9-(4-(5-methyl-3-(trifluoromethyl)-1H-pyrazol-1-yl)benzyl)-9H-purine;
2-(4-cyclopropyl-6-methoxypyrimidin-5-yl)-7-(4-(1-methyl-4-(trifluoromethyl)-1H-imidazol-2-yl)benzyl)-7H-pyrrolo[2,3-d]pyrimidine;
2-(4-cyclopropyl-6-methoxypyrimidin-5-yl)-8-methyl-9-(4-(5-methyl-3-(trifluoromethyl)-1H-pyrazol-1-yl)benzyl)-9H-purine;
5-(4-cyclopropyl-6-methoxypyrimidin-5-yl)-3-(4-(1-methyl-4-(trifluoromethyl)-1H-imidazol-2-yl)benzyl)-3H-[1,2,3]triazolo[4,5-d]pyrimidine;
2-(4-methoxy-6-methylpyrimidin-5-yl)-9-(4-(5-methy]-3-(trifluoromethyl)-1H-pyrazol-1-yl)benzyl)-9H-purine;
2-(4-cyclopropyl-6-methoxypyrimidin-5-yl)-9-(4-(5-methy]-3-(trifluoromethyl)-1H-pyrazol-1-yl)benzyl)-9H-purine;
2-(4-cyclopropyl-6-methoxypyrimidin-5-yl)-9-(4-(1-methyl-4-(trifluoromethyl)-1H-imidazol-2-yl)benzyl)-9H-purine; or
2-(2-isopropylphenyl)-9-(4-(1-methyl-4-(trifluoromethyl)-1H-imidazol-2-yl)benzyl)-9H-purine;
and the compound is not selected from:

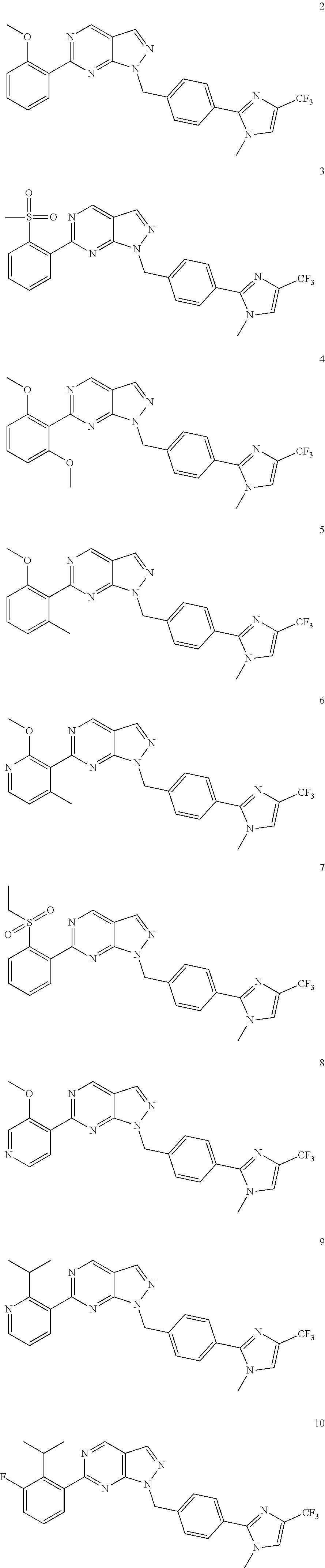

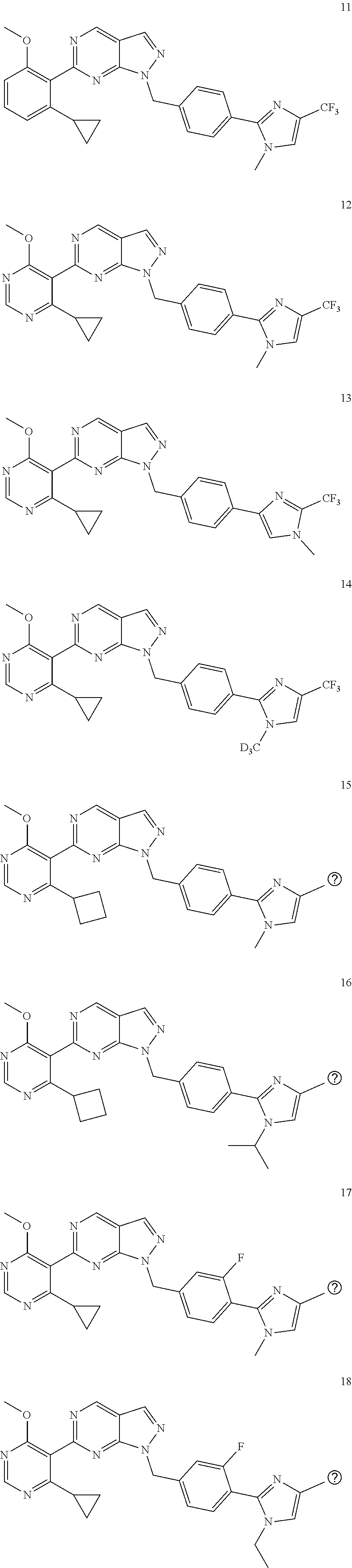

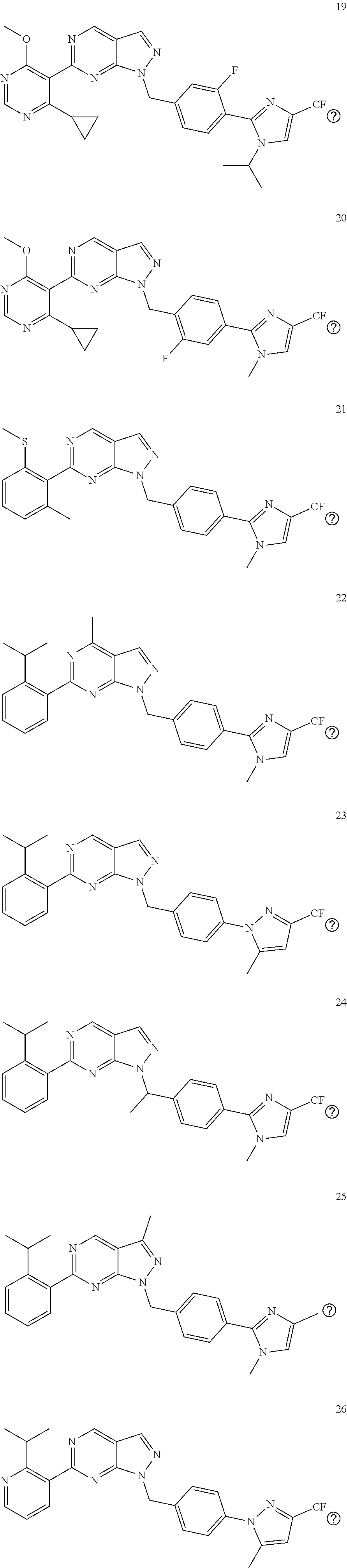

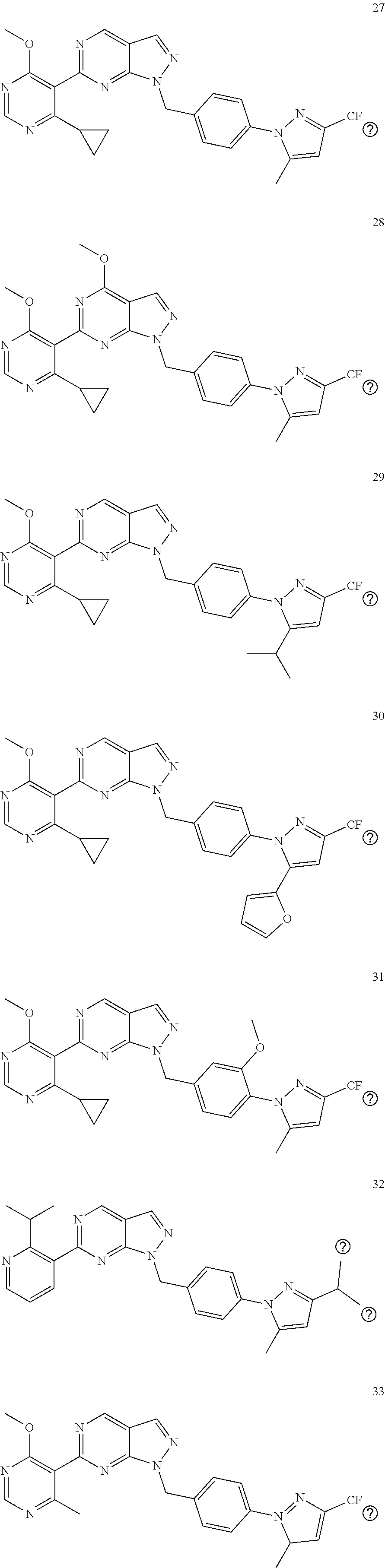

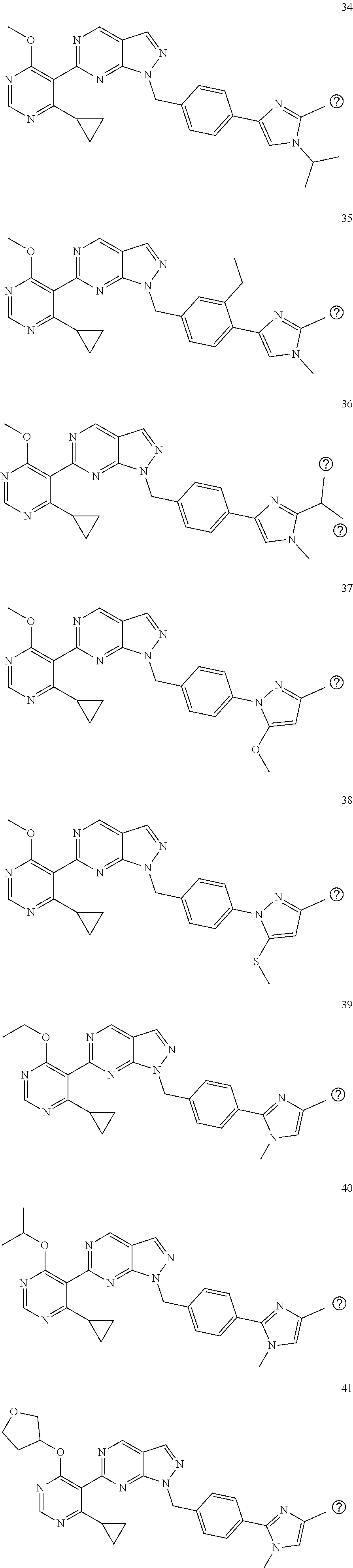

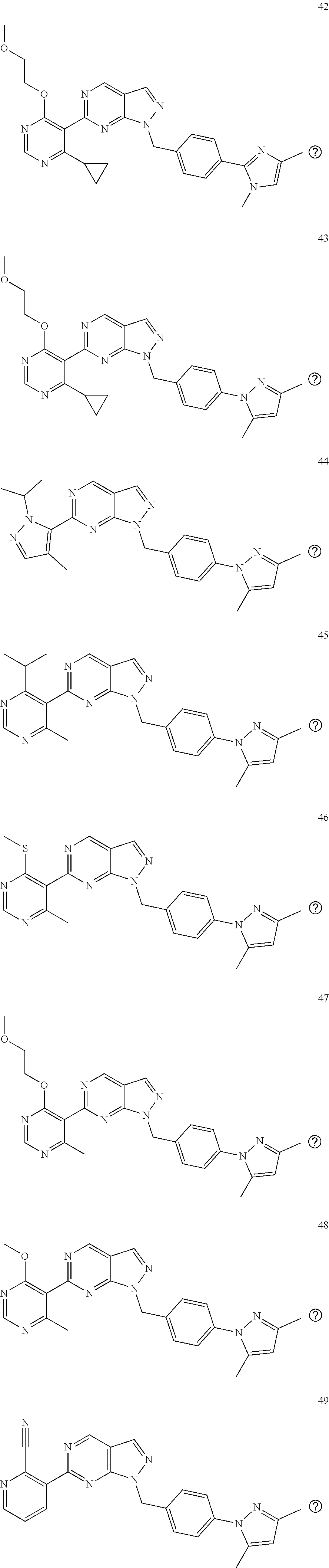

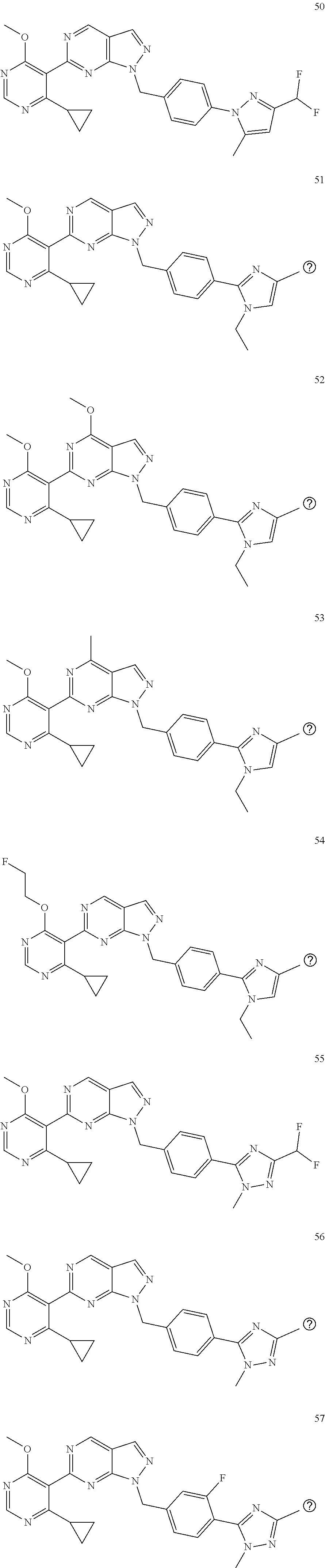

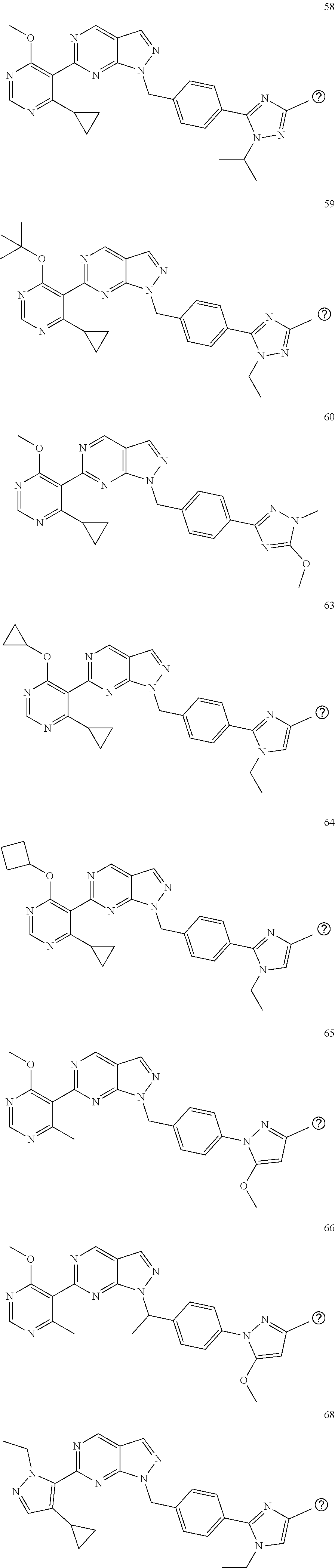

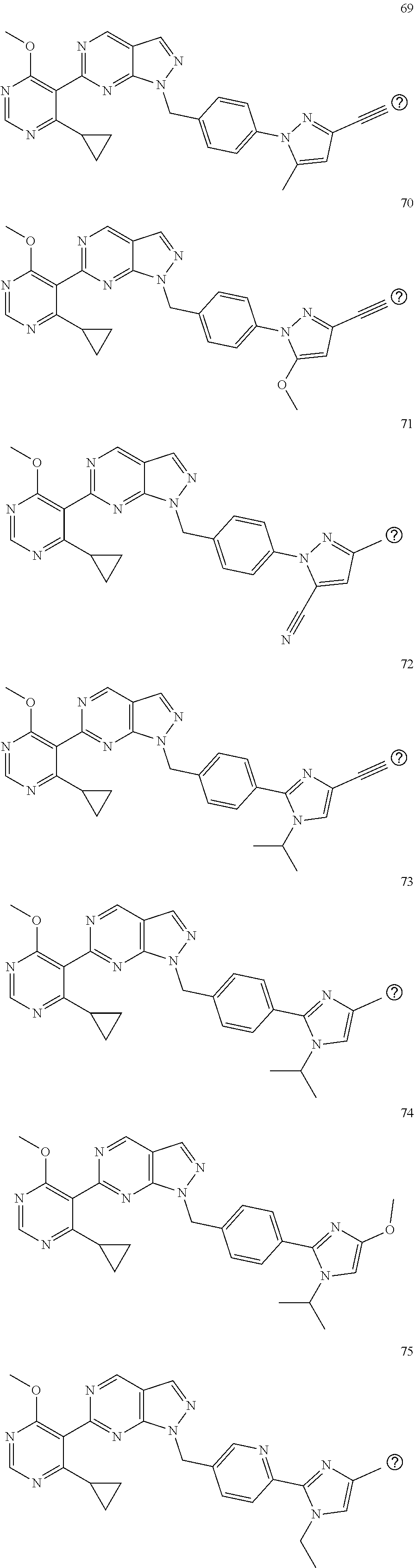

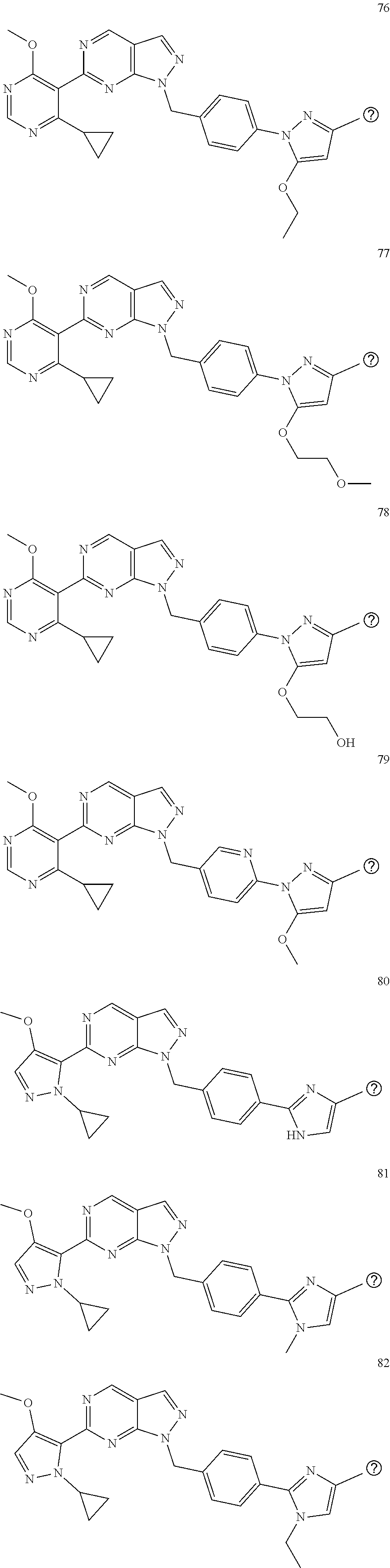

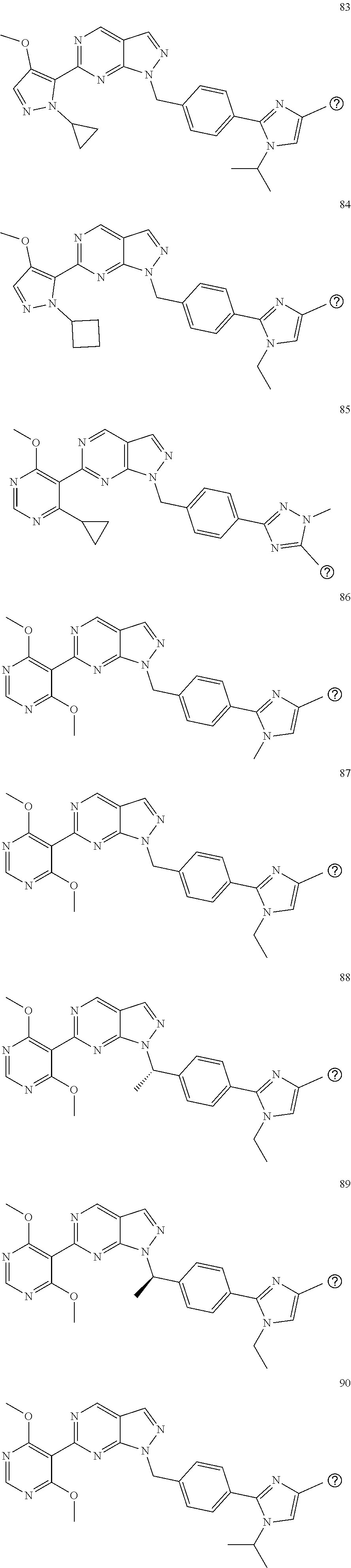

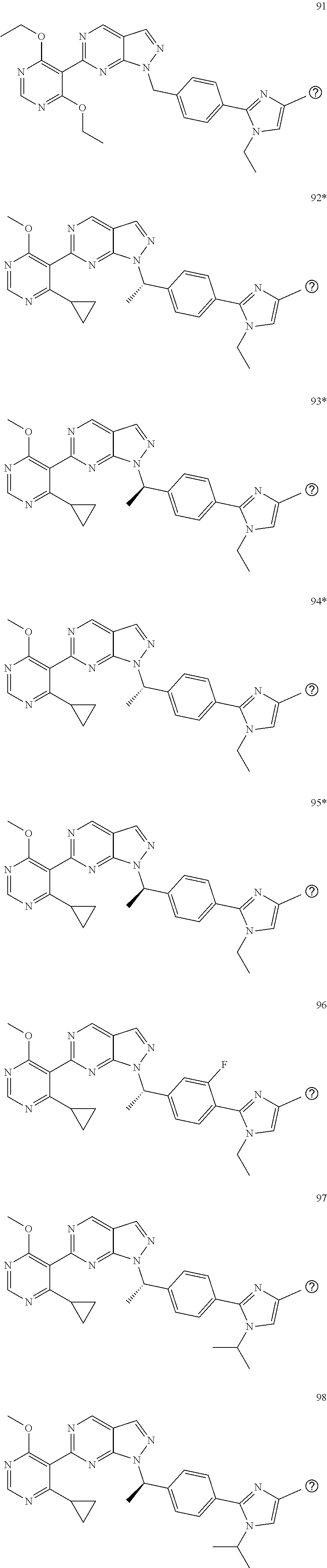

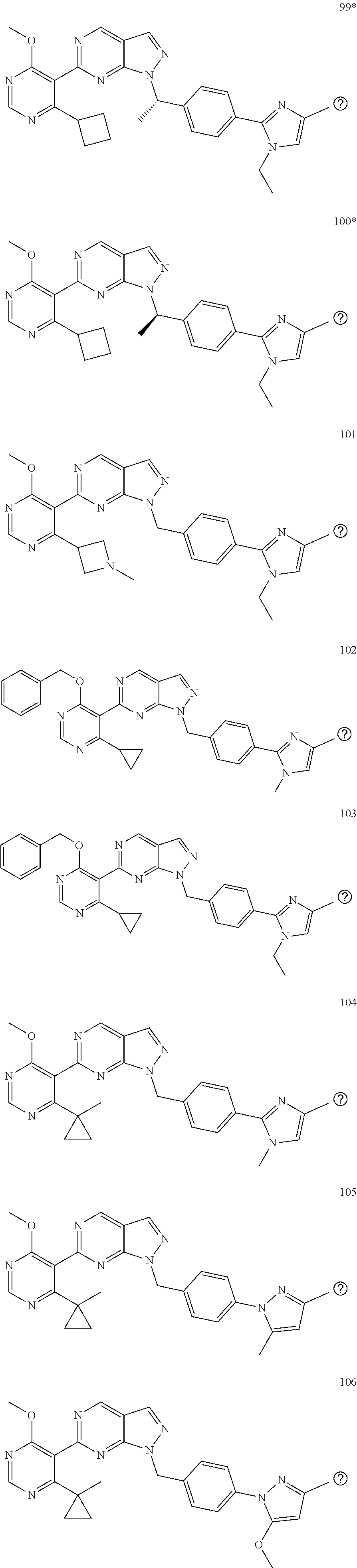

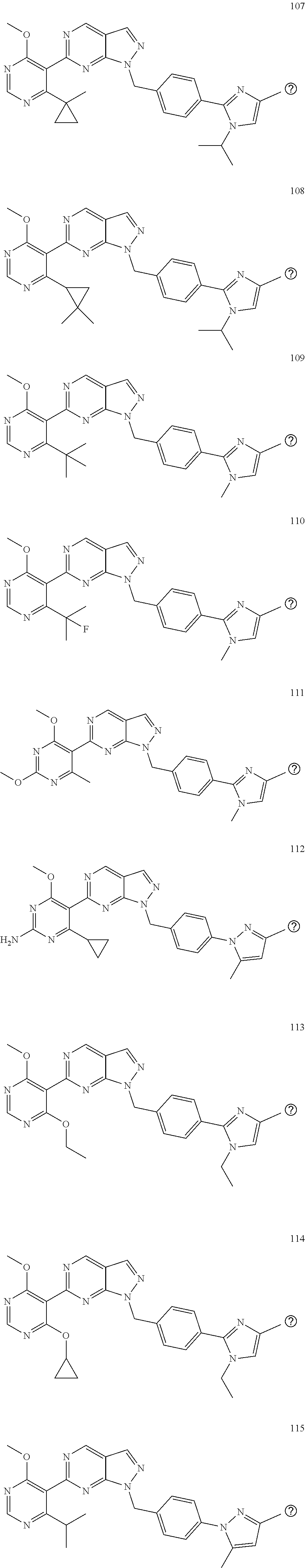

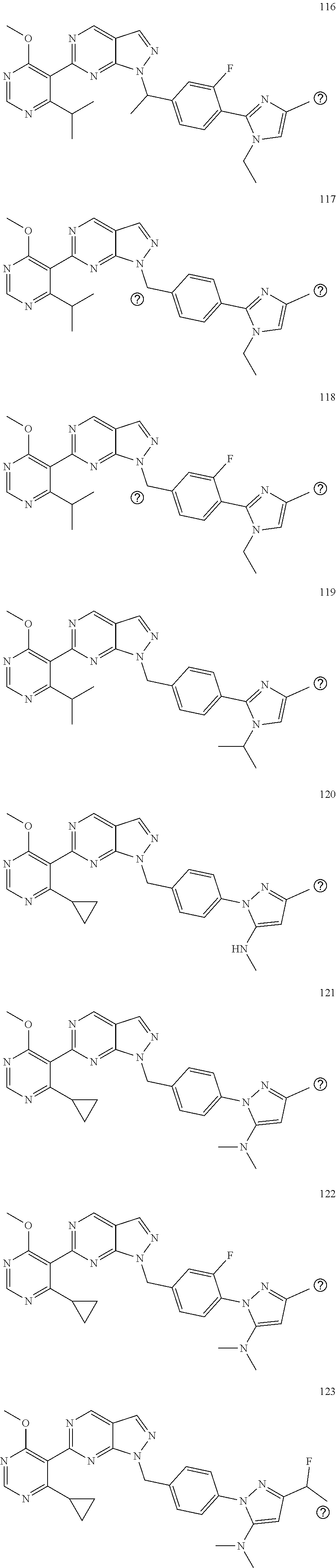

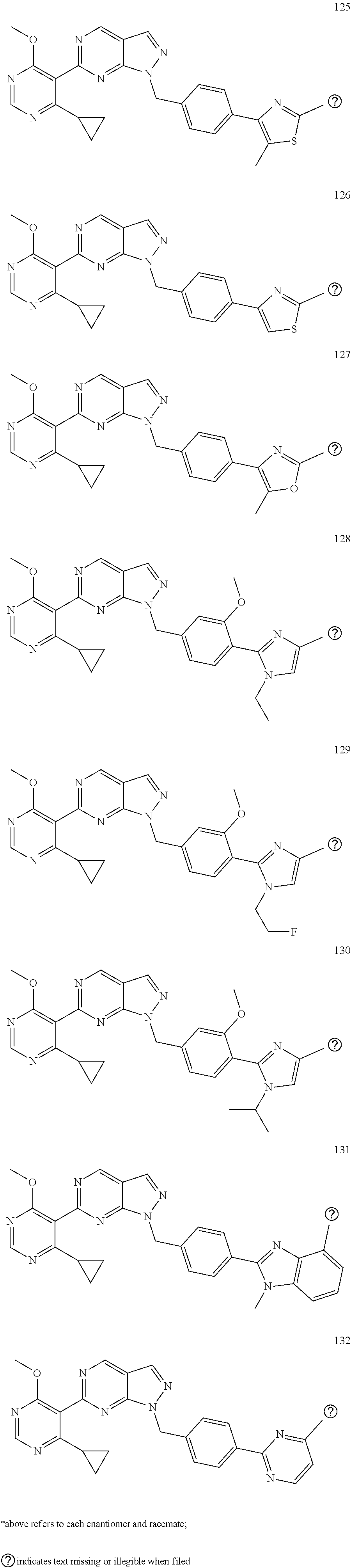

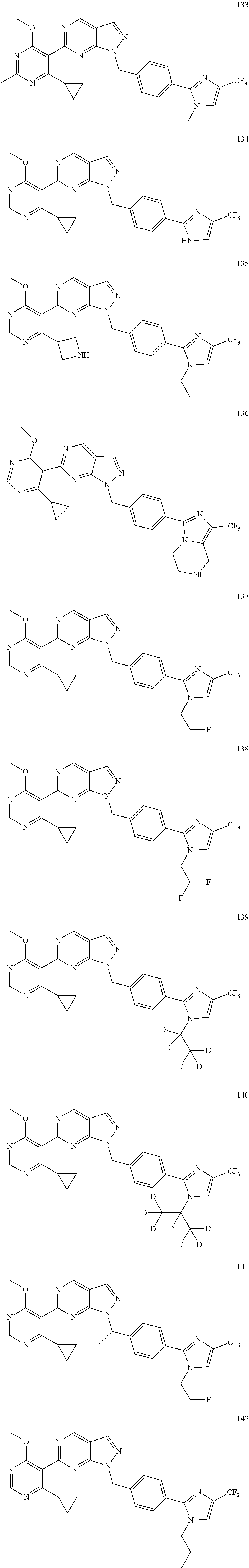

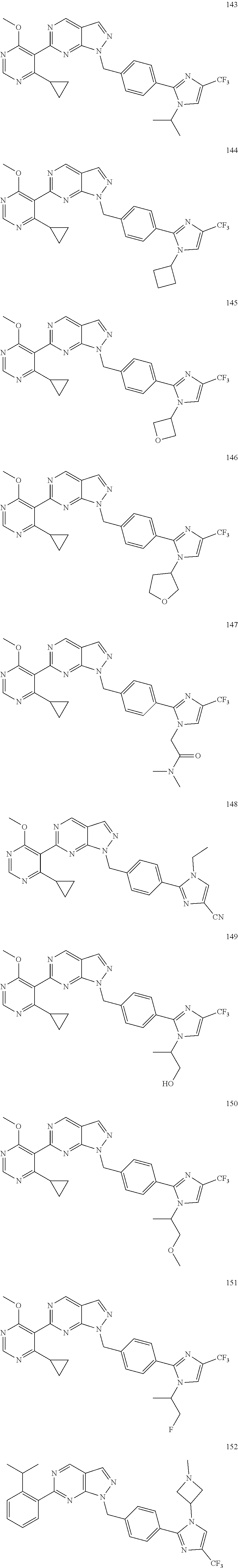

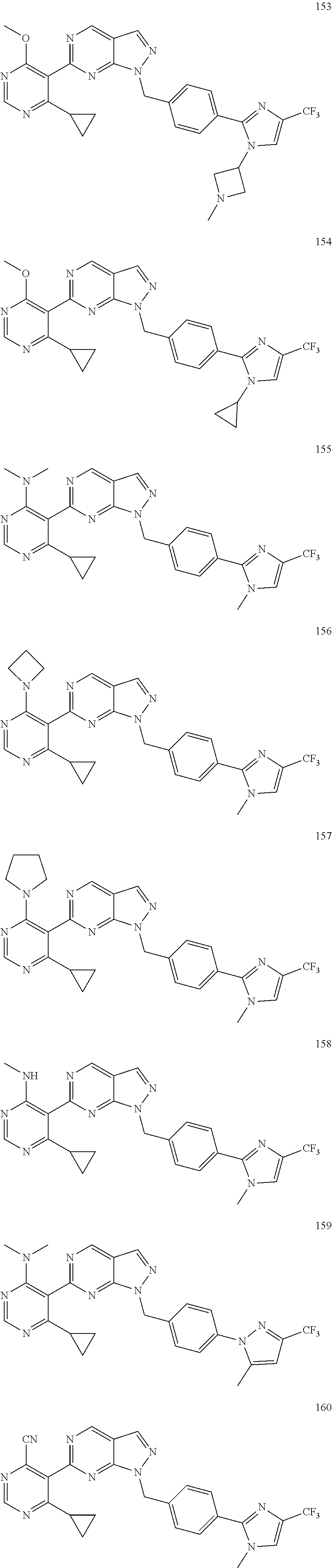

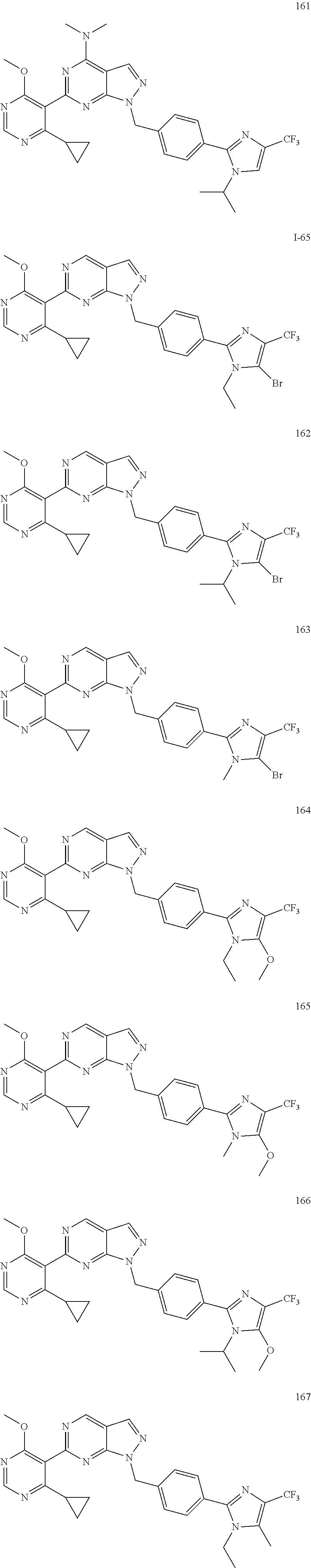

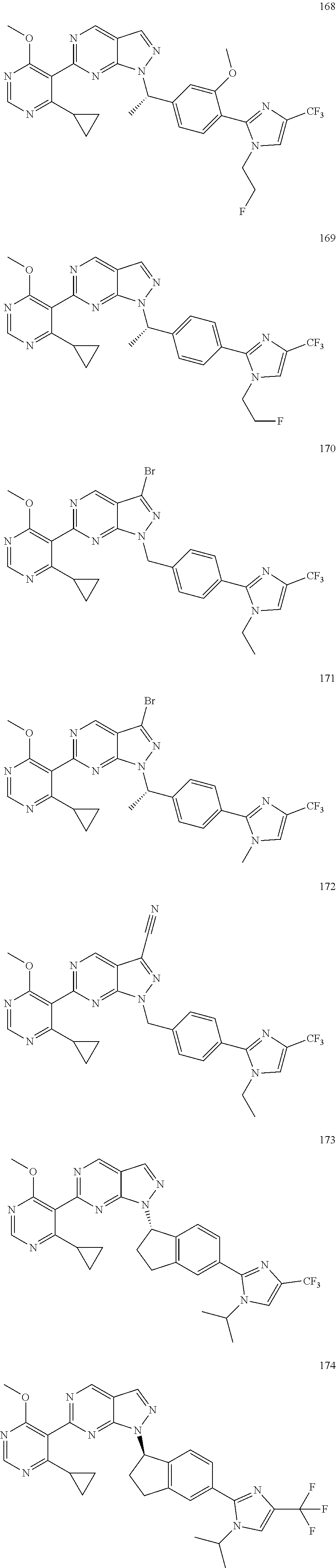

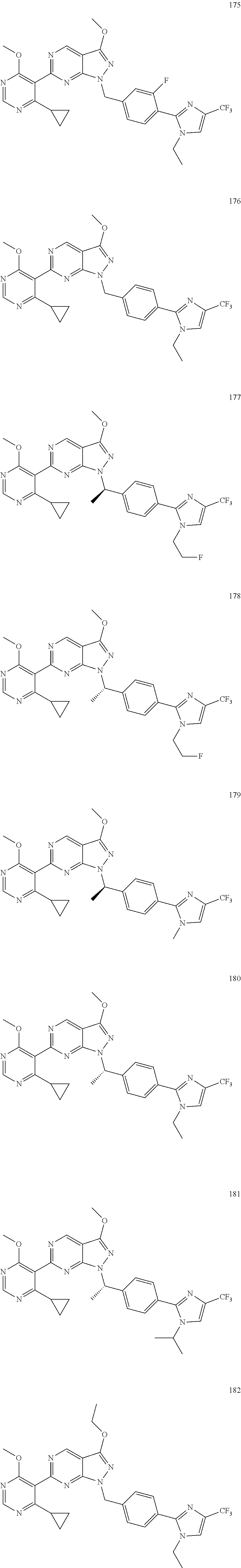

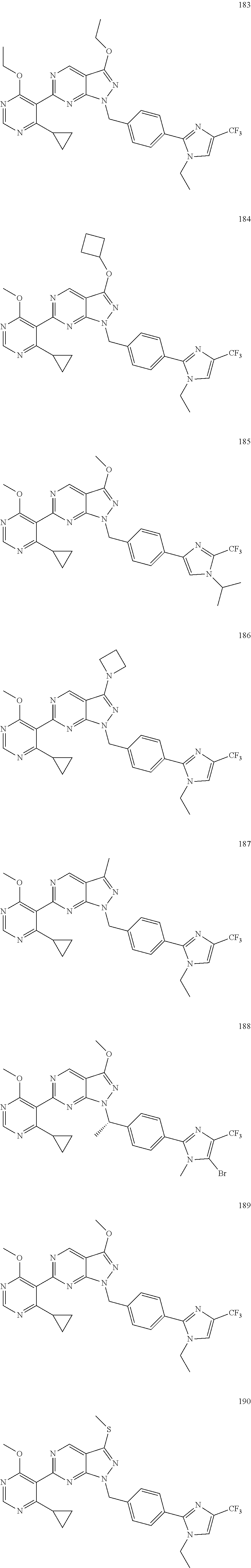

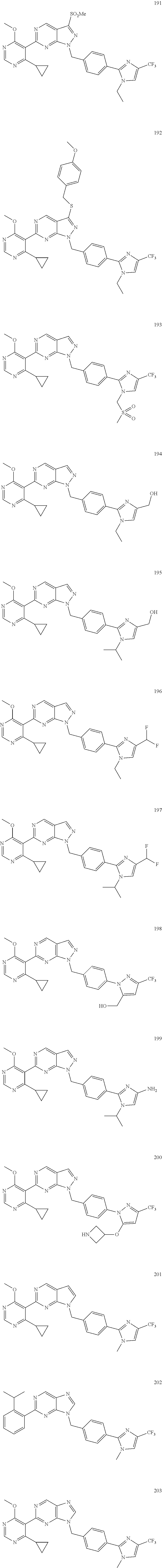

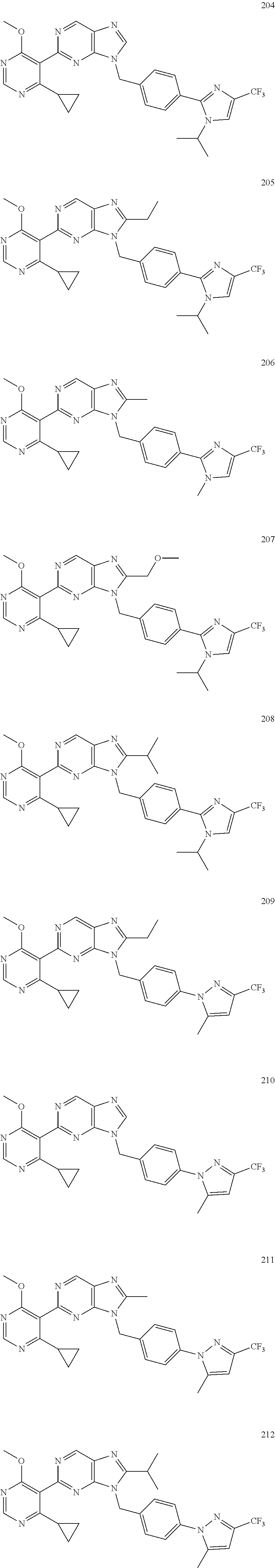

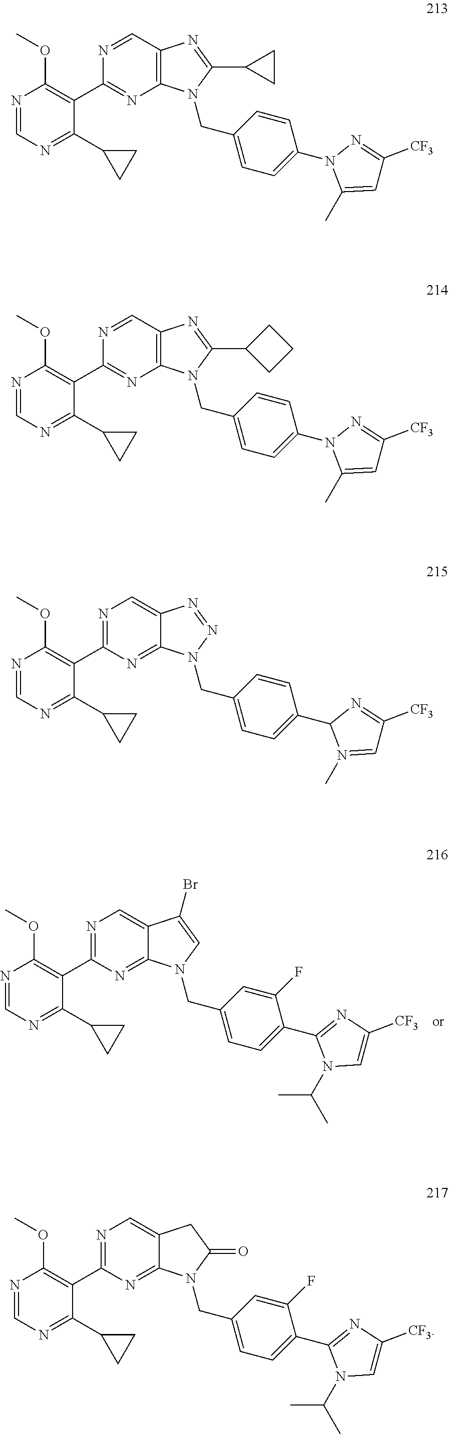

3. The compound of claim 1, wherein the compound is selected from the group consisting of a compound of Formula (IIa), Formula (IIb) and Formula (IIc): 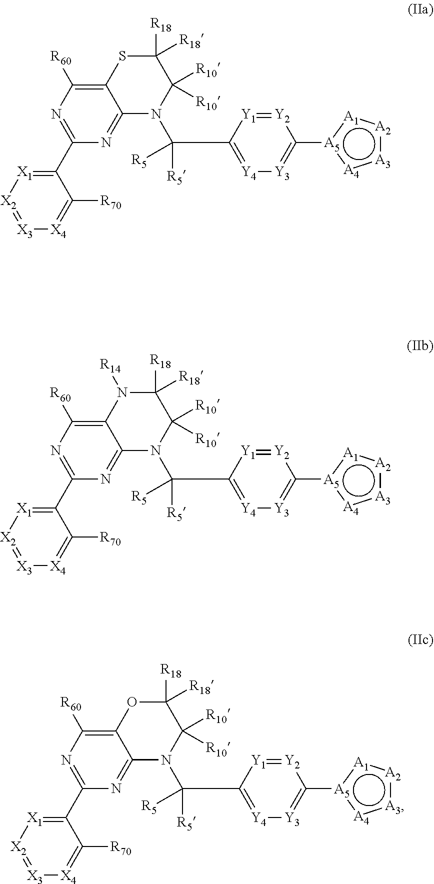
or a pharmaceutically acceptable salt thereof.

4. The compound of claim 1, wherein the compound is selected from the group consisting of a compound of Formula (IIIa), Formula (IIIb) and Formula (IIIc): 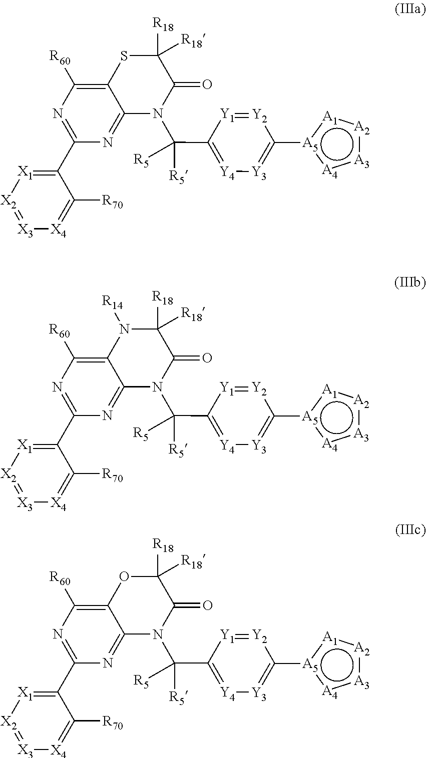
or a pharmaceutically acceptable salt thereof.

5. The compound of claim 1, wherein the compound is selected from the group consisting of a compound of Formula (IVa), Formula (IVb) and Formula (IVc): 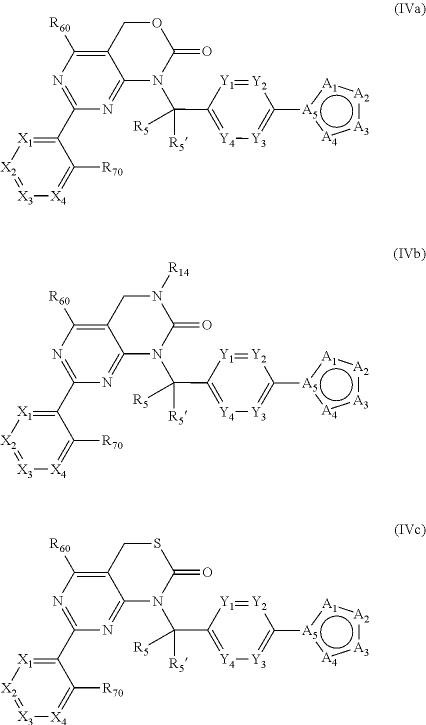
or a pharmaceutically acceptable salt thereof.

6. The compound of claim 1, wherein the compound is selected from the group consisting of a compound of Formula (Va), and Formula (Vb): 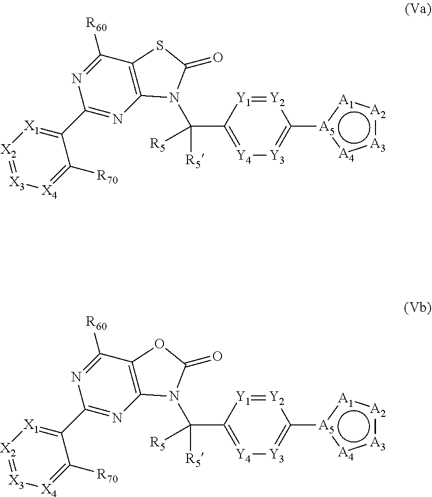
or a pharmaceutically acceptable salt thereof.

7. The compound of claim 1, wherein the compound is selected from the group consisting of a compound of Formula (VIa), and Formula (VIb): 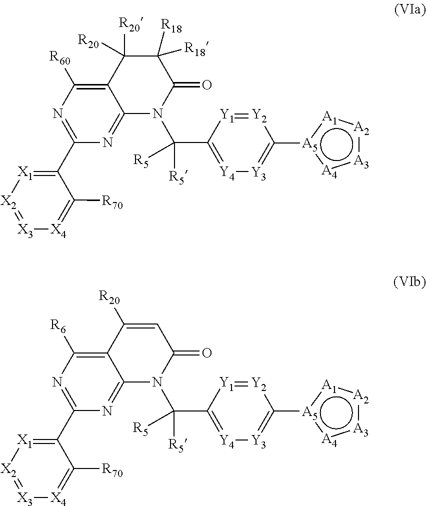
or a pharmaceutically acceptable salt thereof.

8. The compound of claim 1, wherein the compound is a compound of Formula (VII), 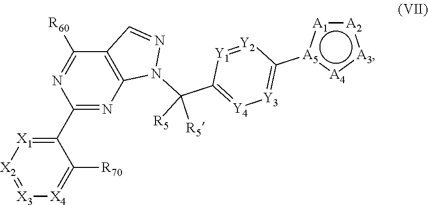
or a pharmaceutically acceptable salt thereof,

9. The compound of claim 1, wherein the compound is selected from the group consisting of a compound of Formula (VIIIa), Formula (VIIIb) and Formula (VIIIc): 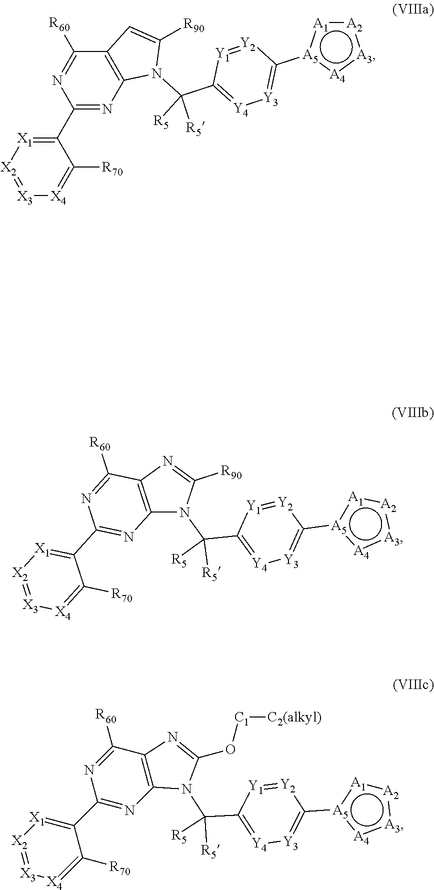
or a pharmaceutically acceptable salt thereof.

10. The compound of claim 1, wherein the compound is a compound of Formula (IX), 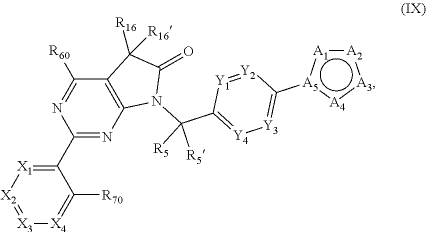
or a pharmaceutically acceptable salt thereof.

11. The compound of claim 1, wherein the compound is selected from the group consisting of a compound of Formula (Xa) and Formula (Xb): 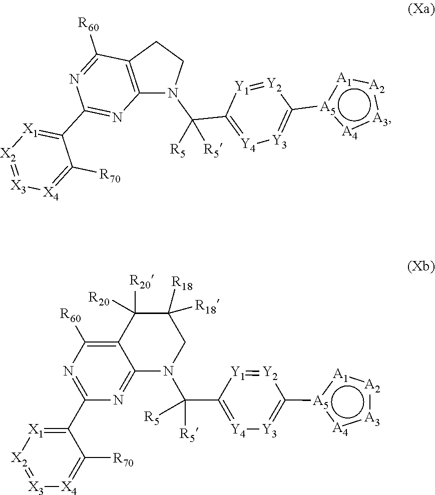
or a pharmaceutically acceptable salt thereof.

12. The compound of any one of claims 1-11, wherein Y₁, Y₂, Y₃ and Y₄ are each CH.

13. The compound of any one of claims 1-12, wherein R₅ and R₅′ are each hydrogen.

14. The compound of any one of claims 1-13, wherein R₇₀ is isopropyl, and cyclopropyl.

15. The compound of any one of claims 1-14, wherein R₆₀ is selected from the group consisting of hydrogen, methyl, methoxy, and halogen.

16. The compound of any one of claims 1-15, wherein A₁ is NR₁, wherein R₁ is a bond.

17. The compound of any one of claims 1-16, wherein A₂ is CR₂, and R₂ is methyl optionally substituted with one or more F.

18. The compound of any one of claims 1-17, wherein A₃ is CH.

19. The compound of any one of claims 1-18, wherein A₄ is NR₁.

20. The compound of claim 19, wherein R₁ is methyl.

21. The compound of claim 19, wherein A₅ is C.

22. The compound of any one of claims 1-17, wherein A₃ is NR₁, wherein R₁ is a bond.

23. The compound of claim 22, wherein A₄ is CR₂.

24. The compound of claim 23, wherein and R₂ is methyl.

25. The compound of claim 24, wherein A₅ is N.

26. The compound of any one of claims 1-16, wherein A₅ is N.

27. The compound of any one of claims 1-16, wherein A₅ is C.

28. The compound of any one of claims 1-17, wherein A₃ is NR₁, wherein R₁ is a bond.

29. The compound of claim 28, wherein A₄ is NR₁.

30. The compound of claim 29, wherein R₁ is methyl.

31. The compound of claim 1, selected from the group consisting of: 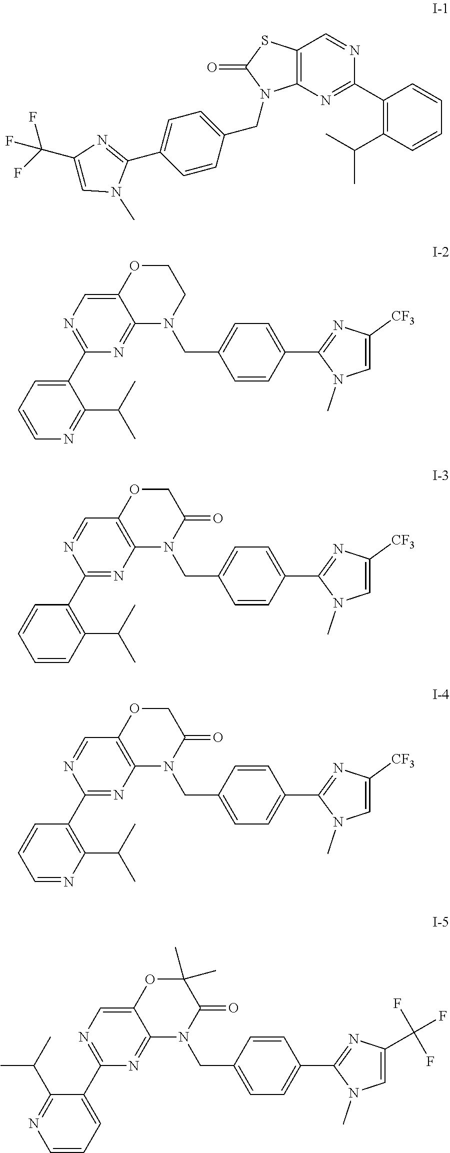 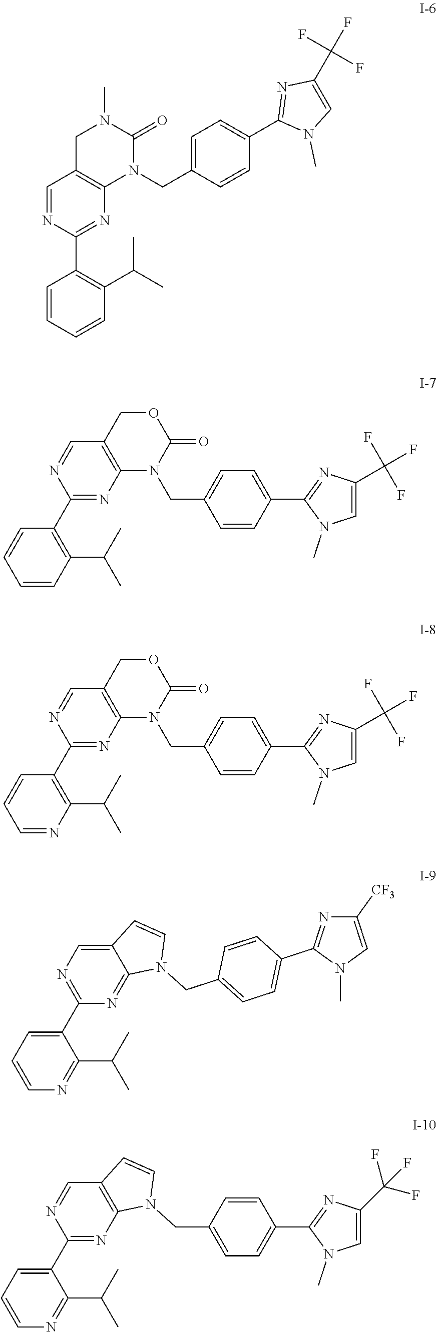 [SMILES: CC(C)c1ccccc1-c1ncc2c(n1)N(Cc1ccc(-c3nc(C(F)(F)F)cn3C)cc1)C(=O)N(C)C2 ; CC(C)c1ccccc1-c1ncc2c(n1)N(Cc1ccc(-c3nc(C(F)(F)F)cn3C)cc1)C(=O)OC2 ; CC(C)c1ncccc1-c1ncc2c(n1)N(Cc1ccc(-c3nc(C(F)(F)F)cn3C)cc1)C(=O)OC2 ; CC(C)c1ncccc1-c1ncc2ccn(Cc3ccc(-c4nc(C(F)(F)F)cn4C)cc3)c2n1 ; CC(C)c1ncccc1-c1ncc2ccn(Cc3ccc(-c4nc(C(F)(F)F)cn4C)cc3)c2n1] 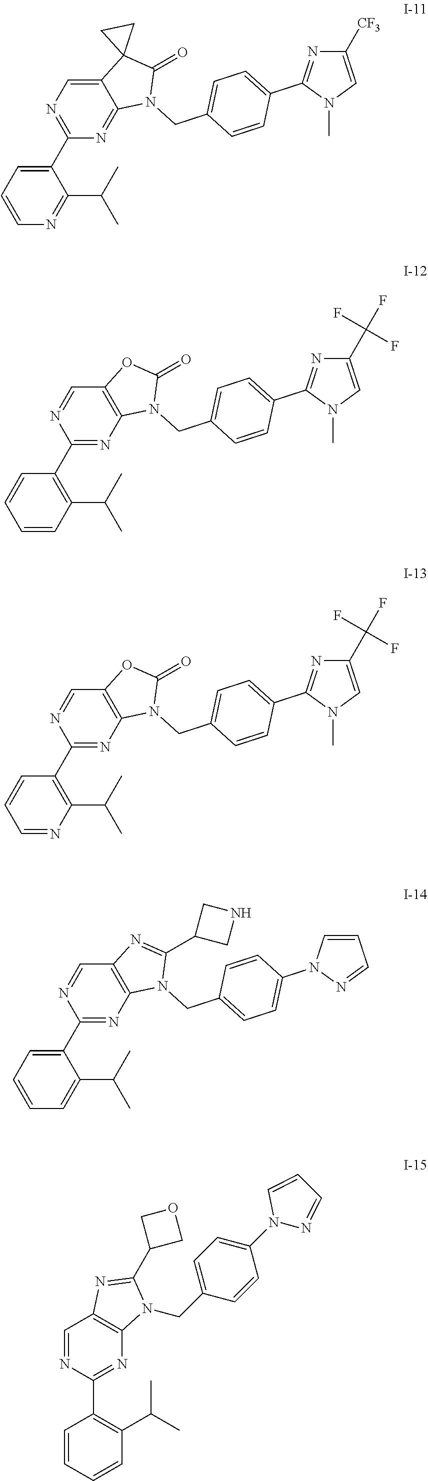 [SMILES: CC(C)c1ncccc1-c1ncc2c(n1)N(Cc1ccc(-c3nc(C(F)(F)F)cn3C)cc1)C(=O)C21CC1 ; CC(C)c1ccccc1-c1ncc2oc(=O)n(Cc3ccc(-c4nc(C(F)(F)F)cn4C)cc3)c2n1 ; CC(C)c1ncccc1-c1ncc2oc(=O)n(Cc3ccc(-c4nc(C(F)(F)F)cn4C)cc3)c2n1 ; CC(C)c1ccccc1-c1ncc2nc(C3CNC3)n(Cc3ccc(-n4cccn4)cc3)c2n1 ; CC(C)c1ccccc1-c1ncc2nc(C3COC3)n(Cc3ccc(-n4cccn4)cc3)c2n1] 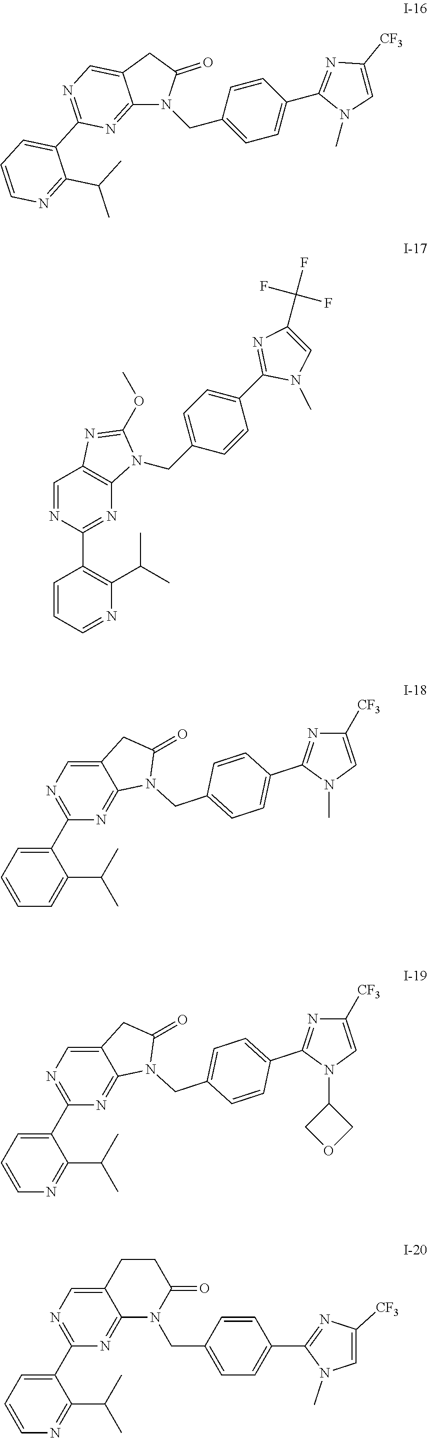 [SMILES: CC(C)c1ncccc1-c1ncc2c(n1)N(Cc1ccc(-c3nc(C(F)(F)F)cn3C)cc1)C(=O)C2 ; COc1nc2cnc(-c3cccnc3C(C)C)nc2n1Cc1ccc(-c2nc(C(F)(F)F)cn2C)cc1 ; CC(C)c1ccccc1-c1ncc2c(n1)N(Cc1ccc(-c3nc(C(F)(F)F)cn3C)cc1)C(=O)C2 ; CC(C)c1ncccc1-c1ncc2c(n1)N(Cc1ccc(-c3nc(C(F)(F)F)cn3C3COC3)cc1)C(=O)C2 ; CC(C)c1ncccc1-c1ncc2c(n1)N(Cc1ccc(-c3nc(C(F)(F)F)cn3C)cc1)C(=O)CC2] 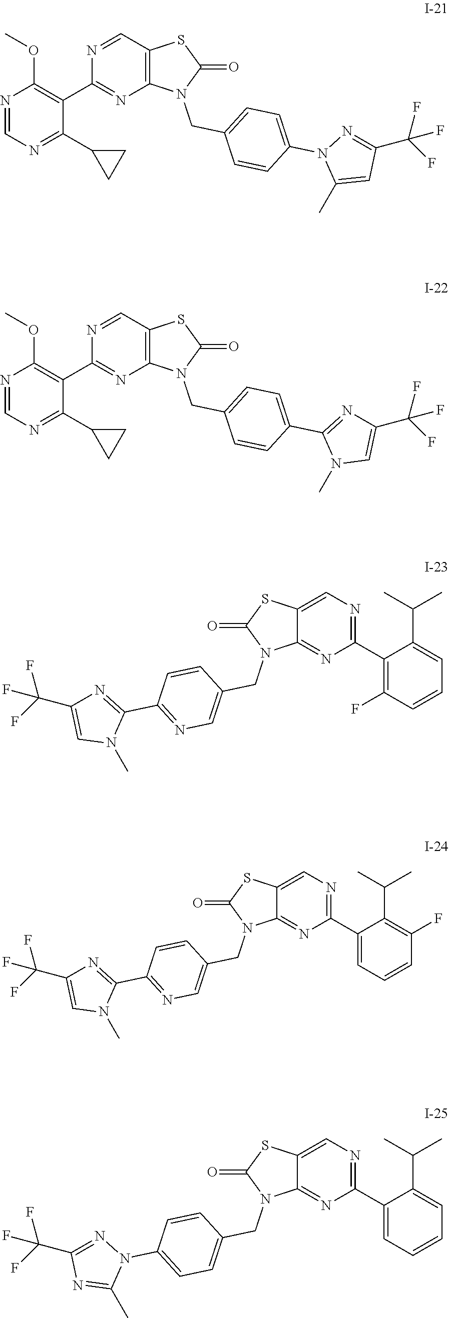 [SMILES: COc1ncnc(C2CC2)c1-c1ncc2sc(=O)n(Cc3ccc(-n4nc(C(F)(F)F)cc4C)cc3)c2n1 ; COc1ncnc(C2CC2)c1-c1ncc2sc(=O)n(Cc3ccc(-c4nc(C(F)(F)F)cn4C)cc3)c2n1 ; CC(C)c1cccc(F)c1-c1ncc2sc(=O)n(Cc3ccc(-c4nc(C(F)(F)F)cn4C)nc3)c2n1 ; CC(C)c1c(F)cccc1-c1ncc2sc(=O)n(Cc3ccc(-c4nc(C(F)(F)F)cn4C)nc3)c2n1 ; Cc1nc(C(F)(F)F)nn1-c1ccc(Cn2c(=O)sc3cnc(-c4ccccc4C(C)C)nc32)cc1] 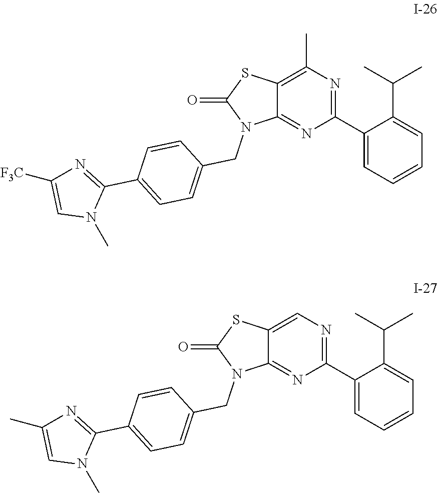
2-(4-cyclopropyl-6-methoxypyrimidin-5-yl)-8-isopropyl-9-(4-(1-isopropyl-4-(trifluoromethyl)-1H-imidazol-2-yl)benzyl)-9H-purine;
3-cyclobutoxy-6-(4-cyclopropyl-6-methoxypyrimidin-5-yl)-1-(4-(1-ethyl-4-(trifluoromethyl)-1H-imidazol-2-yl)benzyl)-1H-pyrazolo[3,4-d]pyrimidine;
6-(4-cyclopropyl-6-methoxypyrimidin-5-yl)-1-(4-(1-isopropyl-2-(trifluoromethyl)-1H-imidazol-4-yl)benzyl)-3-methoxy-1H-pyrazolo[3,4-d]pyrimidine;
2-(4-cyclopropyl-6-methoxypyrimidin-5-yl)-8-ethyl-9-(4-(1-isopropyl-4-(trifluoromethyl)-1H-imidazol-2-yl)benzyl)-9H-purine; (S)-6-(4-cyclopropyl-6-methoxypyrimidin-5-yl)-1-(1-(4-(1-(2-fluoroethyl)-4-(trifluoromethyl)-1H-imidazol-2-yl)phenyl)ethyl)-3-methoxy-1H-pyrazolo[3,4-d]pyrimidine;
3-(azetidin-1-yl)-6-(4-cyclopropyl-6-methoxypyrimidin-5-yl)-1-(4-(1-ethyl-4-(trifluoromethyl)-1H-imidazol-2-yl)benzyl)-1H-pyrazolo[3,4-d]pyrimidine;
1-(4-(5-bromo-1-ethyl-4-(trifluoromethyl)-1H-imidazol-2-yl)benzyl)-6-(4-cyclopropyl-6-methoxypyrimidin-5-yl)-3-methoxy-1H-pyrazolo[3,4-d]pyrimidine;
6-(4-cyclopropyl-6-methoxypyrimidin-5-yl)-1-(1-(4-(1-(2-fluoroethyl)-4-(trifluoromethyl)-1H-imidazol-2-yl)phenyl)ethyl)-3-methoxy-1H-pyrazolo[3,4-d]pyrimidine;
(S)-6-(4-cyclopropyl-6-methoxypyrimidin-5-yl)-1-(1-(4-(1-ethyl-4-(trifluoromethyl)-1H-imidazol-2-yl)phenyl)ethyl)-3-methoxy-1H-pyrazolo[3,4-d]pyrimidine;
(S)-6-(4-cyclopropyl-6-methoxypyrimidin-5-yl)-1-(1-(4-(1-isopropyl-4-(trifluoromethyl)-1H-imidazol-2-yl)phenyl)ethyl)-3-methoxy-1H-pyrazolo[3,4-d]pyrimidine;
6-(4-cyclopropyl-6-ethoxypyrimidin-5-yl)-3-ethoxy-1-(4-(1-ethyl-4-(trifluoromethyl)-1H-imidazol-2-yl)benzyl)-1H-pyrazolo[3,4-d]pyrimidine;
6-(4-cyclopropyl-6-methoxypyrimidin-5-yl)-3-ethoxy-1-(4-(1-ethyl-4-(trifluoromethyl)-1H-imidazol-2-yl)benzyl)-1H-pyrazolo[3,4-d]pyrimidine;
6-(4-cyclopropyl-6-methoxypyrimidin-5-yl)-1-(4-(1-ethyl-4-(trifluoromethyl)-1H-imidazol-2-yl)-3-fluorobenzy{)-3-methoxy-1H-pyrazolo[3,4-d]pyrimidine;
2-(4-cyclopropyl-6-methoxypyrimidin-5-yl)-9-(4-(1-isopropyl-4-(trifluoromethyl)-1H-imidazol-2-yl)benzyl)-9H-purine; 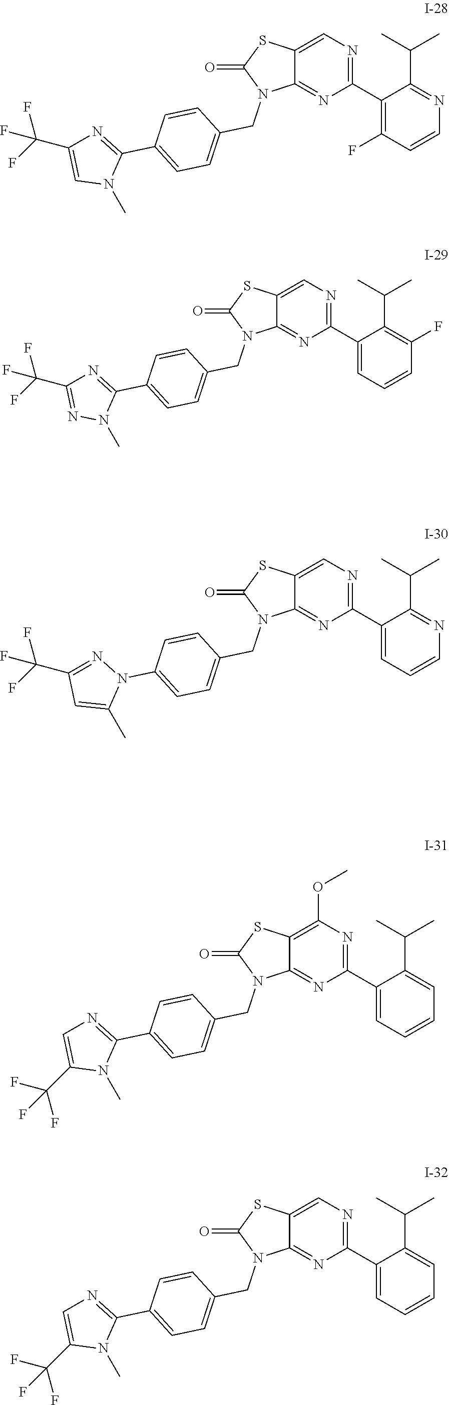 [SMILES: CC(C)c1nccc(F)c1-c1ncc2sc(=O)n(Cc3ccc(-c4nc(C(F)(F)F)cn4C)cc3)c2n1 ; CC(C)c1c(F)cccc1-c1ncc2sc(=O)n(Cc3ccc(-c4nc(C(F)(F)F)nn4C)cc3)c2n1 ; Cc1cc(C(F)(F)F)nn1-c1ccc(Cn2c(=O)sc3cnc(-c4cccnc4C(C)C)nc32)cc1 ; COc1nc(-c2ccccc2C(C)C)nc2c1sc(=O)n2Cc1ccc(-c2ncc(C(F)(F)F)n2C)cc1 ; CC(C)c1ccccc1-c1ncc2sc(=O)n(Cc3ccc(-c4ncc(C(F)(F)F)n4C)cc3)c2n1] 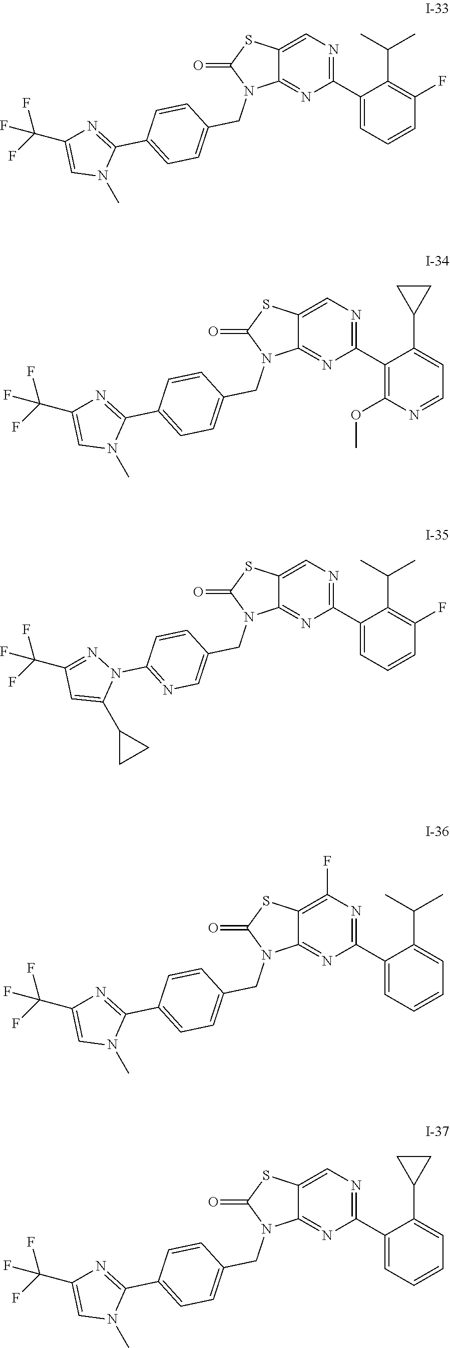 [SMILES: CC(C)c1c(F)cccc1-c1ncc2sc(=O)n(Cc3ccc(-c4nc(C(F)(F)F)cn4C)cc3)c2n1 ; COc1nccc(C2CC2)c1-c1ncc2sc(=O)n(Cc3ccc(-c4nc(C(F)(F)F)cn4C)cc3)c2n1 ; CC(C)c1c(F)cccc1-c1ncc2sc(=O)n(Cc3ccc(-n4nc(C(F)(F)F)cc4C4CC4)nc3)c2n1 ; CC(C)c1ccccc1-c1nc(F)c2sc(=O)n(Cc3ccc(-c4nc(C(F)(F)F)cn4C)cc3)c2n1 ; Cn1cc(C(F)(F)F)nc1-c1ccc(Cn2c(=O)sc3cnc(-c4ccccc4C4CC4)nc32)cc1] 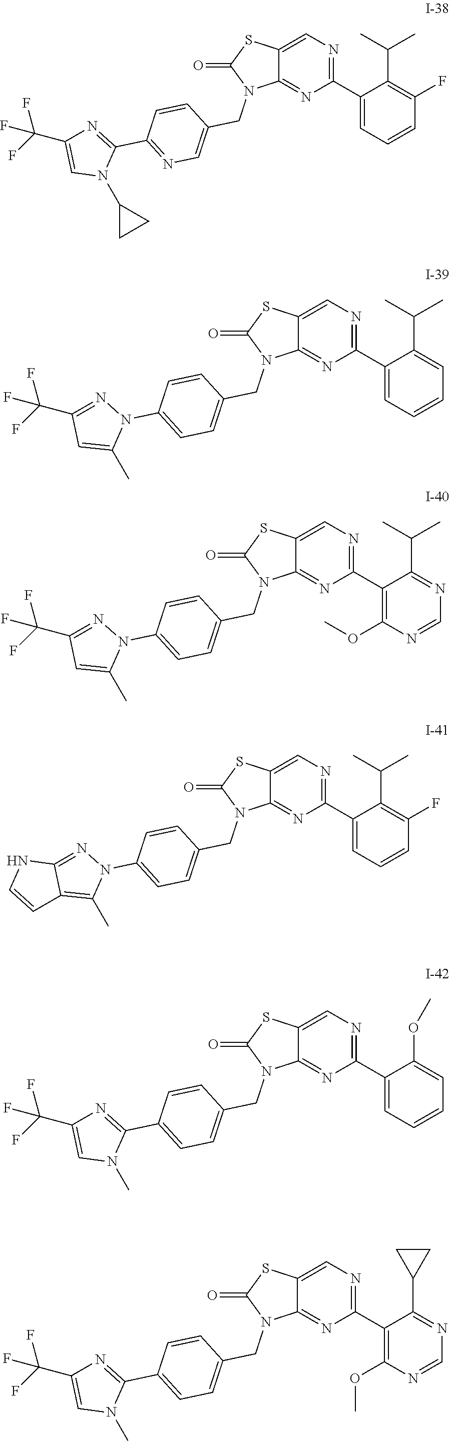 [SMILES: CC(C)c1c(F)cccc1-c1ncc2sc(=O)n(Cc3ccc(-c4nc(C(F)(F)F)cn4C4CC4)nc3)c2n1 ; Cc1cc(C(F)(F)F)nn1-c1ccc(Cn2c(=O)sc3cnc(-c4ccccc4C(C)C)nc32)cc1 ; COc1ncnc(C(C)C)c1-c1ncc2sc(=O)n(Cc3ccc(-n4nc(C(F)(F)F)cc4C)cc3)c2n1 ; Cc1c2cc[nH]c2nn1-c1ccc(Cn2c(=O)sc3cnc(-c4cccc(F)c4C(C)C)nc32)cc1 ; COc1ccccc1-c1ncc2sc(=O)n(Cc3ccc(-c4nc(C(F)(F)F)cn4C)cc3)c2n1 ; COc1ncnc(C2CC2)c1-c1ncc2sc(=O)n(Cc3ccc(-c4nc(C(F)(F)F)cn4C)cc3)c2n1] 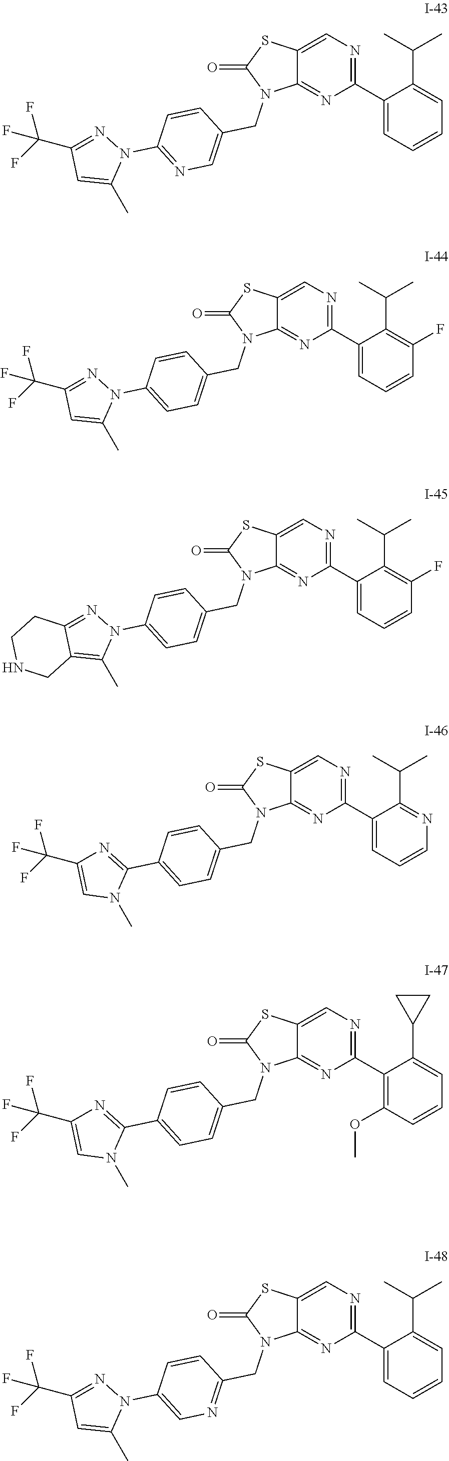 [SMILES: Cc1cc(C(F)(F)F)nn1-c1ccc(Cn2c(=O)sc3cnc(-c4ccccc4C(C)C)nc32)cn1 ; Cc1cc(C(F)(F)F)nn1-c1ccc(Cn2c(=O)sc3cnc(-c4cccc(F)c4C(C)C)nc32)cc1 ; Cc1c2c(nn1-c1ccc(Cn3c(=O)sc4cnc(-c5cccc(F)c5C(C)C)nc43)cc1)CCNC2 ; CC(C)c1ncccc1-c1ncc2sc(=O)n(Cc3ccc(-c4nc(C(F)(F)F)cn4C)cc3)c2n1 ; COc1cccc(C2CC2)c1-c1ncc2sc(=O)n(Cc3ccc(-c4nc(C(F)(F)F)cn4C)cc3)c2n1 ; Cc1cc(C(F)(F)F)nn1-c1ccc(Cn2c(=O)sc3cnc(-c4ccccc4C(C)C)nc32)nc1] 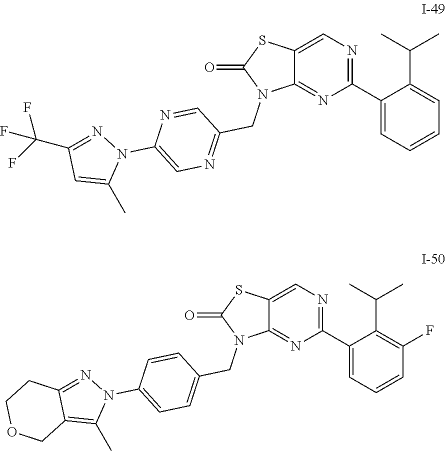 [SMILES: Cc1cc(C(F)(F)F)nn1-c1cnc(Cn2c(=O)sc3cnc(-c4ccccc4C(C)C)nc32)cn1 ; Cc1c2c(nn1-c1ccc(Cn3c(=O)sc4cnc(-c5cccc(F)c5C(C)C)nc43)cc1)CCOC2]
or pharmaceutically acceptable salts thereof.

32. A pharmaceutical composition comprising, a compound of any one of claims 1 to 31, and a pharmaceutically acceptable carrier.

33. A method of treating a disease or disorder associated with DNA damage comprising, administering to a patient in need thereof a therapeutically effective amount of a compound of any one of claims 1 to 31.

34. A method of treating a Poly (ADP-ribose) polymerase (“PARP”) inhibitor refractory or resistant cancer comprising, administering to a patient in need thereof a therapeutically effective amount of a compound of any one of claims 1 to 31.

35. The method of claim 34, wherein the cancer is a PARP inhibitor resistant or refractory BRCA1, BRCA2, or BRCA1 and BRCA2-deficient cancer

36. Use of a compound of any one of claims 1 to 31 in the manufacture of a medicament for inhibiting or reducing DNA repair activity modulated by ubiquitin specific protease 1 (USP1).
